SEQUENCE LISTING (2 sequences)

SEQ ID NO: 1 - protein, 288 aa, Artificial Sequence
  /note=“Description of Artificial Sequence N-terminal capping region”
MDPIRSRTPSPARELLSGPQPDGVQPTADRGVSPPAGGPLDGLPARRTMSRTRLPSPPAPSPAFSADSFSDLLRQFDPSLFNTSLFDSLPPFGAHHTEAATGEWDEVQSGLRAADAPPPTMRVAVTAARPPRAKPAPRRRAAQPSDASPAAQVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLN

SEQ ID NO: 2 - protein, 183 aa, Artificial Sequence
  /note=“Description of Artificial Sequence C-terminal capping region”
RPALESIVAQLSRPDPALAALTNDHLVALACLGGRPALDAVKKGLPHAPALIKRTNRRIPERTSHRVADHAQVVRVLGFFQCHSHPAQAFDDAMTQFGMSRHGLLQLFRRVGVTELEARSGTLPPASQRWDRILQASGMKRAKPSPTSTQTPDQASLHAFADSLERDLDAPSPMHEGDQTRAS

(19) United States
(12) Patent Application Publication
(10) Pub. No.: US 2022/0396777 A1
(43) Pub. Date: Dec. 15, 2022

(54) PATIENT-MATCHED ORGANOID SYSTEMS FOR STUDYING CANCER

(71) Applicants: Dana-Farber Cancer Institute, Inc., Boston, MA (US); Massachusetts Institute of Technology, Cambridge, MA (US)

(72) Inventors: Alexander K. Shalek, Cambridge, MA (US); Peter Winter, Cambridge, MA (US); Andrew Navia, Cambridge, MA (US); Srivatsan Raghavan, Boston, MA (US); William Hahn, Boston, MA (US); Andrew Aguirre, Boston, MA (US); Brian Wolpin, Boston, MA (US); Jennyfer Galvez-Reyes, Cambridge, MA (US)

(21) Appl. No.: 17/770,463

(22) PCT Filed: Oct. 26, 2020
(86) PCT No.: PCT/US2020/057342

§ 371 Date: Apr. 20, 2022

Related U.S. Application Data
(60) Provisional application No. 63/068,907, filed on Aug. 21, 2020.
(60) Provisional application No. 62/984,232, filed on Mar. 2, 2020.
(60) Provisional application No. 62/926,358, filed on Oct. 25, 2019.

(51) Int. Cl. C12N 5/09; G01N 33/50; C12Q 1/6886

(52) U.S. Cl. CPC C12N 5/0693; G01N 33/5011; C12Q 1/6886; C12N 2502/1323; C12N 2501/415; C12N 2501/24; C12Q 2600/158

(57) ABSTRACT

In certain example embodiments, the invention provides a method of generating an ex vivo cell-based system comprising dissociating an original tissue sample obtained from a subject into a single cell population; determining an in vivo phenotype of the tissue sample by conducting single-cell RNA analysis on a first portion of the single cells; establishing an ex vivo cell-based system from a second portion of the single cells; and culturing the ex vivo cell-based system in a medium or conditions selected to maintain the in vivo phenotype. In some embodiments, the original tissue sample is a tumor tissue sample, such as a pancreatic ductal adenocarcinoma (PDAC) tumor sample.

### CROSS-REFERENCE TO RELATED APPLICATIONS

This application claims the benefit of priority to U.S. Provisional Application Ser. No. 62/926,358, filed on 25 Oct. 2019; U.S. Provisional Application Ser. No. 62/984,232, filed on 2 Mar. 2020; and U.S. Provisional Application Ser. No. 63/068,907, filed on 21 Aug. 2020; the entire contents of each of said applications are incorporated herein in their entirety by this reference.

### STATEMENT REGARDING FEDERALLY SPONSORED RESEARCH

This invention was made with government support under Grant No. CA217377 awarded by the National Institutes of Health. The government has certain rights in the invention.

### TECHNICAL FIELD

The subject matter disclosed herein is generally directed to ex vivo cell-based systems that faithfully recapitulate an in vivo phenotype of interest and methods of generating and using the cell-based systems.

### BACKGROUND

Despite extensive preclinical research, pancreatic ductal adenocarcinoma (PDAC) is still associated with a bleak prognosis due to rapid evasion from standard-of-care and experimental treatments and aggressive metastases to other organs (Maitra et al. Annu Rev Pathol 3:157-188 (2008)). Understanding the intrinsic heterogeneity of these tumors and how they interact with their microenvironment could aid both the rational design of novel therapeutic interventions and help nominate patients for the appropriate treatment. The relative homogeny of genetic alterations between patients, coupled with the paucity of therapeutic interventions available for these lesions has motivated an attempt to classify these tumors based on RNA expression (Collisson et al. Nat Rev Gastroenterol Heptol 16:207-220 (2019)). While these efforts were groundbreaking, the use of bulk RNA-seq methods makes distinguishing tumor from non-tumor gene expression particularly challenging (Collisson et al. Nat Rev Gastroenterol Heptol 16:207-220 (2019)). For example, Bailey et al (Nature 531:47-52 (2016)) identified an immunogenic tumor subtype that has recently been hypothesized to be derived mostly from immune infiltrate (Collisson et al. Nat Rev Gastroenterol Heptol 16:207-220 (2019)). Despite these caveats, work by Collison et al. (Nat Med 17:500-503 (2011)), Bailey et al. (Nature 531:47-52 (2016)), and Moffitt et al. (Nat Genet 47:1168-1178 (2015)) defined a total of nine transcriptional subtypes, several with overlapping features. Interestingly, each study identified a subtype with overall poorer prognosis (quasimesenchymal (Bailey et al. Nature 531:47-52 (2016)), squamous (Collison et al. Nat Med 17:500-503 (2011)), basal (Moffitt et al. Nat Genet 47:1168-1178 (2015)) all with similar features-heretofore, the “Basal-like” subtype. Additionally, Collison et al. (Nat Med 17:500-503 (2011)) and Moffitt et al. (Nat Genet 47:1168-1178 (2015)) both identified a “Classical” subtype, again with similar transcriptional signatures. Thus, Classical and Basal-like transcriptional subtypes likely represent the consensus biology across the three studies and are generally accepted by the field (Collisson et al. Nat Rev Gastroenterol Heptol 16:207-220 (2019)).

While this classification scheme has provided a helpful framework for thinking about PDAC tumors transcriptionally, further study is needed to understand, at a cellular level, whether these tumor states are mutually exclusive and how each communicates with their respective microenvironments. Further, it remains an open question how well these expression states are preserved ex vivo as organoid models. Since transcriptional subtype has been shown to influence overall patient survival as well as drug sensitivity (Collisson et al. Nat Rev Gastroenterol Heptol 16:207-220 (2019); Tuveson et al. Science 364:952-955 (2019)), it is imperative that we gain a deeper understanding of these phenotypes in patient tumors and their associated models, especially as the field moves toward using transcriptional state and ex vivo organoid drug-testing to stratify patients (Tuveson et al. Science 364:952-955 (2019)).

Currently, there is a lack of a direct comparison of primary PDAC samples to matched organoid models. If these models are faithful representations of their parent tumors, they would provide a valuable resource for further study and, in theory, personalized medicine (FIG. 2).

### SUMMARY

In certain example embodiments, the invention provides a method of generating an ex vivo cell-based system comprising dissociating an original tissue sample obtained from a subject into a single cell population; determining an in vivo phenotype of the tissue sample by conducting single-cell RNA analysis on a first portion of the single cells; establishing an ex vivo cell-based system from a second portion of the single cells; and culturing the ex vivo cell-based system in a medium or conditions selected to maintain the in vivo phenotype.

In some embodiments, the original tissue sample may be a tumor tissue sample, such as a metastatic tumor tissue sample.

In some embodiments, the method may further comprise conducting a second single-cell RNA analysis on single cells derived from the established ex vivo cell-based system to determine a current phenotype; and if the phenotype has changed, modifying the culture medium or conditions to revert to or decrease the expression space between the current phenotype and the in vivo phenotype.

In some embodiments, selecting or modifying the medium or conditions comprises the addition or subtraction of one or more growth factors or cell signaling molecules, inducing changes in intra-cellular signaling between one or more cell types in the ex vivo cell-based model, inducing changes in cell state of one or more cell types, or changing cellular composition of the ex vivo cell-based model.

In some embodiments, the ex vivo cell-based model is co-cultured with fibroblasts in depleted media.

In some embodiments, the medium comprises one or more growth factors or cell signaling molecules. In some embodiments, the cell signaling molecules comprise WNT7B, WNT10A, or a combination thereof.

In some embodiments, the method may further comprise culturing the cells in a medium which does not contain TGF beta inhibitor.

In some embodiments, the tumor may be a pancreatic ductal adenocarcinoma (PDAC) tumor. In some embodiments, the PDAC is the basal-like subtype, the classical subtype, or a hybrid sub-type including transcriptional phenotypes from both.

In some embodiments, the tumor is a breast cancer tumor. In some embodiments, the tumor is a bladder cancer tumor.

In some embodiments, the organoid may be cultured in a medium comprising IFNγ if the phenotype is a basal phenotype and/or IFNγ phenotype.

In another aspect, the invention provides an ex vivo cell-based system derived by any of the method described herein.

In some embodiments, the ex vivo cell-based system may comprise a tumor microenvironment cell.

In some embodiments, the tumor microenvironment cell may be a tumor infiltrating lymphocyte (TIL) and/or natural killer (NK) cell.

In some embodiments, the ex vivo cell-based system simulates a phenotype from a subject who is responsive to cancer treatment. In some embodiments, the ex vivo cell-based system simulates a phenotype from a subject who is non-responsive to cancer treatment.

In some embodiments, the treatment may be chemotherapy. In some embodiments, the treatment may be immunotherapy. In some embodiments, the treatment may be checkpoint blockade (CPB) therapy.

In some embodiments, the phenotype may be a basal phenotype and/or IFNγ phenotype.

In some embodiments, the system may be an organoid.

In yet another aspect, the invention provides a method for screening therapeutic agents comprising; exposing any of the ex vivo cell-based model systems described herein to one or more therapeutic agents, measuring responsiveness of the ex vivo model to the one or more therapeutic agents; and classifying the one or more therapeutic agents as indicated if the ex vivo model exhibits a responsive phenotype indicating a susceptibility of the model to the one or more therapeutic agents, or contraindicated if the ex vivo model exhibits a non-responsive phenotype indicating a lack of susceptibility of the model to the one or more therapeutic agents.

In some embodiments, the responsive phenotype may be measured by a change in one or more cell types or cell states of the model indicating reduced fitness of the model or cell death of one or more target cell types in the model.

In some embodiments, the non-responsive phenotype may be measured by no change in model phenotype or a change in one or more cell types or cell states indicating increased growth or fitness of the model or one or more cell types in the model.

In some embodiments, the method may further comprise clonally expanding the one or more cell types exhibiting increased growth or fitness and performing single cell RNA analysis of the clonally expanded cells to identify cell type and/or cell state.

In some embodiments, the ex vivo cell-based model may be derived from a subject to be treated.

In some embodiments, the method may further comprise administering the indicated one or more therapeutic agents to the subject.

In some embodiments, the method may further comprise administering one or more therapeutic agents based on the identified cell type and/or cell state of the clonally expanded cells.

In some embodiments, the ex vivo cell-based model system may be a tumor system. In some embodiments, the tumor system may be derived from a pancreatic ductal adenocarcinoma (PDAC) tumor.

In some embodiments, the therapeutic agent may be a chemotherapy.

In some embodiments, the therapeutic agent may be a combination therapy comprising an agent predicted to shift the ex vivo cell model to have increased responsiveness to a chemotherapy and a chemotherapy.

In some embodiments, the therapeutic agent may be an immunotherapy.

In some embodiments, the immunotherapy may be one or more T cells expressing a chimeric antigen receptor (CAR) or T cell receptor (TCR).

In some embodiments, the immunotherapy may be checkpoint blockade (CPB) therapy.

In some embodiments, the therapeutic agent may be a combination therapy comprising an agent predicted to shift the ex vivo cell model to have increased responsiveness to an immunotherapy and an immunotherapy.

In some embodiments, the therapeutic agent may be a targeted therapy.

In some embodiments, the therapeutic agent may be a combination therapy comprising an agent predicted to shift the ex vivo cell model to have increased responsiveness to a targeted therapy and a targeted therapy.

In yet another aspect, the invention provides a method of treating PDAC tumors comprising administering one or more agents that reduce IFNγ expression or interferon response gene expression in the tumor microenvironment.

In yet another aspect, the invention provides a method of treating PDAC tumors comprising administering one or more agents that shift tumor cell phenotype from a basal or IFNγ phenotype to a classical phenotype.

In yet another aspect, the invention provides a method of treating PDAC tumors comprising tumor cells expressing a basal subtype phenotype comprising administering one or more agents capable of interfering with intracellular crosstalk between tumor cells and basal associated tumor associated macrophages (TAM).

In some embodiments, the one or more agents may interfere with CSF1 and/or IL34 from binding to CSF1R. In some embodiments, the one or more agents may bind to CSF1, IL34, and/or CSF1R.

In some embodiments, CSF1R antibodies are administered.

In some embodiments, the method may further comprise administering an immunotherapy, chemotherapy and/or targeted therapy.

In some embodiments, the PDAC may be the basal-like subtype the classical subtype, or a hybrid sub-type including transcriptional phenotypes from both.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1—Illustrates a generalized clinical course involving serial sampling from solid tumors.

FIG. 2—Schematic illustrating precision medicine approaches into clinical care for pancreatic cancer patients. Briefly, the model shows serial biopsies, followed by DNA sequencing and organoid modeling and drug screening to assess whether these are predictive of treatment responses. Single-cell RNA sequencing on patient samples allows for better understanding of tumor cells and their evolution over the course of clinical care.

FIGS. 3A-3E—Schematics illustrating how Seq-Well enables single-cell profiling of PDAC biopsies. (3A) Paucicellular core needle biopsies from pancreatic ductal adenocarcinoma (PDAC) liver metastases are dissociated into single-cell suspensions and split for organoid seeding and scRNA-seq. Organoid samples are subsequently sequenced at an early (1-3) and later (5-7) passage. (3B) Seq-Well, a picowell-based platform is capable of economically interrogating the gene expression of thousands of single cells at once. (3C) t-SNE visualization of tumor and non-tumor cells from 14 patients with metastatic disease. Tumor cells tend to cluster by patient, consistent with previous single-cell studies by our lab and others. Conversely, non-tumor cells are more often admixed between patients. Applicants capture diverse non-tumor cells in the liver microenvironment, each with their own levels of heterogeneity. For example, force-directed visualization of the K-nearest neighbor graph computed over the ˜1,500 most variable genes for the T cells identified in the cohort reveals several transcriptionally distinct T-cell subsets. (3D) Summary for the number of tumor cells by patient and aggregate across patients for the non-tumor cell types identified. (3E) Illustrates initial discovery of cell types—tumor and stromal.

FIGS. 4A-4E—Distinct and hybrid transcriptional states in liver-resident metastatic PDAC cells. (4A) Using only tumor cells from each patient, Applicants computed the average expression of genes defining basal and classical PDAC subtypes as described previously (Moffitt et al. Nat Genet 47:1168-1178 (2015)). Clustering these profiles using Ward's method revealed the expected breakdown for the majority of tumors into basal and classical subtypes. Applicants also observed two that did not fit the expected subtypes and one that co-expresses features of both basal and classical. (4B) Single cell basal vs. classical scores form a representative classical (PANFR0383), basal (PANFR0575) and co-expressing (PANFR0543) tumor. PANFR0543 expresses both basal and classical gene sets within single cells. (4C) Scoring each patient's tumor cells for literature curated gene sets revealed high consistency between the classical subtype identification from different studies. Applicants also confirmed, as previously alluded to (Collisson et al. Nat Rev Gastroenterol Heptol 16:207-220 (2019)), that squamous, basal, and quasimesenchymal subtypes share features of transcriptional state as cells that tend to score high for one, score well for the others. ADEX (Bailey et al. Nature 531:47-52 (2016)) and pancreatic progenitor (Bailey et al. Nature 531:47-52 (2016)) did not score consistently well in these data, possibly reflecting features of the transcriptional states represented in metastatic disease vs the primary site. Immunogenic scores poorly, as expected, since this subtype likely reflects contamination from immune infiltrate in the bulk samples used for its derivation (Collisson et al. Nat Rev Gastroenterol Heptol 16:207-220 (2019)). The “single-cell correlates” are computed by correlating each single cell's basal or classical score (Moffitt et al. Nat Genet 47:1168-1178 (2015)) to the complete expression matrix for all tumor cells. (4D) Applicants found that basal-like tumor cells have high expression of interferon (IFN) response genes, with response to IFNγ being the most prominent. (4E) The basal-like signature correlates with interferon response genes from the mSigDB gene set. Tumor cells from basal-like tumors exhibit high interferon response gene expression.

FIGS. 5A-5B—Genomic instability in cancer enables identification of tumor cells. (5A) Tumor cells cluster by patient, while normal cells cluster by cell type. (5B) Inferred copy number alterations based on arrangement of gene expression geographically by chromosome (right). Copy number variation was summarized for each tumor cell using two metrics, as described in the examples.

FIG. 6—Initial survey of cell types included tumor and stromal/immune cells. As part of Applicants' clinical workflow, they identified one tumor that lacked canonical PDAC alterations (PANFR0580). This tumor was subsequently identified by both RNA sequencing and pathology review as a pancreatic neuroendocrine tumor.

FIG. 7—Tumor cell transcriptomes map to classical vs basal-like subtypes, with one co-expressing tumor.

FIGS. 8A-8C—Transcriptional heterogeneity maps to known subtypes. (8A) Pancreatic neuroendocrine tumors (PanNETs) arise from endocrine cells, not exocrine cells like normal PDAC. They exhibit a distinct biology and disease progression and they metastasize to the liver. (8B and 8C) Moffitt data and others seem to have these subtypes as well but they are currently not recognized as such. The “neither” tumor has distinct PCs that define it, but with a single tumor it's hard to contextualize.

FIG. 9—T cell subsets in the metabolic microenvironment. Basal-like tumors have a higher fraction of cytotoxic T lymphocytes (CTLs).

FIGS. 10A-10B—In silico isolation of tumor cells from 15 patients. (10A) Mutational alterations from targeted DNA sequencing. (10B) t-SNE plot of tumor cells from each patient. One tumor (PANFR0580) lacked any of these mutations and was subsequently identified by single-cell RNA-sequencing and pathology review as a pancreatic neuroendocrine tumor.

FIGS. 11A-11B—Metastatic tumor cell expression states include classical, basal-like, and co-expressor transcriptional subtypes. (11A) Averaged expression for the top 25 Basal and Classical genes (Moffitt et. al. Nat Genet 47:1168-1178 (2015)). (11B) Distribution of single cells across the classical vs. basal-like transcriptional axis for three patient tumors. A subset of cells (PANFR0543) co-express both signatures within single cells.

FIGS. 12A-12G—A phenotypic dampening occurs in organoid culture. (12A) Distribution of classical vs. basal-like transcriptional states scored over single cells from fresh tumor biopsies and matched passage 2 organoid cells for three patient tumors. (12B) InferCNV analysis of single tumor cells from a patient tumor and matched organoid suggests that both transcriptional shifts and sub-clonal selection may be playing a role in differences between organoid models and matched patient tumors. (12C) Summary of cell numbers recovered from the primary sample (P0) and early passage organoids (Early; P1-P3) from patient matched samples as well as proportion of cells cycling by each time point and group. Fitness flips with in vitro culture—classical tumors increase their growth in the organoid culture environment while basal tumors see a decrease in overall “fitness” at early passage. (12D) t-SNE visualization for all primary samples with matched organoid samples. (12E) To confirm the absence of the basal subtype from organoid conditions Applicants compared data from their recent publication of bulk RNA-seq profiles from metastatic PDAC tumors (n=62 cases) with bulk RNA-seq profiles from their organoid cohort (n=70 samples). In this cohort, Applicants confirm several classical, basal, and co-expressing tumors as well as a significant subset that express neither. Applicants also note the general absence of the basal-like subtype from the organoid culture conditions with few—if any—organoids mirroring this more aggressive subtype. (12F) Scatter plot showing scores for both classical and basal subtypes where matched data were available. Here Applicants note a similar loss of the basal phenotype in this patient-matched comparison, supporting the observations made in the single-cell cohort. (12G) Scatter plot showing averaged classical vs. basal-like gene expression shifts in matched organoids and patient tumor cells in single-cell (left) and bulk RNA (right) sequencing cohorts.

FIGS. 13A-13D—T and NK cells from basal-like tumors express high levels of IFNγ. (13A) T and NK cells in the basal metastatic microenvironment produce IFNγ. (13B) T cells from basal-like tumors exhibit high IFNγ gene expression. (13C) Violin plot of IFNγ score for T cells derived from tumors of different transcriptional subtypes. (13D) IFNγ expression score in distinct T cell populations projected over the KNN visualization.

FIG. 14—Serial sampling of pre- and post-treatment metastatic lesions reveals dynamic transcriptional changes.

FIG. 15—Pre- and post-treatment metastatic lesions—different genetic clones.

FIG. 16—Conversion to basal subtype after immunotherapy.

FIG. 17—Schematic illustrating unknowns about whether the microenvironment is important in maintaining transcriptional subtypes.

FIG. 18—Heterogeneity in basal and classical pancreatic cancer subtypes.

FIG. 19—Comparison of organoids to their in vivo counterparts.

FIG. 20—Dependency Map, which shows that pancreas organoids look like gastric tumors.

FIG. 21—Malignant and non-malignant cell types identified in the cohort. Fib=Fibroblast, DC=Dendritic cell, pDC=plasmacytoid dendritic cell, cpDC=cross-presenting dendritic cell, Hep=hepatocyte, Mac=Macrophage/monocyte, Endo=Endothelial, NK=Natural killer.

FIG. 22—Metadata and cell recovery from the 23 cases used in the present study. Mutations and CNVs were assessed by targeted DNA sequencing. Black/Red=“Yes”, White=“No”, Grey=“NA”.

FIG. 23—Shows global transcriptional variation maps to known tumor subtypes.

FIG. 24—Shows tumor-by-tumor transcriptional variation maps to known tumor subtypes.

FIG. 25—Shows results of mapping single-cell data to other published subtyping approaches.

FIG. 26—Basal versus classical axis in metastatic PDAC.

FIG. 27—Heat map and graphs showing results of leveraging single-cell resolution to uncover subtype-associated biology.

FIG. 28—Heat map showing that Wnt signaling, IFN response, and TGF beta signaling correlate with the basal-like state in patients. Top correlated genes (>0.1 Pearson coefficient, >3 s.d. above shuffled) with either basal or classical score.

FIG. 29—Heat map showing that Wnt7B expression correlates with the basal-like state in patients.

FIG. 30—Schematic addressing the question of whether organoid models maintain in vivo transcriptional states ex vivo.

FIG. 31—Heat maps showing changes in transcriptional states between tumor and organoid states.

FIG. 32—Heat maps showing changes along Wnt axis in organoid models.

FIG. 33—Flow cytometry plot and heat maps showing changes along the Wnt axis in organoid models in a classical tumor.

FIG. 34—Flow cytometry plot and heat maps showing changes along the Wnt axis in organoid models in a basal tumor.

FIG. 35—Flow cytometry plot and heat maps showing changes along the Wnt axis in serially sampled organoid models prior to treatment.

FIG. 36—Flow cytometry plot and heat maps showing changes along the Wnt axis in serially sampled organoid models after treatment.

FIG. 37—Micrographs and graph showing results of IFNγ treatment in organoids from classical and basal-like tumors.

FIG. 38—Scatter plots showing differences in clinical outcomes after a six-day or a 10-day drug assay using various drug regimens.

FIG. 39—Graphs showing basal versus classical axis with bulk averages by types on the left and single cell resolution on the right. PDAC transcriptional subtypes are markedly heterogeneous within individual tumors.

FIG. 40—Schematic showing refined basal to classical axis in primary tumor before organoid culture.

FIG. 41—Schematic showing ideally preserved classical and basal states in organoid culture.

FIG. 42—Heat map and graphs showing that stem-like basal phenotype is lost in matched organoid models. Both transcriptional state plasticity and selection are apparent from matched tumor-organoid analysis.

FIG. 43—Heat map showing that basal-like malignant cells co-express WNT7B-driven and immunomodulatory expression programs. Single malignant cells are ranked by their difference in basal and classical scores. WNT7B and epithelial programs are generated using single cell correlation, TGFB and IFN are significantly correlated genes from reference Hallmark gene sets.

FIG. 44—Shows coordinated immune suppression in the basal-like microenvironment. Left panel: Specific cell types identified in all tumors excluding those on active immunotherapy. Top middle portion: General lymphoid and specific T cell skews. Bottom right: Select T cell density images from a basal (left) and classical (right) tumor. Top right: T and NK cells in the basal microenvironment express high levels of IFNγ.

FIG. 45—Shows coordinated immune suppression in the basal-like microenvironment. Left: The major axis of variation in macrophages aligns with an M1 to M2-like continuum. Right: Distribution of monocytes from each tumor along the M1-like to M2-like axis. Strongly basal tumors are dominated by M2-like cells.

FIG. 46—Shows alterations to tumor phenotypes in standard organoid culture conditions. Left: Cycling proportions (pie charts) and take rate for organoids are derived from each transcriptional subtype. Right: Each tumor (grey) and organoid (red) pair is plotted by their average basal and classical score, a line connects each pair.

FIG. 47—Heat maps showing phenotypic differences across organoids and cancer cell lines. Organoids show a skew toward classical phenotypes while pancreatic cancer cell lines from the Cancer Cell Line Encyclopedia (CCLE) exhibit more basal phenotypes. Organoids are initiated in high WNT3A and R-spondin in 3D, CCLE initiated and maintained in serum-containing culture media in 2D.

FIGS. 48A-48E—A clinical pipeline for matched single-cell RNA-578 seq and organoid generation from metastatic biopsies. (48A) Pipeline for collecting patient samples, dissociation and allocation for scRNA-seq, and parallel organoid development. (48B) CNV correlation (to averaged top 5% of altered cells) versus CNV score (mean square) for each single cell in PANFR0575. Cells are colored by their putative class: malignant (light blue) or non-malignant (empty black circles). (48C) Bulk targeted DNA-seq (top) and single-cell (rows, bottom) CNV profiles arranged by chromosome (columns). (48D, 48E) t-distributed stochastic neighbor embedding (t-SNE) visualization for non-malignant (48D) and malignant (48E) single cells in the biopsy cohort. Cells are colored by patient. Endo, Endothelial; Fib, Fibroblast; B, B-cell; Hep, Hepatocyte; DC, Dendritic cell; pDC, Plasmacytoid dendritic cell; Mac, Macrophage; T, T-cell; NK, Natural killer cell.

FIGS. 49A-49G—Patient characteristics and unsupervised cell-type identification across the biopsy cohort. (49A) Clinical and molecular features for all patients included in the dataset (Rx=Therapy; Other=Adrenal (PANFR0637), Omentum (PANFR0635, PANFR0598), Peritoneum (PANFR0588); Org. at P2=Organoid measured at passage 2). Mutations were determined by bulk targeted DNA-seq (Red, Altered; White, wildtype; Grey, Data not available). Number of single cells captured per biopsy and their malignant and non-malignant fraction (see below) is visualized at the right. (49B) Distribution of unique molecules and genes captured in quality cells per biopsy, median values are indicated for each metric (dotted line) and violin plots are colored by patient (top, Log 10(UMIs); bottom, number of genes). (49C) t-SNE visualization of the entire single-cell biopsy dataset (n=23,042 cells) colored by the SNN clusters identified (inset numbers). (49D) Distribution of single cells captured per biopsy across the identified SNN clusters. In general, a patient's malignant cells are expected to form unique clusters driven by CNVs. Owing to this feature, the data are split into putative malignant and non-malignant groups of clusters. (49E) Overview of cell-typing for all cells in the biopsy dataset. Cells are ordered by SNN cluster and separated by cell types. Top heatmap represent expression levels for a subset of select markers (n=73 genes) used to identify cell types. Color bar indicates cell types as labeled in 49F and binarized cell cycle phenotypes are labeled (black, cycling; white, not). CNV scores (mean square of alterations per cell) used to parse malignant from non-malignant are shown using T/NK, endothelial, fibroblasts, and hepatocytes as reference; grey boxes denote normal cell types where we did not compute reference CNV scores. Bottom panel shows biopsy of origin for each cell. The data are split by non-malignant (n=15,302) and malignant (7,740) identity. (49F) t-SNE visualization as in 49C but colored by cell types identified, abbreviations as in FIG. 48D. (49G) Fraction of each cell type contributed by each biopsy sample (color fill, patient ID; as in 49A), cell type totals are noted at the top of each bar.

FIGS. 50A-50B—Copy number variation (CNV) parses malignant from non-malignant cells in each biopsy. (50A) Heatmaps represent select scRNA-seq-derived copy number profiles where expression across the transcriptome is organized by chromosome (columns) for each single putative malignant cell (rows) from a given biopsy. Top bar indicates reference bulk targeted DNA-seq for the same patient and shows strong concordance with the single-cell derived profiles. Overt subclones detected are shown for PANFR0605. (50B) CNV correlation (averaged top 5% of altered cells per biopsy) versus CNV score (mean square of modified expression) for each single putative malignant (colored points) and reference normal cell (empty black circles) within a given biopsy. Only a single sample, PANFR0604, did not contain any malignant cells.

FIGS. 51A-51H—Identifying and contextualizing malignant transcriptional heterogeneity. (51A) Principal component analysis (PCA) and scatter plot for PC1 and PC2 across all malignant cells (n=7,740) separates PANFR0580's malignant cells (n=662) from the rest of the samples. Cells are colored by patient ID (as in FIG. 49A). Heatmap for genes with the strongest negative loading on PC1 (n=30) denote a neuroendocrine identity (TTR, CHGB). This tumor was later classified by histology as a pancreatic neuroendocrine tumor (PanNET). (51B) PCA across verified PDAC cells (n=7,078) showing strong separation over PC1 and PC2 (top). Bottom heatmap shows the top positively and negatively loaded genes (n=50 each) on PC2. Moffitt 50 ID column (left) indicates if that gene (row) is in the Moffitt et al. basal (orange) or classical (blue) gene sets (16) (51C) Principal component (PC) elbow plot showing the standard deviation for the first 20 components calculated over the malignant cell variable genes (Methods). Line is drawn at the putative “elbow” (black versus grey points) as inclusion of additional PCs described overlapping information or quality metrics. Cross-correlational analysis for each single-cell's embeddings across first 9 PCs (black points) and scores for literature curated gene sets describing EMT, classical and basal, and cell cycle phenotypes. PC1 positively correlates with EMT, basal, and to a degree, cycling. Cells with positive embeddings on PC2 are correlated with classical phenotypes and anti-correlated with basal and EMT phenotypes suggesting these phenotypes are anti-correlated across a continuum of expression. PC3 and PC8 describe cells with high cell cycle scores. The other PCs do not associate significantly with these phenotypes. (51D) Heatmap depicts the malignant cell average expression for each gene in the basal and classical gene sets. Hierarchical clustering splits the biopsy cohort into 3 groups, basal (n=7), classical (n=4), and those of apparent mixed, or intermediate identity (n=10). (51E) Summary of transcriptional heterogeneity in the PDAC malignant cells across the biopsy cohort. Main heatmap represents the pairwise correlation of all single malignant cells using the variable genes (n=923 genes) and dendrogram is split by tumors post hierarchical clustering. PCA embeddings for the first 3 PCs (center), literature curated signature scores (center right), and binarized cell cycle program (far right) are indicated. Cell order is maintained across the different heatmaps. (51F) Score difference (basal-classical score) ranked averaged tumor profiles. Where discrete binning is necessary (e.g. when discussing “tumor subtype of origin” for a T cell), Applicants use this strategy to preserve the continuous nature of the phenotype. (51G) Cross-correlational analysis of all signatures measured in the malignant PDAC cells (16, 17, 26, 27). The different signatures give overlapping information and separate into two clades, implying that, in this data, they represent similar properties of each single cell. (51H) Heatmap summarizing the expression of literature-suggested classification and phenotyping gene sets (Pancreatic Progenitor; QM, Quasi-mesenchymal; Immuno, Immunogenic) (16, 17, 26, 27). Left heatmap depicts malignant cells separated by biopsy and are ordered as in 51F. Right heatmap shows expression of the same genes in non-malignant cells, randomly sampled to 450 cells each for visualization. Immunogenic and ADEX are not detected strongly in these metastatic cells. Basal and classical signatures are specific to malignant cells, while EMT and Immunogenic signatures are expressed by fibroblasts and plasma cells respectively. Few genes from the exocrine-like signature were detected in our dataset so it was excluded from the analysis.

FIGS. 52A-52F—Basal, classical, and hybrid transcriptional states in metastatic PDAC. (52A) Heatmap depicts the expression of basal and classical genes (n=30 each, Methods) across all malignant cells. EMT, basal, classical, and cell cycle programs are indicated. (52B) PDAC tumors are arranged by their average classical (x-axis) and basal (y-axis) scores. Points are pie charts summarizing the malignant subtype composition within each biopsy. (52C) Composition of each tumor (% cells) across the three expression subtypes in the primary resection cohort (n=15 cases) determined by multiplexed immunofluorescence (b, basal; m, mixed; c, classical). Representative images for strongly polarized tumors are shown (bottom). (52D) Representative mixed tumor images (top) and corresponding pheno-plots (bottom). Pheno-plot points correspond to cells in the image above and are colored by their subtype, marker negative cells are not visualized. Zoom panel on far right (dotted white box, image; solid black box, pheno-plot) shows juxtaposed hybrid and basal cells. (52E) Pairwise correlation of genes significantly associated with basal or classical expression states. Left bar indicates the subtype association of each gene (orange, basal; blue, classical). (52F) Heatmap shows the relative expression of the indicated basal and classical-associated programs, cells are ordered as in 52A. Left heat bar indicates each gene's correlation to either basal or classical subtypes, and the range for these values is the same as in 52E. Bottom plot indicates each single-cell's biopsy of origin.

FIGS. 53A-53C—Multiplexed immunofluorescence confirms the presence of “hybrid” single cells in primary PDAC. (53A) Heatmap depicting binarized detection (red, detected; white, not detected) for each single cell identified as likely malignant in all 76,092 cells from 15 primary resection cases (Methods). Cells are binned by their classification as basal, classical, hybrid or “No Detection” (cells classified as malignant but with none of the indicated markers detected). (53B) Similar to 53A but using the scRNA-seq data from the biopsy cohort. When considering all markers for basal and classical, only 26 malignant cells had no detection (0.36%) in the scRNA-seq dataset. However, when the markers were restricted to only the 5 used in the mIF cohort Applicants observed a comparable rate of “No Detection” in the metastatic biopsy cohort (39%, mIF; 35%, scRNA-seq). (53C) Representative images (top) and pheno-plots (bottom) for each case in the mIF cohort. In the pheno-plots, marker negative cells are not visualized. Pie charts in the upper right of each pheno-plot represent the distribution of all three subtypes in that sample.

FIGS. 54A-54C—Subtype specific expression signatures. (54A) Tied dot plots depicting the correlation coefficient for each gene (points) to either basal or classical phenotypes from select literature-derived gene sets, indicated at the top of each plot, which summarize aspects of subtype associated biology. Dotted lines represent significance threshold (3 SD above the mean of shuffled data), points and lines are colored if that gene passes the threshold and select genes are indicated. (54B) Heatmap for basal-associated signatures in bulk RNA-seq profiles separated by either TCGA (primary disease) or Panc-Seq (metastatic) cohorts. Tumors are ranked by their score difference along the basal to classical axis and expression for the indicated gene sets is visualized. Basal and classical genes are from Moffitt et al (16). WNT7B and TGFB program genes are the same as in FIG. 52F. The IFN_{Rep} score was negatively and positively associated with ABSOLUTE purity and a general immune/stromal cell contamination score, respectively (right), making it difficult, in bulk profiles, to assign signal specifically to malignant cells as these genes are expected to be highly expressed in any cell type (e.g. macrophages) responding to IFN in the microenvironment. (FIG. 54C) Heatmap for relative expression of core WNT pathway members detected in malignant (left) and randomly sampled non-malignant cells (450 each for visualization, right). Certain WNTs are not detected in either malignant or non-malignant cells (e.g. WNT3A) and are omitted from the plot. Malignant cells are ranked by their WNT7B score. Basal and classical scores are indicated at the top and the basal score correlation coefficient for each gene is indicated at the right.

FIGS. 55A-55G—Asymmetric distribution of immune phenotypes across the basal to classical continuum. (55A) t-SNE visualization of non-malignant cells identified in the metastatic microenvironment, abbreviations are the same as in FIG. 48D (TAM, tumor associated macrophage; Trans, Transition; NKT, natural killer T cell). (55B) Heatmap shows the relative expression for select cell type markers. Top bar indicates the binarized cell cycle program (black, cycling) and the bottom color bar corresponds to the cell type colors noted in 55A. (55C) Cross-correlational heatmap and hierarchical clustering for similarity in the capture frequency of non-malignant cell types from each biopsy. Rainbow coloration in the main heatmap indicates convergence (yellow to red) or divergence (white to blue) across cell types. Right heat bar indicates preferential association for each cell type with either the basal (orange, negative values) or classical (blue, positive values) malignant transcriptional subtypes. Color ranges for both quantities are Pearson's r, white dots indicate P<0.05 for the subtype associations. Top bar chart indicates the total number of cells for each type. (5D) Scatter plot compares each liver biopsy's position on the basal to classical continuum (y-axis, score difference) to the relative abundance of activated NK cells captured from its microenvironment. Points represent individual biopsies and are colored by their discretized transcriptional subtype (n=15). (55E) Distribution (blue heat) of CD8+ T cell phenotype across the progenitor to exhausted/differentiated continuum in each liver-resident biopsy. Biopsies are sorted by the score difference (far left heat bar). The corresponding fractional capture of CD4+ T cells is indicated left of the main heat map for each sample. (55F) Distribution (green heat) of TAM phenotype for the macrophages captured in each liver biopsy. Biopsies with <100 macrophages were excluded. Heatmap is ranked by average monocyte-like to macrophage skew and both average basal and classical scores are indicated (right). (55G) Phenotypic hierarchy for TAM subsets using the expression scores for each phenotype across all TAMs captured in the dataset. The distribution (density; high=more TAMs) across the phenotypic hierarchy is visualized (right) according to malignant transcriptional phenotype as in FIG. 51D.

FIGS. 56A-56M—Identification of T/N 607 K, macrophage, and fibroblast heterogeneity in the metastatic microenvironment. (56A) Force-directed layout visualization (FDL; distance corresponds to similarity) for the KNN graph of T/NK cells in the metastatic cohort. Cells are colored by their separately calculated SNN clusters. (56B) Select cell type marker expression overlaid on the FDL visualization from 56A. The range for each marker is indicated in the bottom left of each plot. (56C) Heatmap for phenotype-specific markers across the T/NK cells. Top bar indicates each cell's assigned cell type and corresponding SNN cluster. (56D) Cells are colored on the FDL by the corresponding cell type color as in 56C. (56E) GZMB expression overlaid on the FDL, similar to 56B, range is indicated in bottom left. (56F) Violin plot for a cytotoxic score (PRF1, GNLY, GZMB, TNF, and IFNG) computed across the subsets identified. The highest cytotoxic activity derives from activated NK cells (FCGR3A+) in the metastatic microenvironment. (56G) Unbiased analysis stratifies the CD8+ T cells across the progenitor to differentiated/exhausted continuum previously identified by several groups (18, 19). This analysis indicates a heavy bias towards the progenitor phenotype (>0 PC1 embedding). Genes highly correlated with this axis were used to construct the expression scores used to bin T cells in FIG. 55E. (56H) PCA identifies 3 major subsets of TAMs in the metastatic niche. PC1 largely separates FCN1+ monocyte-like TAMs from more committed macrophage phenotypes. PC2 separates SPP1+ from C1QC+ macrophage phenotypes. (56I) Heatmap visualization of the gene expression programs specific to each TAM subset identified by the PCA in 56H. Top metadata indicate cell type, SNN cluster, and the discretized transcriptional subtype for each TAM's biopsy of origin. Other indicates either the PNET tumor PANFR0580 or PANFR0604 where Applicants did not recover any tumor cells (black, yes; white, no). (56J) FDL visualization for the TAM phenotypes which reinforces the inferred developmental hierarchy from “monocyte-like” to two different committed macrophage subsets as previously described (12). (56K) Bar chart specifying the number of fibroblasts captured from each biopsy. The distribution is strongly skewed for high frequency recovery of fibroblasts from sites other than the liver or from a different disease (PanNET). (56L) Scatterplot comparing all fibroblasts (n=826) for the expression of previously identified inflammatory cancer-associated fibroblast (iCAF) and myofibroblastic CAF (myCAF) expression signatures. (56M) Heatmap depiction of previously identified iCAF and myCAF signature genes (28). Cells are partitioned into myCAF, iCAF, and co-expression subsets. Top metadata bar indicates the discretized transcriptional subtype for the parent biopsy of each fibroblast (orange, basal; blue, classical; grey, Other). The distribution is also indicated for the fibroblasts originating from the 3 most “fibroblast-rich” biopsies (61% of Fibs).

FIGS. 57A-57G—Differential microenvironmental crosstalk shapes subtype-specific metastatic niches. (57A) Scatterplot comparing differential expression (x-axis) and subtype correlation coefficient (y-axis) for the 219 genes annotated as secreted growth factors, cytokines, or chemokines detected in malignant cells. Genes passing significance are assigned as “subtype specific” for either basal or classical (P<0.05, DE; P<0.003, correlation). (57B) Pathway enrichments for the top genes associated with each subtype. Shared enrichments are in black, orange and blue denote basal and classical-unique terms, respectively. (57C) Scatterplot comparing the fraction of endothelial cells captured and the average expression in malignant cells for CXCL5. Each point represents one biopsy (n=14). (57D) Differential expression between the committed TAM subsets SPP1+ and C1QC+. Genes are colored by their subtype selectivity (P<0.05; Log(Fold Change) >0.5). (57E) TAM phenotypic hierarchy plots for basal subtype tumors (FIG. 55G, bottom), split by biopsy and sorted by decreasing basal score. Heat indicates distribution of total TAMs (bottom right for each plot) per biopsy as in FIG. 55G. (57F) Dot plots for TAM (top, green fill) and malignant (bottom, red fill) expression of the indicated genes sorted by basal classical polarization. Size of the dot indicates fraction of cells expressing a given gene. Left of the dotted line are tumors visualized in 57E; basal tumors high for EMT program expression (PANFR0545 and PANFR0593) are indicated. (57G) Cross-correlation for markers of TAM subsets (green), basal (orange) and classical (blue) markers used in mIF studies, and putative malignant secreted factors (black).

FIGS. 58A-58F—Serial sampling of patient matched organoid models. (58A) Distribution of unique molecules and genes captured in quality cells per organoid sample, median values are indicated for each metric (dotted line) and violin plots are colored by patient ID (top, Log 10(UMIs); bottom, number of genes). (58B) Swimmer's plot for serial sampling of each attempted organoid model. Biopsies are ranked by their score difference and the dotted line denotes the discretized binning depicted in FIG. 51F. X-axis indicates the number of days since initiation (and initial biopsy single-cell profile). Points indicate where samples were taken and their fill color denotes the passage number. Organoids that stopped growing after P2 (e.g. PANFR0545) are indicated with a line and a crossed-out circle. Models that never grew (e.g. PANFR0593) are shown by a crossed-out circle at day 0. Arrows indicate models that survived iterative passages and classifies them as “established models”. 33% and 60% of models reached “establishment” from basal and classical tumors, respectively. (58C) t-SNE visualization of all biopsy and matched organoid cells from iterative passages, colored by patient ID. Dotted circles indicate the only two SNN clusters (4 and 32) with appreciably admixed clusters, the rest were patient specific. Bar chart shows number of organoid cells recovered per biopsy (right). (58D) The same t-SNE visualization as in 58C, but colored by organoid or original biopsy identity. (58E) Relative expression for genes defining cluster 4 (top) and cluster 32 (bottom; 1 versus rest DE with the cells in 58C). Cluster 4 had an ambiguous epithelial identity and low to absent detection of CNVs (see FIG. 60). Cluster 32 cells were defined by canonical fibroblast genes. (58F) Fraction of cluster 4 cells at each passage. These cells did not survive iterative passaging suggesting that they were either untransformed or unfit in organoid culture.

FIGS. 59A-59E—Organoid culture microenvironment selects against the basal state. (59A) Relative expression for basal and classical genes in biopsy cells (left) and their matched, early passage organoid cells (n=13 models; right). (59B) Scatterplot compares the relative contribution of genotypic drift (CNVs, y-axis) versus phenotypic drift (basal/classical gene expression, x-axis). Both quantities represent distance (d=(1−r)/2); higher value=greater distance) derived from Pearson correlation (r). Each point is one organoid/biopsy pair and summarizes the average d between organoid cells and their matched initial biopsy. Dotted lines are P<0.05 comparing average intra-biopsy (biopsy cells to themselves) d across the cohort for both metrics. Fill colors denote classification of original tumor, point outline color is the biopsy identifier. (59C) Line plot for each biopsy and its successive organoid samples (*see Methods). Points represent the sample averaged score at the indicated timepoints, lines tie samples derived from the same initial biopsy. Color indicates if the original biopsy was initially measured as basal (orange) or classical (blue). Colored point outlines denote all samples from the indicated original biopsy. Crossed empty circles indicate when a sample failed to grow. (59D) Representative scatterplots for single-cell basal and classical scores in biopsy (grey) and the indicated organoid passage (red) sample. (59E) Genotype and phenotype evolution in PANFR0575. Cells are sorted first by their subclone (A-F, color bar far left; Methods) and then sample of origin (Biopsy or organoid, right of subclone color bar; Pn, Organoid passage number). Each single cell's corresponding phenotype is shown in the center heatmap and far right expression score bars (Cell cycle, black). The fraction of each subclone in each sample is indicated with pie charts at the bottom, cell numbers per sample are below.

FIGS. 60A-60E—Comparison of genotype and phenotype in matched sample pairs reveals distinct patterns of ex vivo evolution. (60A-60D) Main heatmaps show inferred CNV copy number status for every cell in each biopsy/organoid pair. Cells are ordered by hierarchical clustering of their CNV profiles and letters on the far left indicate subclones that have significant statistical evidence for tree-splitting (Methods). Each cell's origin (biopsy tissue, grey; early passage organoid, red) is also noted (Source). Right metadata bars indicate if that cell came from an admixed SNN cluster (4 or 32; FIG. 58C-58E) as well as each individual cell's expression phenotype (classical, basal, or EMT). Pattern 1 (60A, n=4) consists of models that stopped growing after P2 organoids were measured (3/4 basal). Pattern 2 (60B, n=3) consists of models that showed evidence of selective outgrowth for a clone not seen or seen at very low frequency in the original biopsy (2/3 basal). Pattern 3 (60C, n=5) are all models where neutral evolution occurred, that is, a dominant clone in the biopsy grew out in the organoid (0/5 basal). Pattern 4 (60D) was a single model (PANFR0575, strongest basal in the dataset) that displayed plasticity where at the level of CNVs it fits the pattern of neutral evolution, but phenotypically it completely diverges from its parent biopsy cells. (60E) Genotype and phenotype evolution in PANFR0489R over successive passages in culture (Pn, Passage number), similar to FIG. 59E. The fraction of subclones in each sample are indicated with pie charts at the bottom.

FIGS. 61A-61E—Recovery of the basal state in altered media conditions. (61A) Relative expression for 90 genes representing basal, classical, and WNT7B expression programs across bulk RNA-seq samples from primary resections (TCGA) and metastatic biopsies (Panc-Seq), as well as organoid and cell line (CCLE) models. Phenotype scores are indicated and samples are ranked by their score difference. (61B) Schematic for depleted media experiment. (61C) Single-cell violin plots for basal score in passage matched organoids grown in the indicated media conditions (***P=2.2×10-16). (61D) Dot plot represents the sample average basal score in the indicated conditions. Lines tie samples and color outlines indicate sample identity. Each sample's biopsy basal score is included for reference. (61E) Inferred CNVs, expression scores, and cell cycle status for each cell from either Stripped (grey) or Full (red) organoid media conditions in the PANFR489R experiment.

FIGS. 62A-62C—Alterations to organoid media, but not matrix dimensionality, shift transcriptional phenotype. (62A) Four established models from FIG. 61A were adapted to 2-dimensional culture in standard organoid media and measured via bulk RNA-seq. Rows indicate expression levels of basal and classical genes from Moffitt et al (16). (62B) Scatter plots for each model comparison in the different media conditions, original biopsy tissue (grey) is included for reference. (62C) Brightfield images were obtained for organoids grown in standard organoid media (“Full”) or in media without any growth factors (“Stripped”) at days 1 and 11 after seeding.

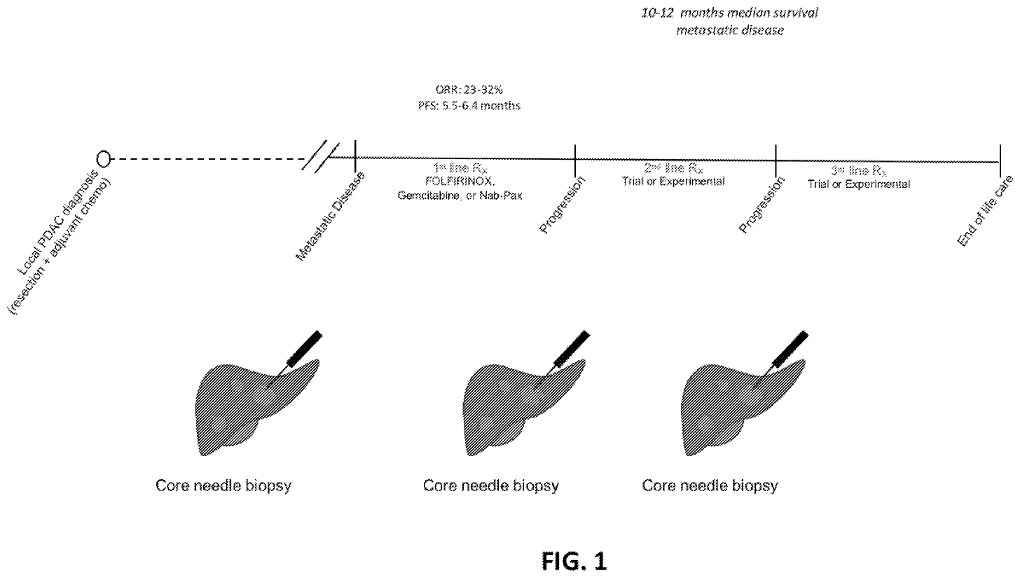

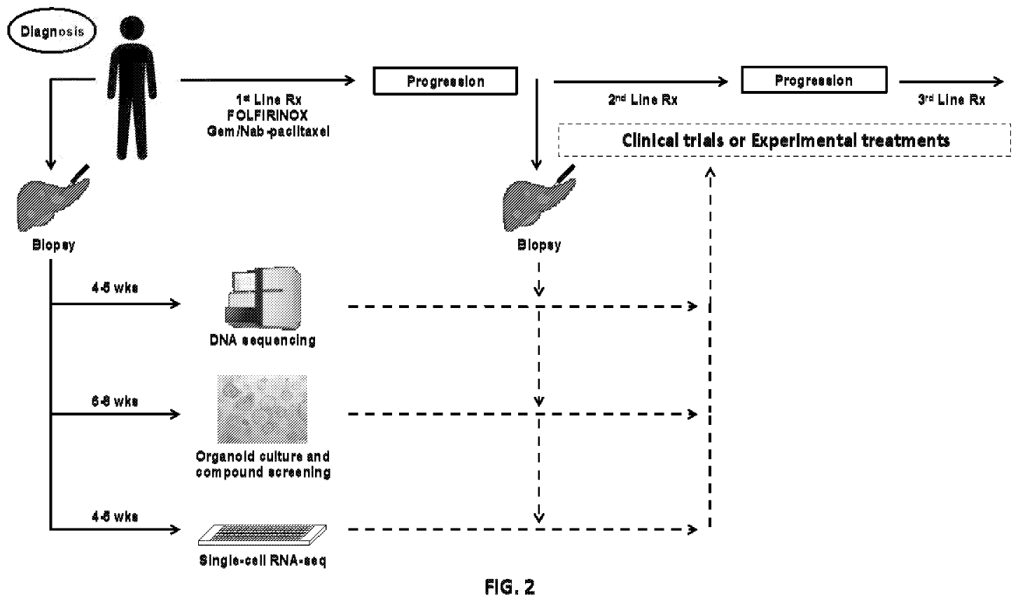

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### Overview

Embodiments disclosed herein provide compositions and methods for generating ex vivo cell-based systems. To date it has been technically challenging to retrieve both a reliable single-cell profile and generate an organoid from a primary patient specimen. Applicants believe a low input single-cell RNA-seq (scRNA-seq) method like Seq-Well (Gierahn et al. Nat Methods 14:395-398 (2017)) is an ideal approach to further interrogate this system. This platform allows one to economically sequence thousands of single cells from a given sample, preserving the information from the microenvironment as well as computationally isolating the tumor cells for further study. Requiring <10K cells, integrating Seq-Well into the organoid generation pipeline is a seamless approach to profiling the exact same single-cell suspension that the resultant organoids are grown from.

### Methods of Generating Ex Vivo Cell-Based Systems

In some embodiments, the invention provides a method of generating an ex vivo cell-based system comprising dissociating an original tissue sample obtained from a subject into a single cell population; determining an in vivo phenotype of the tissue sample by conducting single-cell RNA analysis on a first portion of the single cells; establishing an ex vivo cell-based system from a second portion of the single cells; and culturing the ex vivo cell-based system in a medium or conditions selected to maintain the in vivo phenotype.

An “ex vivo cell-based system” may comprise single cells of a particular type, sub-type or state, or a combination of cells of the same or differing type, sub-type, or state. The ex vivo cell-based system may be a model for screening perturbations to better understand the underlying biology or to identify putative targets for treating a disease, or for screening putative therapeutics, and also include models derived ex vivo but further implanted into a living organism, such as a mouse or pig, prior to perturbation of the model. An ex vivo cell-based system may also be a cell-based therapeutic for delivery to an organism to treat disease, or an implant meant to restore or regenerate damaged tissue. An “in vivo system” may likewise comprise a single cell or a combination of cells of the same or differing type, sub-type, or state. As used herein, “ex vivo” may include, but not be limited to, in vitro systems, unless otherwise specifically indicated. The “in vivo system” may comprise healthy tissue or cells, or tissues or cells in a homeostatic state, or diseased tissue or cells, or diseased tissue or cells in a non-homeostatic state, or tissues or cells within a viable organism, or diseased tissue or cells within a viable organism. A homeostatic state may include cells or tissues demonstrating a physiology and/or structure typically observed in an healthy living organism. In other embodiments, a homeostatic state may be considered the state that a cell or tissue naturally adopts under a given set of growth conditions and absent further defined genetic, chemical, or environmental perturbations.

The ex vivo cell-based system comprises a single cell type or sub-type, a combination of cell types and/or subtypes, a cell-based therapeutic, an explant, or an organoid derived using the methods disclosed herein.

It should be noted that the methods disclosed herein may be used to develop an ex vivo cell-based system de novo from a source starting material, or to improve an existing ex vivo cell-based system. Source starting materials may include cultured cell lines or cells or tissues isolated directly from an in vivo source, including explants and biopsies. The source materials may be pluripotent cells including stem cells.

### Dissociating an Original Tissue Sample

In certain embodiments, a tissue sample is obtained from a subject. As used herein, a “sample”, “tissue sample”, or “biological sample” may contain whole cells and/or live cells and/or cell debris. The sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The tissue sample is dissociated into a single cell population. Standard tissue dissociation techniques may be used. General techniques useful in the practice of this invention in cell culture and media uses are known in the art (e.g., Large Scale Mammalian Cell Culture (Hu et al. 1997. Curr Opin Biotechnol 8: 148); Serum-free Media (K. Kitano. 1991. Biotechnology 17: 73); or Large Scale Mammalian Cell Culture (Curr Opin Biotechnol 2: 375, 1991). The terms “culturing” or “cell culture” are common in the art and broadly refer to maintenance of cells and potentially expansion (proliferation, propagation) of cells in vitro. Typically, animal cells, such as mammalian cells, such as human cells, are cultured by exposing them to (i.e., contacting them with) a suitable cell culture medium in a vessel or container adequate for the purpose (e.g., a 96-, 24-, or 6-well plate, a T-25, T-75, T-150 or T-225 flask, or a cell factory), at art-known conditions conducive to in vitro cell culture, such as temperature of 37° C., 5% v/v CO₂ and >95% humidity.

As used herein, a “population” of cells is any number of cells greater than 1, but is preferably at least 1×10³ cells, at least 1×10⁴ cells, at least at least 1×10⁵ cells, at least 1×10⁶ cells, at least 1×10⁷ cells, at least 1×10⁸ cells, at least 1×10⁹ cells, or at least 1×10¹⁰ cells.

In certain embodiments, the single cell population may comprise a single cell type or subtype or combination of cell types and/or subtypes comprises an immune cell, intestinal cell, liver cell, kidney cell, lung cell, brain cell, epithelial cell, endoderm cell, neuron, ectoderm cell, islet cell, acinar cell, oocyte, sperm, hematopoietic cell, hepatocyte, skin/keratinocyte, melanocyte, bone/osteocyte, hair/dermal papilla cell, cartilage/chondrocyte, fat cell/adipocyte, skeletal muscular cell, endothelium cell, cardiac muscle/cardiomyocyte, trophoblast, tumor cell, or tumor microenvironment (TME) cell.

In some embodiments, the original tissue sample is a tumor tissue sample. The tumor may include, without limitation, solid tumors such as sarcomas and carcinomas. Examples of solid tumors include, but are not limited to fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, epithelial carcinoma, bronchogenic carcinoma, hepatoma, colorectal cancer (e.g., colon cancer, rectal cancer), anal cancer, pancreatic cancer (e.g., pancreatic adenocarcinoma, islet cell carcinoma, neuroendocrine tumors), breast cancer (e.g., ductal carcinoma, lobular carcinoma, inflammatory breast cancer, clear cell carcinoma, mucinous carcinoma), ovarian carcinoma (e.g., ovarian epithelial carcinoma or surface epithelial-stromal tumour including serous tumour, endometrioid tumor and mucinous cystadenocarcinoma, sex-cord-stromal tumor), prostate cancer, liver and bile duct carcinoma (e.g., hepatocellular carcinoma, cholangiocarcinoma, hemangioma), choriocarcinoma, seminoma, embryonal carcinoma, kidney cancer (e.g., renal cell carcinoma, clear cell carcinoma, Wilm's tumor, nephroblastoma), cervical cancer, uterine cancer (e.g., endometrial adenocarcinoma, uterine papillary serous carcinoma, uterine clear-cell carcinoma, uterine sarcomas and leiomyosarcomas, mixed mullerian tumors), testicular cancer, germ cell tumor, lung cancer (e.g., lung adenocarcinoma, squamous cell carcinoma, large cell carcinoma, bronchioloalveolar carcinoma, non-small-cell carcinoma, small cell carcinoma, mesothelioma), bladder carcinoma, signet ring cell carcinoma, cancer of the head and neck (e.g., squamous cell carcinomas), esophageal carcinoma (e.g., esophageal adenocarcinoma), tumors of the brain (e.g., glioma, glioblastoma, medullablastoma, astrocytoma, medulloblastoma, craniopharyngioma, ependymoma, pinealoma, hemangioblastoma, acoustic neuroma, oligodenroglioma, schwannoma, meningioma), neuroblastoma, retinoblastoma, neuroendocrine tumor, melanoma, cancer of the stomach (e.g., stomach adenocarcinoma, gastrointestinal stromal tumor), or carcinoids. Lymphoproliferative disorders are also considered to be proliferative diseases.

In specific embodiments, the tumor is a pancreatic ductal adenocarcinoma (PDAC) tumor.

In other embodiments, the tumor may be a breast cancer tumor.

In yet other embodiments, the tumor may be a bladder cancer tumor.

### Determining an In Vivo Phenotype

As used herein, the term “phenotype” in the context of a tissue sample relates to a set of observable physical characteristics that include one or more cell types, one or more cell states, etc.

In certain embodiments, determining an in vivo phenotype comprises single cell RNA sequencing (see, e.g., Kalisky, T., Blainey, P. & Quake, S. R. Genomic Analysis at the Single-Cell Level. Annual review of genetics 45, 431-445, (2011); Kalisky, T. & Quake, S. R. Single-cell genomics. Nature Methods 8, 311-314 (2011); Islam, S. et al. Characterization of the single-cell transcriptional landscape by highly multiplex RNA-seq. Genome Research, (2011); Tang, F. et al. RNA-Seq analysis to capture the transcriptome landscape of a single cell. Nature Protocols 5, 516-535, (2010); Tang, F. et al. mRNA-Seq whole-transcriptome analysis of a single cell. Nature Methods 6, 377-382, (2009); Ramskold, D. et al. Full-length mRNA-Seq from single-cell levels of RNA and individual circulating tumor cells. Nature Biotechnology 30, 777-782, (2012); and Hashimshony, T., Wagner, F., Sher, N. & Yanai, I. CEL-Seq: Single-Cell RNA-Seq by Multiplexed Linear Amplification. Cell Reports, Cell Reports, Volume 2, Issue 3, p 666-673, 2012).

In certain example embodiments, determining an in vivo phenotype of the tissue sample comprises using single cell RNA sequencing (scRNA-seq) one or more cell (sub)types or one or more cell states in an initial or starting ex vivo cell-based system. Next, differences are identified in the cell (sub)type(s) and/or cell state(s) between the ex vivo cell-based systems a target in vivo system. The cell (sub)type(s) and cell state(s) of the in vivo system may likewise be determined using scRNA-seq. The scRNA-seq analysis may be obtained at the time of running the methods described herein are based on previously archived scRNA-seq analysis. Based on the identified differences, steps to modulate the source material to induce a shift in cell (sub)type(s) and/or cell state(s) that may more closely mimic the target in vivo system may then selected and applied.

In certain embodiments, different methods of single cell sequencing are better suited for sequencing certain samples (e.g., neurons, rare samples may be more optimally sequenced with a plate based method or single nuclei sequencing). In certain embodiments, the invention involves plate based single cell RNA sequencing (see, e.g., Picelli, S. et al., 2014, “Full-length RNA-seq from single cells using Smart-seq2” Nature protocols 9, 171-181, doi:10.1038/nprot.2014.006).

In certain embodiments, the invention involves high-throughput single-cell RNA-seq and/or targeted nucleic acid profiling (for example, sequencing, quantitative reverse transcription polymerase chain reaction, and the like) where the RNAs from different cells are tagged individually, allowing a single library to be created while retaining the cell identity of each read. In this regard reference is made to Macosko et al., 2015, “Highly Parallel Genome-wide Expression Profiling of Individual Cells Using Nanoliter Droplets” Cell 161, 1202-1214; International patent application number PCT/US2015/049178, published as WO2016/040476 on Mar. 17, 2016; Klein et al., 2015, “Droplet Barcoding for Single-Cell Transcriptomics Applied to Embryonic Stem Cells” Cell 161, 1187-1201; International patent application number PCT/US2016/027734, published as WO2016168584A1 on Oct. 20, 2016; Zheng, et al., 2016, “Haplotyping germline and cancer genomes with high-throughput linked-read sequencing” Nature Biotechnology 34, 303-311; Zheng, et al., 2017, “Massively parallel digital transcriptional profiling of single cells” Nat. Commun. 8, 14049 doi: 10.1038/ncomms14049; International patent publication number WO 2014210353 A2; Zilionis, et al., 2017, “Single-cell barcoding and sequencing using droplet microfluidics” Nat Protoc. January; 12(1):44-73; Cao et al., 2017, “Comprehensive single cell transcriptional profiling of a multicellular organism by combinatorial indexing” bioRxiv preprint first posted online Feb. 2, 2017, doi: dx.doi.org/10.1101/104844; Rosenberg et al., 2017, “Scaling single cell transcriptomics through split pool barcoding” bioRxiv preprint first posted online Feb. 2, 2017, doi: dx.doi.org/10.1101/105163; Vitak, et al., “Sequencing thousands of single-cell genomes with combinatorial indexing” Nature Methods, 14(3):302-308, 2017; Cao, et al., Comprehensive single-cell transcriptional profiling of a multicellular organism. Science, 357(6352):661-667, 2017; and Gierahn et al., “Seq-Well: portable, low-cost RNA sequencing of single cells at high throughput” Nature Methods 14, 395-398 (2017), all the contents and disclosure of each of which are herein incorporated by reference in their entirety.

In certain embodiments, the invention involves single nucleus RNA sequencing. In this regard reference is made to Swiech et al., 2014, “In vivo interrogation of gene function in the mammalian brain using CRISPR-Cas9” Nature Biotechnology Vol. 33, pp. 102-106; Habib et al., 2016, “Div-Seq: Single-nucleus RNA-Seq reveals dynamics of rare adult newborn neurons” Science, Vol. 353, Issue 6302, pp. 925-928; Habib et al., 2017, “Massively parallel single-nucleus RNA-seq with DroNc-seq” Nat Methods. 2017 October; 14(10):955-958; and International patent application number PCT/US2016/059239, published as WO2017164936 on Sep. 28, 2017, which are herein incorporated by reference in their entirety.

In certain embodiments, the invention involves the Assay for Transposase Accessible Chromatin using sequencing (ATAC-seq) as described. (see, e.g., Buenrostro, et al., Transposition of native chromatin for fast and sensitive epigenomic profiling of open chromatin, DNA-binding proteins and nucleosome position. Nature methods 2013; 10 (12): 1213-1218; Buenrostro et al., Single-cell chromatin accessibility reveals principles of regulatory variation. Nature 523, 486-490 (2015); Cusanovich, D. A., Daza, R., Adey, A., Pliner, H., Christiansen, L., Gunderson, K. L., Steemers, F. J., Trapnell, C. & Shendure, J. Multiplex single-cell profiling of chromatin accessibility by combinatorial cellular indexing. Science. 2015 May 22; 348(6237):910-4. doi: 10.1126/science.aab1601. Epub 2015 May 7; US20160208323A1; US20160060691A1; and WO2017156336A1).

In specific embodiments, the single-cell RNA sequencing method is Seq-Well.

For further methods of cell culture solutions and systems, see International Patent publication WO2014159356A1, the entirety of which is incorporated by reference herein.

In some embodiments, the methods described herein may further comprise conducting a second single-cell RNA analysis on single cells derived from the established ex vivo cell-based system to determine a current phenotype; and if the phenotype has changed, modifying the culture medium or conditions to revert to or decrease the expression space between the current phenotype and the in vivo phenotype.

Other methods for assessing differences in the ex vivo and in vivo systems may be employed. In certain example embodiments, an assessment of differences in the in vivo and ex vivo proteome may be used to further identify key differences in cell type and sub-types or cell states. For example, isobaric mass tag labeling and liquid chromatography mass spectroscopy may be used to determine relative protein abundances in the ex vivo and in vivo systems.

### Culturing Ex-Vivo Cell-Based-System in Conditions Selected to Maintain the In Vivo Phenotype

In certain example embodiments, a statistically significant shift in the initial ex vivo gene expression distribution toward the gene expression distribution of the in vivo systems is sought post-modulation. A statistically significant shift in gene expression distribution can be at least 10%, at least 11%, at least 12%, at least 13%, at least 14%, at least 15%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%.

In certain example embodiments, statistical shifts may be determined by defining an in vivo score. For example, a gene list of key genes enriched in the in vivo model may be defined. To determine the fractional contribution to a cell's transcriptome to that gene list, the total log (scaled UMI+1) expression values for gene with the list of interest are summed and then divided by the total amount of scaled UMI detected in that cell giving a proportion of a cell's transcriptome dedicated to producing those genes. Thus, statistically significant shifts may be shifts in an initial score for the ex vivo system after modulation towards the in vivo score or after modulation with an aim of moving in a statistically significant fashion towards the in vivo score.

Modification of Cell Culture Systems

The term “modulate” or “modify” broadly denotes a qualitative and/or quantitative alteration, change or variation in that which is being modulated. Where modulation can be assessed quantitatively—for example, where modulation comprises or consists of a change in a quantifiable variable such as a quantifiable property of a cell or where a quantifiable variable provides a suitable surrogate for the modulation—modulation specifically encompasses both increase (e.g., activation) or decrease (e.g., inhibition) in the measured variable. The term encompasses any extent of such modulation, e.g., any extent of such increase or decrease, and may more particularly refer to statistically significant increase or decrease in the measured variable. By means of example, modulation may encompass an increase in the value of the measured variable by at least about 10%, e.g., by at least about 20%, preferably by at least about 30%, e.g., by at least about 40%, more preferably by at least about 50%, e.g., by at least about 75%, even more preferably by at least about 100%, e.g., by at least about 150%, 200%, 250%, 300%, 400% or by at least about 500%, compared to a reference situation without said modulation; or modulation may encompass a decrease or reduction in the value of the measured variable by at least about 10%, e.g., by at least about 20%, by at least about 30%, e.g., by at least about 40%, by at least about 50%, e.g., by at least about 60%, by at least about 70%, e.g., by at least about 80%, by at least about 90%, e.g., by at least about 95%, such as by at least about 96%, 97%, 98%, 99% or even by 100%, compared to a reference situation without said modulation. Preferably, modulation may be specific or selective, hence, one or more desired phenotypic aspects of a cell or cell population may be modulated without substantially altering other (unintended, undesired) phenotypic aspect(s).

Selection of modulating agents will depend on key targets identified by the analysis describe above, and which aspects of gene expression need to be modified to shift expression towards that of the in vivo model. Modulating agents may comprise cytokines, growth factors, hormones, transcription factors, metabolites or small molecules. The modulating agent may also be a genetic modifying agent or an epigenetic modifying agent. The genetic modulating agent may be a CRISPR system, as described further below, a zinc finger nuclease system, a TALEN, or a meganuclease. The epigenetic modifying agent may be a DNA methylation inhibitor, HDAC inhibitor, histone acetylation inhibitor, histone methylation inhibitor, or histone demethylase inhibitor.

TALE Systems

As disclosed herein editing can be made by way of the transcription activator-like effector nucleases (TALENs) system. Transcription activator-like effectors (TALEs) can be engineered to bind practically any desired DNA sequence. Exemplary methods of genome editing using the TALEN system can be found for example in Cermak T. Doyle E L. Christian M. Wang L. Zhang Y. Schmidt C, et al. Efficient design and assembly of custom TALEN and other TAL effector-based constructs for DNA targeting. Nucleic Acids Res. 2011; 39:e82; Zhang F. Cong L. Lodato S. Kosuri S. Church G M. Arlotta P Efficient construction of sequence-specific TAL effectors for modulating mammalian transcription. Nat Biotechnol. 2011; 29:149-153 and U.S. Pat. Nos. 8,450,471, 8,440,431 and 8,440,432, all of which are specifically incorporated by reference.

In advantageous embodiments of the invention, the methods provided herein use isolated, non-naturally occurring, recombinant or engineered DNA binding proteins that comprise TALE monomers as a part of their organizational structure that enable the targeting of nucleic acid sequences with improved efficiency and expanded specificity.

Naturally occurring TALEs or “wild type TALEs” are nucleic acid binding proteins secreted by numerous species of proteobacteria. TALE polypeptides contain a nucleic acid binding domain composed of tandem repeats of highly conserved monomer polypeptides that are predominantly 33, 34 or 35 amino acids in length and that differ from each other mainly in amino acid positions 12 and 13. In advantageous embodiments the nucleic acid is DNA. As used herein, the term “polypeptide monomers”, or “TALE monomers” will be used to refer to the highly conserved repetitive polypeptide sequences within the TALE nucleic acid binding domain and the term “repeat variable di-residues” or “RVD” will be used to refer to the highly variable amino acids at positions 12 and 13 of the polypeptide monomers. As provided throughout the disclosure, the amino acid residues of the RVD are depicted using the IUPAC single letter code for amino acids. A general representation of a TALE monomer which is comprised within the DNA binding domain is X1-11-(X12X13)-X14-33 or 34 or 35, where the subscript indicates the amino acid position and X represents any amino acid. X12X13 indicate the RVDs. In some polypeptide monomers, the variable amino acid at position 13 is missing or absent and in such polypeptide monomers, the RVD consists of a single amino acid. In such cases the RVD may be alternatively represented as X*, where X represents X12 and (*) indicates that X13 is absent. The DNA binding domain comprises several repeats of TALE monomers and this may be represented as (X1-11-(X12X13)-X14-33 or 34 or 35)z, where in an advantageous embodiment, z is at least 5 to 40. In a further advantageous embodiment, z is at least 10 to 26.

The TALE monomers have a nucleotide binding affinity that is determined by the identity of the amino acids in its RVD. For example, polypeptide monomers with an RVD of NI preferentially bind to adenine (A), polypeptide monomers with an RVD of NG preferentially bind to thymine (T), polypeptide monomers with an RVD of HD preferentially bind to cytosine (C) and polypeptide monomers with an RVD of NN preferentially bind to both adenine (A) and guanine (G). In yet another embodiment of the invention, polypeptide monomers with an RVD of IG preferentially bind to T. Thus, the number and order of the polypeptide monomer repeats in the nucleic acid binding domain of a TALE determines its nucleic acid target specificity. In still further embodiments of the invention, polypeptide monomers with an RVD of NS recognize all four base pairs and may bind to A, T, G or C. The structure and function of TALEs is further described in, for example, Moscou et al., Science 326:1501 (2009); Boch et al., Science 326:1509-1512 (2009); and Zhang et al., Nature Biotechnology 29:149-153 (2011), each of which is incorporated by reference in its entirety.

The TALE polypeptides used in methods of the invention are isolated, non-naturally occurring, recombinant or engineered nucleic acid-binding proteins that have nucleic acid or DNA binding regions containing polypeptide monomer repeats that are designed to target specific nucleic acid sequences.

As described herein, polypeptide monomers having an RVD of HN or NH preferentially bind to guanine and thereby allow the generation of TALE polypeptides with high binding specificity for guanine containing target nucleic acid sequences. In a preferred embodiment of the invention, polypeptide monomers having RVDs RN, NN, NK, SN, NH, KN, HN, NQ, HH, RG, KH, RH and SS preferentially bind to guanine. In a much more advantageous embodiment of the invention, polypeptide monomers having RVDs RN, NK, NQ, HH, KH, RH, SS and SN preferentially bind to guanine and thereby allow the generation of TALE polypeptides with high binding specificity for guanine containing target nucleic acid sequences. In an even more advantageous embodiment of the invention, polypeptide monomers having RVDs HH, KH, NH, NK, NQ, RH, RN and SS preferentially bind to guanine and thereby allow the generation of TALE polypeptides with high binding specificity for guanine containing target nucleic acid sequences. In a further advantageous embodiment, the RVDs that have high binding specificity for guanine are RN, NH RH and KH. Furthermore, polypeptide monomers having an RVD of NV preferentially bind to adenine and guanine. In more preferred embodiments of the invention, polypeptide monomers having RVDs of H*, HA, KA, N*, NA, NC, NS, RA, and S* bind to adenine, guanine, cytosine and thymine with comparable affinity.

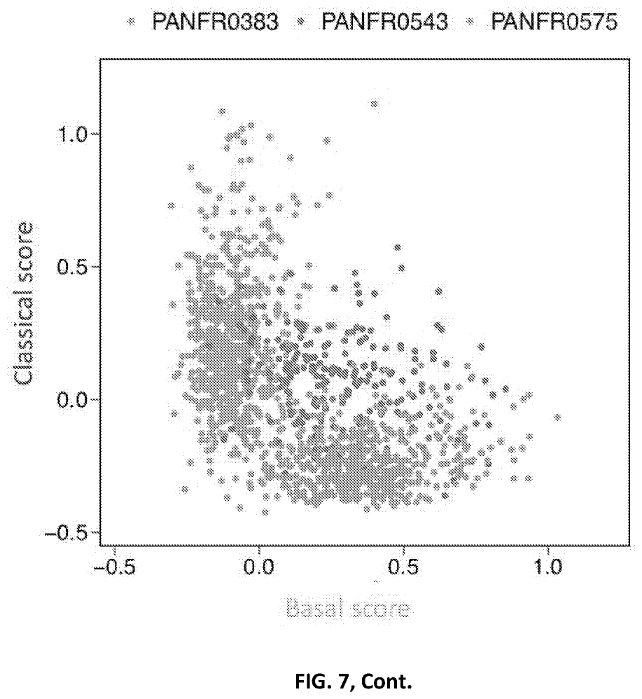

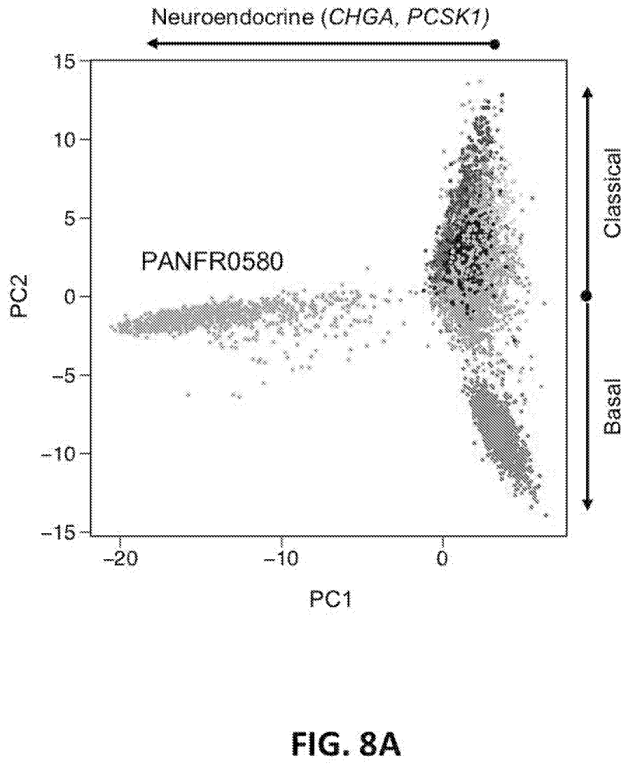

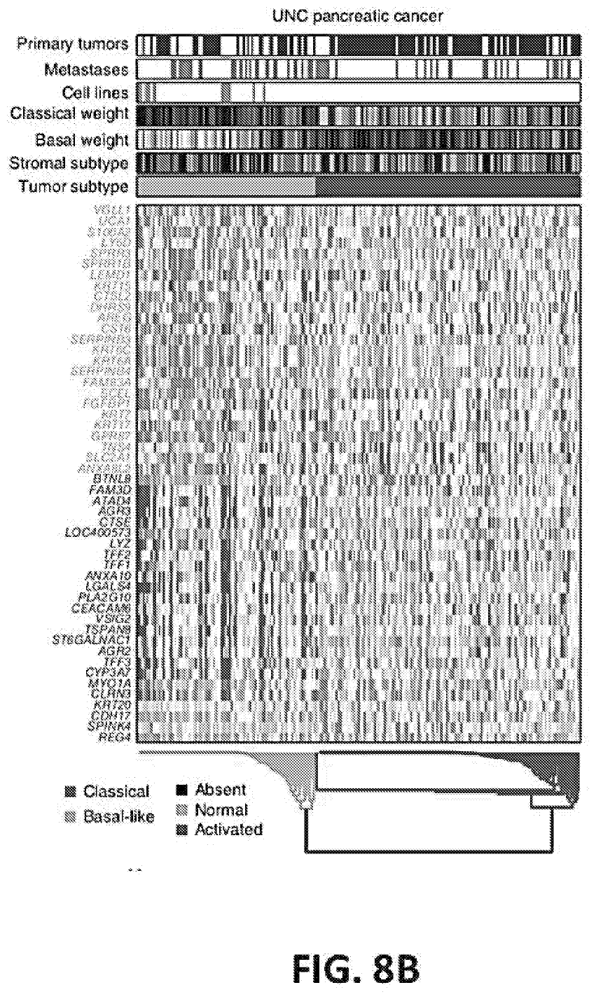

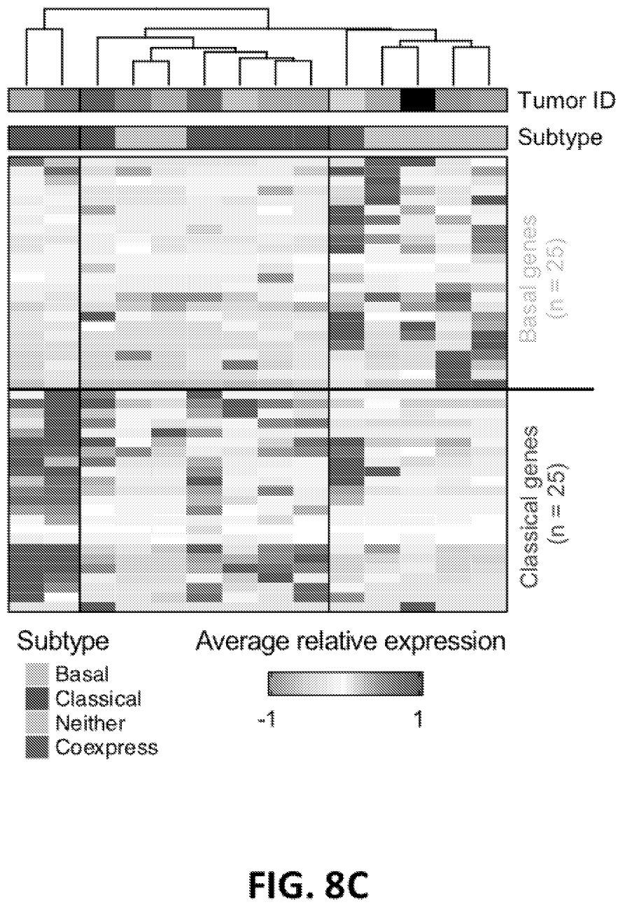

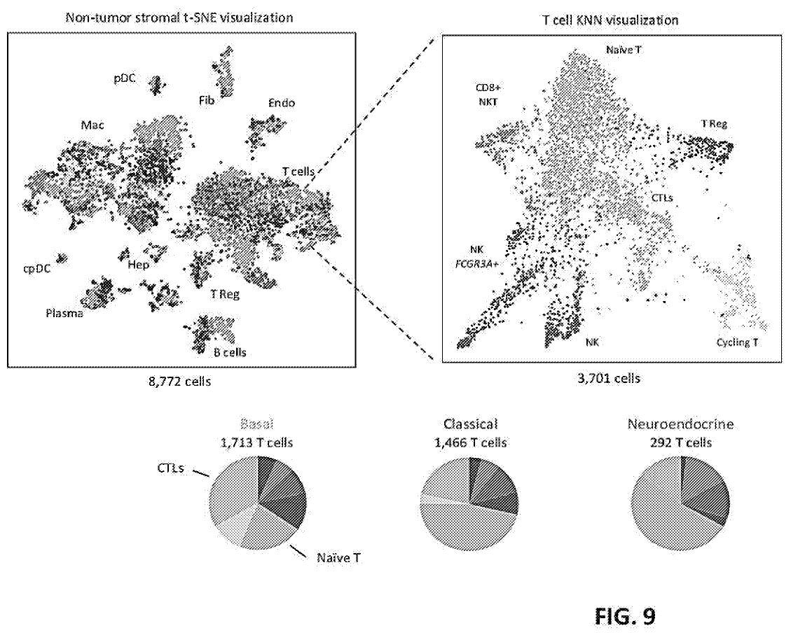

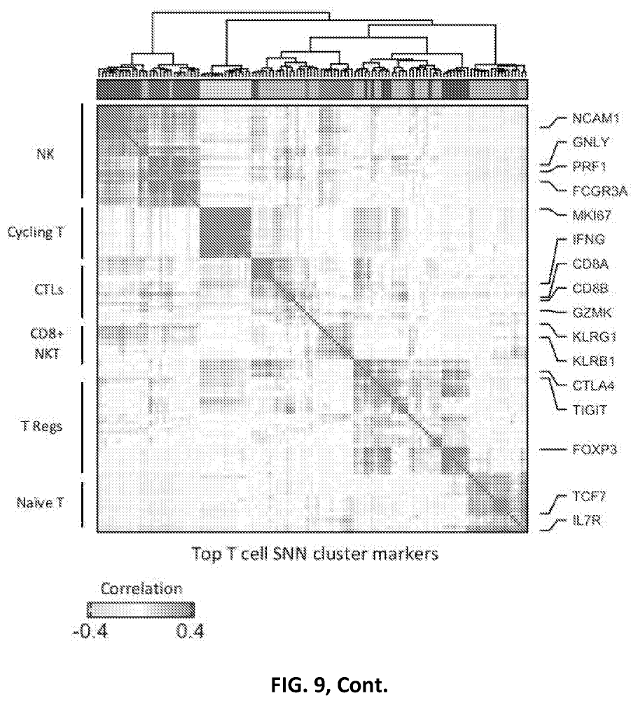

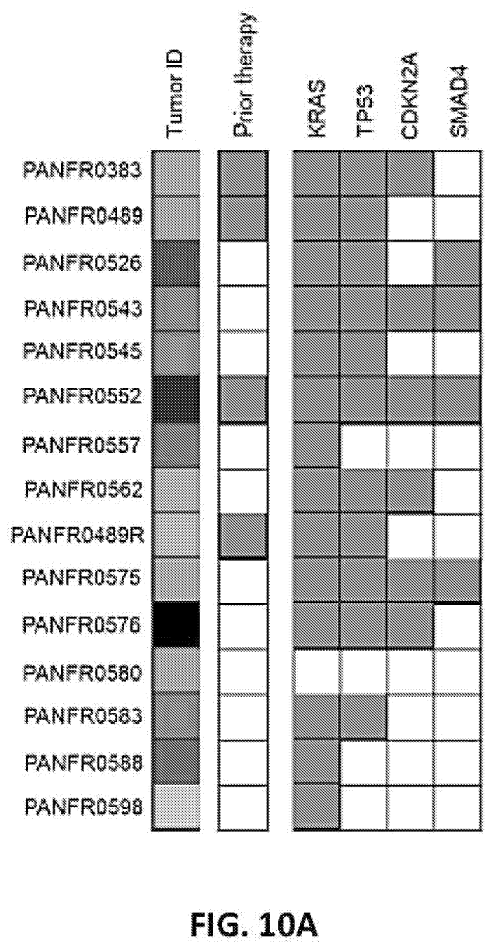

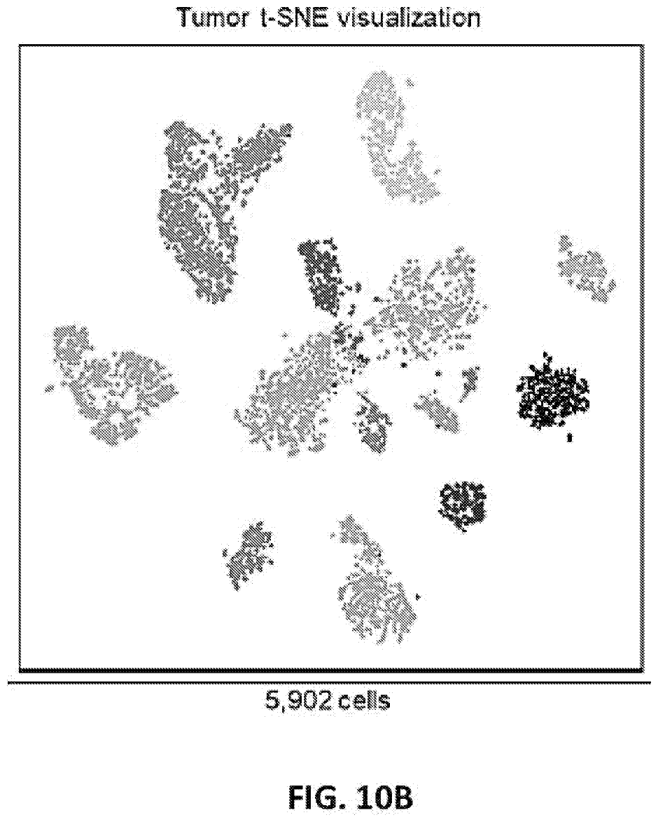

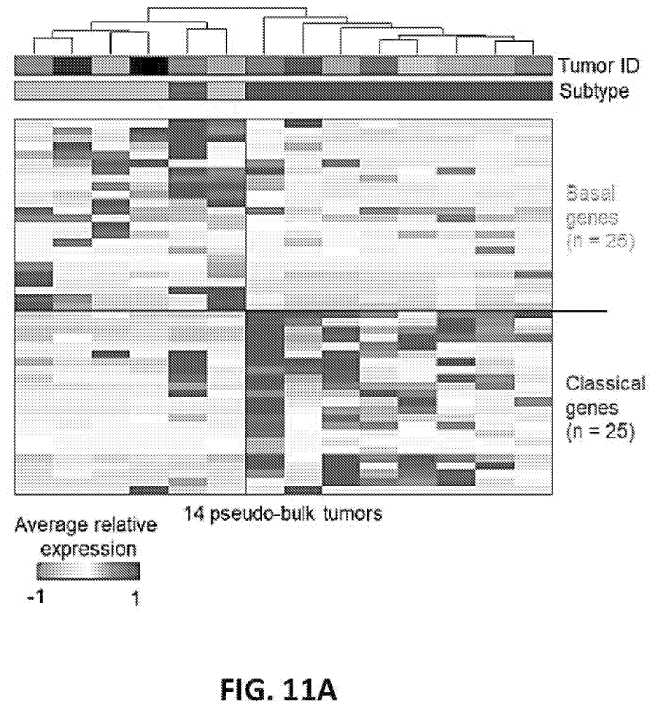

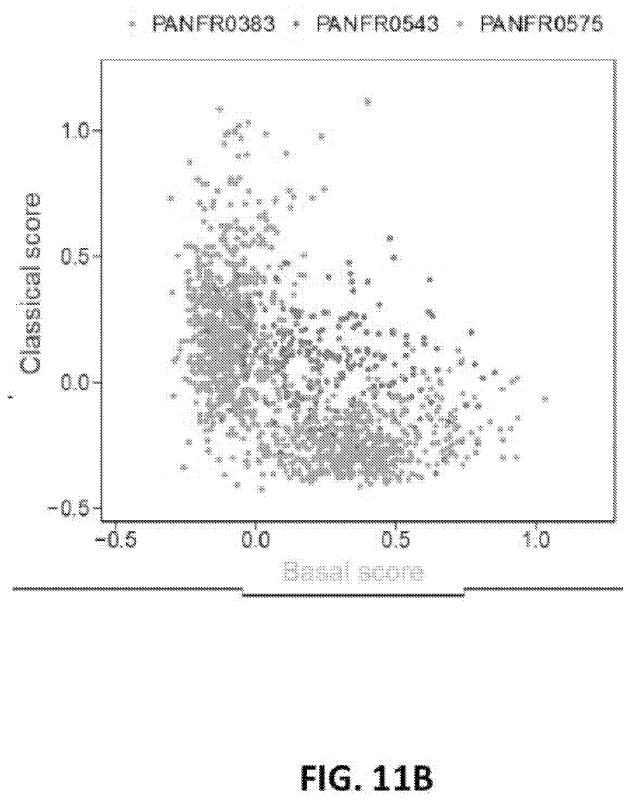

The predetermined N-terminal to C-terminal order of the one or more polypeptide monomers of the nucleic acid or DNA binding domain determines the corresponding predetermined target nucleic acid sequence to which the TALE polypeptides will bind. As used herein the polypeptide monomers and at least one or more half polypeptide monomers are “specifically ordered to target” the genomic locus or gene of interest. In plant genomes, the natural TALE-binding sites always begin with a thymine (T), which may be specified by a cryptic signal within the non-repetitive N-terminus of the TALE polypeptide; in some cases this region may be referred to as repeat 0. In animal genomes, TALE binding sites do not necessarily have to begin with a thymine (T) and TALE polypeptides may target DNA sequences that begin with T, A, G or C. The tandem repeat of TALE monomers always ends with a half-length repeat or a stretch of sequence that may share identity with only the first 20 amino acids of a repetitive full length TALE monomer and this half repeat may be referred to as a half-monomer (FIG. 8), which is included in the term “TALE monomer”. Therefore, it follows that the length of the nucleic acid or DNA being targeted is equal to the number of full polypeptide monomers plus two.

As described in Zhang et al., Nature Biotechnology 29:149-153 (2011), TALE polypeptide binding efficiency may be increased by including amino acid sequences from the “capping regions” that are directly N-terminal or C-terminal of the DNA binding region of naturally occurring TALEs into the engineered TALEs at positions N-terminal or C-terminal of the engineered TALE DNA binding region. Thus, in certain embodiments, the TALE polypeptides described herein further comprise an N-terminal capping region and/or a C-terminal capping region.

(SEQ ID NO: x)
M D P I R S R T P S P A R E L L S G P Q P D G V Q P T A D R G V S P
P A G G P L D G L P A R R T M S R T R L P S P P A P S P A F S A D S
F S D L L R Q F D P S L F N T S L F D S L P P F G A H H T E A A T G
E W D E V Q S G L R A A D A P P P T M R V A V T A A R P P R A K P A
P R R R A A Q P S D A S P A A Q V D L R T L G Y S Q Q Q Q E K I K P
K V R S T V A Q H H E A L V G F I G F T H A H I V A L S Q H P A A L G
T V A V K Y Q D M I A A L P E A T F I E AI V G V G K Q W S G A R A L
E A L L T V A G E L R G P P L Q L D T G Q L L KI A K R G G V T A V
E A V F I A W R N A L T G A P L N

An exemplary amino acid sequence of a N-terminal capping region is:

(SEQ ID NO: X)
R P A L E S I V A Q L S R P D P A L A A L T N D H L V A L A C L G
G R P A L D A V K K G L P H A P A L I K R T N R R I P E R T S H R
V A D H A Q V V R V L G F F Q C H S H P A Q A F D D A M T Q F G M
S R H G L L Q L F R R V G V T E L E A R S G T L P P A S Q R W D R
I L Q A S G M K R A K P S P T S T Q T P D Q A S L H A F A D S I
R D L D A P S P M H E G D Q T R A S

As used herein the predetermined “N-terminus” to “C terminus” orientation of the N-terminal capping region, the DNA binding domain comprising the repeat TALE monomers and the C-terminal capping region provide structural basis for the organization of different domains in the d-TALEs or polypeptides of the invention.

The entire N-terminal and/or C-terminal capping regions are not necessary to enhance the binding activity of the DNA binding region. Therefore, in certain embodiments, fragments of the N-terminal and/or C-terminal capping regions are included in the TALE polypeptides described herein.

In certain embodiments, the TALE polypeptides described herein contain a N-terminal capping region fragment that included at least 10, 20, 30, 40, 50, 54, 60, 70, 80, 87, 90, 94, 100, 102, 110, 117, 120, 130, 140, 147, 150, 160, 170, 180, 190, 200, 210, 220, 230, 240, 250, 260 or 270 amino acids of an N-terminal capping region. In certain embodiments, the N-terminal capping region fragment amino acids are of the C-terminus (the DNA-binding region proximal end) of an N-terminal capping region. As described in Zhang et al., Nature Biotechnology 29:149-153 (2011), N-terminal capping region fragments that include the C-terminal 240 amino acids enhance binding activity equal to the full length capping region, while fragments that include the C-terminal 147 amino acids retain greater than 80% of the efficacy of the full length capping region, and fragments that include the C-terminal 117 amino acids retain greater than 50% of the activity of the full-length capping region.

In some embodiments, the TALE polypeptides described herein contain a C-terminal capping region fragment that included at least 6, 10, 20, 30, 37, 40, 50, 60, 68, 70, 80, 90, 100, 110, 120, 127, 130, 140, 150, 155, 160, 170, 180 amino acids of a C-terminal capping region. In certain embodiments, the C-terminal capping region fragment amino acids are of the N-terminus (the DNA-binding region proximal end) of a C-terminal capping region. As described in Zhang et al., Nature Biotechnology 29:149-153 (2011), C-terminal capping region fragments that include the C-terminal 68 amino acids enhance binding activity equal to the full length capping region, while fragments that include the C-terminal 20 amino acids retain greater than 50% of the efficacy of the full length capping region.

In certain embodiments, the capping regions of the TALE polypeptides described herein do not need to have identical sequences to the capping region sequences provided herein. Thus, in some embodiments, the capping region of the TALE polypeptides described herein have sequences that are at least 50%, 60%, 70%, 80%, 85%, 90%, 91%, 92%, 93%, 94%, 95%, 96%, 97%, 98% or 99% identical or share identity to the capping region amino acid sequences provided herein. Sequence identity is related to sequence homology. Homology comparisons may be conducted by eye, or more usually, with the aid of readily available sequence comparison programs. These commercially available computer programs may calculate percent (%) homology between two or more sequences and may also calculate the sequence identity shared by two or more amino acid or nucleic acid sequences. In some preferred embodiments, the capping region of the TALE polypeptides described herein have sequences that are at least 95% identical or share identity to the capping region amino acid sequences provided herein.

Sequence homologies may be generated by any of a number of computer programs known in the art, which include but are not limited to BLAST or FASTA. Suitable computer program for carrying out alignments like the GCG Wisconsin Bestfit package may also be used. Once the software has produced an optimal alignment, it is possible to calculate % homology, preferably % sequence identity. The software typically does this as part of the sequence comparison and generates a numerical result.

In advantageous embodiments described herein, the TALE polypeptides of the invention include a nucleic acid binding domain linked to the one or more effector domains. The terms “effector domain” or “regulatory and functional domain” refer to a polypeptide sequence that has an activity other than binding to the nucleic acid sequence recognized by the nucleic acid binding domain. By combining a nucleic acid binding domain with one or more effector domains, the polypeptides of the invention may be used to target the one or more functions or activities mediated by the effector domain to a particular target DNA sequence to which the nucleic acid binding domain specifically binds.

In some embodiments of the TALE polypeptides described herein, the activity mediated by the effector domain is a biological activity. For example, in some embodiments the effector domain is a transcriptional inhibitor (i.e., a repressor domain), such as an mSin interaction domain (SID). SID4X domain or a Krappel-associated box (KRAB) or fragments of the KRAB domain. In some embodiments the effector domain is an enhancer of transcription (i.e. an activation domain), such as the VP16, VP64 or p65 activation domain. In some embodiments, the nucleic acid binding is linked, for example, with an effector domain that includes but is not limited to a transposase, integrase, recombinase, resolvase, invertase, protease, DNA methyltransferase, DNA demethylase, histone acetylase, histone deacetylase, nuclease, transcriptional repressor, transcriptional activator, transcription factor recruiting, protein nuclear-localization signal or cellular uptake signal.

In some embodiments, the effector domain is a protein domain which exhibits activities which include but are not limited to transposase activity, integrase activity, recombinase activity, resolvase activity, invertase activity, protease activity, DNA methyltransferase activity, DNA demethylase activity, histone acetylase activity, histone deacetylase activity, nuclease activity, nuclear-localization signaling activity, transcriptional repressor activity, transcriptional activator activity, transcription factor recruiting activity, or cellular uptake signaling activity. Other preferred embodiments of the invention may include any combination the activities described herein.

ZN-Finger Nucleases

Other preferred tools for genome editing for use in the context of this invention include zinc finger systems and TALE systems. One type of programmable DNA-binding domain is provided by artificial zinc-finger (ZF) technology, which involves arrays of ZF modules to target new DNA-binding sites in the genome. Each finger module in a ZF array targets three DNA bases. A customized array of individual zinc finger domains is assembled into a ZF protein (ZFP).

ZFPs can comprise a functional domain. The first synthetic zinc finger nucleases (ZFNs) were developed by fusing a ZF protein to the catalytic domain of the Type IS restriction enzyme FokI. (Kim, Y. G. et al., 1994, Chimeric restriction endonuclease, Proc. Natl. Acad. Sci. U.S.A. 91, 883-887; Kim, Y. G. et al., 1996, Hybrid restriction enzymes: zinc finger fusions to Fok I cleavage domain. Proc. Natl. Acad. Sci. U.S.A. 93, 1156-1160). Increased cleavage specificity can be attained with decreased off target activity by use of paired ZFN heterodimers, each targeting different nucleotide sequences separated by a short spacer. (Doyon, Y. et al., 2011, Enhancing zinc-finger-nuclease activity with improved obligate heterodimeric architectures. Nat. Methods 8, 74-79). ZFPs can also be designed as transcription activators and repressors and have been used to target many genes in a wide variety of organisms. Exemplary methods of genome editing using ZFNs can be found for example in U.S. Pat. Nos. 6,534,261, 6,607,882, 6,746,838, 6,794,136, 6,824,978, 6,866,997, 6,933,113, 6,979,539, 7,013,219, 7,030,215, 7,220,719, 7,241,573, 7,241,574, 7,585,849, 7,595,376, 6,903,185, and 6,479,626, all of which are specifically incorporated by reference.

Meganucleases

As disclosed herein editing can be made by way of meganucleases, which are endodeoxyribonucleases characterized by a large recognition site (double-stranded DNA sequences of 12 to 40 base pairs). Exemplary method for using meganucleases can be found in U.S. Pat. Nos. 8,163,514; 8,133,697; 8,021,867; 8,119,361; 8,119,381; 8,124,369; and 8,129,134, which are specifically incorporated by reference.

In certain embodiments, modulating the ex vivo cell-based system comprises delivering one or more modulating agents that modify expression of one or more cell types or states in the ex vivo cell-based system, delivering an additional cell type or sub-type to the ex vivo cell-based system, or depleting an existing cell type or sub-type from the ex vivo cell-based system. The one or more modulating agents may comprise one or more cytokines, growth factors, hormones, transcription factors, metabolites or small molecules.

Non-limiting examples of hormones include growth hormone (GH), adrenocorticotropic hormone (ACTH), dehydroepiandrosterone (DHEA), cortisol, epinephrine, thyroid hormone, estrogen, progesterone, testosterone, or combinations thereof.

Non-limiting examples of cytokines include lymphokines (e.g., interferon-7 (IFNγ), IL-2, IL-3, IL-4, IL-6, granulocyte-macrophage colony-stimulating factor (GM-CSF), interferon-γ, leukocyte migration inhibitory factors (T-LIF, B-LIF), lymphotoxin-alpha, macrophage-activating factor (MAF), macrophage migration-inhibitory factor (MIF), neuroleukin, immunologic suppressor factors, transfer factors, or combinations thereof), monokines (e.g., IL-1, TNF-alpha, interferon-α, interferon-β, colony stimulating factors, e.g., CSF2, CSF3, macrophage CSF or GM-CSF, or combinations thereof), chemokines (e.g., beta-thromboglobulin, C chemokines, CC chemokines, CXC chemokines, CX3C chemokines, macrophage inflammatory protein (MIP), or combinations thereof), interleukins (e.g., IL-1, IL-2, IL-3, IL-4, IL-5, IL-6, IL-7, IL-8, IL-9, IL-10, IL-11, IL-12, IL-13, IL-14, IL-15, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, IL-36, or combinations thereof), and several related signaling molecules, such as tumour necrosis factor (TNF) and interferons (e.g., interferon-α, interferon-β, interferon-γ, interferon-λ, or combinations thereof).

Non-limiting examples of mitogens include phytohaemagglutinin (PHA), concanavalin A (conA), lipopolysaccharide (LPS), pokeweed mitogen (PWM), phorbol ester such as phorbol myristate acetate (PMA) with or without ionomycin, or combinations thereof.

Non-limiting examples of cell surface receptors the ligands of which may act as immunomodulants include Toll-like receptors (TLRs) (e.g., TLR1, TLR2, TLR3, TLR4, TLR5, TLR6, TLR7, TLR8, TLR9, TLR10, TLR11, TLR12 or TLR13), CD80, CD86, CD40, CCR7, or C-type lectin receptors.

### Small Molecules

In certain embodiments, the one or more agents is a small molecule. The term “small molecule” refers to compounds, preferably organic compounds, with a size comparable to those organic molecules generally used in pharmaceuticals. The term excludes biological macromolecules (e.g., proteins, peptides, nucleic acids, etc.). Preferred small organic molecules range in size up to about 5000 Da, e.g., up to about 4000, preferably up to 3000 Da, more preferably up to 2000 Da, even more preferably up to about 1000 Da, e.g., up to about 900, 800, 700, 600 or up to about 500 Da. In certain embodiments, the small molecule may act as an antagonist or agonist (e.g., blocking an enzyme active site or activating a receptor by binding to a ligand binding site).

One type of small molecule applicable to the present invention is a degrader molecule (see, e.g., Ding, et al., Emerging New Concepts of Degrader Technologies, Trends Pharmacol Sci. 2020 July; 41(7):464-474). The terms “degrader” and “degrader molecule” refer to all compounds capable of specifically targeting a protein for degradation (e.g., ATTEC, AUTAC, LYTAC, or PROTAC, reviewed in Ding, et al. 2020). Proteolysis Targeting Chimera (PROTAC) technology is a rapidly emerging alternative therapeutic strategy with the potential to address many of the challenges currently faced in modern drug development programs. PROTAC technology employs small molecules that recruit target proteins for ubiquitination and removal by the proteasome (see, e.g., Zhou et al., Discovery of a Small-Molecule Degrader of Bromodomain and Extra-Terminal (BET) Proteins with Picomolar Cellular Potencies and Capable of Achieving Tumor Regression. J. Med. Chem. 2018, 61, 462-481; Bondeson and Crews, Targeted Protein Degradation by Small Molecules, Annu Rev Pharmacol Toxicol. 2017 Jan. 6; 57: 107-123; and Lai et al., Modular PROTAC Design for the Degradation of Oncogenic BCR-ABL Angew Chem Int Ed Engl. 2016 Jan. 11; 55(2): 807-810). In certain embodiments, LYTACs are particularly advantageous for cell surface proteins as described herein.

As described herein, small molecules targeting epigenetic proteins are currently being developed and/or used in the clinic to treat disease (see, e.g., Qi et al., HEDD: the human epigenetic drug database. Database, 2016, 1-10; and Ackloo et al., Chemical probes targeting epigenetic proteins: Applications beyond oncology. Epigenetics 2017, VOL. 12, NO. 5, 378-400). In certain embodiments, the one or more agents comprise a histone acetylation inhibitor, histone deacetylase (HDAC) inhibitor, histone lysine methylation inhibitor, histone lysine demethylation inhibitor, DNA methyltransferase (DNMT) inhibitor, inhibitor of acetylated histone binding proteins, inhibitor of methylated histone binding proteins, sirtuin inhibitor, protein arginine methyltransferase inhibitor or kinase inhibitor. In certain embodiments, any small molecule exhibiting the functional activity described above may be used in the present invention. In certain embodiments, the DNA methyltransferase (DNMT) inhibitor is selected from the group consisting of azacitidine (5-azacytidine), decitabine (5-aza-2′-deoxycytidine), EGCG (epigallocatechin-3-gallate), zebularine, hydralazine, and procainamide. In certain embodiments, the histone acetylation inhibitor is C646. In certain embodiments, the histone deacetylase (HDAC) inhibitor is selected from the group consisting of vorinostat, givinostat, panobinostat, belinostat, entinostat, CG-1521, romidepsin, ITF-A, ITF-B, valproic acid, OSU-HDAC-44, HC-toxin, magnesium valproate, plitidepsin, tasquinimod, sodium butyrate, mocetinostat, carbamazepine, SB939, CHR-2845, CHR-3996, JNJ-26481585, sodium phenylbutyrate, pivanex, abexinostat, resminostat, dacinostat, droxinostat, and trichostatin A (TSA). In certain embodiments, the histone lysine demethylation inhibitor is selected from the group consisting of pargyline, clorgyline, bizine, GSK2879552, GSK-J4, KDM5-C70, JIB-04, and tranylcypromine. In certain embodiments, the histone lysine methylation inhibitor is selected from the group consisting of EPZ-6438, GSK126, CPI-360, CPI-1205, CPI-0209, DZNep, GSK343, EI1, BIX-01294, UNC0638, EPZ004777, GSK343, UNC1999 and UNC0224. In certain embodiments, the inhibitor of acetylated histone binding proteins is selected from the group consisting of AZD5153 (see e.g., Rhyasen et al., AZD5153: A Novel Bivalent BET Bromodomain Inhibitor Highly Active against Hematologic Malignancies, Mol Cancer Ther. 2016 November; 15(11):2563-2574. Epub 2016 Aug. 29), PFI-1, CPI-203, CPI-0610, RVX-208, OTX015, I-BET151, I-BET762, I-BET-726, dBET1, ARV-771, ARV-825, BETd-260/ZBC260 and MZ1. In certain embodiments, the inhibitor of methylated histone binding proteins is selected from the group consisting of UNC669 and UNC1215. In certain embodiments, the sirtuin inhibitor comprises nicotinamide.

In some embodiments, the ex vivo cell-based system may be cultured in a medium comprising IFNγ if the phenotype is a basal phenotype and/or a IFNγ phenotype.

Modulation may be monitored in a number of ways. For example, expression of one or more key marker genes identified as described above may be measured at regular levels to assess increases in expression levels. Shifting of the ex vivo system to that of the in vivo system may also be measured phenotypically. For example, imaging an immunocytochemistry for key in vivo markers may be assessed at regular intervals to detect increased expression of the key in vivo markers. Likewise, flow cytometry may be used in a similar manner. In addition to detecting key in vivo markers, imaging modalities such as those described above may be used to further detect changes in cell morphology of the ex vivo system to more closely resemble the target in vivo system.

In certain embodiments, differentiation promoting agents may be used to obtain particular types of target cells. Differentiation promoting agents include anticoagulants, chelating agents, and antibiotics. Examples of such agents may be one or more of the following: vitamins and minerals or derivatives thereof, such as A (retinol), B3, C (ascorbate), ascorbate 2-phosphate, D such as D2 or D3, K, retinoic acid, nicotinamide, zinc or zinc compound, and calcium or calcium compounds; natural or synthetic hormones such as hydrocortisone, and dexamethasone; amino acids or derivatives thereof, such as L-glutamine (L-glu), ethylene glycol tetracetic acid (EGTA), proline, and non-essential amino acids (NEAA); compounds or derivatives thereof, such as β-mercaptoethal, dibutyl cyclic adenosine monophosphate (db-cAMP), monothioglycerol (MTG), putrescine, dimethyl sulfoxide (DMSO), hypoxanthine, adenine, forskolin, cilostamide, and 3-isobutyl-1-methylxanthine; nucleosides and analogues thereof, such as 5-azacytidine; acids or salts thereof, such as ascorbic acid, pyruvate, okadic acid, linoleic acid, ethylenediaminetetraacetic acid (EDTA), anticoagulant citrate dextrose formula A (ACDA), disodium EDTA, sodium butyrate, and glycerophosphate; antibiotics or drugs, such as G418, gentamycine, Pentoxifylline (1-(5-oxohexyl)-3,7-dimethylxanthine), and indomethacin; and proteins such as tissue plasminogen activator (TPA).

In certain example embodiments, the ex vivo system may be further modulated to not only more faithfully recapitulate a target in vivo system, but the ex vivo system may be further modulated to induce a gain of function. For example, one or more genes, gene expression cassettes (modules), or gene expression signature associated with the gain of function may be induced. Example gain of functions include, but are not limited to, increased anti-apoptotic activity or improved anti-microbial secretion.

In certain embodiments, gene signatures are modulated to shift an ex vivo system to more faithfully recapitulate an in vivo system. As used herein a “signature” may encompass any gene or genes, protein or proteins, or epigenetic element(s) whose expression profile or whose occurrence is associated with a specific cell type, subtype, or cell state of a specific cell type or subtype within a population of cells. For ease of discussion, when discussing gene expression, any of gene or genes, protein or proteins, or epigenetic element(s) may be substituted. As used herein, the terms “signature”, “expression profile”, or “expression program” may be used interchangeably. It is to be understood that also when referring to proteins (e.g. differentially expressed proteins), such may fall within the definition of “gene” signature. Levels of expression or activity or prevalence may be compared between different cells in order to characterize or identify for instance signatures specific for cell (sub)populations. Increased or decreased expression or activity or prevalence of signature genes may be compared between different cells in order to characterize or identify for instance specific cell (sub)populations. The detection of a signature in single cells may be used to identify and quantitate for instance specific cell (sub)populations. A signature may include a gene or genes, protein or proteins, or epigenetic element(s) whose expression or occurrence is specific to a cell (sub)population, such that expression or occurrence is exclusive to the cell (sub)population. A gene signature as used herein, may thus refer to any set of up- and down-regulated genes that are representative of a cell type or subtype. A gene signature as used herein, may also refer to any set of up- and down-regulated genes between different cells or cell (sub)populations derived from a gene-expression profile. For example, a gene signature may comprise a list of genes differentially expressed in a distinction of interest.

The signature as defined herein (be it a gene signature, protein signature or other genetic or epigenetic signature) can be used to indicate the presence of a cell type, a subtype of the cell type, the state of the microenvironment of a population of cells, a particular cell type population or subpopulation, and/or the overall status of the entire cell (sub)population. Furthermore, the signature may be indicative of cells within a population of cells in vivo. The signature may also be used to suggest for instance particular therapies, or to follow up treatment, or to suggest ways to modulate immune systems. The signatures of the present invention may be discovered by analysis of expression profiles of single cells within a population of cells from isolated samples (e.g. tumor samples), thus allowing the discovery of novel cell subtypes or cell states that were previously invisible or unrecognized. The presence of subtypes or cell states may be determined by subtype specific or cell state specific signatures. The presence of these specific cell (sub)types or cell states may be determined by applying the signature genes to bulk sequencing data in a sample. Not being bound by a theory the signatures of the present invention may be microenvironment specific, such as their expression in a particular spatio-temporal context. Not being bound by a theory, signatures as discussed herein are specific to a particular pathological context. Not being bound by a theory, a combination of cell subtypes having a particular signature may indicate an outcome. Not being bound by a theory, the signatures can be used to deconvolute the network of cells present in a particular pathological condition. Not being bound by a theory the presence of specific cells and cell subtypes are indicative of a particular response to treatment, such as including increased or decreased susceptibility to treatment. The signature may indicate the presence of one particular cell type. In one embodiment, the novel signatures are used to detect multiple cell states or hierarchies that occur in subpopulations of cancer cells that are linked to particular pathological condition (e.g. cancer grade), or linked to a particular outcome or progression of the disease (e.g. metastasis), or linked to a particular response to treatment of the disease.

The signature according to certain embodiments of the present invention may comprise or consist of one or more genes, proteins and/or epigenetic elements, such as for instance 1, 2, 3, 4, 5, 6, 7, 8, 9, 10 or more. In certain embodiments, the signature may comprise or consist of two or more genes, proteins and/or epigenetic elements, such as for instance 2, 3, 4, 5, 6, 7, 8, 9, 10 or more. In certain embodiments, the signature may comprise or consist of three or more genes, proteins and/or epigenetic elements, such as for instance 3, 4, 5, 6, 7, 8, 9, 10 or more. In certain embodiments, the signature may comprise or consist of four or more genes, proteins and/or epigenetic elements, such as for instance 4, 5, 6, 7, 8, 9, 10 or more. In certain embodiments, the signature may comprise or consist of five or more genes, proteins and/or epigenetic elements, such as for instance 5, 6, 7, 8, 9, 10 or more. In certain embodiments, the signature may comprise or consist of six or more genes, proteins and/or epigenetic elements, such as for instance 6, 7, 8, 9, 10 or more. In certain embodiments, the signature may comprise or consist of seven or more genes, proteins and/or epigenetic elements, such as for instance 7, 8, 9, 10 or more. In certain embodiments, the signature may comprise or consist of eight or more genes, proteins and/or epigenetic elements, such as for instance 8, 9, 10 or more. In certain embodiments, the signature may comprise or consist of nine or more genes, proteins and/or epigenetic elements, such as for instance 9, 10 or more. In certain embodiments, the signature may comprise or consist of ten or more genes, proteins and/or epigenetic elements, such as for instance 10, 11, 12, 13, 14, 15, or more. It is to be understood that a signature according to the invention may for instance also include genes or proteins as well as epigenetic elements combined.

In certain embodiments, a signature is characterized as being specific for a particular cell or cell (sub)population if it is upregulated or only present, detected or detectable in that particular cell or cell (sub)population, or alternatively is downregulated or only absent, or undetectable in that particular cell or cell (sub)population. In this context, a signature consists of one or more differentially expressed genes/proteins or differential epigenetic elements when comparing different cells or cell (sub)populations, including comparing different tumor cells or tumor cell (sub)populations, as well as comparing tumor cells or tumor cell (sub)populations with non-tumor cells or non-tumor cell (sub)populations. It is to be understood that “differentially expressed” genes/proteins include genes/proteins which are up- or down-regulated as well as genes/proteins which are turned on or off. When referring to up- or down-regulation, in certain embodiments, such up- or down-regulation is preferably at least two-fold, such as two-fold, three-fold, four-fold, five-fold, or more, such as for instance at least ten-fold, at least 20-fold, at least 30-fold, at least 40-fold, at least 50-fold, or more. Alternatively, or in addition, differential expression may be determined based on common statistical tests, as is known in the art.

As discussed herein, differentially expressed genes/proteins, or differential epigenetic elements may be differentially expressed on a single cell level, or may be differentially expressed on a cell population level. Preferably, the differentially expressed genes/proteins or epigenetic elements as discussed herein, such as constituting the gene signatures as discussed herein, when as to the cell population level, refer to genes that are differentially expressed in all or substantially all cells of the population (such as at least 80%, preferably at least 90%, such as at least 95% of the individual cells). This allows one to define a particular subpopulation of cells. As referred to herein, a “subpopulation” of cells preferably refers to a particular subset of cells of a particular cell type which can be distinguished or are uniquely identifiable and set apart from other cells of this cell type. The cell subpopulation may be phenotypically characterized, and is preferably characterized by the signature as discussed herein. A cell (sub)population as referred to herein may constitute of a (sub)population of cells of a particular cell type characterized by a specific cell state.

When referring to induction, or alternatively suppression of a particular signature, preferable is meant induction or alternatively suppression (or upregulation or downregulation) of at least one gene/protein and/or epigenetic element of the signature, such as for instance at least two, at least three, at least four, at least five, at least six, or all genes/proteins and/or epigenetic elements of the signature.

In further aspects, the invention relates to gene signatures, protein signatures, and/or other genetic or epigenetic signatures of particular tumor cell subpopulations, as defined herein elsewhere. The invention hereto also further relates to particular tumor cell subpopulations, which may be identified based on the methods according to the invention as discussed herein; as well as methods to obtain such cell (sub)populations and screening methods to identify agents capable of inducing or suppressing particular tumor cell (sub)populations.

As will be clear to the skilled person, “modulating” or “modifying” can also involve effecting a change (which can either be an increase or a decrease) in affinity, avidity, specificity and/or selectivity of a target or antigen, for one or more of its targets compared to the same conditions but without the presence of a modulating agent. Again, this can be determined in any suitable manner and/or using any suitable assay known per se, depending on the target. In particular, an action as an inhibitor/antagonist or activator/agonist can be such that an intended biological or physiological activity is increased or decreased, respectively, by at least 5%, at least 10%, at least 25%, at least 50%, at least 60%, at least 70%, at least 80%, or 90% or more, compared to the biological or physiological activity in the same assay under the same conditions but without the presence of the inhibitor/antagonist agent or activator/agonist agent. Modulating can also involve activating the target or antigen or the mechanism or pathway in which it is involved.

The terms “high,” “higher,” “increased,” “elevated,” or “elevation” refer to increases above basal levels, e.g., as compared to a control. The terms “low,” “lower,” “reduced,” “decreased” or “reduction” refer to decreases below basal levels, e.g., as compared to a control.

The term “control” refers to any reference standard suitable to provide a comparison to the expression products in the test sample. In one embodiment, the control comprises obtaining a “control sample” from which expression product levels are detected and compared to the expression product levels from the test sample. Such a control sample may comprise any suitable sample, including but not limited to a sample from a control patient (can be stored sample or previous sample measurement) with a known outcome; normal tissue, fluid, or cells isolated from a subject, such as a normal patient or the patient having a condition of interest.

In certain embodiments, the cell culture medium may be modified to revert to or decrease the expression space between the current phenotype and the in vivo phenotype.

In certain embodiments, the gene expression space comprises 10 or more genes, 20 or more genes, 30 or more genes, 40 or more genes, 50 or more genes, 100 or more genes, 500 or more genes, or 1000 or more genes. In certain embodiments, the expression space defines one or more cell pathways. In certain embodiments, the expression space is a transcriptome of the target in vivo system.

In certain embodiments, the shift in cell type and/or cell states that reduces the distance in gene expression space in the initial cell-based system is a statistically significant shift in the gene expression distribution of the initial cell-based system toward that of the in vivo system. The statistically significant shift may be at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90′%, at least 95%. The statistical shift may include the overall transcriptional identity or the transcriptional identity of one or more genes, gene expression cassettes, or gene expression signatures of the ex vivo system compared to the in vivo system (i.e., at least 10%, at least 15%, at least 20%, at least 25%, at least 30%, at least 35%, at least 40%, at least 45%, at least 50%, at least 55%, at least 60%, at least 65%, at least 70%, at least 75%, at least 80° %, at least 85%, at least 90%, at least 95% of the genes, gene expression cassettes, or gene expression signatures are statistically shifted in a gene expression distribution). A shift of 0% means that there is no difference to the in vivo system. A gene distribution may be the average or range of expression of particular genes, gene expression cassettes, or gene expression signatures in the ex vivo or in vivo system (e.g., a plurality of a cell of interest from an in vivo subject may be sequenced and a distribution is determined for the expression of genes, gene expression cassettes, or gene expression signatures). In certain embodiments, the distribution is a count-based metric for the number of transcripts of each gene present in a cell. A statistical difference between the distributions indicates a shift. The one or more genes, gene expression cassettes, or gene expression signatures may be selected to compare transcriptional identity based on the one or more genes, gene expression cassettes, or gene expression signatures having the most variance as determined by methods of dimension reduction (e.g., tSNE analysis). In certain embodiments, comparing a gene expression distribution comprises comparing the initial cells with the lowest statistically significant shift as compared to the in vivo system (e.g., determining shifts when comparing only the ex vivo cells with a shift of less than 95%, less than 90%, less than 85%, less than 80%, less than 75%, less than 70%, less than 65%, less than 60%, less than 55%, less than 50%, less than 45%, less than 40%, less than 35%, less than 30%, less than 25%, less than 20%, less than 15%, less than 10% to the in vivo system).

In some embodiments, selecting or modifying the medium or conditions comprises the addition of one or more growth factors or cell signaling molecules.

Non-limiting examples of growth factors include those of fibroblast growth factor (FGF) family, bone morphogenic protein (BMP) family, platelet derived growth factor (PDGF) family, transforming growth factor beta (TGFbeta) family, nerve growth factor (NGF) family, epidermal growth factor (EGF) family, insulin related growth factor (IGF) family, hepatocyte growth factor (HGF) family, hematopoietic growth factors (HeGFs), platelet-derived endothelial cell growth factor (PD-ECGF), angiopoietin, vascular endothelial growth factor (VEGF) family, glucocorticoids, or combinations thereof.

In specific embodiments, the method may further comprise culturing the cells in a medium which does not contain TGF beta inhibitor.

In other embodiments, the method may further comprise modulating NFKB signaling. Nuclear factor-κB (NF-κB)/Rel proteins include NF-κB2 p52/p100, NF-κB1 p50/p105, c-Rel, RelA/p65, and RelB. These proteins function as dimeric transcription factors that regulate the expression of genes influencing a broad range of biological processes including innate and adaptive immunity, inflammation, stress responses, B-cell development, and lymphoid organogenesis. In the classical (or canonical) pathway, NF-κB/Rel proteins are bound and inhibited by IκB proteins. Proinflammatory cytokines, LPS, growth factors, and antigen receptors activate an IKK complex (IKKβ, IKKα, and NEMO), which phosphorylates IκB proteins. Phosphorylation of 1 KB leads to its ubiquitination and proteasomal degradation, freeing NF-κB/Rel complexes. Active NF-κB/Rel complexes are further activated by post-translational modifications (phosphorylation, acetylation, glycosylation) and translocate to the nucleus where, either alone or in combination with other transcription factors including AP-1, Ets, and Stat, they induce target gene expression. In the alternative (or noncanonical) NF-κB pathway, NF-κB2 p100/RelB complexes are inactive in the cytoplasm. Signaling through a subset of receptors, including LTβR, CD40, and BR3, activates the kinase NIK, which in turn activates IKKα complexes that phosphorylate C-terminal residues in NF-κB2 p100. Phosphorylation of NF-κB2 p100 leads to its ubiquitination and proteasomal processing to NF-κB2 p52. This creates transcriptionally competent NF-κB p52/RelB complexes that translocate to the nucleus and induce target gene expression.

In other embodiments, the method may further comprise modulating WNT signaling. “Wnt signaling” refers to the series of biochemical events that ensues following binding of a stimulatory ligand (e.g., a Wnt protein) to a receptor for a Wnt family member, ultimately leading to changes in gene transcription and, if in vivo, often leading to a characteristic biological effect in an organism.

In other embodiments, the method may further comprise interfering with pancreatic progenitor phenotypes. In pancreatic development, Pdx1 is expressed by a population of cells in the posterior foregut region of the definitive endoderm, and Pdx1-positive epithelial cells give rise to the developing pancreatic buds, and eventually, the whole of the pancreas-its exocrine, endocrine, and ductal cell populations. Pancreatic Pdx1-positive cells first arise at mouse embryonic day 8.5-9.0, and Pdx1 expression continues until embryonic day 12.0-12.5. Homozygous Pdx1 knockout mice form pancreatic buds but fail to develop a pancreas, and transgenic mice in which tetracycline application results in death of Pdx1-positive cells are almost completely a pancreatic if doxycycline (tetracycline derivative) is administered throughout the pregnancy of these transgenic mice, illustrating the necessity of Pdx1-positive cells in pancreatic development.

Pdx1 is accepted as the earliest marker for pancreatic differentiation, with the fates of pancreatic cells controlled by downstream transcription factors. The initial pancreatic bud is composed of Pdx1-positive pancreatic progenitor cells that co-express Hlxb9, Hnf6, Ptf1a and NKX6-1. These cells further proliferate and branch in response to FGF-10 signaling. Afterwards, fating of the pancreatic cells begins; a population of cells has Notch signaling inhibited, and subsequently, expresses Ngn3. This Ngn3-positive population is a transient population of pancreatic endocrine progenitors that gives rise to the a, p, A, PP, and E cells of the islets of Langerhans. Other cells will give rise to the exocrine and ductal pancreatic cell populations.

In specific embodiments, the method may further comprise modulating Pdx1, Hxb9, Hnf6, Ptf1a, and/or NKX6-1.

In other embodiments, the method may further comprise modulating the extracellular matrix. Cells typically require a surface for attachment to grow and proliferate. Specialized growth matrices along with specific culture media conditions may be needed to maintain certain cells in an undifferentiated state. A gelatinous protein mixture derived from mouse tumor cells and commercialized as Matrigel is commonly used as a basement membrane matrix for stem cells because it retains the stem cells in an undifferentiated state.

In some embodiments, selecting or modifying the medium or conditions comprises inducing changes in intra-cellular signaling between one or more cell types in the ex vivo cell-based model, inducing changes in cell state of one or more cell types, or changing cellular composition of the ex vivo cell-based model.

In some embodiments, the ex vivo cell-based model may be co-cultured with fibroblasts in depleted media, as described in the examples. In specific embodiments, incorporation of additional cell types such as fibroblasts may aid in maintaining basal-like cell phenotypes.

In some embodiments, the ex vivo cell-based model may be co-cultured with T cells. In some embodiments, the ex vivo cell-based model may be co-cultured with macrophages.

In preferred embodiments, the growth factors or cell signaling molecules are added to the medium at the time when the ex vivo cell-based system is established. EX VIVO CELL-BASED SYSTEMS

In some embodiments, the invention provides an ex vivo cell-based system derived by the example methods described herein.

In some embodiments, the ex vivo cell-based system comprises a tumor microenvironment cell. The tumor microenvironment (TME) is the cellular environment in which the tumor exists, including surrounding blood vessels, immune cells, cancer associated fibroblasts (CAFs), bone marrow-derived inflammatory cells, lymphocytes, signaling molecules and the extracellular matrix (ECM).

The tumor microenvironment cell may be a tumor infiltrating lymphocyte (TIL) and/or natural killer (NK) cell. Tumor infiltrating lymphocytes (TILs) are white blood cells that have left the bloodstream and migrated toward a tumor. They include T cells and B cells and are part of the larger category of ‘tumor-infiltrating immune cells’, which consist of both mononuclear and polymorphonuclear immune cells, such as T cells, B cells, natural killer cells, macrophages, neutrophils, dendritic cells, mast cells, eosinophils, basophils, etc., in variable proportions. Their abundance varies in different types of tumors and stages and in some cases relate to disease prognosis.

Immune cells may be obtained using any method known in the art. In one embodiment T cells that have infiltrated a tumor are isolated. T cells may be removed during surgery. T cells may be isolated after removal of tumor tissue by biopsy. T cells may be isolated by any means known in the art. In one embodiment, the method may comprise obtaining a bulk population of T cells from a tumor sample by any suitable method known in the art. For example, a bulk population of T cells can be obtained from a tumor sample by dissociating the tumor sample into a cell suspension from which specific cell populations can be selected. Suitable methods of obtaining a bulk population of T cells may include, but are not limited to, any one or more of mechanically dissociating (e.g., mincing) the tumor, enzymatically dissociating (e.g., digesting) the tumor, and aspiration (e.g., as with a needle).

The bulk population of T cells obtained from a tumor sample may comprise any suitable type of T cell. Preferably, the bulk population of T cells obtained from a tumor sample comprises tumor infiltrating lymphocytes (TILs).

T cells can be obtained from a number of sources, including peripheral blood mononuclear cells, bone marrow, lymph node tissue, spleen tissue, and tumors. In certain embodiments of the present invention, T cells can be obtained from a unit of blood collected from a subject using any number of techniques known to the skilled artisan, such as Ficoll separation. In one preferred embodiment, cells from the circulating blood of an individual are obtained by apheresis or leukapheresis. The apheresis product typically contains lymphocytes, including T cells, monocytes, granulocytes, B cells, other nucleated white blood cells, red blood cells, and platelets. In one embodiment, the cells collected by apheresis may be washed to remove the plasma fraction and to place the cells in an appropriate buffer or media for subsequent processing steps. In one embodiment of the invention, the cells are washed with phosphate buffered saline (PBS). In an alternative embodiment, the wash solution lacks calcium and may lack magnesium or may lack many if not all divalent cations. Initial activation steps in the absence of calcium lead to magnified activation. As those of ordinary skill in the art would readily appreciate a washing step may be accomplished by methods known to those in the art, such as by using a semi-automated “flow-through” centrifuge (for example, the Cobe 2991 cell processor) according to the manufacturer's instructions. After washing, the cells may be resuspended in a variety of biocompatible buffers, such as, for example, Ca-free, Mg-free PBS. Alternatively, the undesirable components of the apheresis sample may be removed and the cells directly resuspended in culture media.

In another embodiment, T cells are isolated from peripheral blood lymphocytes by lysing the red blood cells and depleting the monocytes, for example, by centrifugation through a PERCOLL™ gradient.

A specific subpopulation of T cells can be further isolated by positive or negative selection techniques. For example, in one preferred embodiment, T cells are isolated by incubation with antibody-conjugated beads (e.g., specific for any marker described herein), such as DYNABEADS® for a time period sufficient for positive selection of the desired T cells. In one embodiment, the time period is about 30 minutes. In a further embodiment, the time period ranges from 30 minutes to 36 hours or longer and all integer values there between. In a further embodiment, the time period is at least 1, 2, 3, 4, 5, or 6 hours. In yet another preferred embodiment, the time period is 10 to 24 hours. In one preferred embodiment, the incubation time period is 24 hours. For isolation of T cells from patients with leukemia, use of longer incubation times, such as 24 hours, can increase cell yield. Longer incubation times may be used to isolate T cells in any situation where there are few T cells as compared to other cell types, such in isolating tumor infiltrating lymphocytes (TIL) from tumor tissue or from immunocompromised individuals. Further, use of longer incubation times can increase the efficiency of capture of CD8+ T cells.

Enrichment of a T cell population by negative selection can be accomplished with a combination of antibodies directed to surface markers unique to the negatively selected cells. A preferred method is cell sorting and/or selection via negative magnetic immunoadherence or flow cytometry that uses a cocktail of monoclonal antibodies directed to cell surface markers present on the cells negatively selected.

Further, monocyte populations (i.e., CD14+ cells) may be depleted from blood preparations by a variety of methodologies, including anti-CD14 coated beads or columns, or utilization of the phagocytotic activity of these cells to facilitate removal. Accordingly, in one embodiment, the invention uses paramagnetic particles of a size sufficient to be engulfed by phagocytotic monocytes. In certain embodiments, the paramagnetic particles are commercially available beads, for example, those produced by Life Technologies under the trade name Dynabeads™. In one embodiment, other non-specific cells are removed by coating the paramagnetic particles with “irrelevant” proteins (e.g., serum proteins or antibodies). Irrelevant proteins and antibodies include those proteins and antibodies or fragments thereof that do not specifically target the T cells to be isolated. In certain embodiments the irrelevant beads include beads coated with sheep anti-mouse antibodies, goat anti-mouse antibodies, and human serum albumin.

In brief, such depletion of monocytes is performed by preincubating T cells isolated from whole blood, apheresed peripheral blood, or tumors with one or more varieties of irrelevant or non-antibody coupled paramagnetic particles at any amount that allows for removal of monocytes (approximately a 20:1 bead:cell ratio) for about 30 minutes to 2 hours at 22 to 37 degrees C., followed by magnetic removal of cells which have attached to or engulfed the paramagnetic particles. Such separation can be performed using standard methods available in the art. For example, any magnetic separation methodology may be used including a variety of which are commercially available, (e.g., DYNAL® Magnetic Particle Concentrator (DYNAL MPC®)). Assurance of requisite depletion can be monitored by a variety of methodologies known to those of ordinary skill in the art, including flow cytometric analysis of CD14 positive cells, before and after depletion.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion. Further, use of high cell concentrations allows more efficient capture of cells that may weakly express target antigens of interest or from samples where there are many tumor cells present (i.e., leukemic blood, tumor tissue, etc). Such populations of cells may have therapeutic value and would be desirable to obtain.

In a related embodiment, it may be desirable to use lower concentrations of cells. By significantly diluting the mixture of T cells and surface (e.g., particles such as beads), interactions between the particles and cells is minimized. This selects for cells that express high amounts of desired antigens to be bound to the particles. In one embodiment, the concentration of cells used is 5×106/ml. In other embodiments, the concentration used can be from about 1×105/ml to 1×106/ml, and any integer value in between.

In certain embodiments, T cells can also be frozen. Wishing not to be bound by theory, the freeze and subsequent thaw step provides a more uniform product by removing granulocytes and to some extent monocytes in the cell population. After a washing step to remove plasma and platelets, the cells may be suspended in a freezing solution. While many freezing solutions and parameters are known in the art and will be useful in this context, one method involves using PBS containing 20% DMSO and 8% human serum albumin, or other suitable cell freezing media, the cells then are frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

T cells for use in the present invention may also be antigen-specific T cells. For example, tumor-specific T cells can be used. In certain embodiments, antigen-specific T cells can be isolated from a patient of interest, such as a patient afflicted with a cancer or an infectious disease. In one embodiment neoepitopes are determined for a subject and T cells specific to these antigens are isolated. Antigen-specific cells for use in expansion may also be generated in vitro using any number of methods known in the art, for example, as described in U.S. Patent Publication No. US 20040224402 entitled, Generation and Isolation of Antigen-Specific T Cells, or in U.S. Pat. No. 6,040,177. Antigen-specific cells for use in the present invention may also be generated using any number of methods known in the art, for example, as described in Current Protocols in Immunology, or Current Protocols in Cell Biology, both published by John Wiley & Sons, Inc., Boston, Mass.

In a related embodiment, it may be desirable to sort or otherwise positively select (e.g. via magnetic selection) the antigen specific cells prior to or following one or two rounds of expansion. Sorting or positively selecting antigen-specific cells can be carried out using peptide-MHC tetramers (Altman, et al., Science. 1996 Oct. 4; 274(5284):94-6). In another embodiment the adaptable tetramer technology approach is used (Andersen et al., 2012 Nat Protoc. 7:891-902). Tetramers are limited by the need to utilize predicted binding peptides based on prior hypotheses, and the restriction to specific HLAs. Peptide-MHC tetramers can be generated using techniques known in the art and can be made with any MHC molecule of interest and any antigen of interest as described herein. Specific epitopes to be used in this context can be identified using numerous assays known in the art. For example, the ability of a polypeptide to bind to MHC class I may be evaluated indirectly by monitoring the ability to promote incorporation of 1251 labeled p2-microglobulin (p2m) into MHC class I/p2m/peptide heterotrimeric complexes (see Parker et al., J. Immunol. 152:163, 1994).

In one embodiment cells are directly labeled with an epitope-specific reagent for isolation by flow cytometry followed by characterization of phenotype and TCRs. In one T cells are isolated by contacting the T cell specific antibodies. Sorting of antigen-specific T cells, or generally any cells of the present invention, can be carried out using any of a variety of commercially available cell sorters, including, but not limited to, MoFlo sorter (DakoCytomation, Fort Collins, Colo.), FACSAria™, FACSArray™, FACSVantage6m, BD™ LSR II, and FACSCalibur™ (BD Biosciences, San Jose, Calif.).

In a preferred embodiment, the method comprises selecting cells that also express CD3. The method may comprise specifically selecting the cells in any suitable manner. Preferably, the selecting is carried out using flow cytometry. The flow cytometry may be carried out using any suitable method known in the art. The flow cytometry may employ any suitable antibodies and stains. Preferably, the antibody is chosen such that it specifically recognizes and binds to the particular biomarker being selected. For example, the specific selection of CD3, CD8, TIM-3, LAG-3, 4-1BB, or PD-1 may be carried out using anti-CD3, anti-CD8, anti-TIM-3, anti-LAG-3, anti-4-1BB, or anti-PD-1 antibodies, respectively. The antibody or antibodies may be conjugated to a bead (e.g., a magnetic bead) or to a fluorochrome. Preferably, the flow cytometry is fluorescence-activated cell sorting (FACS). TCRs expressed on T cells can be selected based on reactivity to autologous tumors. Additionally, T cells that are reactive to tumors can be selected for based on markers using the methods described in patent publication Nos. WO2014133567 and WO2014133568, herein incorporated by reference in their entirety. Additionally, activated T cells can be selected for based on surface expression of CD107a.

In certain embodiments, the ex vivo cell-based system simulates a phenotype from a subject who is responsive to cancer treatment. In other embodiments, the ex vivo cell-based system simulates a phenotype from a subject who is non-responsive to cancer treatment.

As used in this context, to “treat” means to cure, ameliorate, stabilize, prevent, or reduce the severity of at least one symptom or a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. It is understood that treatment, while intended to cure, ameliorate, stabilize, or prevent a disease, pathological condition, or disorder, need not actually result in the cure, amelioration, stabilization or prevention. The effects of treatment can be measured or assessed as described herein and as known in the art as is suitable for the disease, pathological condition, or disorder involved. Such measurements and assessments can be made in qualitative and/or quantitative terms. Thus, for example, characteristics or features of a disease, pathological condition, or disorder and/or symptoms of a disease, pathological condition, or disorder can be reduced to any effect or to any amount.

The term “in need of treatment” as used herein refers to a judgment made by a caregiver (e.g. physician, nurse, nurse practitioner, or individual in the case of humans; veterinarian in the case of animals, including non-human animals) that a subject requires or will benefit from treatment. This judgment is made based on a variety of factors that are in the realm of a caregiver's experience, but that include the knowledge that the subject is ill, or will be ill, as the result of a condition that is treatable by the compositions and therapeutic agents described herein.

In some embodiments, the cancer treatment may comprise chemotherapy. In some embodiments the cancer treatment may comprise immunotherapy.

### Immunotherapy

Immunotherapy is the treatment of disease by activating (activation immunotherapies) or suppressing the immune system (suppression immunotherapies). Immunotherapy is particularly suitable for treating various forms of cancer. Immunomodulatory regimens often have fewer side effects than existing drugs, including a decreased potential for creating resistance when treating microbial disease. Cell-based immunotherapies are effective for some cancers. Immune effector cells such as lymphocytes, macrophages, dendritic cells, natural killer (NK) cells, cytotoxic T lymphocytes (CTL), etc., work together to defend the body against cancer by targeting abnormal antigens expressed on the surface of tumor cells. Therapies such as granulocyte colony-stimulating factor (G-CSF), interferons, imiquimod and cellular membrane fractions from bacteria are licensed for medical use. Others including IL-2, IL-7, IL-12, various chemokines, synthetic cytosine phosphate-guanosine (CpG) oligodeoxynucleotides and glucans are involved in clinical and preclinical studies.

“Anti-immune checkpoint” or “immune checkpoint inhibitor or “immune checkpoint blockade” therapy refers to the use of agents that inhibit immune checkpoint nucleic acids and/or proteins. Immune checkpoints share the common function of providing inhibitory signals that suppress immune response and inhibition of one or more immune checkpoints can block or otherwise neutralize inhibitory signaling to thereby upregulate an immune response in order to more efficaciously treat cancer. Exemplary agents useful for inhibiting immune checkpoints include antibodies, small molecules, peptides, peptidomimetics, natural ligands, and derivatives of natural ligands, that can either bind and/or inactivate or inhibit immune checkpoint proteins, or fragments thereof; as well as RNA interference, antisense, nucleic acid aptamers, etc. that can downregulate the expression and/or activity of immune checkpoint nucleic acids, or fragments thereof. Exemplary agents for upregulating an immune response include antibodies against one or more immune checkpoint proteins block the interaction between the proteins and its natural receptor(s); a non-activating form of one or more immune checkpoint proteins (e.g., a dominant negative polypeptide); small molecules or peptides that block the interaction between one or more immune checkpoint proteins and its natural receptor(s); fusion proteins (e.g. the extracellular portion of an immune checkpoint inhibition protein fused to the Fc portion of an antibody or immunoglobulin) that bind to its natural receptor(s); nucleic acid molecules that block immune checkpoint nucleic acid transcription or translation; and the like. Such agents can directly block the interaction between the one or more immune checkpoints and its natural receptor(s) (e.g., antibodies) to prevent inhibitory signaling and upregulate an immune response. Alternatively, agents can indirectly block the interaction between one or more immune checkpoint proteins and its natural receptor(s) to prevent inhibitory signaling and upregulate an immune response. For example, a soluble version of an immune checkpoint protein ligand such as a stabilized extracellular domain can bind to its receptor to indirectly reduce the effective concentration of the receptor to bind to an appropriate ligand. In one embodiment, anti-PD-1 antibodies, anti-PD-L1 antibodies, and/or anti-PD-L2 antibodies, either alone or in combination, are used to inhibit immune checkpoints. These embodiments are also applicable to specific therapy against particular immune checkpoints, such as the PD-1 pathway (e.g., anti-PD-1 pathway therapy, otherwise known as PD-1 pathway inhibitor therapy). Numerous immune checkpoint inhibitors are known and publicly available including, for example, Keytruda® (pembrolizumab; anti-PD-1 antibody), Opdivo® (nivolumab; anti-PD-1 antibody), Tecentriq® (atezolizumab; anti-PD-L1 antibody), durvalumab (anti-PD-L1 antibody), and the like.

The present invention also contemplates use of the CRISPR-Cas system described herein, e.g. C2c1 effector protein systems, to modify cells for adoptive therapies.

As used herein, “ACT”, “adoptive cell therapy” and “adoptive cell transfer” may be used interchangeably. In certain embodiments, Adoptive cell therapy (ACT) can refer to the transfer of cells to a patient with the goal of transferring the functionality and characteristics into the new host by engraftment of the cells (see, e.g., Mettananda et al., Editing an α-globin enhancer in primary human hematopoietic stem cells as a treatment for β-thalassemia, Nat Commun. 2017 Sep. 4; 8(1):424). As used herein, the term “engraft” or “engrafiment” refers to the process of cell incorporation into a tissue of interest in vivo through contact with existing cells of the tissue. Adoptive cell therapy (ACT) can refer to the transfer of cells, most commonly immune-derived cells, back into the same patient or into a new recipient host with the goal of transferring the immunologic functionality and characteristics into the new host. If possible, use of autologous cells helps the recipient by minimizing graft-versus-host disease (GVHD) issues. The adoptive transfer of autologous tumor infiltrating lymphocytes (TIL) (Besser et al., (2010) Clin. Cancer Res 16 (9) 2646-55, Dudley et al., (2002) Science 298 (5594): 850-4; and Dudley et al., (2005) Journal of Clinical Oncology 23 (10): 2346-57.) or genetically re-directed peripheral blood mononuclear cells (Johnson et al., (2009) Blood 114 (3): 535-46; and Morgan et al., (2006) Science 314(5796) 126-9) has been used to successfully treat patients with advanced solid tumors, including melanoma and colorectal carcinoma, as well as patients with CD19-expressing hematologic malignancies (Kalos et al., (2011) Science Translational Medicine 3 (95): 95ra73). In certain embodiments, allogenic cells immune cells are transferred (see, e.g., Ren et al., (2017) Clin Cancer Res 23 (9) 2255-2266). As described further herein, allogenic cells can be edited to reduce alloreactivity and prevent graft-versus-host disease. Thus, use of allogenic cells allows for cells to be obtained from healthy donors and prepared for use in patients as opposed to preparing autologous cells from a patient after diagnosis.

In some embodiments, the invention described herein relates to a method for adoptive immunotherapy, in which T cells are edited ex vivo by CRISPR to modulate at least one gene and subsequently administered to a patient in need thereof. In some embodiments, the CRISPR editing comprising knocking-out or knocking-down the expression of at least one target gene in the edited T cells. In some embodiments, in addition to modulating the target gene, the T cells are also edited ex vivo by CRISPR to (1) knock-in an exogenous gene encoding a chimeric antigen receptor (CAR) or a T-cell receptor (TCR), (2) knock-out or knock-down expression of an immune checkpoint receptor, (3) knock-out or knock-down expression of an endogenous TCR, (4) knock-out or knock-down expression of a human leukocyte antigen class I (HLA-I) proteins, and/or (5) knock-out or knock-down expression of an endogenous gene encoding an antigen targeted by an exogenous CAR or TCR.

In some embodiments, the T cells are contacted ex vivo with an adeno-associated virus (AAV) vector encoding a CRISPR effector protein, and a guide molecule comprising a guide sequence hybridizable to a target sequence, a tracr mate sequence, and a tracr sequence hybridizable to the tracr mate sequence. In some embodiments, the T cells are contacted ex vivo (e.g., by electroporation) with a ribonucleoprotein (RNP) comprising a CRISPR effector protein complexed with a guide molecule, wherein the guide molecule comprising a guide sequence hybridizable to a target sequence, a tracr mate sequence, and a tracr sequence hybridizable to the tracr mate sequence. See Rupp et al., Scientific Reports 7:737 (2017); Liu et al., Cell Research 27:154-157 (2017). In some embodiments, the T cells are contacted ex vivo (e.g., by electroporation) with an mRNA encoding a CRISPR effector protein, and a guide molecule comprising a guide sequence hybridizable to a target sequence, a tracr mate sequence, and a tracr sequence hybridizable to the tracr mate sequence. See Eyquem et al., Nature 543:113-117 (2017). In some embodiments, the T cells are not contacted ex vivo with a lentivirus or retrovirus vector.

In some embodiments, the method comprises editing T cells ex vivo by CRISPR to knock-in an exogenous gene encoding a CAR, thereby allowing the edited T cells to recognize cancer cells based on the expression of specific proteins located on the cell surface. In some embodiments, T cells are edited ex vivo by CRISPR to knock-in an exogenous gene encoding a TCR, thereby allowing the edited T cells to recognize proteins derived from either the surface or inside of the cancer cells. In some embodiments, the method comprising providing an exogenous CAR-encoding or TCR-encoding sequence as a donor sequence, which can be integrated by homology-directed repair (HDR) into a genomic locus targeted by a CRISPR guide sequence. In some embodiments, targeting the exogenous CAR or TCR to an endogenous TCR a constant (TRAC) locus can reduce tonic CAR signaling and facilitate effective internalization and re-expression of the CAR following single or repeated exposure to antigen, thereby delaying effector T-cell differentiation and exhaustion. See Eyquem et al., Nature 543:113-117 (2017).

In some embodiments, the method comprises editing T cells ex vivo by CRISPR to block one or more immune checkpoint receptors to reduce immunosuppression by cancer cells. In some embodiments, T cells are edited ex vivo by CRISPR to knock-out or knock-down an endogenous gene involved in the programmed death-1 (PD-1) signaling pathway, such as PD-1 and PD-L1. In some embodiments, T cells are edited ex vivo by CRISPR to mutate the Pdcd1 locus or the CD274 locus. In some embodiments, T cells are edited ex vivo by CRISPR using one or more guide sequences targeting the first exon of PD-1. See Rupp et al., Scientific Reports 7:737 (2017); Liu et al., Cell Research 27:154-157 (2017).

In some embodiments, the method comprises editing T cells ex vivo by CRISPR to eliminate potential alloreactive TCRs to allow allogeneic adoptive transfer. In some embodiments, T cells are edited ex vivo by CRISPR to knock-out or knock-down an endogenous gene encoding a TCR (e.g., an αβ TCR) to avoid graft-versus-host-disease (GVHD). In some embodiments, T cells are edited ex vivo by CRISPR to mutate the TRAC locus. In some embodiments, T cells are edited ex vivo by CRISPR using one or more guide sequences targeting the first exon of TRAC. See Liu et al., Cell Research 27:154-157 (2017). In some embodiments, the method comprises use of CRISPR to knock-in an exogenous gene encoding a CAR or a TCR into the TRAC locus, while simultaneously knocking-out the endogenous TCR (e.g., with a donor sequence encoding a self-cleaving P2A peptide following the CAR cDNA). See Eyquem et al., Nature 543:113-117 (2017). In some embodiments, the exogenous gene comprises a promoter-less CAR-encoding or TCR-encoding sequence which is inserted operably downstream of an endogenous TCR promoter.

In some embodiments, the method comprises editing T cells ex vivo by CRISPR to knock-out or knock-down an endogenous gene encoding an HLA-I protein to minimize immunogenicity of the edited T cells. In some embodiments, T cells are edited ex vivo by CRISPR to mutate the beta-2 microglobulin (B2M) locus. In some embodiments, T cells are edited ex vivo by CRISPR using one or more guide sequences targeting the first exon of B2M. See Liu et al., Cell Research 27:154-157 (2017). In some embodiments, the method comprises use of CRISPR to knock-in an exogenous gene encoding a CAR or a TCR into the B2M locus, while simultaneously knocking-out the endogenous B2M (e.g., with a donor sequence encoding a self-cleaving P2A peptide following the CAR cDNA). See Eyquem et al., Nature 543:113-117 (2017). In some embodiments, the exogenous gene comprises a promoter-less CAR-encoding or TCR-encoding sequence which is inserted operably downstream of an endogenous B2M promoter.

In some embodiments, the method comprises editing T cells ex vivo by CRISPR to knock-out or knock-down an endogenous gene encoding an antigen targeted by an exogenous CAR or TCR. In some embodiments, the T cells are edited ex vivo by CRISPR to knock-out or knock-down the expression of a tumor antigen selected from human telomerase reverse transcriptase (hTERT), survivin, mouse double minute 2 homolog (MDM2), cytochrome P450 1B 1 (CYP1B), HER2/neu, Wilms' tumor gene 1 (WT1), livin, alphafetoprotein (AFP), carcinoembryonic antigen (CEA), mucin 16 (MUC16), MUC1, prostate-specific membrane antigen (PSMA), p53 or cyclin (DI) (see WO2016/011210). In some embodiments, the T cells are edited ex vivo by CRISPR to knock-out or knock-down the expression of an antigen selected from B cell maturation antigen (BCMA), transmembrane activator and CAML Interactor (TACI), or B-cell activating factor receptor (BAFF-R), CD38, CD138, CS-1, CD33, CD26, CD30, CD53, CD92, CD100, CD148, CD150, CD200, CD261, CD262, or CD362 (see WO2017/011804).

Aspects of the invention accordingly involve the adoptive transfer of immune system cells, such as T cells, specific for selected antigens, such as tumor associated antigens (see Maus et al., 2014, Adoptive Immunotherapy for Cancer or Viruses, Annual Review of Immunology, Vol. 32: 189-225; Rosenberg and Restifo, 2015, Adoptive cell transfer as personalized immunotherapy for human cancer, Science Vol. 348 no. 6230 pp. 62-68; and, Restifo et al., 2015, Adoptive immunotherapy for cancer: harnessing the T cell response. Nat. Rev. Immunol. 12(4): 269-281; and Jenson and Riddell, 2014, Design and implementation of adoptive therapy with chimeric antigen receptor-modified T cells. Immunol Rev. 257(1): 127-144). Various strategies may for example be employed to genetically modify T cells by altering the specificity of the T cell receptor (TCR) for example by introducing new TCR α and β chains with selected peptide specificity (see U.S. Pat. No. 8,697,854; PCT Patent Publications: WO2003020763, WO2004033685, WO2004044004, WO2005114215, WO2006000830, WO2008038002, WO2008039818, WO2004074322, WO2005113595, WO2006125962, WO2013166321, WO2013039889, WO2014018863, WO2014083173; U.S. Pat. No. 8,088,379).

As an alternative to, or addition to, TCR modifications, chimeric antigen receptors (CARs) may be used in order to generate immunoresponsive cells, such as T cells, specific for selected targets, such as malignant cells, with a wide variety of receptor chimera constructs having been described (see U.S. Pat. Nos. 5,843,728; 5,851,828; 5,912,170; 6,004,811; 6,284,240; 6,392,013; 6,410,014; 6,753,162; 8,211,422; and, PCT Publication WO9215322). Autologous T cells engineered to express chimeric antigen receptors (CARs) against leukemia antigens such as CD19 on B cells have shown promising results for the treatment of relapsed or refractory B-cell malignancies. However, a subset of cancer patients especially heavily pretreated cancer patients could be unable to receive this highly active therapy because of failed expansion. Moreover, it is still a challenge to manufacture an effective therapeutic product for infant cancer patients due to their small blood volume. On the other hand, the inherent characters of autologous CAR-T cell therapy including personalized autologous T cell manufacturing and widely “distributed” approach result in the difficulty of industrialization of autologous CAR-T cell therapy. Universal CD19-specific CAR-T cell (UCART019), derived from one or more healthy unrelated donors but could avoid graft-versus-host-disease (GVHD) and minimize their immunogenicity, is undoubtedly an alternative option to address above-mentioned issues. Alternative CAR constructs may be characterized as belonging to successive generations. First-generation CARs typically consist of a single-chain variable fragment of an antibody specific for an antigen, for example comprising a VL linked to a VH of a specific antibody, linked by a flexible linker, for example by a CD8a hinge domain and a CD8a transmembrane domain, to the transmembrane and intracellular signaling domains of either CD3ζ or FcRγ (scFv-CD3ζ or scFv-FcRγ; see U.S. Pat. Nos. 7,741,465; 5,912,172; 5,906,936). Second-generation CARs incorporate the intracellular domains of one or more costimulatory molecules, such as CD28, OX40 (CD134), or 4-1BB (CD137) within the endodomain (for example scFv-CD28/OX40/4-1BB-CD3ζ; see U.S. Pat. Nos. 8,911,993; 8,916,381; 8,975,071; 9,101,584; 9,102,760; 9,102,761). Third-generation CARs include a combination of costimulatory endodomains, such a CD3ζ-chain, CD97, GD11a-CD18, CD2, ICOS, CD27, CD154, CDS, OX40, 4-1BB, or CD28 signaling domains (for example scFv-CD28-4-1BB-CD3ζ or scFv-CD28-OX40-CD3ζ; see U.S. Pat. Nos. 8,906,682; 8,399,645; 5,686,281; PCT Publication No. WO2014134165; PCT Publication No. WO2012079000). Alternatively, costimulation may be orchestrated by expressing CARs in antigen-specific T cells, chosen so as to be activated and expanded following engagement of their native αβTCR, for example by antigen on professional antigen-presenting cells, with attendant costimulation. In addition, additional engineered receptors may be provided on the immunoresponsive cells, for example to improve targeting of a T-cell attack and/or minimize side effects. Han et. al (clinicaltrials, A Study Evaluating UCART019 in Patients with Relapsed or Refractory CD19+ Leukemia and Lymphoma) have generated gene-disrupted allogeneic CD19-directed BBζ CAR-T cells (termed UCART019) by combining the lentiviral delivery of CAR and CRISPR RNA electroporation to disrupt endogenous TCR and B2M genes simultaneously and will test whether it can evade host-mediated immunity and deliver antileukemic effects without GVHD.

Alternative techniques may be used to transform target immunoresponsive cells, such as protoplast fusion, lipofection, transfection or electroporation. A wide variety of vectors may be used, such as retroviral vectors, lentiviral vectors, adenoviral vectors, adeno-associated viral vectors, plasmids or transposons, such as a Sleeping Beauty transposon (see U.S. Pat. Nos. 6,489,458; 7,148,203; 7,160,682; 7,985,739; 8,227,432), may be used to introduce CARs, for example using 2nd generation antigen-specific CARs signaling through CD3ζ and either CD28 or CD137. Viral vectors may for example include vectors based on HIV, SV40, EBV, HSV or BPV.

Cells that are targeted for transformation may for example include T cells, Natural Killer (NK) cells, cytotoxic T lymphocytes (CTL), regulatory T cells, human embryonic stem cells, tumor-infiltrating lymphocytes (TIL) or a pluripotent stem cell from which lymphoid cells may be differentiated. T cells expressing a desired CAR may for example be selected through co-culture with γ-irradiated activating and propagating cells (AaPC), which co-express the cancer antigen and co-stimulatory molecules. The engineered CAR T-cells may be expanded, for example by co-culture on AaPC in presence of soluble factors, such as IL-2 and IL-21. This expansion may for example be carried out so as to provide memory CAR+ T cells (which may for example be assayed by non-enzymatic digital array and/or multi-panel flow cytometry). In this way, CAR T cells may be provided that have specific cytotoxic activity against antigen-bearing tumors (optionally in conjunction with production of desired chemokines such as interferon-γ). CAR T cells of this kind may for example be used in animal models, for example to threat tumor xenografts.

In general, CARs are comprised of an extracellular domain, a transmembrane domain, and an intracellular domain, wherein the extracellular domain comprises an antigen-binding domain that is specific for a predetermined target. While the antigen-binding domain of a CAR is often an antibody or antibody fragment (e.g., a single chain variable fragment, scFv), the binding domain is not particularly limited so long as it results in specific recognition of a target. For example, in some embodiments, the antigen-binding domain may comprise a receptor, such that the CAR is capable of binding to the ligand of the receptor. Alternatively, the antigen-binding domain may comprise a ligand, such that the CAR is capable of binding the endogenous receptor of that ligand.

The antigen-binding domain of a CAR is generally separated from the transmembrane domain by a hinge or spacer. The spacer is also not particularly limited, and it is designed to provide the CAR with flexibility. For example, a spacer domain may comprise a portion of a human Fc domain, including a portion of the CH3 domain, or the hinge region of any immunoglobulin, such as IgA, IgD, IgE, IgG, or IgM, or variants thereof. Furthermore, the hinge region may be modified so as to prevent off-target binding by FcRs or other potential interfering objects. For example, the hinge may comprise an IgG4 Fc domain with or without a S228P, L235E, and/or N297Q mutation (according to Kabat numbering) in order to decrease binding to FcRs. Additional spacers/hinges include, but are not limited to, CD4, CD8, and CD28 hinge regions.

The transmembrane domain of a CAR may be derived either from a natural or from a synthetic source. Where the source is natural, the domain may be derived from any membrane bound or transmembrane protein. Transmembrane regions of particular use in this disclosure may be derived from CD8, CD28, CD3, CD45, CD4, CD5, CDS, CD9, CD 16, CD22, CD33, CD37, CD64, CD80, CD86, CD 134, CD137, CD 154, TCR. Alternatively, the transmembrane domain may be synthetic, in which case it will comprise predominantly hydrophobic residues such as leucine and valine. Preferably a triplet of phenylalanine, tryptophan and valine will be found at each end of a synthetic transmembrane domain. Optionally, a short oligo- or polypeptide linker, preferably between 2 and 10 amino acids in length may form the linkage between the transmembrane domain and the cytoplasmic signaling domain of the CAR. A glycine-serine doublet provides a particularly suitable linker.

Alternative CAR constructs may be characterized as belonging to successive generations. First-generation CARs typically consist of a single-chain variable fragment of an antibody specific for an antigen, for example comprising a VL linked to a VH of a specific antibody, linked by a flexible linker, for example by a CD8a hinge domain and a CD8a transmembrane domain, to the transmembrane and intracellular signaling domains of either CD3ζ or FcRγ (scFv-CD3ζ or scFv-FcRγ; see U.S. Pat. Nos. 7,741,465; 5,912,172; 5,906,936). Second-generation CARs incorporate the intracellular domains of one or more costimulatory molecules, such as CD28, OX40 (CD134), or 4-1BB (CD137) within the endodomain (for example scFv-CD28/OX40/4-1BB-CD3ζ; see U.S. Pat. Nos. 8,911,993; 8,916,381; 8,975,071; 9,101,584; 9,102,760; 9,102,761). Third-generation CARs include a combination of costimulatory endodomains, such a CD3ζ-chain, CD97, GD11a-CD18, CD2, ICOS, CD27, CD154, CDS, OX40, 4-1BB, CD2, CD7, LIGHT, LFA-1, NKG2C, B7-H3, CD30, CD40, PD-1, or CD28 signaling domains (for example scFv-CD28-4-1BB-CD3 or scFv-CD28-OX40-CD3ζ; see U.S. Pat. Nos. 8,906,682; 8,399,645; 5,686,281; PCT Publication No. WO2014134165; PCT Publication No. WO2012079000). In certain embodiments, the primary signaling domain comprises a functional signaling domain of a protein selected from the group consisting of CD3 zeta, CD3 gamma, CD3 delta, CD3 epsilon, common FcR gamma (FCERIG), FcR beta (Fc Epsilon R1b), CD79a, CD79b, Fc gamma RIIa, DAP10, and DAP12. In certain preferred embodiments, the primary signaling domain comprises a functional signaling domain of CD3ζ or FcR7. In certain embodiments, the one or more costimulatory signaling domains comprise a functional signaling domain of a protein selected, each independently, from the group consisting of: CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD160, CD19, CD4, CD8 alpha, CD8 beta, IL2R beta, IL2R gamma, IL7R alpha, ITGA4, VLA 1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Lyl08), SLAM (SLAMFI, CD150, IPO-3), BLAME (SLAMF8), SELPLG (CD162), LTBR, LAT, GADS, SLP-76, PAG/Cbp, NKp44, NKp30, NKp46, and NKG2D. In certain embodiments, the one or more costimulatory signaling domains comprise a functional signaling domain of a protein selected, each independently, from the group consisting of: 4-1BB, CD27, and CD28. In certain embodiments, a chimeric antigen receptor may have the design as described in U.S. Pat. No. 7,446,190, comprising an intracellular domain of CD3ζ chain (such as amino acid residues 52-163 of the human CD3 zeta chain, as shown in SEQ ID NO: 14 of U.S. Pat. No. 7,446,190), a signaling region from CD28 and an antigen-binding element (or portion or domain; such as scFv). The CD28 portion, when between the zeta chain portion and the antigen-binding element, may suitably include the transmembrane and signaling domains of CD28 (such as amino acid residues 114-220 of SEQ ID NO: 10, full sequence shown in SEQ ID NO: 6 of U.S. Pat. No. 7,446,190; these can include the following portion of CD28 as set forth in Genbank identifier NM_006139 (sequence version 1, 2 or 3): IEVMYPPPYLDNEKSNGTIIHVKGKHLCPSPLFPGPSKPFWVLVVVGGVLACYSLLV TVAFIIFWVRSKRSRLLHSDYMNMTPRRPGPTRKHYQPYAPPRDFAAYRS)).

Alternatively, when the zeta sequence lies between the CD28 sequence and the antigen-binding element, intracellular domain of CD28 can be used alone (such as amino sequence set forth in SEQ ID NO: 9 of U.S. Pat. No. 7,446,190). Hence, certain embodiments employ a CAR comprising (a) a zeta chain portion comprising the intracellular domain of human CD3ζ chain, (b) a costimulatory signaling region, and (c) an antigen-binding element (or portion or domain), wherein the costimulatory signaling region comprises the amino acid sequence encoded by SEQ ID NO: 6 of U.S. Pat. No. 7,446,190.

Alternatively, costimulation may be orchestrated by expressing CARs in antigen-specific T cells, chosen so as to be activated and expanded following engagement of their native αβTCR, for example by antigen on professional antigen-presenting cells, with attendant costimulation. In addition, additional engineered receptors may be provided on the immunoresponsive cells, for example to improve targeting of a T-cell attack and/or minimize side effects

By means of an example and without limitation, Kochenderfer et al., (2009) J Immunother. 32 (7): 689-702 described anti-CD19 chimeric antigen receptors (CAR). FMC63-28Z CAR contained a single chain variable region moiety (scFv) recognizing CD19 derived from the FMC63 mouse hybridoma (described in Nicholson et al., (1997) Molecular Immunology 34: 1157-1165), a portion of the human CD28 molecule, and the intracellular component of the human TCR-ζ molecule. FMC63-CD828BBZ CAR contained the FMC63 scFv, the hinge and transmembrane regions of the CD8 molecule, the cytoplasmic portions of CD28 and 4-1BB, and the cytoplasmic component of the TCR-ζ molecule. The exact sequence of the CD28 molecule included in the FMC63-28Z CAR corresponded to Genbank identifier NM_006139; the sequence included all amino acids starting with the amino acid sequence IEVMYPPPY and continuing all the way to the carboxy-terminus of the protein. To encode the anti-CD19 scFv component of the vector, the authors designed a DNA sequence which was based on a portion of a previously published CAR (Cooper et al., (2003) Blood 101: 1637-1644). This sequence encoded the following components in frame from the 5′ end to the 3′ end: an XhoI site, the human granulocyte-macrophage colony-stimulating factor (GM-CSF) receptor α-chain signal sequence, the FMC63 light chain variable region (as in Nicholson et al., supra), a linker peptide (as in Cooper et al., supra), the FMC63 heavy chain variable region (as in Nicholson et al., supra), and a NotI site. A plasmid encoding this sequence was digested with XhoI and NotI. To form the MSGV-FMC63-28Z retroviral vector, the XhoI and NotI-digested fragment encoding the FMC63 scFv was ligated into a second XhoI and NotI-digested fragment that encoded the MSGV retroviral backbone (as in Hughes et al., (2005) Human Gene Therapy 16: 457-472) as well as part of the extracellular portion of human CD28, the entire transmembrane and cytoplasmic portion of human CD28, and the cytoplasmic portion of the human TCR-ζ molecule (as in Maher et al., 2002) Nature Biotechnology 20: 70-75). The FMC63-28Z CAR is included in the KTE-C19 (axicabtagene ciloleucel) anti-CD19 CAR-T therapy product in development by Kite Pharma, Inc. for the treatment of inter alia patients with relapsed/refractory aggressive B-cell non-Hodgkin lymphoma (NHL). Accordingly, in certain embodiments, cells intended for adoptive cell therapies, more particularly immunoresponsive cells such as T cells, may express the FMC63-28Z CAR as described by Kochenderfer et al. (supra). Hence, in certain embodiments, cells intended for adoptive cell therapies, more particularly immunoresponsive cells such as T cells, may comprise a CAR comprising an extracellular antigen-binding element (or portion or domain; such as scFv) that specifically binds to an antigen, an intracellular signaling domain comprising an intracellular domain of a CD3ζ chain, and a costimulatory signaling region comprising a signaling domain of CD28. Preferably, the CD28 amino acid sequence is as set forth in Genbank identifier NM_006139 (sequence version 1, 2 or 3) starting with the amino acid sequence IEVMYPPPY and continuing all the way to the carboxy-terminus of the protein. The sequence is reproduced herein: IEVMYPPPYLDNEKSNGTIIHVKGKHLCPSPLFPGPSKPFWVLVVVGGVLACYSLLV TVAFIIFWVRSKRSRLLHSDYMNMTPRRPGPTRKHYQPYAPPRDFAAYRS. Preferably, the antigen is CD19, more preferably the antigen-binding element is an anti-CD19 scFv, even more preferably the anti-CD19 scFv as described by Kochenderfer et al. (supra).

Additional anti-CD19 CARs are further described in WO2015187528. More particularly Example 1 and Table 1 of WO2015187528, incorporated by reference herein, demonstrate the generation of anti-CD19 CARs based on a fully human anti-CD19 monoclonal antibody (47G4, as described in US20100104509) and murine anti-CD19 monoclonal antibody (as described in Nicholson et al. and explained above). Various combinations of a signal sequence (human CD8-alpha or GM-CSF receptor), extracellular and transmembrane regions (human CD8-alpha) and intracellular T-cell signaling domains (CD28-CD3ζ; 4-1BB-CD3ζ; CD27-CD3ζ; CD28-CD27-CD3ζ, 4-1BB-CD27-CD3ζ; CD27-4-1BB-CD3ζ; CD28-CD27-FcεRI gamma chain; or CD28-FcεRI gamma chain) were disclosed. Hence, in certain embodiments, cells intended for adoptive cell therapies, more particularly immunoresponsive cells such as T cells, may comprise a CAR comprising an extracellular antigen-binding element that specifically binds to an antigen, an extracellular and transmembrane region as set forth in Table 1 of WO2015187528 and an intracellular T-cell signaling domain as set forth in Table 1 of WO2015187528. Preferably, the antigen is CD19, more preferably the antigen-binding element is an anti-CD19 scFv, even more preferably the mouse or human anti-CD19 scFv as described in Example 1 of WO2015187528. In certain embodiments, the CAR comprises, consists essentially of or consists of an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, or SEQ ID NO: 13 as set forth in Table 1 of WO2015187528.

By means of an example and without limitation, chimeric antigen receptor that recognizes the CD70 antigen is described in WO2012058460A2 (see also, Park et al., CD70 as a target for chimeric antigen receptor T cells in head and neck squamous cell carcinoma, Oral Oncol. 2018 March; 78:145-150; and Jin et al., CD70, a novel target of CAR T-cell therapy for gliomas, Neuro Oncol. 2018 Jan. 10; 20(1):55-65). CD70 is expressed by diffuse large B-cell and follicular lymphoma and also by the malignant cells of Hodgkins lymphoma, Waldenstrom's macroglobulinemia and multiple myeloma, and by HTLV-1- and EBV-associated malignancies. (Agathanggelou et al. Am. J. Pathol. 1995; 147: 1152-1160; Hunter et al., Blood 2004, 104:4881. 26; Lens et al., J Immunol. 2005; 174:6212-6219; Baba et al., J Virol. 2008; 82:3843-3852.) In addition, CD70 is expressed by non-hematological malignancies such as renal cell carcinoma and glioblastoma. (Junker et al., J Urol. 2005; 173:2150-2153; Chahlavi et al., Cancer Res 2005; 65:5428-5438) Physiologically, CD70 expression is transient and restricted to a subset of highly activated T, B, and dendritic cells.

By means of an example and without limitation, chimeric antigen receptor that recognizes BCMA has been described (see, e.g., US20160046724A1; WO2016014789A2; WO2017211900A1; WO2015158671A1; US20180085444A1; WO2018028647A1; US20170283504A1; and WO2013154760A1).

The CRISPR systems disclosed herein may be used for targeting an antigen to be targeted in adoptive cell therapy. In certain embodiments, an antigen (such as a tumor antigen) to be targeted in adoptive cell therapy (such as TIL, CAR, or TCR T-cell therapy) of a disease (such as particularly of tumor or cancer) may be selected from a group consisting of: B cell maturation antigen (BCMA) (see, e.g., Friedman et al., Effective Targeting of Multiple BCMA-Expressing Hematological Malignancies by Anti-BCMA CAR T Cells, Hum Gene Ther. 2018 Mar. 8; Berdeja J G, et al. Durable clinical responses in heavily pretreated patients with relapsed/refractory multiple myeloma: updated results from a multicenter study of bb2121 anti-Bcma CAR T cell therapy. Blood. 2017; 130:740; and Mouhieddine and Ghobrial, Immunotherapy in Multiple Myeloma: The Era of CAR T Cell Therapy, Hematologist, May-June 2018, Volume 15, issue 3); PSA (prostate-specific antigen); prostate-specific membrane antigen (PSMA); PSCA (Prostate stem cell antigen); Tyrosine-protein kinase transmembrane receptor ROR1; fibroblast activation protein (FAP); Tumor-associated glycoprotein 72 (TAG72); Carcinoembryonic antigen (CEA); Epithelial cell adhesion molecule (EPCAM); Mesothelin; Human Epidermal growth factor Receptor 2 (ERBB2 (Her2/neu)); Prostase; Prostatic acid phosphatase (PAP); elongation factor 2 mutant (ELF2M); Insulin-like growth factor 1 receptor (IGF-1R); gplOO, BCR-ABL (breakpoint cluster region-Abelson); tyrosinase; New York esophageal squamous cell carcinoma 1 (NY-ESO-1); κ-light chain, LAGE (L antigen); MAGE (melanoma antigen); Melanoma-associated antigen 1 (MAGE-A1); MAGE A3; MAGE A6; legumain; Human papillomavirus (HPV) E6; HPV E7; prostein; survivin; PCTA1 (Galectin 8); Melan-A/MART-1; Ras mutant; TRP-1 (tyrosinase related protein 1, or gp75); Tyrosinase-related Protein 2 (TRP2); TRP-2/INT2 (TRP-2/intron 2); RAGE (renal antigen); receptor for advanced glycation end products 1 (RAGE1); Renal ubiquitous 1, 2 (RU1, RU2); intestinal carboxyl esterase (iCE); Heat shock protein 70-2 (HSP70-2) mutant; thyroid stimulating hormone receptor (TSHR); CD123; CD171; CD19; CD20; CD22; CD26; CD30; CD33; CD44v7/8 (cluster of differentiation 44, exons 7/8); CD53; CD92; CD100; CD148; CD150; CD200; CD261; CD262; CD362; CS-1 (CD2 subset 1, CRACC, SLAMF7, CD319, and 19A24); C-type lectin-like molecule-1 (CLL-1); ganglioside GD3 (aNeu5Ac(2-8)aNeu5Ac(2-3)bDGalp(1-4)bDGlcp(1-1)Cer); Tn antigen (Tn Ag); Fms-Like Tyrosine Kinase 3 (FLT3); CD38; CD138; CD44v6; B7H3 (CD276); KIT (CD117); Interleukin-13 receptor subunit alpha-2 (IL-13Ra2); Interleukin 11 receptor alpha (IL-11Ra); prostate stem cell antigen (PSCA); Protease Serine 21 (PRSS21); vascular endothelial growth factor receptor 2 (VEGFR2); Lewis(Y) antigen; CD24; Platelet-derived growth factor receptor beta (PDGFR-beta); stage-specific embryonic antigen-4 (SSEA-4); Mucin 1, cell surface associated (MUC1); mucin 16 (MUC16); epidermal growth factor receptor (EGFR); epidermal growth factor receptor variant III (EGFRvIII); neural cell adhesion molecule (NCAM); carbonic anhydrase IX (CAIX); Proteasome (Prosome, Macropain) Subunit, Beta Type, 9 (LMP2); ephrin type-A receptor 2 (EphA2); Ephrin B2; Fucosyl GM1; sialyl Lewis adhesion molecule (sLe); ganglioside GM3 (aNeu5Ac(2-3)bDGalp(1-4)bDGlcp(1-1)Cer); TGS5; high molecular weight-melanoma-associated antigen (HMWMAA); o-acetyl-GD2 ganglioside (OAcGD2); Folate receptor alpha; Folate receptor beta; tumor endothelial marker 1 (TEM1/CD248); tumor endothelial marker 7-related (TEM7R); claudin 6 (CLDN6); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); ETS translocation-variant gene 6, located on chromosome 12p (ETV6-AML); sperm protein 17 (SPA17); X Antigen Family, Member 1A (XAGE1), angiopoietin-binding cell surface receptor 2 (Tie 2); CT (cancer/testis (antigen)); melanoma cancer testis antigen-1 (MAD-CT-1); melanoma cancer testis antigen-2 (MAD-CT-2); Fos-related antigen 1; p53; p53 mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; Cyclin D1; v-myc avian myelocytomatosis viral oncogene neuroblastoma derived homolog (MYCN); Ras Homolog Family Member C (RhoC); Cytochrome P450 1B1 (CYP1B1); CCCTC-Binding Factor (Zinc Finger Protein)-Like (BORIS); Squamous Cell Carcinoma Antigen Recognized By T Cells-1 or 3 (SART1, SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint-1, -2, -3 or -4 (SSX1, SSX2, SSX3, SSX4); CD79a; CD79b; CD72; Leukocyte-associated immunoglobulin-like receptor 1 (LAIR1); Fc fragment of IgA receptor (FCAR); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); mouse double minute 2 homolog (MDM2); livin; alphafetoprotein (AFP); transmembrane activator and CAML Interactor (TACI); B-cell activating factor receptor (BAFF-R); V-Ki-ras2 Kirsten rat sarcoma viral oncogene homolog (KRAS); immunoglobulin lambda-like polypeptide 1 (IGLL 1); 707-AP (707 alanine proline); ART-4 (adenocarcinoma antigen recognized by T4 cells); BAGE (B antigen; b-catenin/m, b-catenin/mutated); CAMEL (CTL-recognized antigen on melanoma); CAP1 (carcinoembryonic antigen peptide 1); CASP-8 (caspase-8); CDC27m (cell-division cycle 27 mutated); CDK4/m (cycline-dependent kinase 4 mutated); Cyp-B (cyclophilin B); DAM (differentiation antigen melanoma); EGP-2 (epithelial glycoprotein 2); EGP-40 (epithelial glycoprotein 40); Erbb2, 3, 4 (erythroblastic leukemia viral oncogene homolog-2, -3, 4); FBP (folate binding protein); fAchR (Fetal acetylcholine receptor); G250 (glycoprotein 250); GAGE (G antigen); GnT-V (N-acetylglucosaminyltransferase V); HAGE (helicose antigen); ULA-A (human leukocyte antigen-A); HST2 (human signet ring tumor 2); KIAA0205; KDR (kinase insert domain receptor); LDLR/FUT (low density lipid receptor/GDP L-fucose: b-D-galactosidase 2-α-L fucosyltransferase); L1CAM (L1 cell adhesion molecule); MC1R (melanocortin 1 receptor); Myosin/m (myosin mutated); MUM-1, -2, -3 (melanoma ubiquitous mutated 1, 2, 3); NA88-A (NA cDNA clone of patient M88); KG2D (Natural killer group 2, member D) ligands; oncofetal antigen (h5T4); p190 minor bcr-abl (protein of 190KD bcr-abl); Pml/RARa (promyelocytic leukaemia/retinoic acid receptor a); PRAME (preferentially expressed antigen of melanoma); SAGE (sarcoma antigen); TEL/AML1 (translocation Ets-family leukemia/acute myeloid leukemia 1); TPI/m (triosephosphate isomerase mutated); CD70; trophoblast glycoprotein (TPBG); αvβó integrin, B7-H3; B7-H6; CD20; CD44; chondroitin sulfate proteoglycan 4 (CSPG4), bDGalpNAc(1-4)[aNeu5Ac(2-8)aNeu5Ac(2-3)]bDGalp(1-4)bDGlcp(1-1)Cer (GD2), aNeu5Ac(2-8)aNeu5Ac(2-3)bDGalp(1-4)bDGlcp(1-1)Cer (GD3); human leukocyte antigen A1 MAGE family member A1 (HLA-A1+MAGEA1); human leukocyte antigen A2 MAGE family member A1 (HLA-A2+MAGEA1); human leukocyte antigen A3 MAGE family member A1 (HLA-A3+MAGEA1); MAGEA1; human leukocyte antigen A1 New York Esophageal Squamous Cell Carcinoma 1 (FILA-A1+NY-ESO-1); human leukocyte antigen A2 New York Esophageal Squamous Cell Carcinoma 1 (HLA-A2+NY-ESO-1), lambda light chain, kappa light chain, tumor endothelial marker 5 (TEM5), tumor endothelial marker 7 (TEM7), tumor endothelial marker 8 (TEM8), TEM5, TEM7, TEM8, IFN-inducible p78, melanotransferrin (p97), human kallikrein (huK2), Axl, ROR2, FKBP11, KAMP3, ITGA8, FCRL5, LAGA-1, CD133, cD34, EBV nuclear antigen-1 (EBNA1), latent membrane protein 1 (LMP1) and LMP2A, CD75, gp100, MICA, MICB, MART1, carcinoembryonic antigen, CA-125, MAGEC2, CTAG2, CTAG1, pd-12, CLA, CD142, CD73, CD49c, CD66c, CD104, CD318, TSPAN8, CLEC14, human immunodeficiency virus 1 (HIV-1) reverse transcriptase (RT), Cd16, BLTA, IL-2, IL-7, IL-15, IL-21, IL-12, CCR4, CCR2b, Heparanase, CD137L, LEM, and Bcl-2, Msln, Cd8, IL-15, 4-1BBL, OX40L, 4-IBB, cd95, cd27, HVENM, CXCR4; and any combination thereof. In some example, the antigen to be targeted may be CXCR. In some examples, the antigen to be targeted may be PD-1.

In certain embodiments, an antigen to be targeted in adoptive cell therapy (such as particularly CAR or TCR T-cell therapy) of a disease (such as particularly of tumor or cancer) is a tumor-specific antigen (TSA).

In certain embodiments, an antigen to be targeted in adoptive cell therapy (such as particularly CAR or TCR T-cell therapy) of a disease (such as particularly of tumor or cancer) is a neoantigen.

In certain embodiments, an antigen to be targeted in adoptive cell therapy (such as particularly CAR or TCR T-cell therapy) of a disease (such as particularly of tumor or cancer) is a tumor-associated antigen (TAA).

In certain embodiments, an antigen to be targeted in adoptive cell therapy (such as particularly CAR or TCR T-cell therapy) of a disease (such as particularly of tumor or cancer) is a universal tumor antigen. In certain preferred embodiments, the universal tumor antigen is selected from the group consisting of: a human telomerase reverse transcriptase (hTERT), survivin, mouse double minute 2 homolog (MDM2), cytochrome P450 1B 1 (CYP1B), HER2/neu, Wilms' tumor gene 1 (WT1), livin, alphafetoprotein (AFP), carcinoembryonic antigen (CEA), mucin 16 (MUC16), MUC1, prostate-specific membrane antigen (PSMA), p53, cyclin (D1), and any combinations thereof.

In certain embodiments, an antigen (such as a tumor antigen) to be targeted in adoptive cell therapy (such as particularly CAR or TCR T-cell therapy) of a disease (such as particularly of tumor or cancer) may be selected from a group consisting of: CD19, BCMA, CD70, CLL-1, MAGE A3, MAGE A6, HPV E6, HPV E7, WT1, CD22, CD171, ROR1, MUC16, and SSX2. In certain preferred embodiments, the antigen may be CD19. For example, CD19 may be targeted in hematologic malignancies, such as in lymphomas, more particularly in B-cell lymphomas, such as without limitation in diffuse large B-cell lymphoma, primary mediastinal b-cell lymphoma, transformed follicular lymphoma, marginal zone lymphoma, mantle cell lymphoma, acute lymphoblastic leukemia including adult and pediatric ALL, non-Hodgkin lymphoma, indolent non-Hodgkin lymphoma, or chronic lymphocytic leukemia. For example, BCMA may be targeted in multiple myeloma or plasma cell leukemia (see, e.g., 2018 American Association for Cancer Research (AACR) Annual meeting Poster: Allogeneic Chimeric Antigen Receptor T Cells Targeting B Cell Maturation Antigen). For example, CLL1 may be targeted in acute myeloid leukemia. For example, MAGE A3, MAGE A6, SSX2, and/or KRAS may be targeted in solid tumors. For example, HPV E6 and/or HPV E7 may be targeted in cervical cancer or head and neck cancer. For example, WT1 may be targeted in acute myeloid leukemia (AML), myelodysplastic syndromes (MDS), chronic myeloid leukemia (CML), non-small cell lung cancer, breast, pancreatic, ovarian or colorectal cancers, or mesothelioma. For example, CD22 may be targeted in B cell malignancies, including non-Hodgkin lymphoma, diffuse large B-cell lymphoma, or acute lymphoblastic leukemia. For example, CD171 may be targeted in neuroblastoma, glioblastoma, or lung, pancreatic, or ovarian cancers. For example, ROR1 may be targeted in ROR1+ malignancies, including non-small cell lung cancer, triple negative breast cancer, pancreatic cancer, prostate cancer, ALL, chronic lymphocytic leukemia, or mantle cell lymphoma. For example, MUC16 may be targeted in MUC16ecto+ epithelial ovarian, fallopian tube or primary peritoneal cancer. For example, CD70 may be targeted in both hematologic malignancies as well as in solid cancers such as renal cell carcinoma (RCC), gliomas (e.g., GBM), and head and neck cancers (HNSCC). CD70 is expressed in both hematologic malignancies as well as in solid cancers, while its expression in normal tissues is restricted to a subset of lymphoid cell types (see, e.g., 2018 American Association for Cancer Research (AACR) Annual meeting Poster: Allogeneic CRISPR Engineered Anti-CD70 CAR-T Cells Demonstrate Potent Preclinical Activity Against Both Solid and Hematological Cancer Cells).

In some embodiments, the target antigen is a viral antigen. Many viral antigen targets have been identified and are known, including peptides derived from viral genomes in HIV, HTLV and other viruses (see e.g., Addo et al. (2007) PLoS ONE, 2, e321; Tsomides et al. (1994) J Exp Med, 180, 1283-93; Utz et al. (1996) J Virol, 70, 843-51). Exemplary viral antigens include, but are not limited to, an antigen from hepatitis A, hepatitis B (e.g., HBV core and surface antigens (HBVc, HBVs)), hepatitis C (HCV), Epstein-Ban* virus (e.g. EBVA), human papillomavirus (HPV; e.g. E6 and E7), human immunodeficiency type-1 virus (HIV1), Kaposi's sarcoma herpes virus (KSHV), human papilloma virus (HPV), influenza virus, Lassa virus, HTLN-i, HIN-1, HIN-IL CMN, EBN or HPN. In some embodiments, the target protein is a bacterial antigen or other pathogenic antigen, such as Mycobacterium tuberculosis (MT) antigens, trypanosome, e.g., Trypanosoma cruzi (T. cruzi), antigens such as surface antigen (TSA), or malaria antigens. Specific viral antigen or epitopes or other pathogenic antigens or peptide epitopes are known (see e.g., Addo et al. (2007) PLoS ONE, 2, e321; Anikeeva et al. (2009) Clin Immunol, 130, 98-109). [0133] In some embodiments, the antigen is an antigen derived from a virus associated with cancer, such as an oncogenic virus. For example, an oncogenic virus is one in which infection from certain viruses are known to lead to the development of different types of cancers, for example, hepatitis A, hepatitis B (HB V), hepatitis C (HCV), human papilloma virus (HPV), hepatitis viral infections, Epstein-Barr virus (EBV), human herpes virus 8 (HHV-8), human T-cell leukemia virus-1 (HTLV-1), human T-cell leukemia virus-2 (HTLV-2), or a cytomegalovirus (CMV) antigen. In some embodiments, the viral antigen is an HPV antigen, which, in some cases, can lead to a greater risk of developing cervical and/or head and neck cancers. In some embodiments, the antigen can be a HPV-16 antigen, and HPV-18 antigen, and HPV-31 antigen, an HPV-33 antigen or an HPV-35 antigen. In some embodiments, the viral antigen is an HPV-16 antigens (e.g., seroreactive regions of the E1, E2, E6 or E7 proteins of HPV-16, see e.g. U.S. Pat. No. 6,531,127) or an HPV-18 antigens (e.g., seroreactive regions of the L1 and/or L2 proteins of HPV-18, such as described in U.S. Pat. No. 5,840,306).

In some embodiments, the viral antigen is a HBV or HCV antigen, which, in some cases, can lead to a greater risk of developing liver cancer than HBV or HCV negative subjects. For example, in some embodiments, the heterologous antigen is an HBV antigen, such as a hepatitis B core antigen or an hepatitis B envelope antigen (US2012/0308580).

In some embodiments, the viral antigen is an EBV antigen, which, in some cases, can lead to a greater risk for developing Burkitt's lymphoma, nasopharyngeal carcinoma and Hodgkin's disease than EBV negative subjects. For example, EBV is a human herpes virus that, in some cases, is found associated with numerous human tumors of diverse tissue origin. While primarily found as an asymptomatic infection, EBV-positive tumors can be characterized by active expression of viral gene products, such as EBNA-1, LMP-1 and LMP-2A. In some embodiments, the heterologous antigen is an EBV antigen that can include Epstein-Barr nuclear antigen (EBNA)-1, EBNA-2, EBNA-3A, EBNA-3B, EBNA-3C, EBNA-leader protein (EBNA-LP), latent membrane proteins LMP-1, LMP-2A and LMP-2B, EBV-EA, EBV-MA or EBV-VCA. [0137] In some embodiments, the viral antigen is an HTLV-1 or HTLV-2 antigen, which, in some cases, can lead to a greater risk for developing T-cell leukemia than HTLV-1 or HTLV-2 negative subjects. For example, in some embodiments, the heterologous antigen is an HTLV-antigen, such as TAX.

In some embodiments, the viral antigen is a HHV-8 antigen, which, in some cases, can lead to a greater risk for developing Kaposi's sarcoma than HHV-8 negative subjects. In some embodiments, the heterologous antigen is a CMV antigen, such as pp65 or pp64 (see U.S. Pat. No. 8,361,473).

In some embodiments, the viral antigen is a virus-specific surface antigen such as an HIV-specific antigen (such as HIV gp120); an EBV-specific antigen, a CMV-specific antigen, a HPV-specific antigen, a Lasse Virus-specific antigen, an Influenza Virus-specific antigen as well as any derivate or variant of these surface markers.

Approaches such as the foregoing may be adapted to provide methods of treating and/or increasing survival of a subject having a disease, such as a neoplasia, for example by administering an effective amount of an immunoresponsive cell comprising an antigen recognizing receptor that binds a selected antigen, wherein the binding activates the immunoresponsive cell, thereby treating or preventing the disease (such as a neoplasia, a pathogen infection, an autoimmune disorder, or an allogeneic transplant reaction). Dosing in CAR T cell therapies may for example involve administration of from 106 to 109 cells/kg, with or without a course of lymphodepletion, for example with cyclophosphamide.

A person with ordinary skill in the art may use the CRISPR system disclosed in this invention in a similar system as described above. With respect to the specific CRISPR nucleases, the CRISPR system may recognize a PAM sequence that is a T-rich sequence. In some embodiments, the PAM sequence is 5′ TTN 3′ or 5′ ATTN 3′, wherein N is any nucleotide. In some embodiments, the CRISPR system introduces one or more staggered double strand breaks (DSBs) with a 5′ overhang to the target gene. In particular embodiments, the 5′ overhang is 7 nt. In some embodiments, the CRISPR system introduces a template DNA sequence at the staggered DSB via HR or NHEJ. In some particular embodiments, the CRISPR system comprises a catalytically inactivated protein associated with a functional domain that modifies the target gene. In a particular embodiment, the CRISPR system introduces a single mutation. In another particular embodiment, the CRISPR system introduces a single nucleotide modification to the transcript of the target gene.

In one embodiment, the treatment can be administrated into patients undergoing an immunosuppressive treatment. The cells or population of cells, may be made resistant to at least one immunosuppressive agent due to the inactivation of a gene encoding a receptor for such immunosuppressive agent. Not being bound by a theory, the immunosuppressive treatment should help the selection and expansion of the immunoresponsive or T cells according to the invention within the patient.

In certain embodiments, the treatment can be administered before primary treatment (e.g., surgery or radiation therapy) to shrink a tumor before the primary treatment. In another embodiment, the treatment can be administered after primary treatment to remove any remaining cancer cells.

In certain embodiments, immunometabolic barriers can be targeted therapeutically prior to and/or during ACT to enhance responses to ACT or CAR T-cell therapy and to support endogenous immunity (see, e.g., Irving et al., Engineering Chimeric Antigen Receptor T-Cells for Racing in Solid Tumors: Don't Forget the Fuel, Front. Immunol., 3 Apr. 2017, doi.org/10.3389/fimmu.2017.00267).

The administration of the cells or population of cells according to the present invention may be carried out in any convenient manner, including by aerosol inhalation, injection, ingestion, transfusion, implantation or transplantation. The cells or population of cells may be administered to a patient subcutaneously, intradermally, intratumorally, intranodally, intramedullary, intramuscularly, by intravenous or intralymphatic injection, or intraperitoneally. In one embodiment, the cell compositions of the present invention are preferably administered by intravenous injection.

The administration of the cells or population of cells can consist of the administration of 104-109 cells per kg body weight, preferably 105 to 106 cells/kg body weight including all integer values of cell numbers within those ranges. Dosing in CAR T cell therapies may for example involve administration of from 106 to 109 cells/kg, with or without a course of lymphodepletion, for example with cyclophosphamide. The cells or population of cells can be administrated in one or more doses. In another embodiment, the effective amount of cells are administrated as a single dose. In another embodiment, the effective amount of cells are administrated as more than one dose over a period time. Timing of administration is within the judgment of managing physician and depends on the clinical condition of the patient. The cells or population of cells may be obtained from any source, such as a blood bank or a donor. While individual needs vary, determination of optimal ranges of effective amounts of a given cell type for a particular disease or conditions are within the skill of one in the art. An effective amount means an amount which provides a therapeutic or prophylactic benefit. The dosage administrated will be dependent upon the age, health and weight of the recipient, kind of concurrent treatment, if any, frequency of treatment and the nature of the effect desired.

In another embodiment, the effective amount of cells or composition comprising those cells are administrated parenterally. The administration can be an intravenous administration. The administration can be directly done by injection within a tumor.

To guard against possible adverse reactions, engineered immunoresponsive cells may be equipped with a transgenic safety switch, in the form of a transgene that renders the cells vulnerable to exposure to a specific signal. For example, the herpes simplex viral thymidine kinase (TK) gene may be used in this way, for example by introduction into allogeneic T lymphocytes used as donor lymphocyte infusions following stem cell transplantation (Greco, et al., Improving the safety of cell therapy with the TK-suicide gene. Front. Pharmacol. 2015; 6: 95). In such cells, administration of a nucleoside prodrug such as ganciclovir or acyclovir causes cell death. Alternative safety switch constructs include inducible caspase 9, for example triggered by administration of a small-molecule dimerizer that brings together two nonfunctional icasp9 molecules to form the active enzyme. A wide variety of alternative approaches to implementing cellular proliferation controls have been described (see U.S. Patent Publication No. 20130071414; PCT Patent Publication WO2011146862; PCT Patent Publication WO2014011987; PCT Patent Publication WO2013040371; Zhou et al. BLOOD, 2014, 123/25:3895-3905; Di Stasi et al., The New England Journal of Medicine 2011; 365:1673-1683; Sadelain M, The New England Journal of Medicine 2011; 365:1735-173; Ramos et al., Stem Cells 28(6):1107-15 (2010)).

In a further refinement of adoptive therapies, genome editing with a CRISPR-Cas system as described herein may be used to tailor immunoresponsive cells to alternative implementations, for example providing edited CAR T cells (see Poirot et al., 2015, Multiplex genome edited T-cell manufacturing platform for “off-the-shelf” adoptive T-cell immunotherapies, Cancer Res 75 (18): 3853). For example, immunoresponsive cells may be edited to delete expression of some or all of the class of HLA type II and/or type I molecules, or to knockout selected genes that may inhibit the desired immune response, such as the PD1 gene.

Cells may be edited using any CRISPR system and method of use thereof as described herein. CRISPR systems may be delivered to an immune cell by any method described herein. In preferred embodiments, cells are edited ex vivo and transferred to a subject in need thereof. Immunoresponsive cells, CAR T cells or any cells used for adoptive cell transfer may be edited. Editing may be performed to eliminate potential alloreactive T-cell receptors (TCR), disrupt the target of a chemotherapeutic agent, block an immune checkpoint, activate a T cell, and/or increase the differentiation and/or proliferation of functionally exhausted or dysfunctional CD8+ T-cells (see PCT Patent Publications: WO2013176915, WO2014059173, WO2014172606, WO2014184744, and WO2014191128). Editing may result in inactivation of a gene.

By inactivating a gene it is intended that the gene of interest is not expressed in a functional protein form. In a particular embodiment, the CRISPR system specifically catalyzes cleavage in one targeted gene thereby inactivating said targeted gene. The nucleic acid strand breaks caused are commonly repaired through the distinct mechanisms of homologous recombination or non-homologous end joining (NHEJ). However, NHEJ is an imperfect repair process that often results in changes to the DNA sequence at the site of the cleavage. Repair via non-homologous end joining (NHEJ) often results in small insertions or deletions (Indel) and can be used for the creation of specific gene knockouts. Cells in which a cleavage induced mutagenesis event has occurred can be identified and/or selected by well-known methods in the art.

T cell receptors (TCR) are cell surface receptors that participate in the activation of T cells in response to the presentation of antigen. The TCR is generally made from two chains, α and β, which assemble to form a heterodimer and associates with the CD3-transducing subunits to form the T cell receptor complex present on the cell surface. Each α and β chain of the TCR consists of an immunoglobulin-like N-terminal variable (V) and constant (C) region, a hydrophobic transmembrane domain, and a short cytoplasmic region. As for immunoglobulin molecules, the variable region of the α and β chains are generated by V(D)J recombination, creating a large diversity of antigen specificities within the population of T cells. However, in contrast to immunoglobulins that recognize intact antigen, T cells are activated by processed peptide fragments in association with an MHC molecule, introducing an extra dimension to antigen recognition by T cells, known as MHC restriction. Recognition of MHC disparities between the donor and recipient through the T cell receptor leads to T cell proliferation and the potential development of graft versus host disease (GVHD). The inactivation of TCRα or TCRβ can result in the elimination of the TCR from the surface of T cells preventing recognition of alloantigen and thus GVHD. However, TCR disruption generally results in the elimination of the CD3 signaling component and alters the means of further T cell expansion.

Allogeneic cells are rapidly rejected by the host immune system. It has been demonstrated that, allogeneic leukocytes present in non-irradiated blood products will persist for no more than 5 to 6 days (Boni, Muranski et al. 2008 Blood 1; 112(12):4746-54). Thus, to prevent rejection of allogeneic cells, the host's immune system usually has to be suppressed to some extent. However, in the case of adoptive cell transfer the use of immunosuppressive drugs also have a detrimental effect on the introduced therapeutic T cells. Therefore, to effectively use an adoptive immunotherapy approach in these conditions, the introduced cells would need to be resistant to the immunosuppressive treatment. Thus, in a particular embodiment, the present invention further comprises a step of modifying T cells to make them resistant to an immunosuppressive agent, preferably by inactivating at least one gene encoding a target for an immunosuppressive agent. An immunosuppressive agent is an agent that suppresses immune function by one of several mechanisms of action. An immunosuppressive agent can be, but is not limited to a calcineurin inhibitor, a target of rapamycin, an interleukin-2 receptor α-chain blocker, an inhibitor of inosine monophosphate dehydrogenase, an inhibitor of dihydrofolic acid reductase, a corticosteroid or an immunosuppressive antimetabolite. The present invention allows conferring immunosuppressive resistance to T cells for immunotherapy by inactivating the target of the immunosuppressive agent in T cells. As non-limiting examples, targets for an immunosuppressive agent can be a receptor for an immunosuppressive agent such as: CD52, glucocorticoid receptor (GR), a FKBP family gene member and a cyclophilin family gene member.

Immune checkpoints are inhibitory pathways that slow down or stop immune reactions and prevent excessive tissue damage from uncontrolled activity of immune cells. In certain embodiments, the immune checkpoint targeted is the programmed death-1 (PD-1 or CD279) gene (PDCD1). In other embodiments, the immune checkpoint targeted is cytotoxic T-lymphocyte-associated antigen (CTLA-4). In additional embodiments, the immune checkpoint targeted is another member of the CD28 and CTLA4 Ig superfamily such as BTLA, LAG3, ICOS, PDL1 or KIR. In further additional embodiments, the immune checkpoint targeted is a member of the TNFR superfamily such as CD40, OX40, CD137, GITR, CD27 or TIM-3.

Additional immune checkpoints include Src homology 2 domain-containing protein tyrosine phosphatase 1 (SHP-1) (Watson H A, et al., SHP-1: the next checkpoint target for cancer immunotherapy? Biochem Soc Trans. 2016 Apr. 15; 44(2):356-62). SHP-1 is a widely expressed inhibitory protein tyrosine phosphatase (PTP). In T-cells, it is a negative regulator of antigen-dependent activation and proliferation. It is a cytosolic protein, and therefore not amenable to antibody-mediated therapies, but its role in activation and proliferation makes it an attractive target for genetic manipulation in adoptive transfer strategies, such as chimeric antigen receptor (CAR) T cells. Immune checkpoints may also include T cell immunoreceptor with Ig and ITIM domains (TIGITNstm3/WUCAM/VSIG9) and VISTA (Le Mercier I, et al., (2015) Beyond CTLA-4 and PD-1, the generation Z of negative checkpoint regulators. Front. Immunol. 6:418).

WO2014172606 relates to the use of MT1 and/or MT1 inhibitors to increase proliferation and/or activity of exhausted CD8+ T-cells and to decrease CD8+ T-cell exhaustion (e.g., decrease functionally exhausted or unresponsive CD8+ immune cells). In certain embodiments, metallothioneins are targeted by gene editing in adoptively transferred T cells.

In certain embodiments, targets of gene editing may be at least one targeted locus involved in the expression of an immune checkpoint protein. Such targets may include, but are not limited to CTLA4, PPP2CA, PPP2CB, PTPN6, PTPN22, PDCD1, ICOS (CD278), PDL1, KIR, LAG3, HAVCR2, BTLA, CD160, TIGIT, CD96, CRTAM, LAIR1, SIGLEC7, SIGLEC9, CD244 (2B4), TNFRSF10B, TNFRSF10A, CASP8, CASP10, CASP3, CASP6, CASP7, FADD, FAS, TGFBRII, TGFRBRI, SMAD2, SMAD3, SMAD4, SMAD10, SKI, SKIL, TGIF1, IL10RA, IL10RB, HMOX2, IL6R, IL6ST, EIF2AK4, CSK, PAG1, SIT1, FOXP3, PRDM1, BATF, VISTA, GUCYIA2, GUCYIA3, GUCYIB2, GUCYIB3, MT1, MT2, CD40, OX40, CD137, GITR, CD27, SHP-1, TIM-3, CEACAM-1, CEACAM-3, or CEACAM-5. In preferred embodiments, the gene locus involved in the expression of PD-1 or CTLA-4 genes is targeted. In other preferred embodiments, combinations of genes are targeted, such as but not limited to PD-1 and TIGIT.

In other embodiments, at least two genes are edited. Pairs of genes may include, but are not limited to PD1 and TCRα, PD1 and TCRs, CTLA-4 and TCRα, CTLA-4 and TCRβ, LAG3 and TCRα, LAG3 and TCRβ, Tim3 and TCRα, Tim3 and TCRβ, BTLA and TCRα, BTLA and TCRβ, BY55 and TCRα, BY55 and TCRs, TIGIT and TCRα, TIGIT and TCRβ, B7H5 and TCRα, B7H5 and TCRβ3, LAIR1 and TCRα, LAIR1 and TCRβ, SIGLEC10 and TCRα, SIGLEC10 and TCRs, 2B4 and TCRα, 2B4 and TCRβ.

Whether prior to or after genetic modification of the T cells, the T cells can be activated and expanded generally using methods as described, for example, in U.S. Pat. Nos. 6,352,694; 6,534,055; 6,905,680; 5,858,358; 6,887,466; 6,905,681; 7,144,575; 7,232,566; 7,175,843; 5,883,223; 6,905,874; 6,797,514; 6,867,041; and 7,572,631. T cells can be expanded in vitro or in vivo.

The practice of the present invention employs, unless otherwise indicated, conventional techniques of immunology, biochemistry, chemistry, molecular biology, microbiology, cell biology, genomics and recombinant DNA, which are within the skill of the art. See MOLECULAR CLONING: A LABORATORY MANUAL, 2nd edition (1989) (Sambrook, Fritsch and Maniatis); MOLECULAR CLONING: A LABORATORY MANUAL, 4th edition (2012) (Green and Sambrook); CURRENT PROTOCOLS IN MOLECULAR BIOLOGY (1987) (F. M. Ausubel, et al. eds.); the series METHODS IN ENZYMOLOGY (Academic Press, Inc.); PCR 2: A PRACTICAL APPROACH (1995)(M. J. MacPherson, B. D. Hames and G. R. Taylor eds.); ANTIBODIES, A LABORATORY MANUAL (1988) (Harlow and Lane, eds.); ANTIBODIES A LABORATORY MANUAL, 2nd edition (2013) (E. A. Greenfield ed.); and ANIMAL CELL CULTURE (1987) (R. I. Freshney, ed.).

The practice of the present invention employs, unless otherwise indicated, conventional techniques for generation of genetically modified mice. See Marten H. Hofker and Jan van Deursen, TRANSGENIC MOUSE METHODS AND PROTOCOLS, 2nd edition (2011).

In some embodiments, the treatment in a subject who is either responsive or non-responsive to cancer treatment, is checkpoint blockade therapy. In specific exemplary embodiments, the checkpoint blockade therapy may include anti-PD-1, anti-CTLA4, anti-PDL1, anti-TIM-3 and/or anti-LAG3, as described above.

In some embodiments, the phenotype of the subject is a basal phenotype and/or IFNγ phenotype.

In some embodiments, the ex vivo cell-based system is an organoid.

As used herein, the term “organoid” or “epithelial organoid” refers to a cell cluster or aggregate that resembles an organ, or part of an organ, and possesses cell types relevant to that particular organ. Organoid technology has been previously described for example, for brain, retinal, stomach, lung, thyroid, small intestine, colon, liver, kidney, pancreas, prostate, mammary gland, fallopian tube, taste buds, salivary glands, and esophagus (see, e.g., Clevers, Modeling Development and Disease with Organoids, Cell. 2016 Jun. 16; 165(7):1586-1597).

### Methods for Screening Therapeutic Agents

In some embodiments, the invention provides a method for screening therapeutic agents, comprising exposing the ex vivo cell-based model system as described herein to one or more therapeutic agents, measuring responsiveness of the ex vivo model to the one or more therapeutic agents; and classifying the one or more therapeutic agents as indicated if the ex vivo model exhibits a responsive phenotype indicating a susceptibility of the model to the one or more therapeutic agents, or contraindicated if the ex vivo model exhibits a non-responsive phenotype indicating a lack of susceptibility of the model to the one or more therapeutic agents.

### Exposing Ex Vivo Cell-Based System

The compositions and methods described herein comprise exposing the cell-based model system to one or more therapeutic agents. Embodiments may comprise exposing to a single agent or a combination of multiple agents, for example two, three, four, five, six or more agents. Exposing the agents may comprise administering multiple agents together, separately, or over different time courses. The ex vivo cell based system derived from a subject to be treated and the agents screened are to select for the best treatment or treatment combination. Accordingly, a variety of permutations of single or multiple agents administered, time course of exposing the cell-based system, dose of agents and varying combinations of agents can be utilized to optimize selection of treatment. In certain embodiments, a library of agents is tested in combination with a standard treatment to identify agents that make a tumor system more or less responsive to the standard treatment (e.g., chemotherapy, immunotherapy, or targeted therapy).

The terms “therapeutic agent”, “therapeutic capable agent” or “treatment agent” are used interchangeably and refer to a molecule or compound that confers some beneficial effect upon administration to a subject. The beneficial effect includes enablement of diagnostic determinations; amelioration of a disease, symptom, disorder, or pathological condition; reducing or preventing the onset of a disease, symptom, disorder or condition; and generally counteracting a disease, symptom, disorder or pathological condition.

In certain embodiments, libraries are screened. A combinatorial library contains a large number of potential therapeutic compounds. A combinatorial chemical library may be a collection of diverse chemical compounds generated by either chemical synthesis or biological synthesis, by combining a number of chemical “building blocks” such as reagents. For example, a linear combinatorial chemical library, such as a polypeptide library, is formed by combining a set of chemical building blocks (amino acids) in every possible way for a given compound length (for example, the number of amino acids in a polypeptide compound). Millions of chemical compounds can be synthesized through such combinatorial mixing of chemical building blocks.

Appropriate agents can be contained in libraries, for example, synthetic or natural compounds in a combinatorial library. Numerous libraries are commercially available or can be readily produced; means for random and directed synthesis of a wide variety of organic compounds and biomolecules, including expression of randomized oligonucleotides, such as antisense oligonucleotides and oligopeptides, also are known. Alternatively, libraries of natural compounds in the form of bacterial, fungal, plant and animal extracts are available or can be readily produced. Additionally, natural or synthetically produced libraries and compounds are readily modified through conventional chemical, physical and biochemical means, and may be used to produce combinatorial libraries. Such libraries are useful for the screening of a large number of different compounds.

Preparation and screening of combinatorial libraries is well known to those of skill in the art. Libraries (such as combinatorial chemical libraries) useful in the disclosed methods include, but are not limited to, peptide libraries (see, e.g., U.S. Pat. No. 5,010,175; Furka, Int. J. Pept. Prot. Res., 37:487-493, 1991; Houghton et al, Nature, 354:84-88, 1991; PCT Publication No. WO 91/19735), (see, e.g., Lam et al., Nature, 354:82-84, 1991, Houghten et al., Nature, 354:84-86, 1991), and combinatorial chemistry-derived molecular library made of D- and/or L-configuration amino acids, phosphopeptides (including, but not limited to, members of random or partially degenerate, directed phosphopeptide libraries; see, e.g., Songyang et al., Cell, 72:767-778, 1993), antibodies (including, but not limited to, polyclonal, monoclonal, humanized, anti-idiotypic, chimeric or single chain antibodies, and Fab, F(ab′)2 and Fab expression library fragments, and epitope-binding fragments thereof), small organic or inorganic molecules (such as, so-called natural products or members of chemical combinatorial libraries), molecular complexes (such as protein complexes), or nucleic acids, encoded peptides (e.g., PCT Publication WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Natl Acad. Sa. USA, 90:6909-6913, 1993), vinylogous polypeptides (Hagihara et al., J. Am. Chem. Soc, 114:6568, 1992), nonpeptidal peptidomimetics with glucose scaffolding (Hirschmann et al., J. Am. Chem. Soc, 114:9217-9218, 1992), analogous organic syntheses of small compound libraries (Chen et al., J. Am. Chem. Soc, 116:2661, 1994), oligo carbamates (Cho et al., Science, 261:1303, 1003), and/or peptidyl phosphonates (Campbell et al., J. Org. Chem., 59:658, 1994), nucleic acid libraries (see Sambrook et al. Molecular Cloning, A Laboratory Manual, Cold Springs Harbor Press, N Y., 1989; Ausubel et al., Current Protocols m Molecular Biology, Green Publishing Associates and Wiley Interscience, N. Y., 1989), peptide nucleic acid libraries (see, e.g., U.S. Pat. No. 5,539,083), antibody libraries (see, e.g., Vaughn et al., Nat. Biotechnol, 14:309-314, 1996; PCT App. No. PCT/US96/10287), carbohydrate libraries (see, e.g., Liang et al., Science, 274:1520-1522, 1996; U.S. Pat. No. 5,593,853), small organic molecule libraries (see, e.g., benzodiazepines, Baum, C&EN, January 18, page 33, 1993; isoprenoids, U.S. Pat. No. 5,569,588; thiazolidionones and methathiazones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514) and the like.

Libraries useful for the disclosed screening methods can be produced in a variety of manners including, but not limited to, spatially arrayed multipin peptide synthesis (Geysen, et al., Proc. Natl. Acad. Sci., 81(13):3998-4002, 1984), “tea bag” peptide synthesis (Houghten, Proc. Natl. Acad. Sci., 82(15):5131-5135, 1985), phage display (Scott and Smith, Science, 249:386-390, 1990), spot or disc synthesis (Dittrich et al., Bworg. Med. Chem. Lett., 8(17):2351-2356, 1998), or split and mix solid phase synthesis on beads (Furka et al., Int. J. Pept. Protein Res., 37(6):487-493, 1991; Lam et al., Chem. Rev., 97 (2):411-448, 1997).

Devices for the preparation of combinatorial libraries are also commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, for example, ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md., etc.).

Libraries can include a varying number of compositions (members), such as up to about 100 members, such as up to about 1,000 members, such as up to about 5,000 members, such as up to about 10,000 members, such as up to about 100,000 members, such as up to about 500,000 members, or even more than 500,000 members. In one example, the methods can involve providing a combinatorial chemical or peptide library containing a large number of potential therapeutic compounds. Such combinatorial libraries are then screened by the methods disclosed herein to identify those library members (particularly chemical species or subclasses) that display a desired characteristic activity.

The compounds identified using the methods disclosed herein can serve as conventional “lead compounds” or can themselves be used as potential or actual therapeutics. In some instances, pools of candidate agents can be identified and further screened to determine which individual or subpools of agents in the collective have a desired activity. Compounds identified by the disclosed methods can be used as therapeutics or lead compounds for drug development for a variety of conditions.

Control reactions can be performed in combination with the libraries. Such optional control reactions are appropriate and can increase the reliability of the screening. Accordingly, disclosed methods can include such a control reaction.

Phenotyping small molecules can be used to identify pathways and biological programs that the small molecule affect or modulate. This information can be used to treat diseases where important biological programs are discovered to be shifted in the disease and where a small molecule is shown to also modulate the same program. Phenotyping small molecules can be used to identify off-target effects of small molecules. Phenotyping small molecules can be used to establish genome-wide transcriptional expression data for each small molecule. The phenotyping can use cultured human cells treated with the small molecules to identify bioactive small molecules. The method can be used for any cell type. Thus, the effects of the small molecules on different cell types can be determined. Simple pattern-matching algorithms can be used that together enable the discovery of functional connections between drugs, genes and diseases through the transitory feature of common gene-expression changes. The method is a general method for phenotyping any small molecule known or subsequently known. The present invention advantageously can be used to determine the effects on phenotypes of many small molecules in parallel.

In certain embodiments, agents for screening are selected from a group of compounds predicted to modulate an identified pathway or cell state in a tumor. In certain embodiments, the present invention provides for gene signature screening. The concept of signature screening was introduced by Stegmaier et al. (Gene expression-based high-throughput screening (GE-HTS) and application to leukemia differentiation. Nature Genet. 36, 257-263 (2004)), who realized that if a gene-expression signature was the proxy for a phenotype of interest, it could be used to find small molecules that effect that phenotype without knowledge of a validated drug target. The signatures or biological programs of the present invention may be used to screen for drugs that reduce the signature or biological program in cells as described herein. The signature or biological program may be used for GE-HTS. In certain embodiments, pharmacological screens may be used to identify drugs that are selectively toxic to cells having a signature.

The Connectivity Map (cmap) is a collection of genome-wide transcriptional expression data from cultured human cells treated with bioactive small molecules and simple pattern-matching algorithms that together enable the discovery of functional connections between drugs, genes and diseases through the transitory feature of common gene-expression changes (see, Lamb et al., The Connectivity Map: Using Gene-Expression Signatures to Connect Small Molecules, Genes, and Disease. Science 29 Sep. 2006: Vol. 313, Issue 5795, pp. 1929-1935, DOI: 10.1126/science. 1132939; and Lamb, J., The Connectivity Map: a new tool for biomedical research. Nature Reviews Cancer January 2007: Vol. 7, pp. 54-60). In certain embodiments, Cmap can be used to screen for small molecules capable of modulating a signature or biological program of the present invention in silico.

Preferably, the therapeutic agent may be administered in a therapeutically effective amount of the active components. The term “therapeutically effective amount” refers to an amount which can elicit a biological or medicinal response in a tissue, system, animal or human that is being sought by a researcher, veterinarian, medical doctor or other clinician, and in particular can prevent or alleviate one or more of the local or systemic symptoms or features of a disease or condition being treated.

For example, in methods for treating cancer in a subject, an effective amount of a combination of inhibitors is any amount that provides an anti-cancer effect, such as reduces or prevents proliferation of a cancer cell or is cytotoxic towards a cancer cell.

### Measuring Responsiveness of the Ex Vivo Model

Responsiveness in the ex vivo model may be measured in a number of ways. In some embodiments, the responsive phenotype is measured by a change in one or more cell types or cell states of the model. The change in one or more cell types of cell states of the model can, in embodiments, indicate reduced fitness of the model or cell death of one or more target cell types in the model. The responsiveness of the model may be performed according to the single-cell RNA analysis on single cells derived from the established system to determine a current phenotype.

In some embodiments, the non-responsive phenotype is measured by no change in model phenotype or a change in one or more cell types or cell states indicating increased growth or fitness of the model or one or more cell types in the model.

In some embodiments, the method of screening may further comprise clonally expanding the one or more cell types exhibiting increased growth or fitness and performing single cell RNA analysis of the clonally expanded cells to identify cell type and/or cell state.

Such methods are described in U.S. Pat. No. 8,637,307 and is herein incorporated by reference in its entirety. For example, the number of T cells may be increased at least about 3-fold (or 4-, 5-, 6-, 7-, 8-, or 9-fold), more preferably at least about 10-fold (or 20-, 30-, 40-, 50-, 60-, 70-, 80-, or 90-fold), more preferably at least about 100-fold, more preferably at least about 1,000 fold, or most preferably at least about 100,000-fold. The numbers of T cells may be expanded using any suitable method known in the art. Exemplary methods of expanding the numbers of cells are described in patent publication No. WO 2003057171, U.S. Pat. No. 8,034,334, and U.S. Patent Application Publication No. 2012/0244133, each of which is incorporated herein by reference.

In one embodiment, ex vivo T cell expansion can be performed by isolation of T cells and subsequent stimulation or activation followed by further expansion. In one embodiment of the invention, the T cells may be stimulated or activated by a single agent. In another embodiment, T cells are stimulated or activated with two agents, one that induces a primary signal and a second that is a co-stimulatory signal. Ligands useful for stimulating a single signal or stimulating a primary signal and an accessory molecule that stimulates a second signal may be used in soluble form. Ligands may be attached to the surface of a cell, to an Engineered Multivalent Signaling Platform (EMSP), or immobilized on a surface. In a preferred embodiment both primary and secondary agents are co-immobilized on a surface, for example a bead or a cell. In one embodiment, the molecule providing the primary activation signal may be a CD3 ligand, and the co-stimulatory molecule may be a CD28 ligand or 4-1BB ligand.

In some embodiments, the ex vivo cell-based model is derived from a subject to be treated.

In some embodiments, the method may further comprise administering the indicated one or more therapeutic agents to the subject. The administration of the one or more therapeutic agents according to the present invention may be carried out in any convenient manner, including by aerosol inhalation, injection, ingestion, transfusion, implantation or transplantation. The therapeutic agent(s) may be administered to a patient subcutaneously, intradermally, intratumorally, intranodally, intramedullary, intramuscularly, by intravenous or intralymphatic injection, or intraperitoneally. In one embodiment, the one or more therapeutic agents of the present invention are preferably administered by intravenous injection.

In some embodiments, the method of therapeutic agents may further comprise administering one or more therapeutic agents based on the identified cell type and/or cell state of the clonally expanded cells.

### Methods for Treating Tumors

In certain embodiments, the present invention provides for one or more therapeutic agents against any one or more targets identified. In certain embodiments, the agents are used to modulate cell types. For example, the agents may be used to modulate cells for adoptive cell transfer or to modulate tumors. In certain embodiments, the one or more agents comprises a small molecule inhibitor, small molecule degrader (e.g., ATTEC, AUTAC, LYTAC, or PROTAC), genetic modifying agent, antibody, antibody fragment, antibody-like protein scaffold, aptamer, protein, or any combination thereof. With regards to methods of treating tumors, mention is made of Benci et al. (Cell 178(4):933-948 (2019)), the contents of which are herein incorporated by reference in their entirety.

The terms “therapeutic agent”, “therapeutic capable agent” or “treatment agent” are used interchangeably and refer to a molecule or compound that confers some beneficial effect upon administration to a subject. The beneficial effect includes enablement of diagnostic determinations; amelioration of a disease, symptom, disorder, or pathological condition; reducing or preventing the onset of a disease, symptom, disorder or condition; and generally counteracting a disease, symptom, disorder or pathological condition.

As used herein, “treatment” or “treating,” or “palliating” or “ameliorating” are used interchangeably. These terms refer to an approach for obtaining beneficial or desired results including but not limited to a therapeutic benefit and/or a prophylactic benefit. By therapeutic benefit is meant any therapeutically relevant improvement in or effect on one or more diseases, conditions, or symptoms under treatment. For prophylactic benefit, the compositions may be administered to a subject at risk of developing a particular disease, condition, or symptom, or to a subject reporting one or more of the physiological symptoms of a disease, even though the disease, condition, or symptom may not have yet been manifested. As used herein “treating” includes ameliorating, curing, preventing it from becoming worse, slowing the rate of progression, or preventing the disorder from re-occurring (i.e., to prevent a relapse).

The term “effective amount” or “therapeutically effective amount” refers to the amount of an agent that is sufficient to effect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. The term also applies to a dose that will provide an image for detection by any one of the imaging methods described herein. The specific dose may vary depending on one or more of: the particular agent chosen, the dosing regimen to be followed, whether it is administered in combination with other compounds, timing of administration, the tissue to be imaged, and the physical delivery system in which it is carried.

### Standard of Care for Pancreatic Cancer

Aspects of the invention involve modifying the therapy within a standard of care based on the detection of any of the biomarkers or tumor subtypes as described herein. In one embodiment, therapy comprising an agent is administered within a standard of care where addition of the agent is synergistic within the steps of the standard of care. In one embodiment, the therapy targets and/or shifts a tumor to a treatment responder phenotype. In one embodiment, the therapy targets tumor cells expressing a gene program. The term “standard of care” as used herein refers to the current treatment that is accepted by medical experts as a proper treatment for a certain type of disease and that is widely used by healthcare professionals. Standard of care is also called best practice, standard medical care, and standard therapy. Standards of care for cancer generally include surgery, lymph node removal, radiation, chemotherapy, targeted therapies, antibodies targeting the tumor, and immunotherapy. Immunotherapy can include checkpoint blockers (CBP), chimeric antigen receptors (CARs), and adoptive T-cell therapy. The standards of care for the most common cancers can be found on the website of National Cancer Institute (www.cancer.gov/cancertopics). A treatment clinical trial is a research study meant to help improve current treatments or obtain information on new treatments for patients with cancer. When clinical trials show that a new treatment is better than the standard treatment, the new treatment may be considered the new standard treatment.

The standard of care for pancreatic cancer includes, surgery, ablation or embolization treatments, radiation therapy, chemotherapy, targeted therapy, immunotherapy, and pain control. Surgery includes potentially curative surgery and palliative surgery. Ablation refers to treatments that destroy tumors, usually with extreme heat or cold. During embolization, substances are injected into an artery to try to block the blood flow to cancer cells, causing them to die. Radiation might be given after surgery (known as adjuvant treatment) to try to lower the chance of the cancer coming back. The radiation is typically given along with chemotherapy, which is together known as chemoradiation or chemoradiotherapy. For borderline resectable tumors, radiation might be given along with chemotherapy before surgery (neoadjuvant treatment) to try to shrink the tumor and make it easier to remove completely. Radiation therapy combined with chemotherapy may be used as part of the main treatment in people whose cancers have grown beyond the pancreas and cannot be removed by surgery (locally advanced/unresectable cancers). Chemotherapy is often part of the treatment for pancreatic cancer and may be used at any stage. Chemotherapy can include Gemcitabine (Gemzar), 5-fluorouracil (5-FU), Oxaliplatin (Eloxatin), Albumin-bound paclitaxel (Abraxane), Capecitabine (Xeloda), Cisplatin, Irinotecan (Camptosar), Paclitaxel (Taxol), and Docetaxel (Taxotere). Targeted therapies specifically target pancreatic cancer specific mutations or changes as compared to normal cells. Targeted therapies include, but are not limited to, EGFR inhibitors (e.g., Erlotinib (Tarceva)), PARP inhibitors (e.g., Olaparib (Lynparza)), and NTRK inhibitors (e.g., larotrectinib (Vitrakvi) and entrectinib (Rozlytrek)). Standard immunotherapy includes checkpoint inhibitors (e.g., PD-1 inhibitors).

### Interferon Signaling

Also within the scope of the invention is a method of treating PDAC tumors comprising administering one or more agents that reduce IFNγ expression in the tumor microenvironment. The term “reduce” refers to decreases below basal levels, e.g., as compared to a control. The term “control” refers to any reference standard suitable to provide a comparison to the expression products in the test sample. In one embodiment, the control comprises obtaining a “control sample” from which expression product levels are detected and compared to the expression product levels from the test sample. Such a control sample may comprise any suitable sample, including but not limited to a sample from a control patient (can be stored sample or previous sample measurement) with a known outcome; normal tissue, fluid, or cells isolated from a subject, such as a normal patient or the patient having a condition of interest.

In some embodiments, the invention also comprises a method of treating PDAC tumors comprising administering one or more agents that shift tumor cell phenotype from a basal or IFNγ phenotype to a classical phenotype, as described herein.

In certain embodiments, reduced IFNγ expression by the tumor microenvironment (e.g., immune cells) results in reduced expression of interferon stimulated genes in the tumor. In certain embodiments, treating PDAC tumors comprises administering one or more agents that reduce interferon response gene expression (e.g., IFI44L, ISG15, IDO1, MT2A, CD274). An interferon-stimulated gene (ISG) is a gene whose expression is stimulated by interferon. Interferon activates the JAK-STAT signaling pathway to induce transcription of ISGs. ISGs can be divided based on what class of interferon they are activated by: type I, type II, or type III interferon. The type II interferon class only has one cytokine (IFN-γ), which has some antiviral activity, but is more important in establishing cellular immunity through activating macrophages and promoting major histocompatibility complex (MHC) class II.

### IDO1 Inhibitors

In certain embodiments, treatment comprises an IDO1 inhibitor. In certain embodiments an IDO1 inhibitor is administered in combination with a CPB therapy (e.g., anti-PDL1 (CD274) or -PD-1). IDO1 modulates immune cell function to a suppressive phenotype and is therefore partially accountable for tumor escape from host immune surveillance. The enzyme indoleamine 2,3-dioxygenase 1 (IDO1) degrades the essential amino acid tryptophan into kynurenine and other metabolites. These metabolites and the paucity of tryptophan leads to suppression of effector T-cell function and augmented differentiation of regulatory T cells.

In certain embodiments, the IDO1 inhibitor includes, but is not limited to indoximod, epacadostat, navoximod, PF-06840003, BMS-986205, and microRNA-153 (miR-153) (see, e.g., Liu, M., Wang, X., Wang, L. et al. Targeting the IDO1 pathway in cancer: from bench to bedside. J Hematol Oncol 11, 100 (2018)). IDO1 inhibitors may also include any inhibitors as described in US patent publication US20170037125A1.

CSF1R Signaling inhibitors

In certain embodiments, CSF1R signaling is inhibited. In certain embodiments CSF1R-blocking antibodies are administered to a PDAC patient (see, e.g., Wang, Q., Lu, Y., Li, R. et al. Therapeutic effects of CSF1R-blocking antibodies in multiple myeloma. Leukemia 32, 176-183 (2018)). A variety of small molecules and monoclonal antibodies (mAbs) directed at CSF1R or its ligand CSF1 are in clinical development both as monotherapy and in combination with standard treatment modalities such as chemotherapy as well as other cancer-immunotherapy approaches (see, e.g., Cannarile, M. A., Weisser, M., Jacob, W. et al. Colony-stimulating factor 1 receptor (CSF1R) inhibitors in cancer therapy.j. immunotherapy cancer 5, 53 (2017). IL34 inhibitors have also been described (see, e.g., Ge, Yun et al. “Immunomodulation of Interleukin-34 and its Potential Significance as a Disease Biomarker and Therapeutic Target.” International journal of biological sciences vol. 15, 9 1835-1845. 20 Jul. 2019; and Noy R, Pollard J W Tumor-associated macrophages; from mechanisms to therapy. Immunity. 2014; 41(1):49-61).

### Combination Treatments

In certain embodiments, targeting combinations may provide for enhanced or otherwise previously unknown activity in the treatment of disease. In certain embodiments, an agent against one of the targets in a combination may already be known or used clinically. In certain embodiments, targeting the combination may require less of the agent as compared to the current standard of care and provide for less toxicity and improved treatment.

In certain embodiments, one or more agents are administered in a combination therapy. In certain embodiments, treatment with an agent that interferes with CSF1R signaling may alter the tumor microenvironment, such that it is less tumor supportive and anti-inflammatory, thus providing for more sensitivity to an immunotherapy (e.g., ACT, checkpoint blockade therapy), chemotherapy, or targeted therapies.

In certain embodiments, reducing IFNγ expression or ISG expression may make a tumor more responsive to a therapy, such as immunotherapy, chemotherapy, or targeted therapies.

### Pharmaceutical Compositions

Pharmaceutical compositions are also contemplated within the scope of the disclosure. In some cases, one or more modulating agents may be comprised in a pharmaceutical composition or formulation.

A “pharmaceutical composition” refers to a composition that usually contains an excipient, such as a pharmaceutically acceptable carrier that is conventional in the art and that is suitable for administration to cells or to a subject. Pharmaceutically acceptable as used throughout this specification is consistent with the art and means compatible with the other ingredients of a pharmaceutical composition and not deleterious to the recipient thereof.

As used herein, “carrier” or “excipient” includes any and all solvents, diluents, buffers (such as, e.g., neutral buffered saline or phosphate buffered saline), solubilisers, colloids, dispersion media, vehicles, fillers, chelating agents (such as, e.g., EDTA or glutathione), amino acids (such as, e.g., glycine), proteins, disintegrants, binders, lubricants, wetting agents, emulsifiers, sweeteners, colorants, flavourings, aromatisers, thickeners, agents for achieving a depot effect, coatings, antifungal agents, preservatives, stabilisers, antioxidants, tonicity controlling agents, absorption delaying agents, and the like. The use of such media and agents for pharmaceutical active components is well known in the art. Such materials should be non-toxic and should not interfere with the activity of the cells or active components.

The precise nature of the carrier or excipient or other material will depend on the route of administration. For example, the composition may be in the form of a parenterally acceptable aqueous solution, which is pyrogen-free and has suitable pH, isotonicity and stability. For general principles in medicinal formulation, the reader is referred to Cell Therapy: Stem Cell Transplantation, Gene Therapy, and Cellular Immunotherapy, by G. Morstyn & W. Sheridan eds., Cambridge University Press, 1996; and Hematopoietic Stem Cell Therapy, E. D. Ball, J. Lister & P. Law, Churchill Livingstone, 2000.

It will be appreciated that administration of therapeutic entities in accordance with the invention will be administered with suitable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences (15th ed, Mack Publishing Company, Easton, Pa. (1975)), particularly Chapter 87 by Blaug, Seymour, therein. These formulations include, for example, powders, pastes, ointments, jellies, waxes, oils, lipids, lipid (cationicoranionic) containing vesicles (such as Lipofectin™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. Any of the foregoing mixtures may be appropriate in treatments and therapies in accordance with the present invention, provided that the active ingredient in the formulation is not inactivated by the formulation and the formulation is physiologically compatible and tolerable with the route of administration. See also Baldrick P. “Pharmaceutical excipient development: the need for preclinical guidance.” Regul. Toxicol Pharmacol. 32(2):210-8(2000), Wang W. “Lyophilization and development of solid protein pharmaceuticals.” Int. J. Pharm. 203(1-2):1-60(2000), Charman, W N “Lipids, lipophilic drugs, and oral drug delivery-some emerging concepts.” J Pharm Sci. 89(8):967-78(2000), Powell et al. “Compendium of excipients for parenteral formulations” PDAJ Pharm Sci Technol. 52:238-311(1998) and the citations therein for additional information related to formulations, excipients and carriers well known to pharmaceutical chemists.

The medicaments are prepared in a manner known to those skilled in the art, for example, by means of conventional dissolving, lyophilizing, mixing, granulating or confectioning processes. Methods well known in the art for making formulations are found, for example, in Remington: The Science and Practice of Pharmacy, 20th ed., ed. A. R. Gennaro, 2000, Lippincott Williams & Wilkins, Philadelphia, and Encyclopedia of Pharmaceutical Technology, eds. J. Swarbrick and J. C. Boylan, 1988-1999, Marcel Dekker, New York.

Administration of medicaments of the invention may be by any suitable means that results in a compound concentration that is effective for treating or inhibiting (e.g., by delaying) the development of a disease. The compound is admixed with a suitable carrier substance, e.g., a pharmaceutically acceptable excipient that preserves the therapeutic properties of the compound with which it is administered. One exemplary pharmaceutically acceptable excipient is physiological saline. The suitable carrier substance is generally present in an amount of 1-95% by weight of the total weight of the medicament. The medicament may be provided in a dosage form that is suitable for administration. Thus, the medicament may be in form of, e.g., tablets, capsules, pills, powders, granulates, suspensions, emulsions, solutions, gels including hydrogels, pastes, ointments, creams, plasters, drenches, delivery devices, injectables, implants, sprays, or aerosols.

The modulating agents may be used in a pharmaceutical composition when combined with a pharmaceutically acceptable carrier. Such compositions comprise a therapeutically-effective amount of the agent and a pharmaceutically acceptable carrier. Such a composition may also further comprise (in addition to an agent and a carrier) diluents, fillers, salts, buffers, stabilizers, solubilizers, and other materials well known in the art. Compositions comprising the agent can be administered in the form of salts provided the salts are pharmaceutically acceptable. Salts may be prepared using standard procedures known to those skilled in the art of synthetic organic chemistry.

The term “pharmaceutically acceptable salts” refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like. The term “pharmaceutically acceptable salt” further includes all acceptable salts such as acetate, lactobionate, benzenesulfonate, laurate, benzoate, malate, bicarbonate, maleate, bisulfate, mandelate, bitartrate, mesylate, borate, methylbromide, bromide, methylnitrate, calcium edetate, methylsulfate, camsylate, mucate, carbonate, napsylate, chloride, nitrate, clavulanate, N-methylglucamine, citrate, ammonium salt, dihydrochloride, oleate, edetate, oxalate, edisylate, pamoate (embonate), estolate, palmitate, esylate, pantothenate, fumarate, phosphate/diphosphate, gluceptate, polygalacturonate, gluconate, salicylate, glutamate, stearate, glycollylarsanilate, sulfate, hexylresorcinate, subacetate, hydrabamine, succinate, hydrobromide, tannate, hydrochloride, tartrate, hydroxynaphthoate, teoclate, iodide, tosylate, isothionate, triethiodide, lactate, panoate, valerate, and the like which can be used as a dosage form for modifying the solubility or hydrolysis characteristics or can be used in sustained release or pro-drug formulations. It will be understood that, as used herein, references to specific agents (e.g., neuromedin U receptor agonists or antagonists), also include the pharmaceutically acceptable salts thereof.

Methods of administrating the pharmacological compositions, including agonists, antagonists, antibodies or fragments thereof, to an individual include, but are not limited to, intradermal, intrathecal, intramuscular, intraperitoneal, intravenous, subcutaneous, intranasal, epidural, by inhalation, and oral routes. The compositions can be administered by any convenient route, for example by infusion or bolus injection, by absorption through epithelial or mucocutaneous linings (for example, oral mucosa, rectal and intestinal mucosa, and the like), ocular, and the like and can be administered together with other biologically-active agents. Administration can be systemic or local. In addition, it may be advantageous to administer the composition into the central nervous system by any suitable route, including intraventricular and intrathecal injection. Pulmonary administration may also be employed by use of an inhaler or nebulizer, and formulation with an aerosolizing agent. It may also be desirable to administer the agent locally to the area in need of treatment; this may be achieved by, for example, and not by way of limitation, local infusion during surgery, topical application, by injection, by means of a catheter, by means of a suppository, or by means of an implant.

Various delivery systems are known and can be used to administer the pharmacological compositions including, but not limited to, encapsulation in liposomes, microparticles, microcapsules; minicells; polymers; capsules; tablets; and the like. In one embodiment, the agent may be delivered in a vesicle, in particular a liposome. In a liposome, the agent is combined, in addition to other pharmaceutically acceptable carriers, with amphipathic agents such as lipids which exist in aggregated form as micelles, insoluble monolayers, liquid crystals, or lamellar layers in aqueous solution. Suitable lipids for liposomal formulation include, without limitation, monoglycerides, diglycerides, sulfatides, lysolecithin, phospholipids, saponin, bile acids, and the like. Preparation of such liposomal formulations is within the level of skill in the art, as disclosed, for example, in U.S. Pat. Nos. 4,837,028 and 4,737,323. In yet another embodiment, the pharmacological compositions can be delivered in a controlled release system including, but not limited to: a delivery pump (See, for example, Saudek, et al., New Engl. J. Med. 321: 574 (1989) and a semi-permeable polymeric material (See, for example, Howard, et al., J. Neurosurg. 71: 105 (1989)). Additionally, the controlled release system can be placed in proximity of the therapeutic target (e.g., a tumor or infected tissue), thus requiring only a fraction of the systemic dose. See, for example, Goodson, In: Medical Applications of Controlled Release, 1984. (CRC Press, Boca Raton, Fla.).

The amount of the agents which will be effective in the treatment of a particular disorder or condition will depend on the nature of the disorder or condition, and may be determined by standard clinical techniques by those of skill within the art. In addition, in vitro assays may optionally be employed to help identify optimal dosage ranges. The precise dose to be employed in the formulation will also depend on the route of administration, and the overall seriousness of the disease or disorder, and should be decided according to the judgment of the practitioner and each patient's circumstances. Ultimately, the attending physician will decide the amount of the agent with which to treat each individual patient. In certain embodiments, the attending physician will administer low doses of the agent and observe the patient's response. Larger doses of the agent may be administered until the optimal therapeutic effect is obtained for the patient, and at that point the dosage is not increased further. In general, the daily dose range of a drug lie within the range known in the art for a particular drug or biologic. Effective doses may be extrapolated from dose-response curves derived from in vitro or animal model test systems. Ultimately the attending physician will decide on the appropriate duration of therapy using compositions of the present invention. Dosage will also vary according to the age, weight and response of the individual patient.

Methods for administering antibodies for therapeutic use is well known to one skilled in the art. In certain embodiments, small particle aerosols of antibodies or fragments thereof may be administered (see e.g., Piazza et al., J. Infect. Dis., Vol. 166, pp. 1422-1424, 1992; and Brown, Aerosol Science and Technology, Vol. 24, pp. 45-56, 1996). In certain embodiments, antibodies are administered in metered-dose propellant driven aerosols. In certain embodiments, antibodies may be administered in liposomes, i.e., immunoliposomes (see, e.g., Maruyama et al., Biochim. Biophys. Acta, Vol. 1234, pp. 74-80, 1995). In certain embodiments, immunoconjugates, immunoliposomes or immunomicrospheres containing an agent of the present invention is administered by inhalation.

In certain embodiments, antibodies may be topically administered to mucosa, such as the oropharynx, nasal cavity, respiratory tract, gastrointestinal tract, eye such as the conjunctival mucosa, vagina, urogenital mucosa, or for dermal application. In certain embodiments, antibodies are administered to the nasal, bronchial or pulmonary mucosa. In order to obtain optimal delivery of the antibodies to the pulmonary cavity in particular, it may be advantageous to add a surfactant such as a phosphoglyceride, e.g. phosphatidylcholine, and/or a hydrophilic or hydrophobic complex of a positively or negatively charged excipient and a charged antibody of the opposite charge.

Other excipients suitable for pharmaceutical compositions intended for delivery of antibodies to the respiratory tract mucosa may be a) carbohydrates, e.g., monosaccharides such as fructose, galactose, glucose. D-mannose, sorbiose, and the like; disaccharides, such as lactose, trehalose, cellobiose, and the like; cyclodextrins, such as 2-hydroxypropyl-β-cyclodextrin; and polysaccharides, such as raffinose, maltodextrins, dextrans, and the like; b) amino acids, such as glycine, arginine, aspartic acid, glutamic acid, cysteine, lysine and the like; c) organic salts prepared from organic acids and bases, such as sodium citrate, sodium ascorbate, magnesium gluconate, sodium gluconate, tromethamine hydrochloride, and the like: d) peptides and proteins, such as aspartame, human serum albumin, gelatin, and the like; e) alditols, such mannitol, xylitol, and the like, and f) polycationic polymers, such as chitosan or a chitosan salt or derivative.

Examples of solvents are e.g. water, alcohols, vegetable or marine oils (e.g. edible oils like almond oil, castor oil, cacao butter, coconut oil, corn oil, cottonseed oil, linseed oil, olive oil, palm oil, peanut oil, poppy seed oil, rapeseed oil, sesame oil, soybean oil, sunflower oil, and tea seed oil), mineral oils, fatty oils, liquid paraffin, polyethylene glycols, propylene glycols, glycerol, liquid polyalkylsiloxanes, and mixtures thereof.

Examples of buffering agents are e.g. citric acid, acetic acid, tartaric acid, lactic acid, hydrogenphosphoric acid, diethyl amine etc. Suitable examples of preservatives for use in compositions are parabenes, such as methyl, ethyl, propyl p-hydroxybenzoate, butylparaben, isobutylparaben, isopropylparaben, potassium sorbate, sorbic acid, benzoic acid, methyl benzoate, phenoxyethanol, bronopol, bronidox, MDM hydantoin, iodopropynyl butylcarbamate, EDTA, benzalconium chloride, and benzylalcohol, or mixtures of preservatives.

Examples of humectants are glycerin, propylene glycol, sorbitol, lactic acid, urea, and mixtures thereof.

Examples of antioxidants are butylated hydroxy anisole (BHA), ascorbic acid and derivatives thereof, tocopherol and derivatives thereof, cysteine, and mixtures thereof.

Examples of emulsifying agents are naturally occurring gums, e.g. gum acacia or gum tragacanth; naturally occurring phosphatides, e.g. soybean lecithin, sorbitan monooleate derivatives: wool fats; wool alcohols; sorbitan esters; monoglycerides; fatty alcohols; fatty acid esters (e.g. triglycerides of fatty acids); and mixtures thereof.

Examples of suspending agents are e.g. celluloses and cellulose derivatives such as, e.g., carboxymethyl cellulose, hydroxyethylcellulose, hydroxypropylcellulose, hydroxypropylmethylcellulose, carraghenan, acacia gum, arabic gum, tragacanth, and mixtures thereof.

Examples of gel bases, viscosity-increasing agents or components which are able to take up exudate from a wound are: liquid paraffin, polyethylene, fatty oils, colloidal silica or aluminum, zinc soaps, glycerol, propylene glycol, tragacanth, carboxyvinyl polymers, magnesium-aluminum silicates, Carbopol®, hydrophilic polymers such as, e.g. starch or cellulose derivatives such as, e.g., carboxymethylcellulose, hydroxyethylcellulose and other cellulose derivatives, water-swellable hydrocolloids, carragenans, hyaluronates (e.g. hyaluronate gel optionally containing sodium chloride), and alginates including propylene glycol alginate.

Examples of ointment bases are e.g. beeswax, paraffin, cetanol, cetyl palmitate, vegetable oils, sorbitan esters of fatty acids (Span), polyethylene glycols, and condensation products between sorbitan esters of fatty acids and ethylene oxide, e.g. polyoxyethylene sorbitan monooleate (Tween).

Examples of hydrophobic or water-emulsifying ointment bases are paraffins, vegetable oils, animal fats, synthetic glycerides, waxes, lanolin, and liquid polyalkylsiloxanes. Examples of hydrophilic ointment bases are solid macrogols (polyethylene glycols). Other examples of ointment bases are triethanolamine soaps, sulphated fatty alcohol and polysorbates.

Examples of other excipients are polymers such as carmelose, sodium carmelose, hydroxypropylmethylcellulose, hydroxyethylcellulose, hydroxypropylcellulose, pectin, xanthan gum, locust bean gum, acacia gum, gelatin, carbomer, emulsifiers like vitamin E, glyceryl stearates, cetanyl glucoside, collagen, carrageenan, hyaluronates and alginates and chitosans.

There are a variety of techniques available for introducing nucleic acids into viable cells. The techniques vary depending upon whether the nucleic acid is transferred into cultured cells in vitro, or in vivo in the cells of the intended host. Techniques suitable for the transfer of nucleic acid into mammalian cells in vitro include the use of liposomes, electroporation, microinjection, cell fusion, DEAE-dextran, the calcium phosphate precipitation method, etc. The currently preferred in vivo gene transfer techniques include transduction with viral (typically lentivirus, adeno associated virus (AAV) and adenovirus) vectors.

The pharmaceutical composition can be applied parenterally, rectally, orally or topically. Preferably, the pharmaceutical composition may be used for intravenous, intramuscular, subcutaneous, peritoneal, peridural, rectal, nasal, pulmonary, mucosal, or oral application. In a preferred embodiment, the pharmaceutical composition according to the invention is intended to be used as an infuse. The skilled person will understand that compositions which are to be administered orally or topically will usually not comprise cells, although it may be envisioned for oral compositions to also comprise cells, for example when gastro-intestinal tract indications are treated. Each of the cells or active components (e.g., modulants, immunomodulants, antigens) as discussed herein may be administered by the same route or may be administered by a different route. By means of example, and without limitation, cells may be administered parenterally and other active components may be administered orally.

Liquid pharmaceutical compositions may generally include a liquid carrier such as water or a pharmaceutically acceptable aqueous solution. For example, physiological saline solution, tissue or cell culture media, dextrose or other saccharide solution or glycols such as ethylene glycol, propyleneglycol or polyethylene glycol may be included. The composition may include one or more cell protective molecules, cell regenerative molecules, growth factors, anti-apoptotic factors or factors that regulate gene expression in the cells. Such substances may render the cells independent of their environment. Such pharmaceutical compositions may contain further components ensuring the viability of the cells therein. For example, the compositions may comprise a suitable buffer system (e.g., phosphate or carbonate buffer system) to achieve desirable pH, more usually near neutral pH, and may comprise sufficient salt to ensure isoosmotic conditions for the cells to prevent osmotic stress. For example, suitable solution for these purposes may be phosphate-buffered saline (PBS), sodium chloride solution, Ringer's Injection or Lactated Ringer's Injection, as known in the art. Further, the composition may comprise a carrier protein, e.g., albumin (e.g., bovine or human albumin), which may increase the viability of the cells.

Further suitably pharmaceutically acceptable carriers or additives are well known to those skilled in the art and for instance may be selected from proteins such as collagen or gelatine, carbohydrates such as starch, polysaccharides, sugars (dextrose, glucose and sucrose), cellulose derivatives like sodium or calcium carboxymethylcellulose, hydroxypropyl cellulose or hydroxypropylmethyl cellulose, pregeletanized starches, pectin agar, carrageenan, clays, hydrophilic gums (acacia gum, guar gum, arabic gum and xanthan gum), alginic acid, alginates, hyaluronic acid, polyglycolic and polylactic acid, dextran, pectins, synthetic polymers such as water-soluble acrylic polymer or polyvinylpyrrolidone, proteoglycans, calcium phosphate and the like.

If desired, cell preparation can be administered on a support, scaffold, matrix or material to provide improved tissue regeneration. For example, the material can be a granular ceramic, or a biopolymer such as gelatine, collagen, or fibrinogen. Porous matrices can be synthesized according to standard techniques (e.g., Mikos et al., Biomaterials 14: 323, 1993; Mikos et al., Polymer 35:1068, 1994; Cook et al., J. Biomed. Mater. Res. 35:513, 1997). Such support, scaffold, matrix or material may be biodegradable or non-biodegradable. Hence, the cells may be transferred to and/or cultured on suitable substrate, such as porous or non-porous substrate, to provide for implants.

For example, cells that have proliferated, or that are being differentiated in culture dishes, can be transferred onto three-dimensional solid supports in order to cause them to multiply and/or continue the differentiation process by incubating the solid support in a liquid nutrient medium of the invention, if necessary. Cells can be transferred onto a three-dimensional solid support, e.g. by impregnating the support with a liquid suspension containing the cells. The impregnated supports obtained in this way can be implanted in a human subject. Such impregnated supports can also be re-cultured by immersing them in a liquid culture medium, prior to being finally implanted. The three-dimensional solid support needs to be biocompatible so as to enable it to be implanted in a human. It may be biodegradable or non-biodegradable.

The cells or cell populations can be administered in a manner that permits them to survive, grow, propagate and/or differentiate towards desired cell types (e.g. differentiation) or cell states. The cells or cell populations may be grafted to or may migrate to and engraft within the intended organ. In certain embodiments, a pharmaceutical cell preparation as taught herein may be administered in a form of liquid composition. In embodiments, the cells or pharmaceutical composition comprising such can be administered systemically, topically, within an organ or at a site of organ dysfunction or lesion.

The isolated cells, cells, or populations thereof as disclosed throughout this specification may be suitably cultured or cultivated in vitro. The term “in vitro” generally denotes outside, or external to, a body, e.g., an animal or human body. The term encompasses “ex vivo”. The terms “culturing” or “cell culture” are common in the art and broadly refer to maintenance of cells and potentially expansion (proliferation, propagation) of cells in vitro. Typically, animal cells, such as mammalian cells, such as human cells, are cultured by exposing them to (i.e., contacting them with) a suitable cell culture medium in a vessel or container adequate for the purpose (e.g., a 96-, 24-, or 6-well plate, a T-25, T-75, T-150 or T-225 flask, or a cell factory), at art-known conditions conducive to in vitro cell culture, such as temperature of 37° C., 5% v/v C02 and >95% humidity. The term “medium” as used herein broadly encompasses any cell culture medium conducive to maintenance of cells, preferably conducive to proliferation of cells. Typically, the medium will be a liquid culture medium, which facilitates easy manipulation (e.g., decantation, pipetting, centrifugation, filtration, and such) thereof.

Further embodiments are illustrated in the following Examples which are given for illustrative purposes only and are not intended to limit the scope of the invention.

### EXAMPLES

### Example 1—Organoid Modeling and Single-Cell Sequencing from Metastatic Biopsies

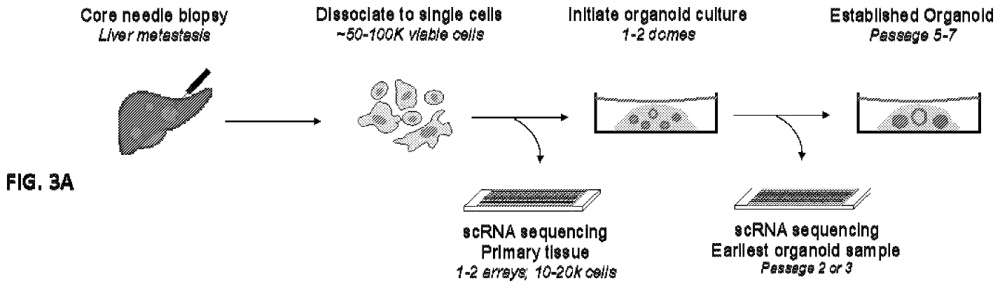

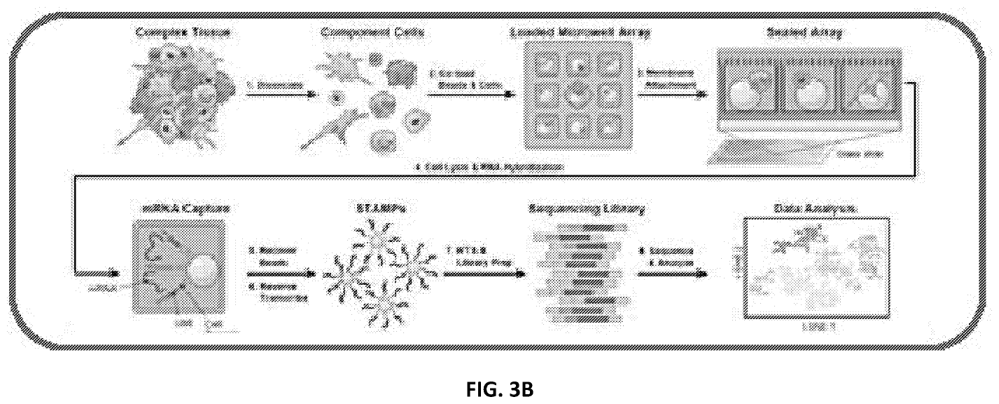

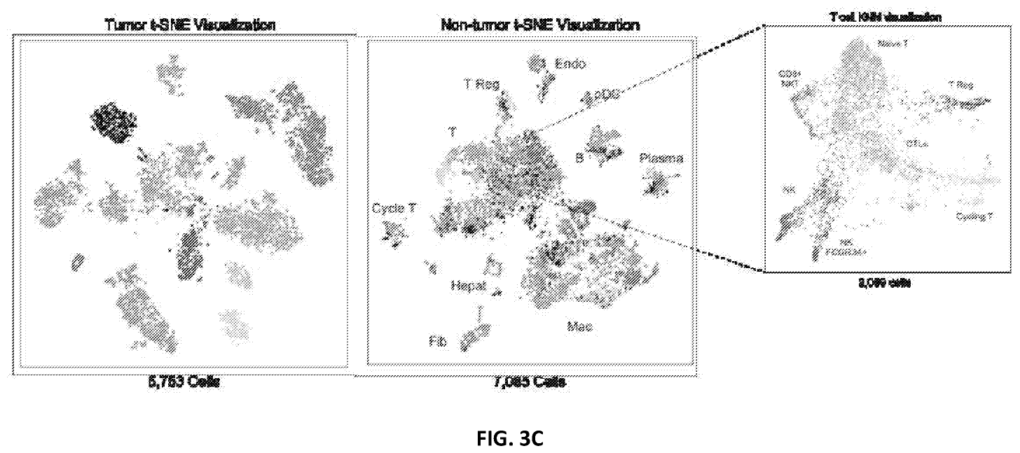

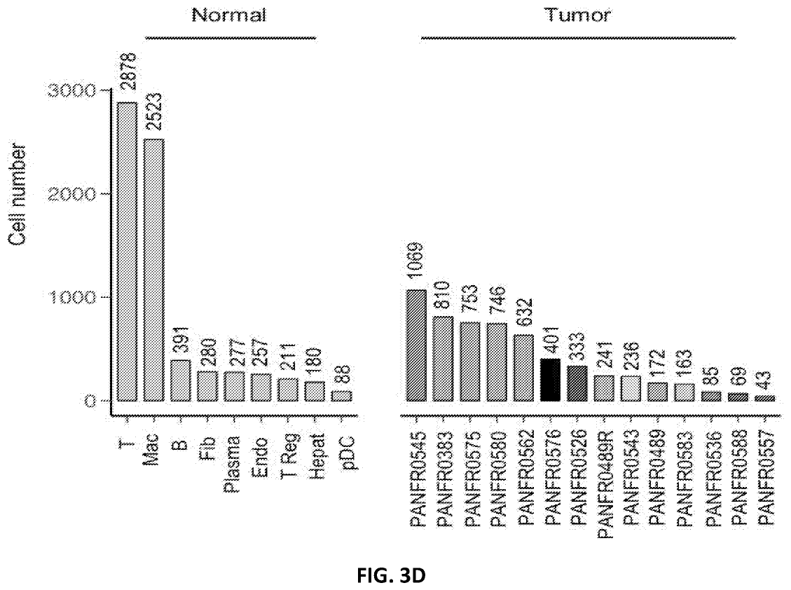

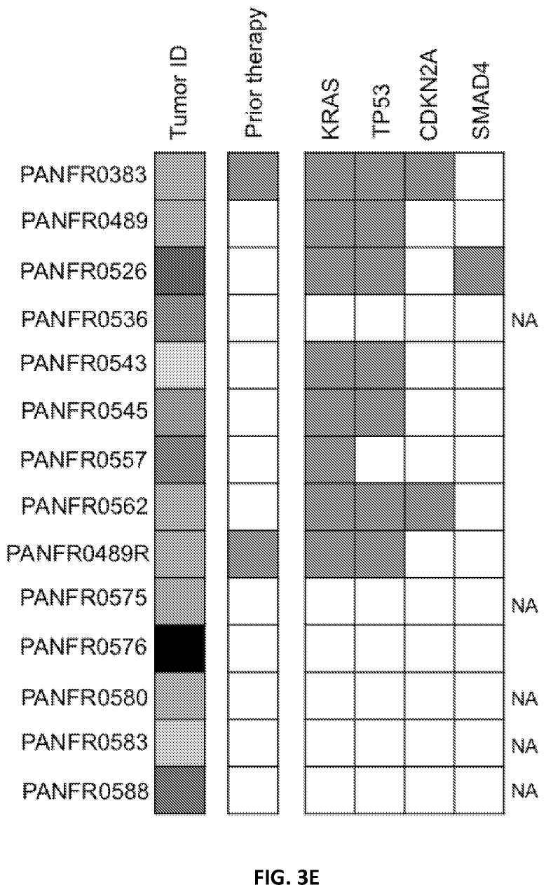

Applicants studied the microenvironment of core-needle biopsies from the liver metastatic site of PDAC patients. Samples were split for scRNA-seq using Seq-Well (Gierahn et al. Nat Methods 14:395-398 (2017)) and organoid generation using tissue specific dissociation and culturing techniques developed in the Hahn lab. Organoid samples were periodically dissociated and profiled at early (P1-3) and late (P5-7) passages to examine model fidelity (FIG. 3A). Applicants profiled samples from 14 patients, with successful organoids seeded from 10. The dataset includes over 20,000 cells including primary tumor (FIG. 3C, left graph), immune cells and fibroblasts as well as organoid samples (FIG. 3C, middle graph and magnified portion on the right).

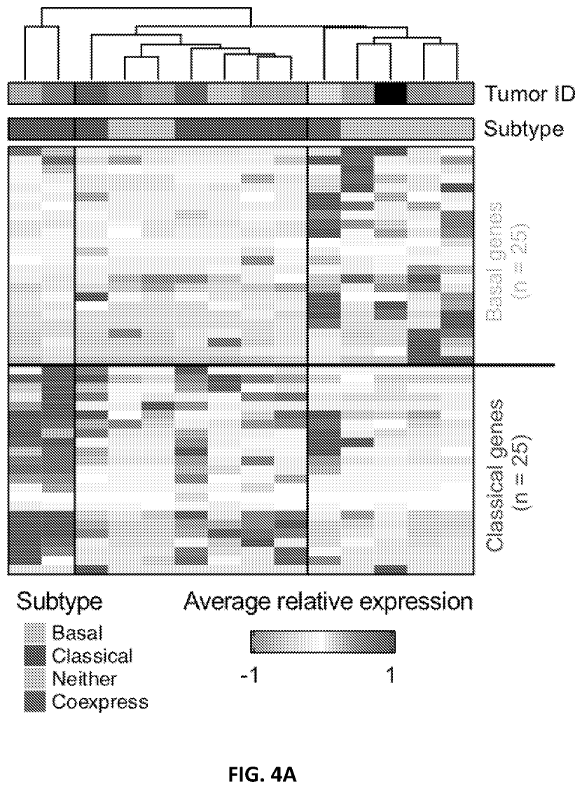

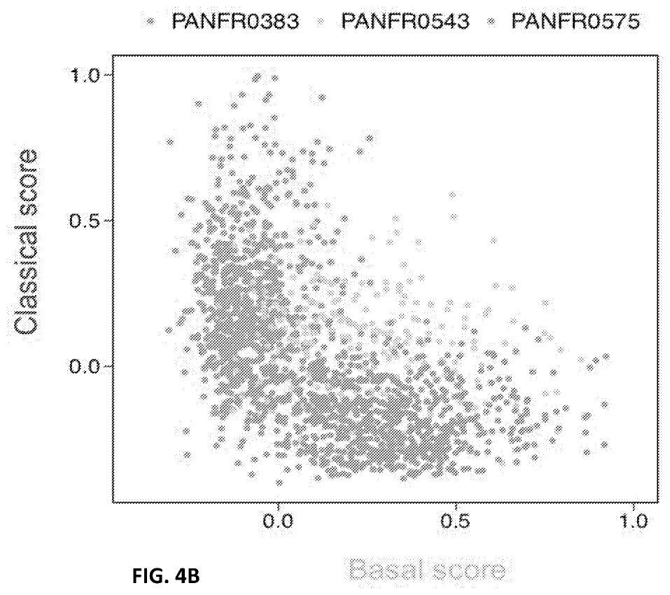

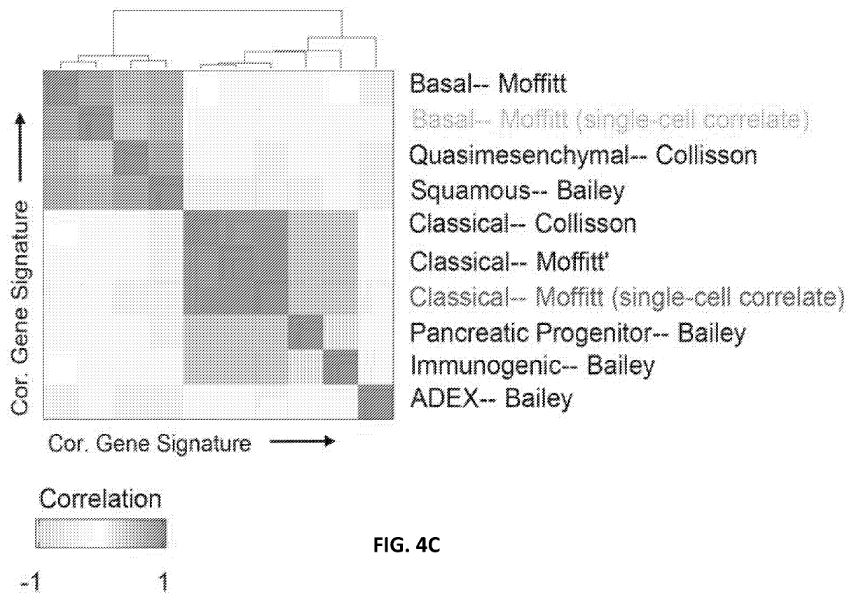

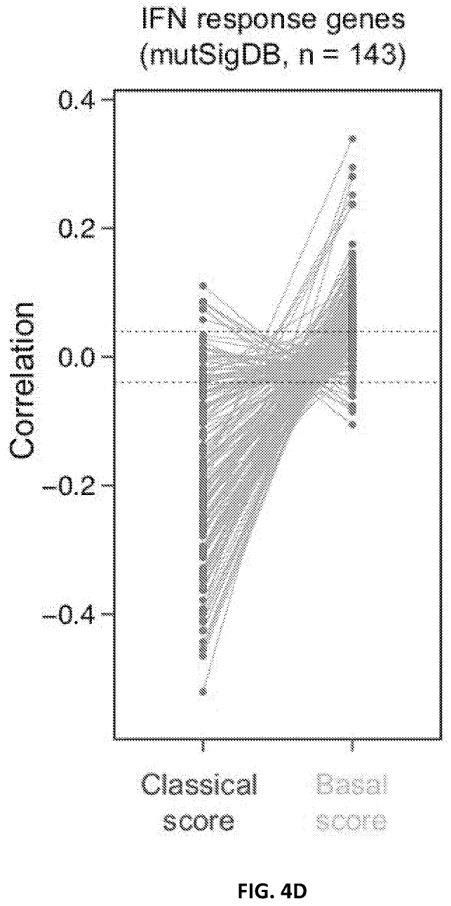

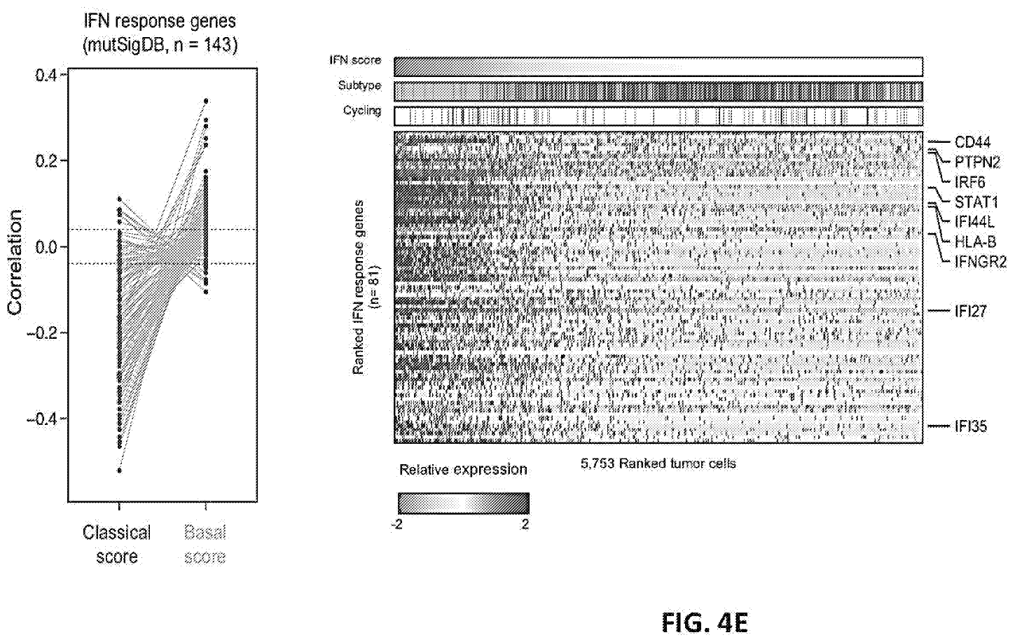

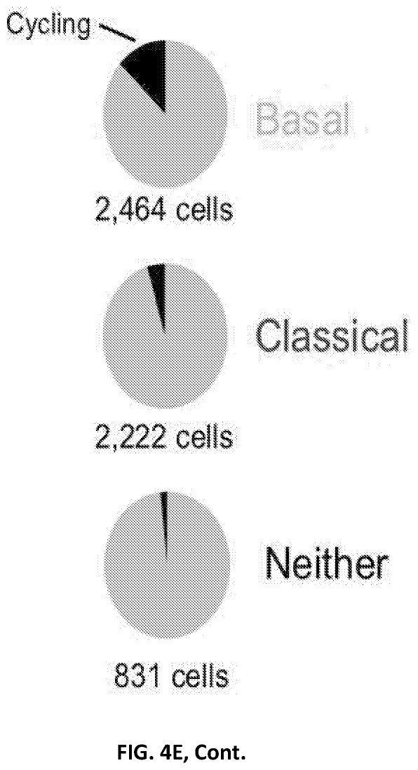

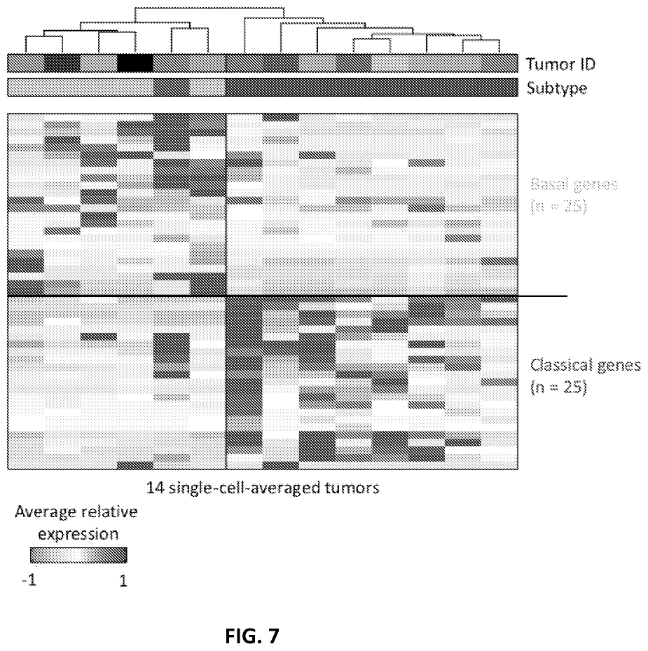

After sequencing, cells were trimmed based on gene (500) and transcript count (800) thresholds. Cell types were then called using SNN clustering and analysis of cluster marker genes. Applicants found that tumor cells tend to cluster by patient (FIG. 3C, left) whereas non-tumor cells are admixed and cluster by cell type (FIG. 3C, middle). To confirm tumor assignments, Applicants leveraged the instability in cancer genomes to distinguish malignant epithelial cells by using InferCNV, a package that arranges gene expression geographically by chromosome to identify large regions of increased or decreased expression to infer large chromosomal gains and losses (Patel et al. Science 344:1396-1401 (2014)). Further unbiased analysis (PCA) of the tumor cells revealed that patient samples largely fall into the basal and classical spectrum described in the Moffitt paper (Nat Genet 47:1168-1178 (2015)) (FIG. 7). Clustering these profiles using Ward's method revealed the expected breakdown for the majority of tumors into basal and classical subtypes. Applicants also observed two that did not fit the expected subtypes and one that co-expresses features of both basal and classical (FIG. 4A). Applicants also confirmed, as previously alluded to (Collisson et al. Nat Rev Gastroenterol Heptol 16:207-220 (2019)), that squamous, basal, and quasimesenchymal subtypes share features of transcriptional state as cells that tend to score high for one, score well for the others. ADEX (Bailey et al. Nature 531:47-52 (2016)) and pancreatic progenitor (Bailey et al. Nature 531:47-52 (2016)) did not score consistently well in these data, possibly reflecting features of the transcriptional states represented in metastatic disease vs the primary site. Immunogenic scores poorly, as expected, since this subtype likely reflects contamination from immune infiltrate in the bulk samples used for its derivation (Collisson et al. Nat Rev Gastroenterol Heptol 16:207-220 (2019)). The “single-cell correlates” are computed by correlating each single cell's basal or classical score (Moffitt et al. Nat Genet 47:1168-1178 (2015)) to the complete expression matrix for all tumor cells (FIG. 4C).

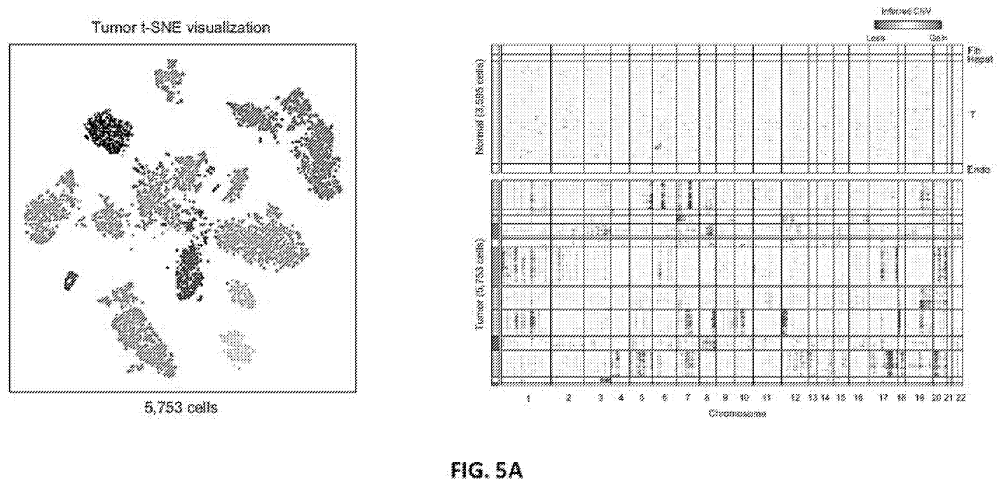

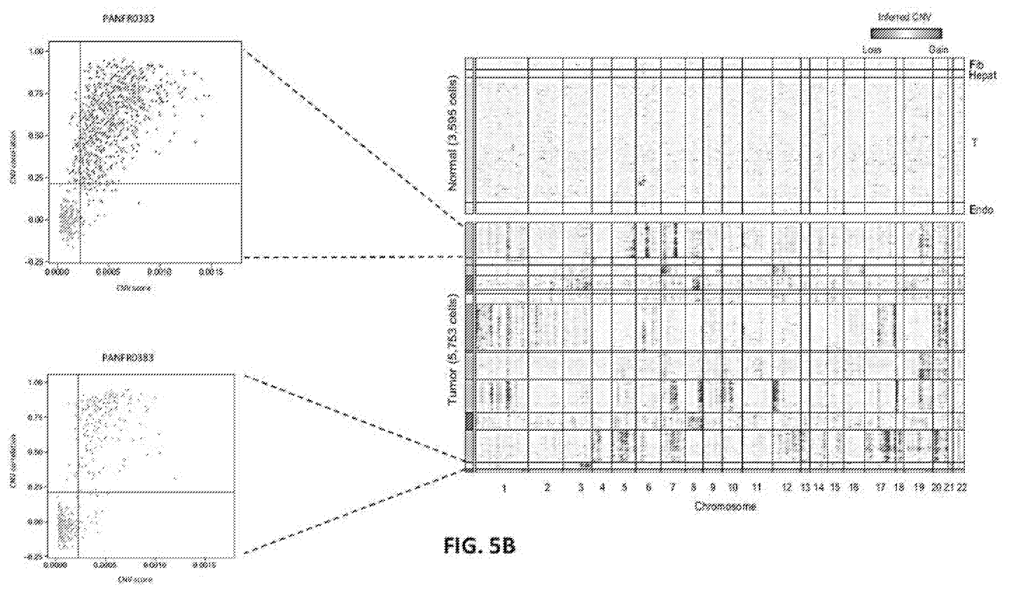

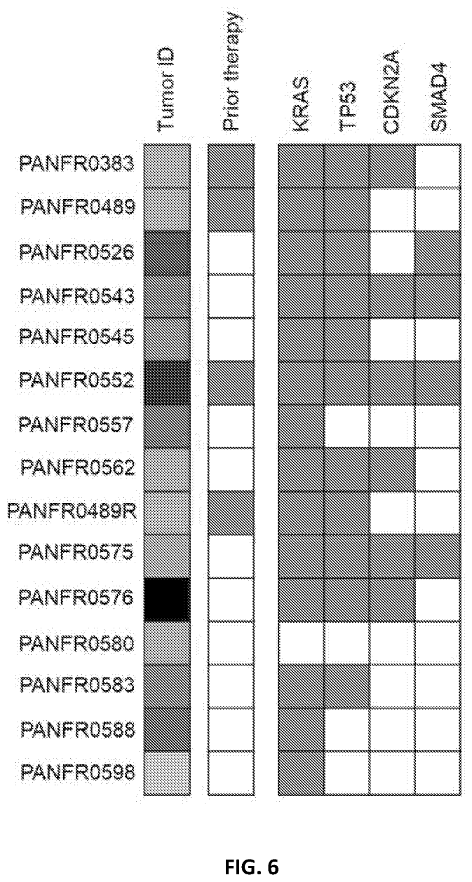

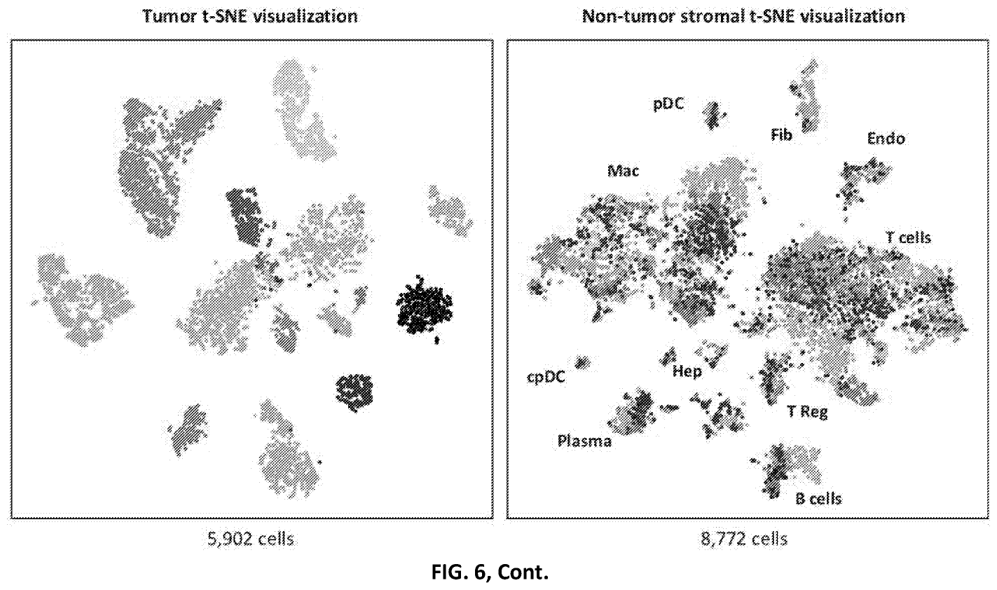

To confirm tumor cell identity in heterogeneous samples, Applicants arranged gene expression geographically by chromosome. Large regions of increased or decreased expression relative to normal cells (FIG. 5B, top) helped to infer large chromosomal gains and losses in each patient's cells (tumor cells, FIG. 5B, bottom). To summarize the copy number variation (CNV) analysis for each tumor cell, Applicants used two metrics. First, the sum of each putative malignant cell's gene-by-gene mean-squared divergence in CNV signal (CNV score) was computed. Next, within each tumor, Applicants identified the top 5% of altered cells by CNV score and averaged their profiles to create a reference score of “high-confidence malignant” for each tumor. Tumor and non-tumor stromal cells were then correlated to this representative score giving an assessment of each cell's relation to the most altered cells in each tumor. Two representative patients are shown in FIG. 5A, PANF0383 (810 cells, top) and PANF0583 (163 cells, bottom).

### Example 2—Matched Primary Tumor Samples and Early Passage Organoids Reveal Significant Divergence in Transcriptional Phenotype

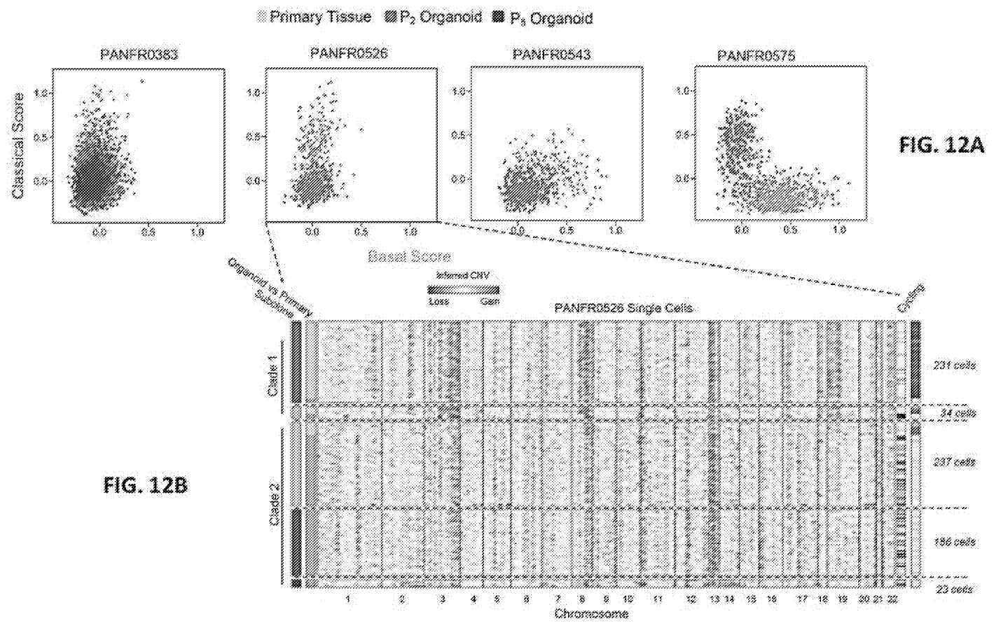

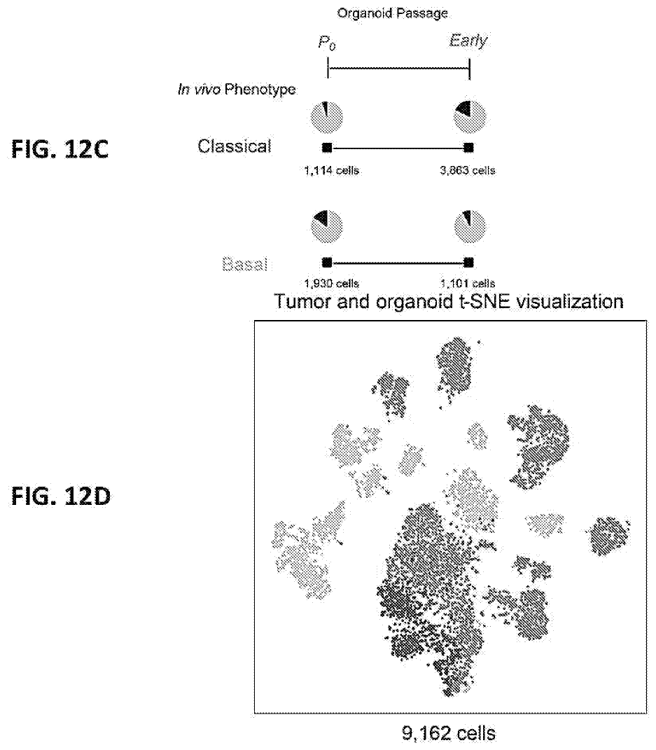

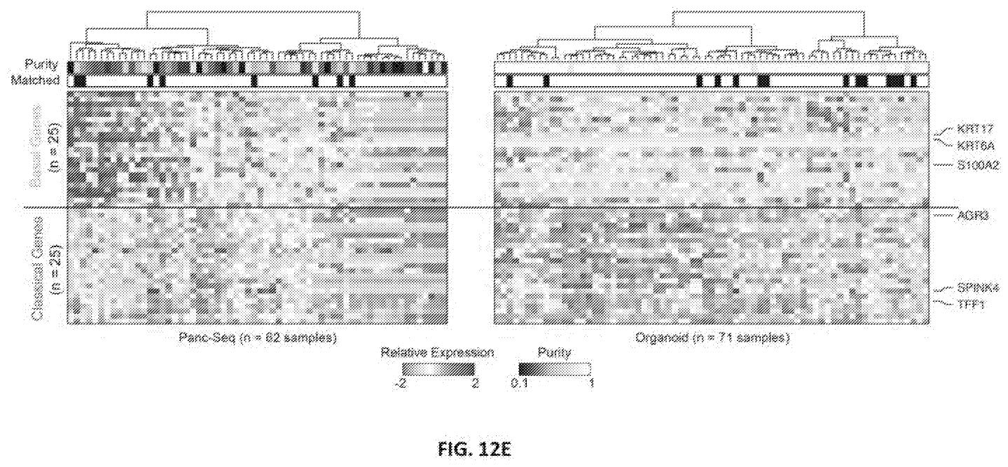

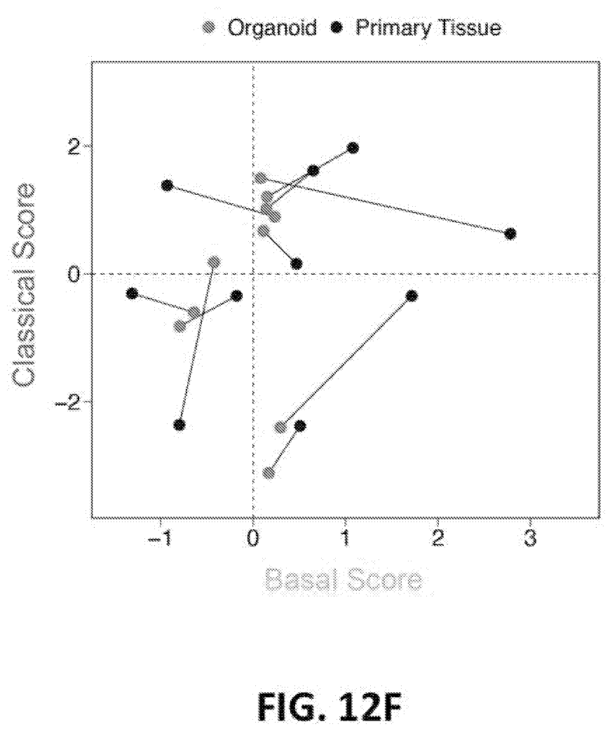

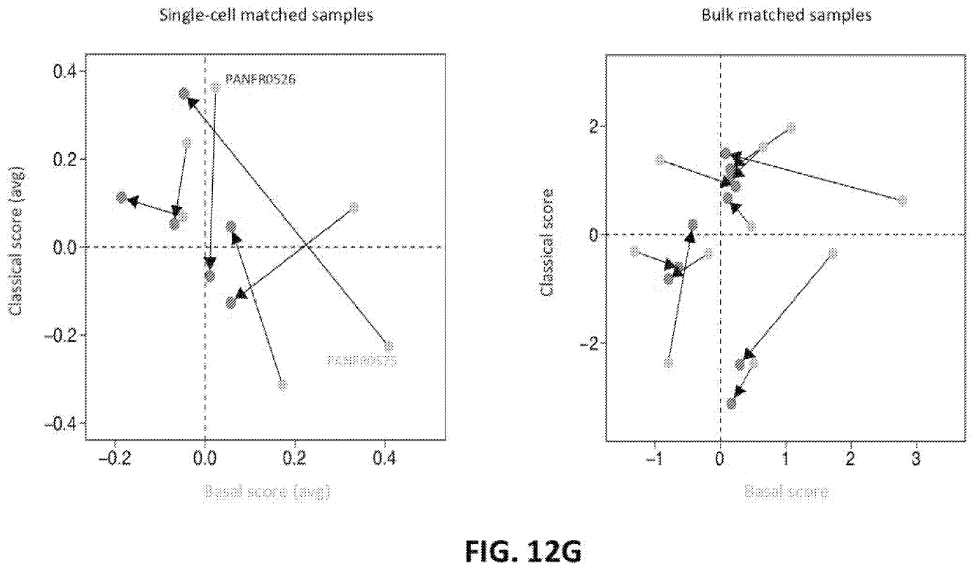

While these tumor classes can be seen in primary patient samples, much of the expression that defines the in vivo tumor state is dampened or disappears in the organoid model system (FIGS. 12A-12G). Basal-like tumors, in particular, have organoid transcriptional profiles that diverge from their in vivo counterparts as early as P2 (FIG. 12A, PANFR0575). InferCNV analysis of single tumor cells from a patient tumor and matched organoid suggests that both transcriptional shifts and sub-clonal selection may be playing a role in differences between organoid models and matched patient tumors (FIG. 12B). FIG. 12C shows a summary of cell numbers recovered from the primary sample (P0) and early passage organoids (Early; P1-P3) from patient matched samples as well as proportion of cells cycling by each time point and group. Fitness flips with in vitro culture—classical tumors increase their growth in the organoid culture environment while basal tumors see a decrease in overall “fitness” at early passage. To confirm the absence of the basal subtype from organoid conditions Applicants compared data from their recent publication of bulk RNA-seq profiles from metastatic PDAC tumors (n=62 cases) with bulk RNA-seq profiles from their organoid cohort (n=70 samples). In this cohort, Applicants confirm several classical, basal, and co-expressing tumors as well as a significant subset that express neither. Applicants also note the general absence of the basal-like subtype from the organoid culture conditions with few—if any—organoids mirroring this more aggressive subtype (FIG. 12E). Here Applicants note a similar loss of the basal phenotype in this patient-matched comparison, supporting the observations made in the single-cell cohort (FIG. 12F).

Applicants hypothesized that there are two main reasons for this lack of fidelity: 1) an inappropriate mix of growth factors and inhibitors in the media and 2) omission of other cell types or cues in the microenvironment critical for determining tumor cell fate.

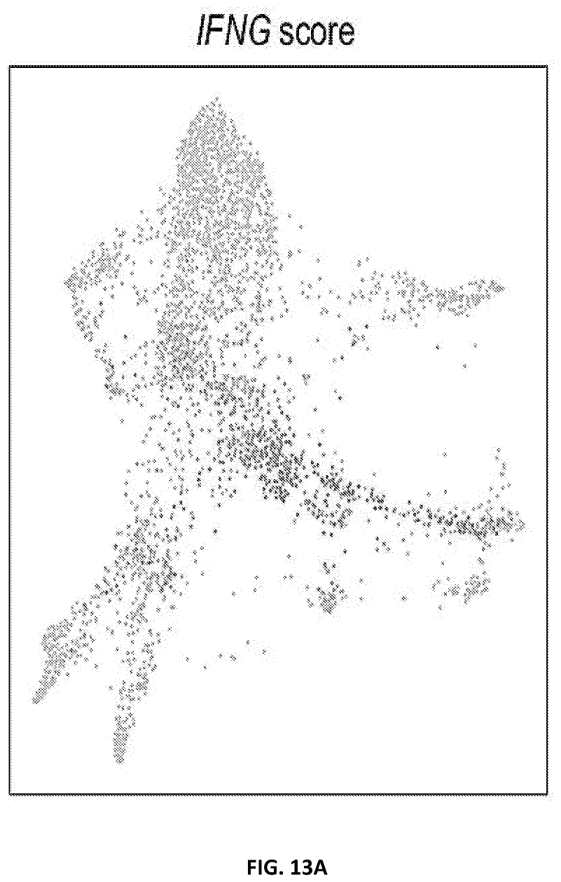

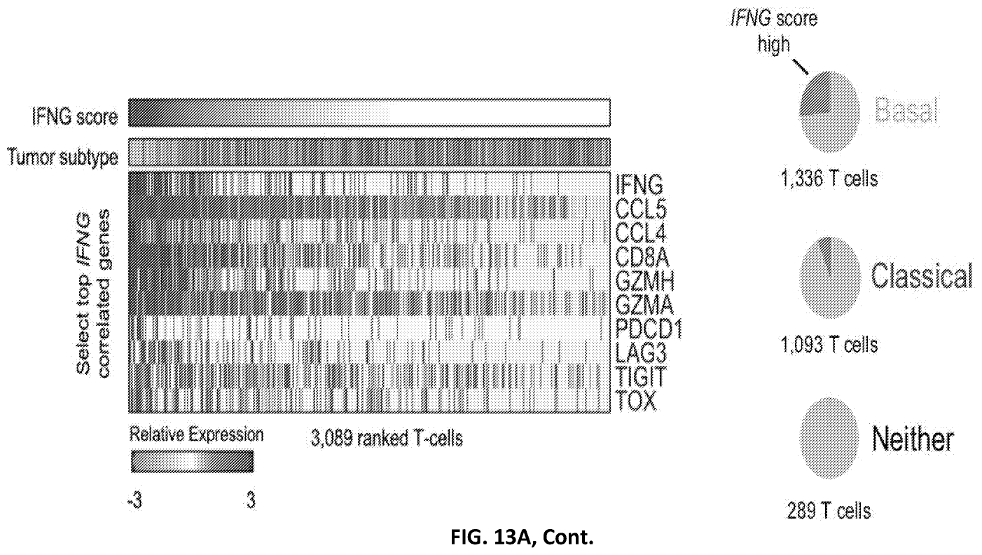

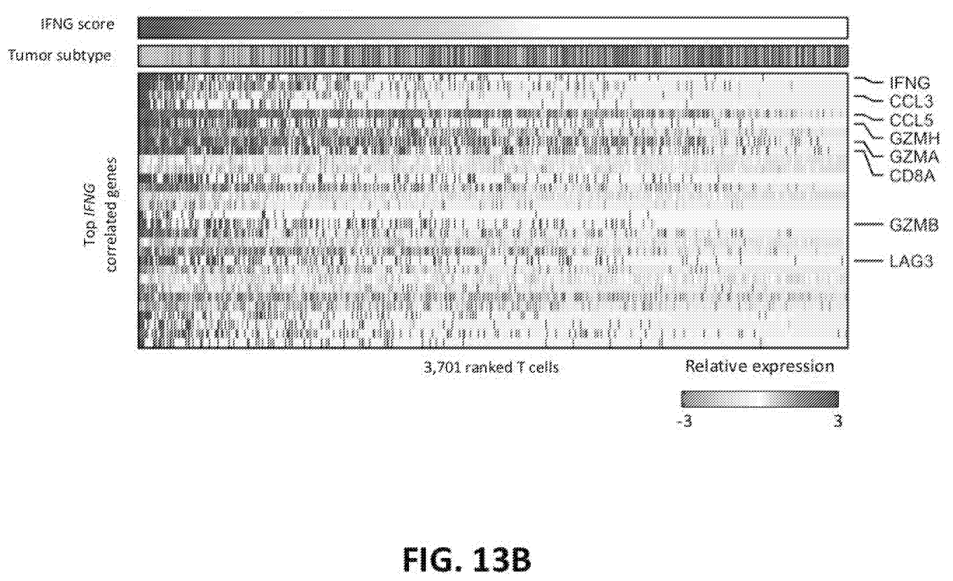

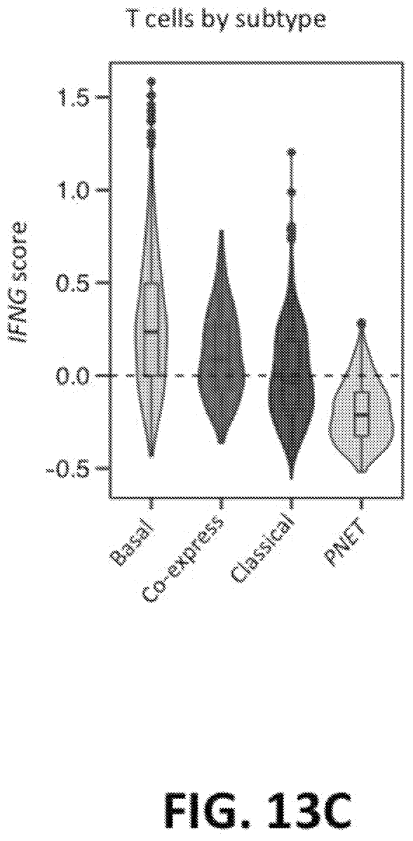

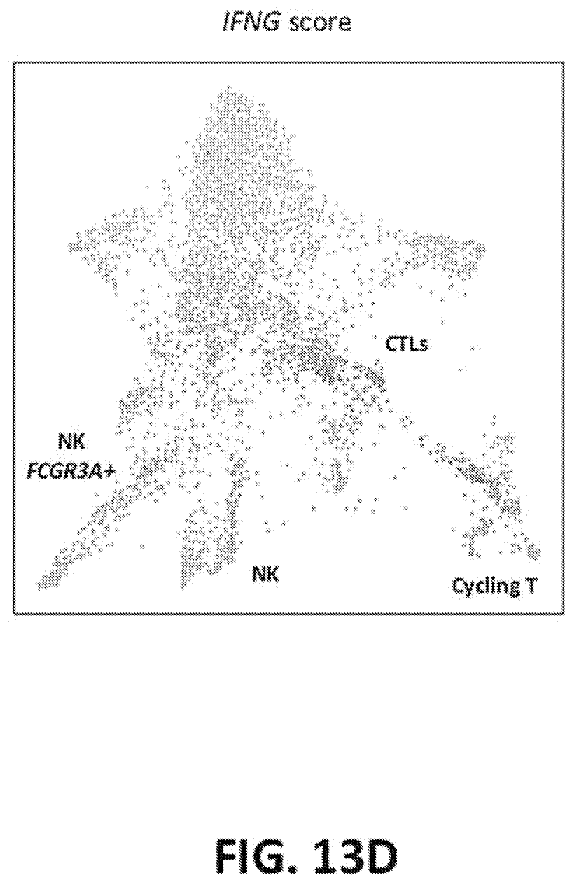

Deeper analysis of tumor phenotypes supported the hypothesis that traditional organoid media may dampen tumor gene expression. The basal-like state expresses genes associated with the TGFB pathway signaling pathway and the media for culturing organoids contains a TGFB inhibitor. This could dampen or even select against the basal state. There was also evidence of missing microenvironmental factors. For example, the basal-like primary tumors express a number of genes associated with the IFNγ response, but are cycling more often. Interestingly, the T-cells from basal-like samples have higher expression of IFNγ, suggestive of crosstalk between tumor cells and T/NK cells in this transcriptional context (FIGS. 4D and 13A-13D). This expression has not been well defined previously in basal-like tumors yet may be critical in defining the in vivo basal state. This altered microenvironment ultimately may lead to a shift in the expression state of the resultant organoids.

Given these findings, the organoid media and growth conditions may be modified to attempt to better match the in vivo microenvironment. The emergent phenotypes of PDAC cells when they are initiated as organoids may also be explored. Initial efforts to change growth media are underway, with a focus on systematically removing many of the growth supplements including TGFB inhibitors. These efforts are guided by novel expression patterns seen in the primary patient data, and by protocols developed in Applicants' lab to specifically use scRNA-seq to compare in vivo and in vitro cell types and states (Mead et al. BMC Biol 16 (2018)). Additionally, Applicants plan to continue to co-culture mouse fibroblasts with depleted media to better replicate the tumor microenvironment (Patel et al. Science 344:1396-1401 (2014)). Finally, Applicants will test the hypothesis that IFNγ supports tumor cell expression state by both initiating organoid cultures with IFNγ and by shifting late passage cultures into new IFNγ media. In light of Applicants' in vivo observations, it will be interesting to compare how tumors that were either classical or basal in situ tolerate exposure to this cytokine. All of these perturbations aim to both test the plasticity of the tumor cells and more faithfully mirror the in vivo tumor state.

In conclusion, Applicants' work provides an important window into the biology of metastatic PDAC to 1) discover therapeutically exploitable tumor phenotypes defined by and reliant on intricate microenvironmental interactions and 2) further empower the field by directly comparing tumor clonality and transcriptional phenotypes across in vivo specimens and their in vitro organoid counterparts.

### Example 3—Assessment of Tumor Heterogeneity and the Tumor Microenvironment in Metastatic Pancreatic Ductal Adenocarcinoma (PDAC) and Matched Patient-Derived Organoids

Despite relatively low incidence, pancreatic cancer has the 3rd highest rate of mortality. It is difficult to detect early and 50% of patients present with metastatic disease. Key oncogenic drivers are well-established, but there are still no approved targeted therapies. There is a critical need for new models and therapeutic approaches.

Several recent studies have proposed PDAC transcriptional classification schemes based on bulk RNA sequencing of patient tumors. These studies have broadly identified at least two transcriptional subtypes with differences in clinical outcomes—a basal-like subtype which has poorer survival and a more classical subtype which appears more responsive to chemotherapy. There are ongoing efforts to assess whether these transcriptional subtypes can be used to guide first-line chemotherapeutic selection. Applicants applied single-cell RNA sequencing of clinical liver metastases to determine transcriptional heterogeneity within tumor cell populations and to characterize the tumor microenvironment. In parallel, RNA expression patterns in matched patient-derived organoid models were investigated.

When studying genomic classification in PDAC, there are few targetable genomic alterations. Only 15-20% of patients have therapeutic decisions altered by genomic data. Tumors often do not respond or develop rapid resistance to targeted inhibitors. Applicants employed the following two major efforts to address these limitations: (1) transcriptional profiling to identify expression-based subtypes; and (2) organoid modeling and personalized functional testing.

Regarding PDAC transcriptional classification and organoid modeling, Applicants asked the following key questions: (1) Are the basal and classical classifications maintained in the metastatic niche and at the single-cell level? (2) Do they change as a function of therapeutic treatment? (3) Are these features preserved in organoid culture?

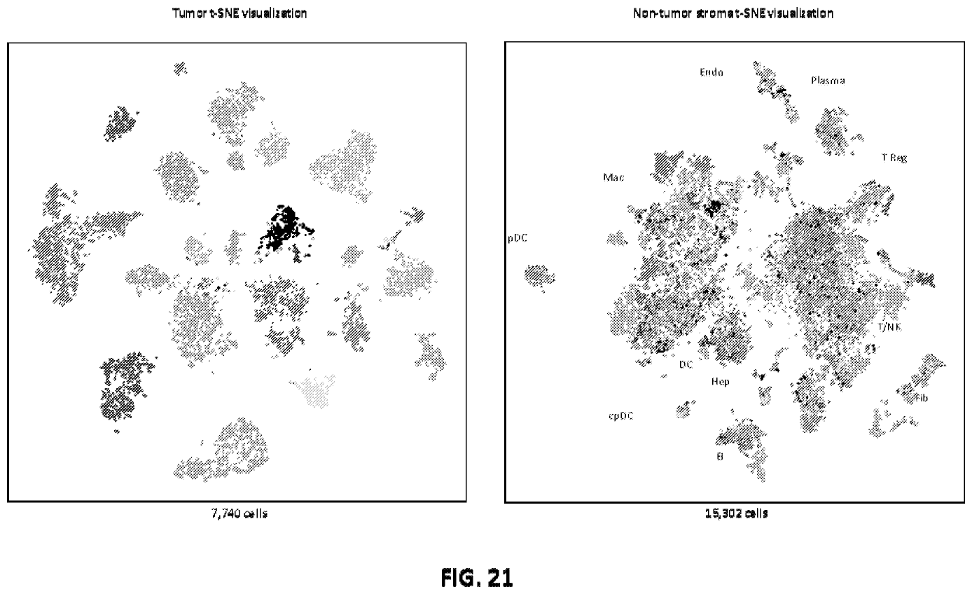

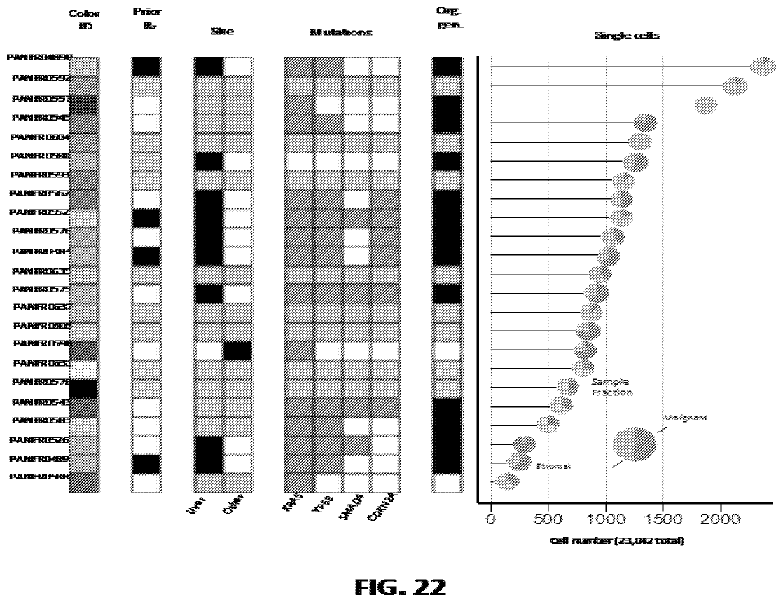

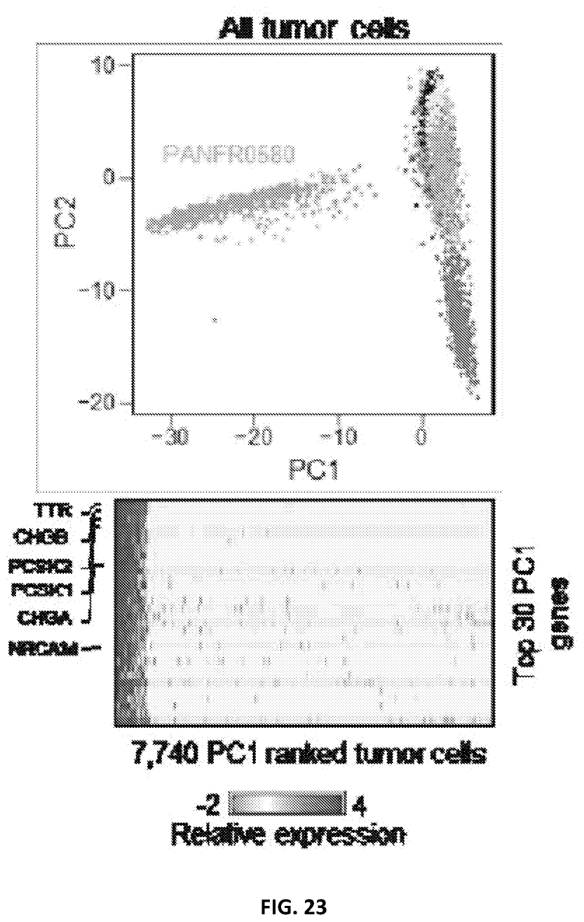

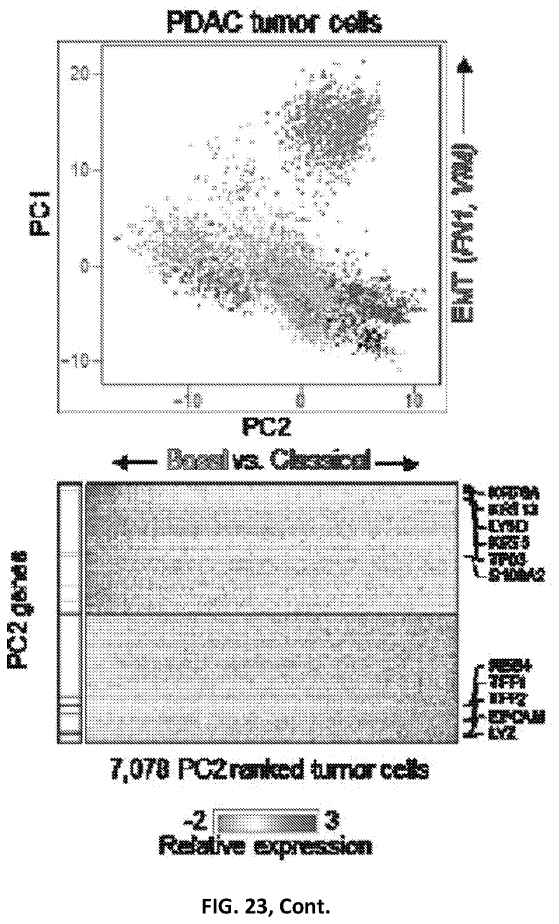

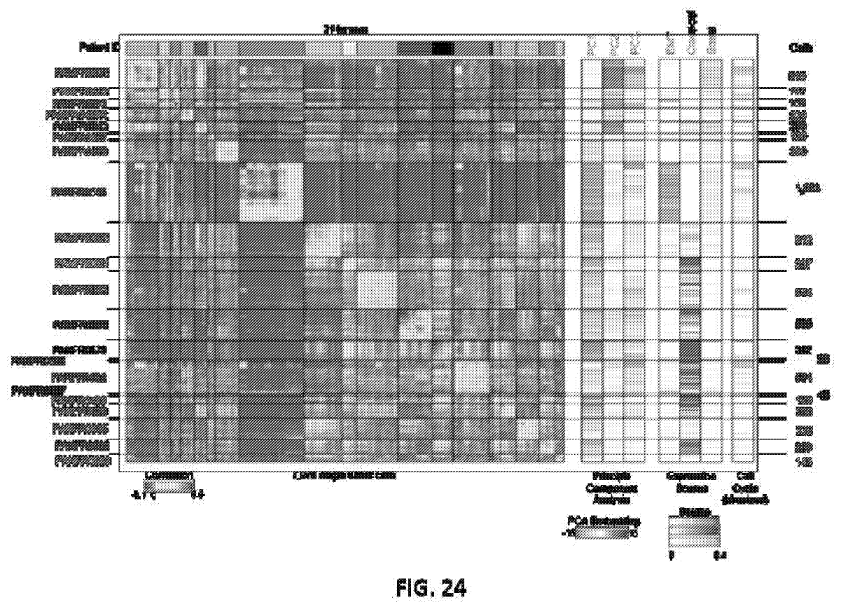

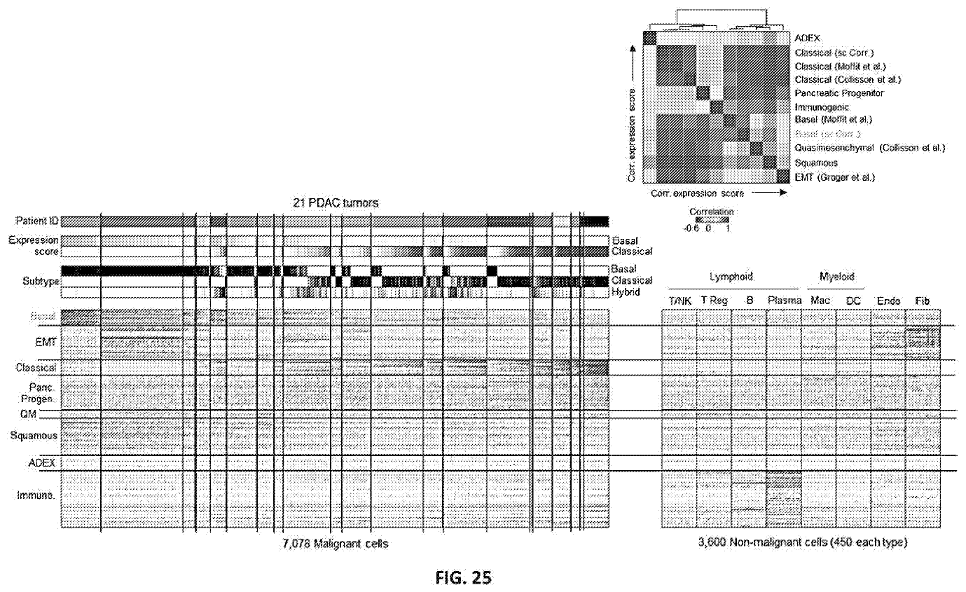

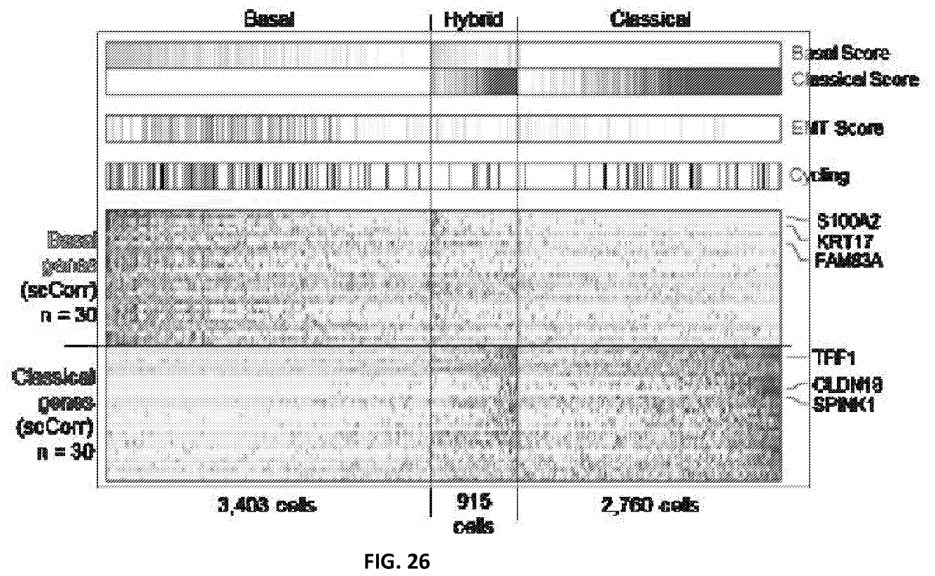

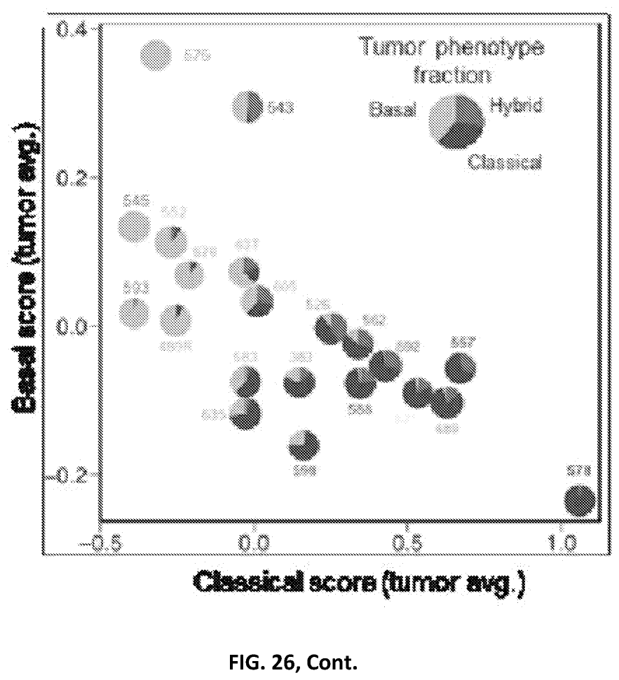

An initial survey of cell types—tumor and stromal/immune cells, is shown in FIGS. 21 and 22. Malignant cells cluster by patient (the colors are the same in FIGS. 21 and 22). Malignant identity is confirmed by single-cell CNV analysis (FIG. 21, left panel). Non-malignant cells cluster by cell type, irrespective of donor (FIG. 21, right panel). To address the question of whether basal and classical classifications are maintained in the metastatic niche and at the single-cell level, Applicants generated transcriptional variation maps to known subtypes. Global maps are shown in FIG. 23 and tumor-by-tumor maps are shown in FIG. 24. FIG. 25 shows maps of single-cell data compared to other published subtyping approaches. Applicants found that tumor cell transcriptomes map to classical vs. basal-like subtypes and that tumor cells exhibit classical, basal-like, and hybrid phenotypes (FIG. 26). Single malignant cells exist along a continuum of basal to classical and are capable of co-expressing both transcriptional programs in the same cells. Basal cells are more mesenchymal and proliferate more aggressively than classical cells. Of note, the EMT phenotype, basal vs. classical and cycling programs are all identified in the first three principal components of the malignant cells. Genes displayed in the left panel of FIG. 26 are the top 30 correlated genes to either basal or classical scores from Moffitt et al. (2015). In the right panel of FIG. 26, tumors are plotted by their average basal and classical scores and pie charts represent the fraction of each type within each tumor.

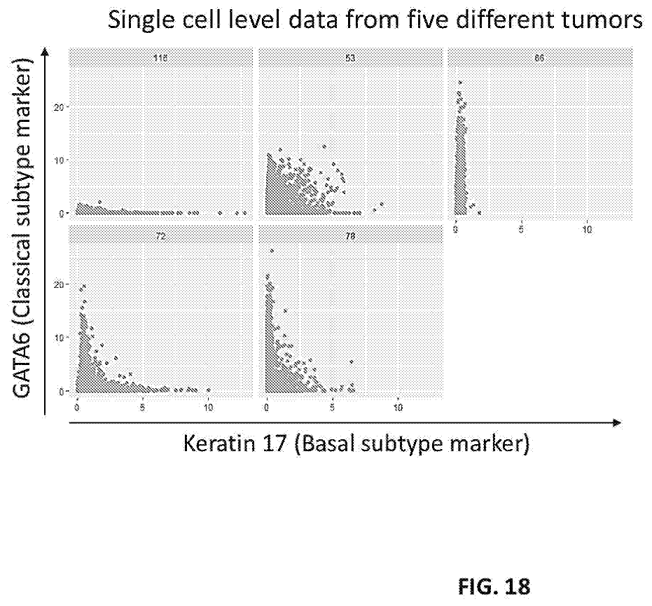

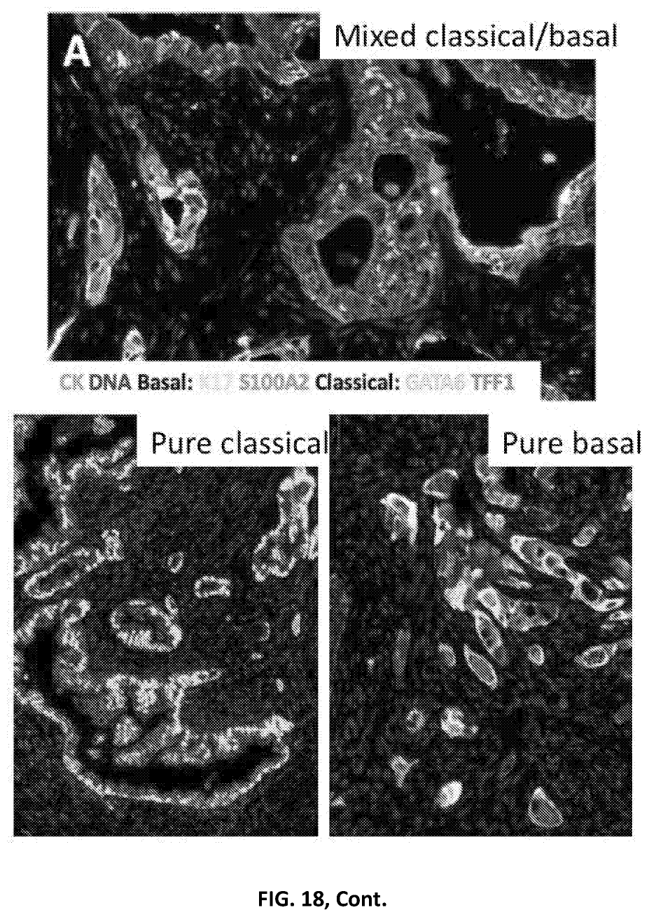

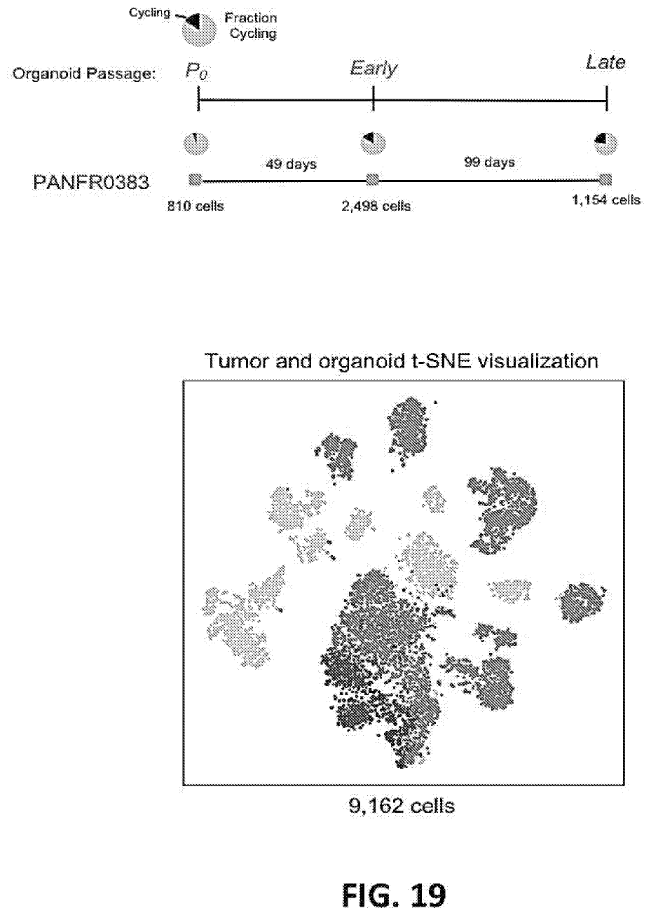

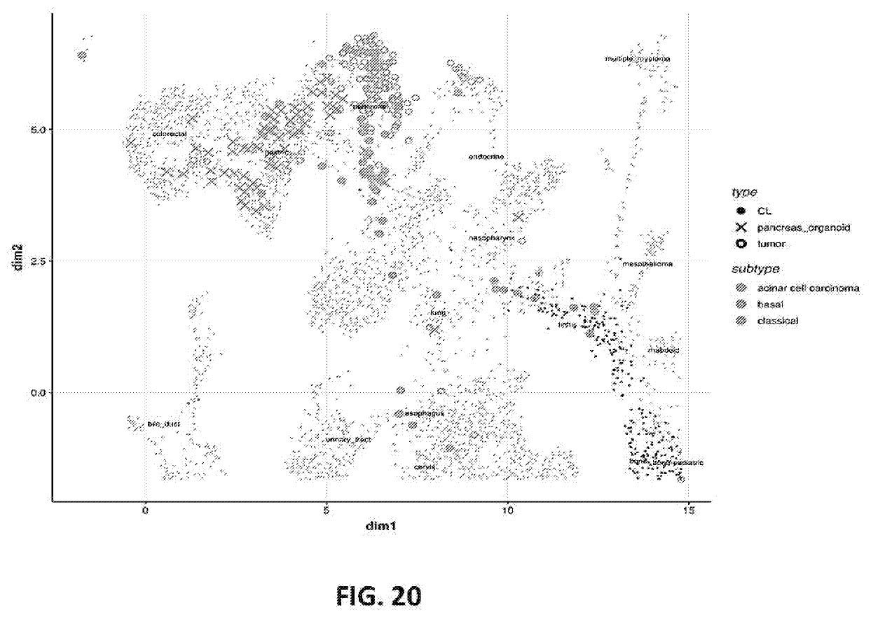

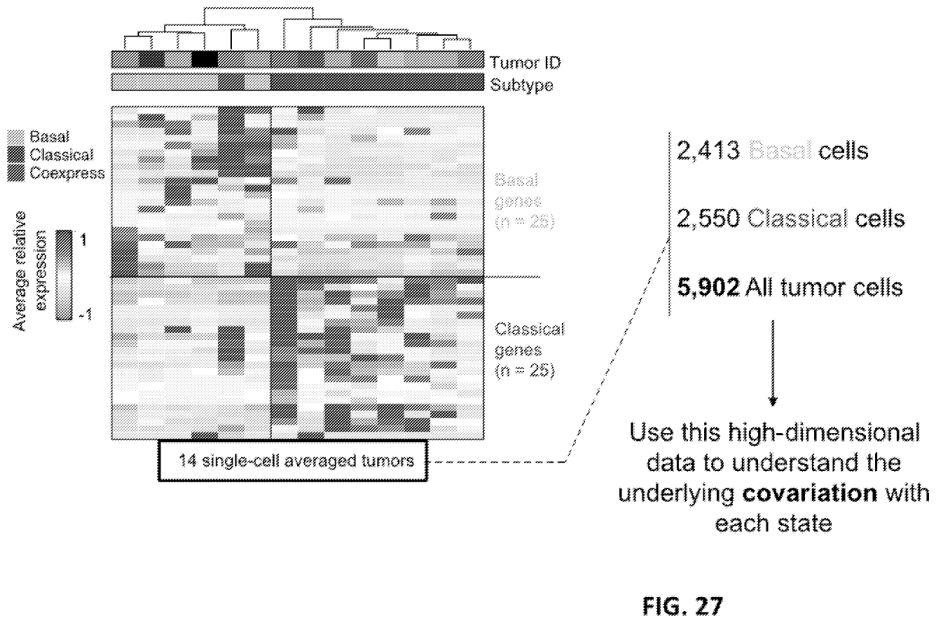

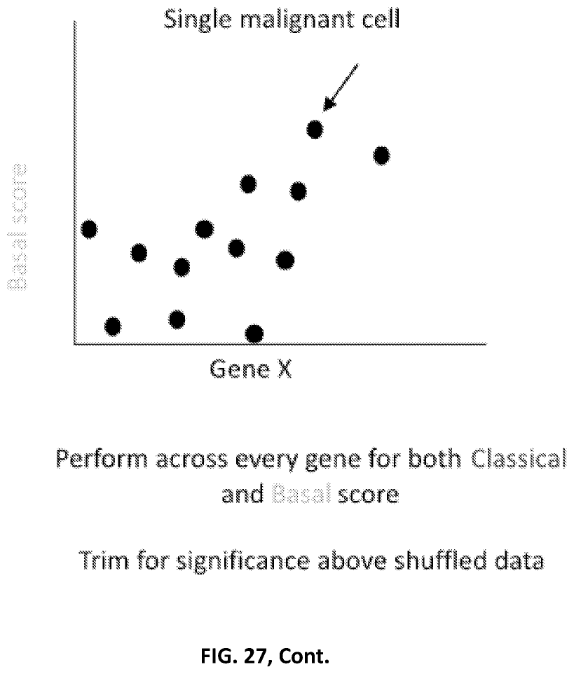

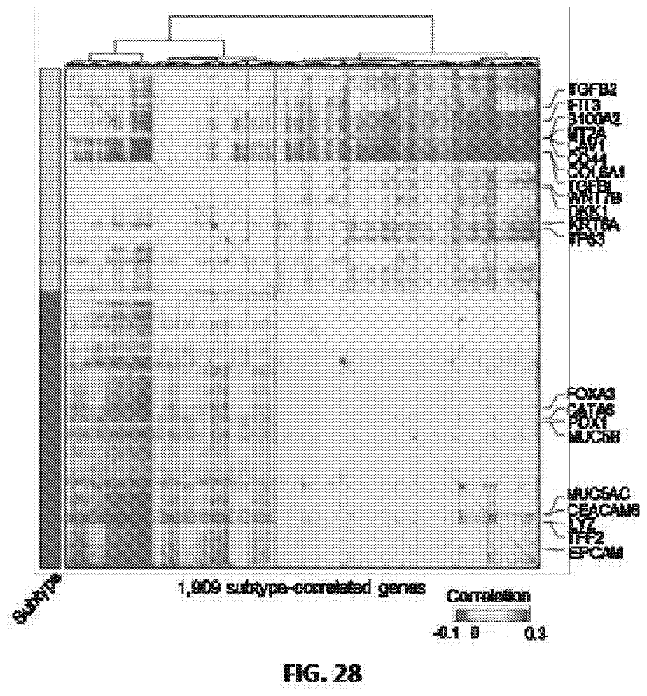

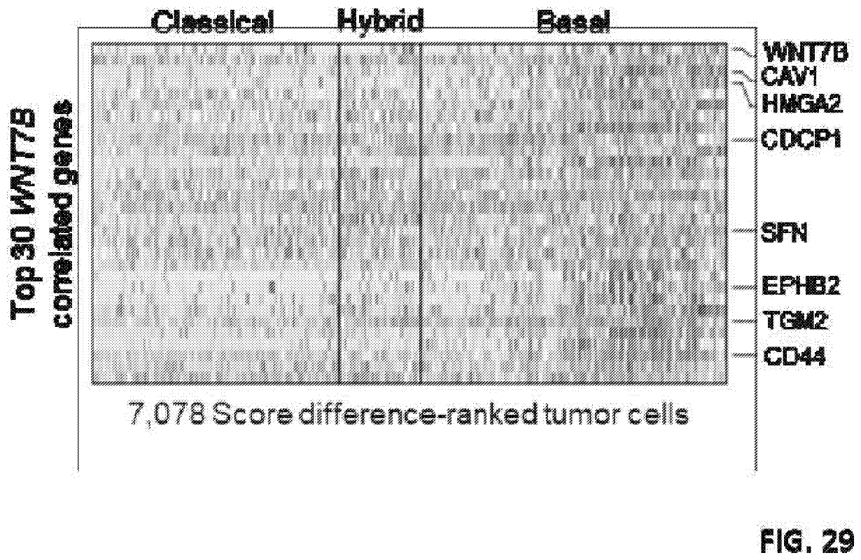

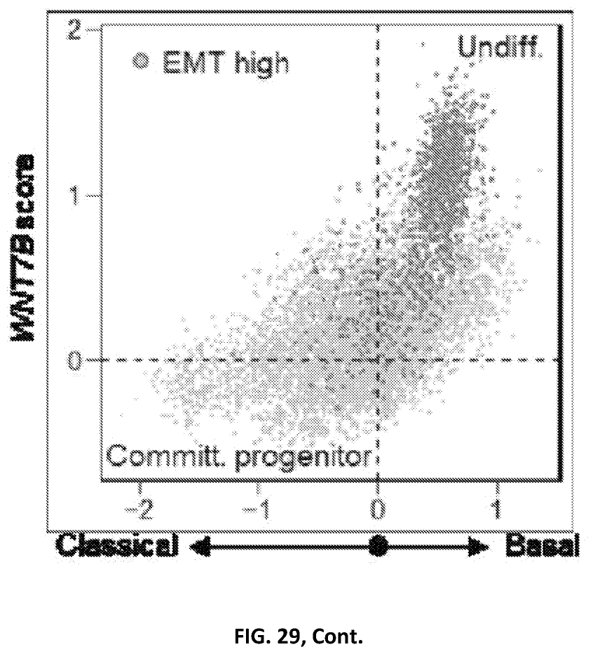

Multiplexed fluorescence confirmed classical, basal, and co-expressor subtypes (FIG. 18). Applicants leveraged single-cell resolution to uncover biology associated with different subtypes (FIG. 27). This raised the question of whether there exist additional transcriptional modules that co-vary with basal versus classical states. FIG. 28 shows that Wnt signaling, IFN response, and TGF beta signaling correlate with the basal-like state in vivo; in particular, Wnt7B signaling (FIG. 29), IFNG crosstalk, and TGF-beta signaling. Overall, Applicants found that basal and classical classifications are maintained in the metastatic niche and at the single-cell level, with evidence for additional (co-expressor) subtypes and microenvironmental contributions.

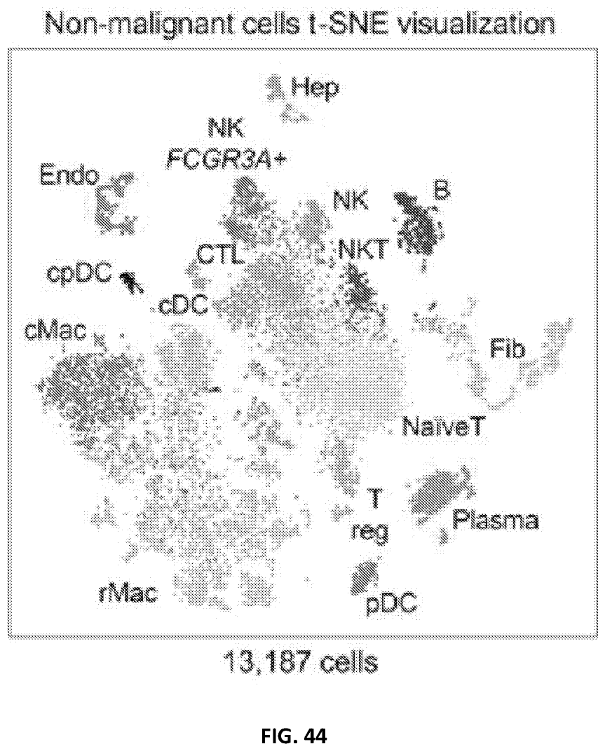

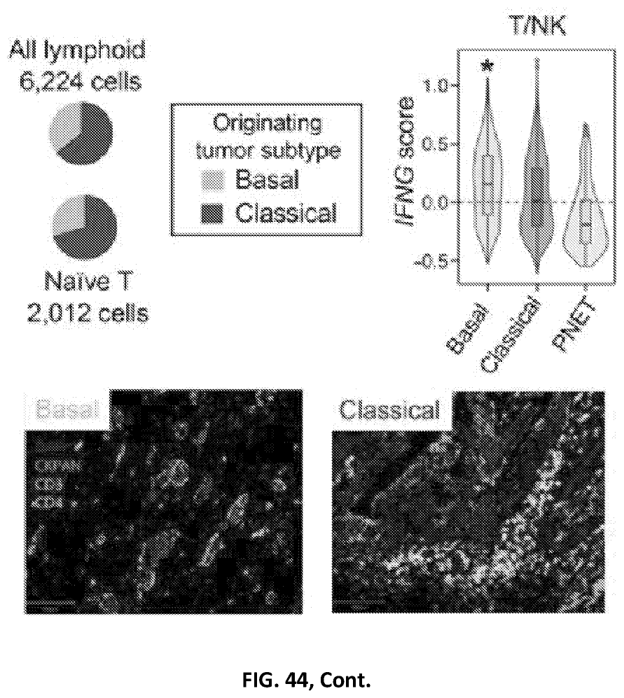

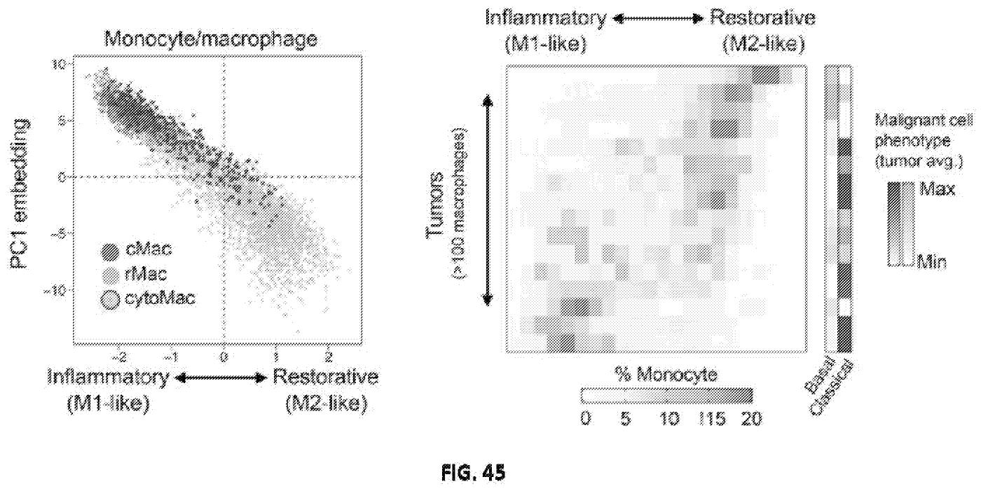

Applicants also found that the basal-like microenvironment excludes T cells and enriches for supportive macrophage phenotypes. FIG. 44 shows the specific cell types identified in all tumors excluding those on active immunotherapy.

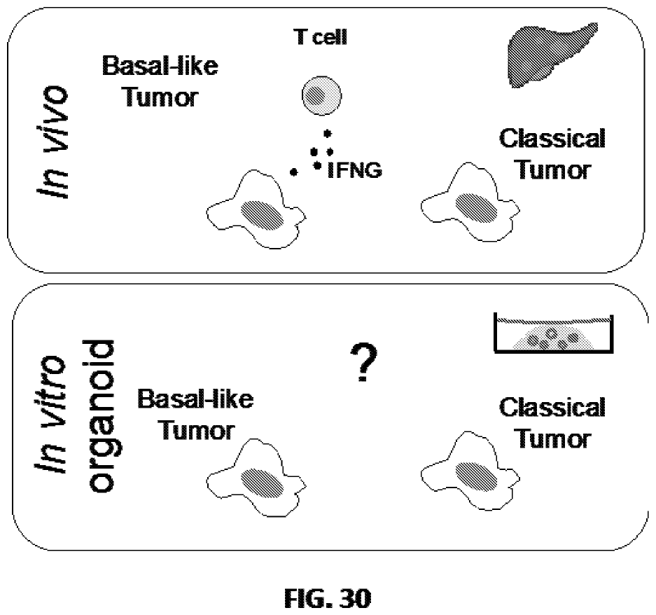

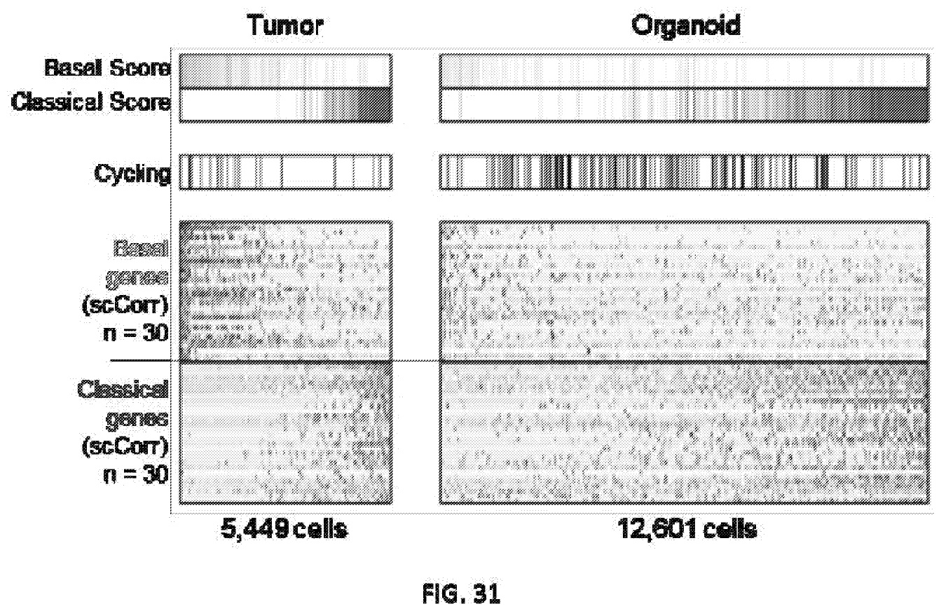

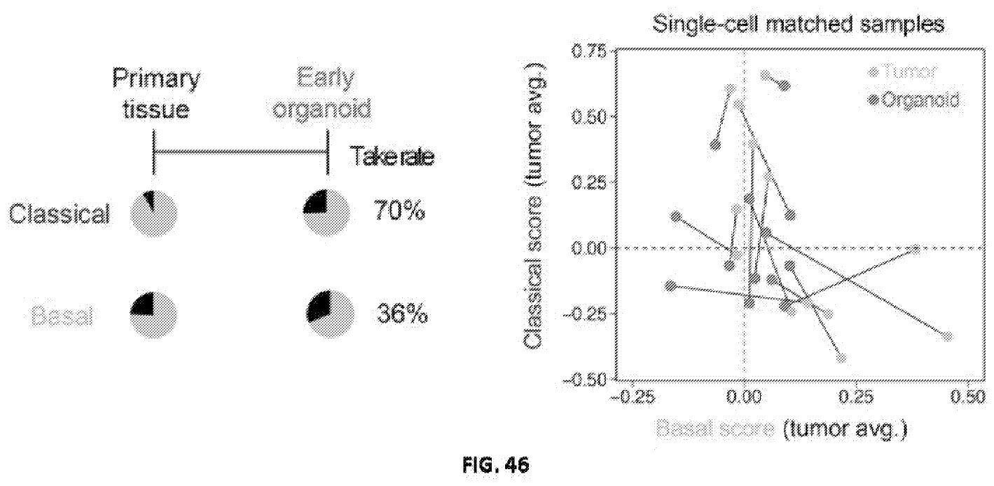

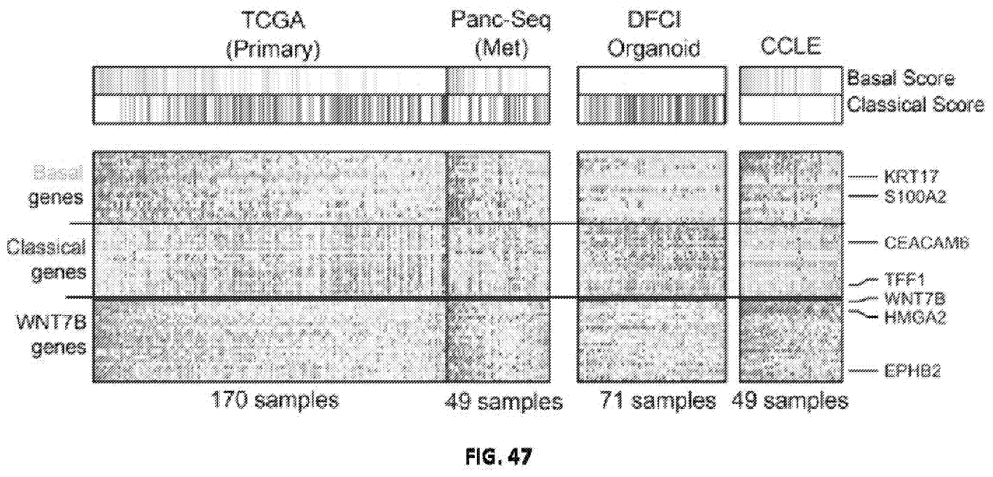

Bulk RNA sequencing of metastatic PDAC tumors shows a distribution of basal-like and classical phenotypes. Organoid models are over-represented in the classical state, with few basal-like models. Applicants next address the question of whether basal and classical features are preserved in organoid culture, where high levels of Wnt3A, R-spondin (R-spo1), TGF-beta inhibitors are present. As it turns out, organoid models alter their transcriptional phenotypes early, with multiple mechanisms of drift involved. Such mechanisms may include sub-clonal selection and expansion, or transcriptional plasticity with maintenance of genomic identity (FIGS. 12A, 12C, and 12G). Some organoid cultures exhibit transcriptional shifts relative to matched primary specimens. Tumor cells that are basal-like in patients appear to lose most of these features as organoids in vitro. In FIG. 46, each organoid represents the earliest possible sample measured for each. Organoids derived from basal tumors lose their phenotype and sometimes switch to classical phenotypes. In other words, organoid models lack basal-like transcriptional features (FIG. 31). Overall, there appears to be selection away from basal-like subtypes in organoid culture. Studies to assess organoid plasticity and conditions that might preserve the basal-like state are ongoing.

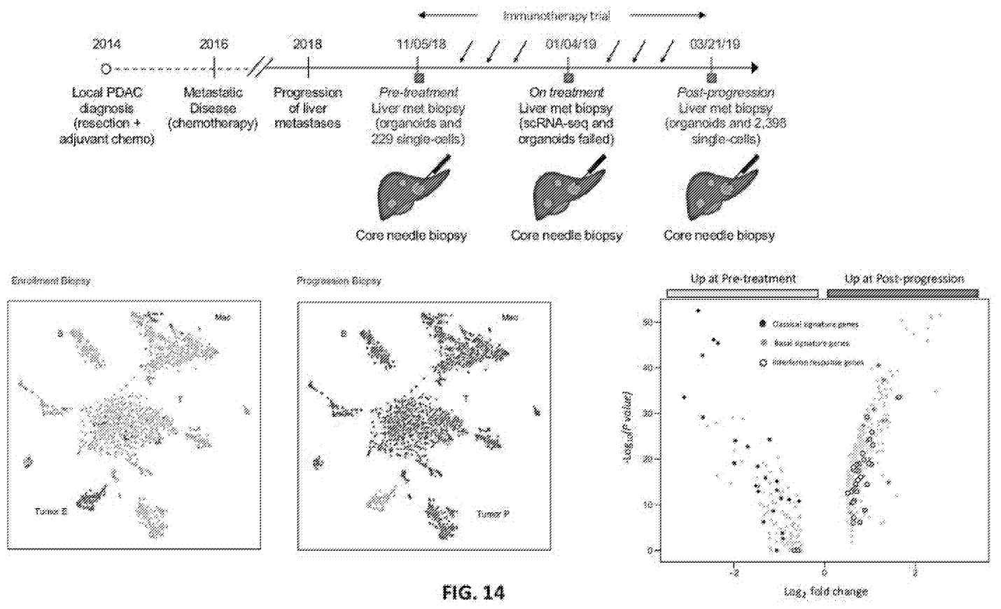

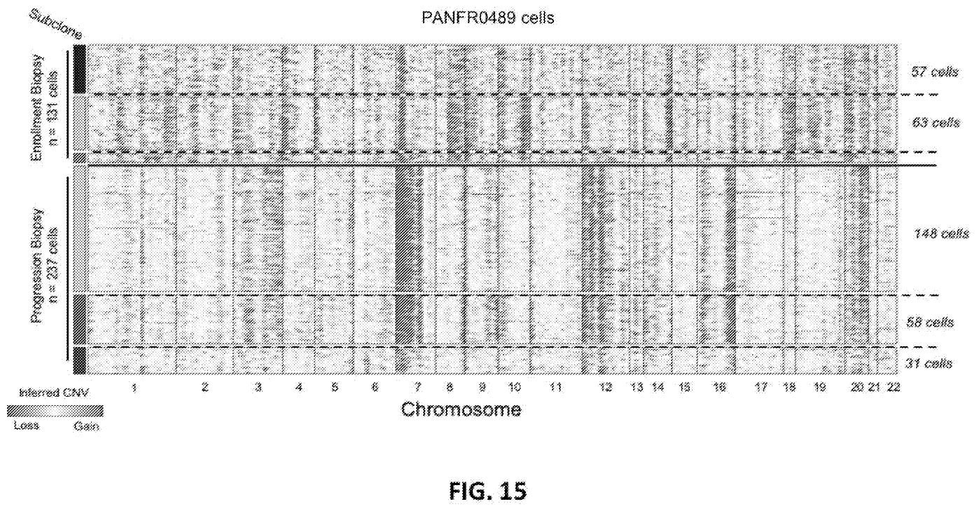

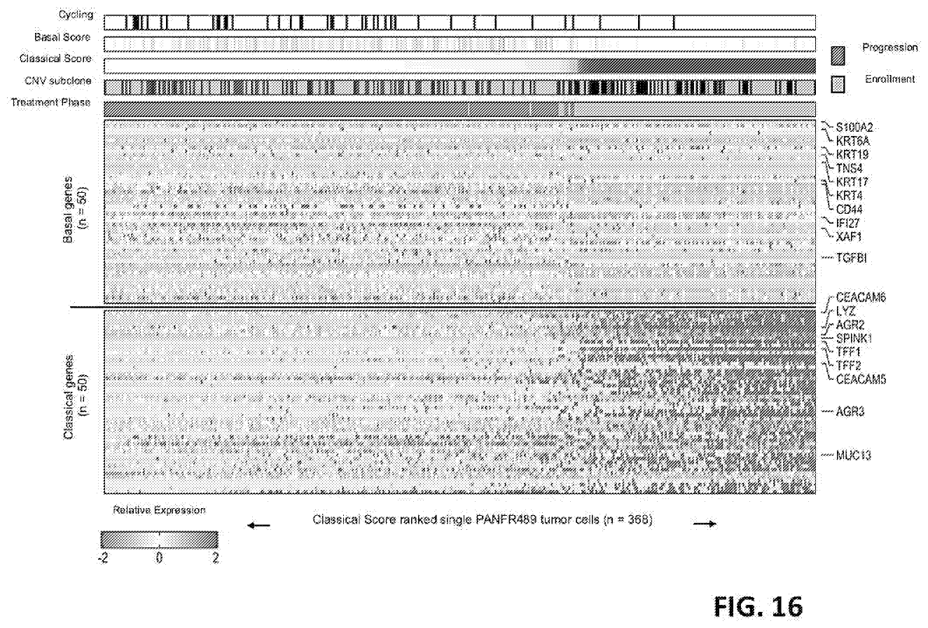

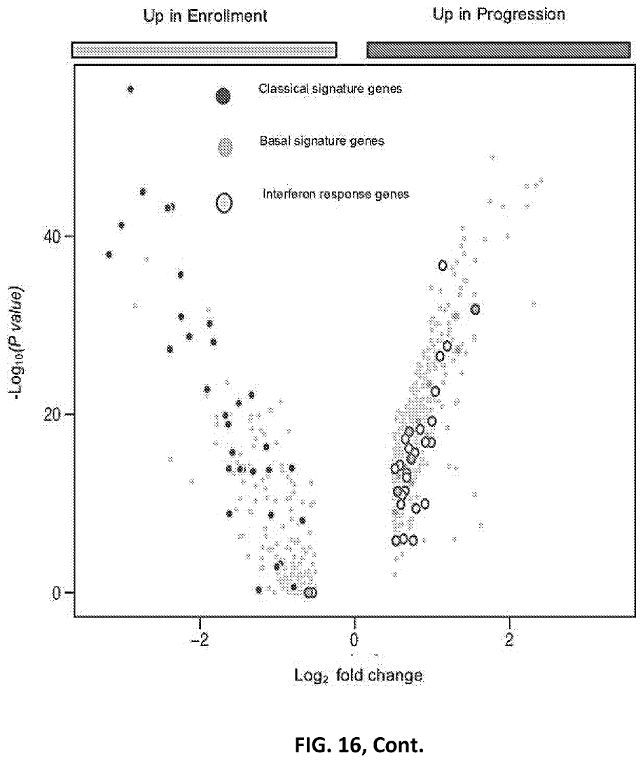

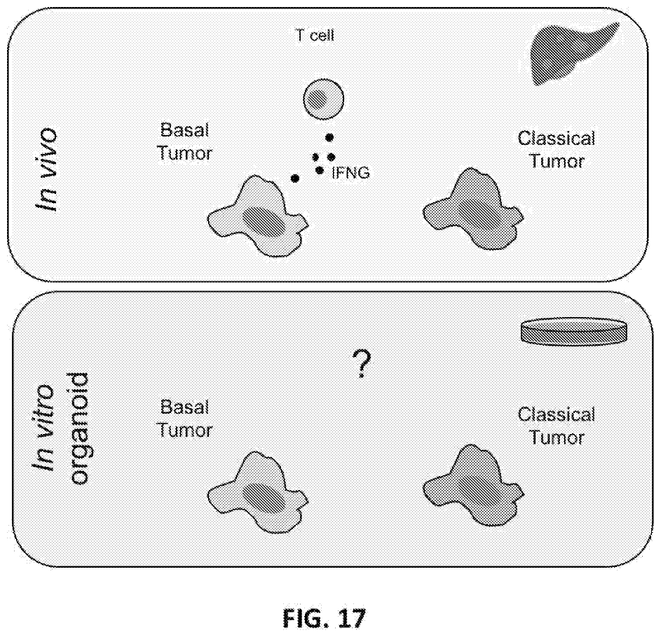

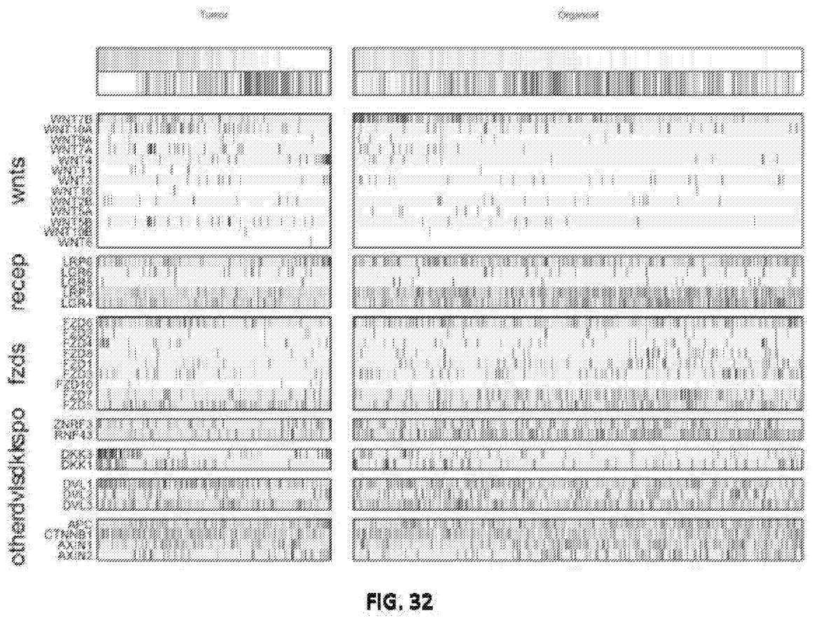

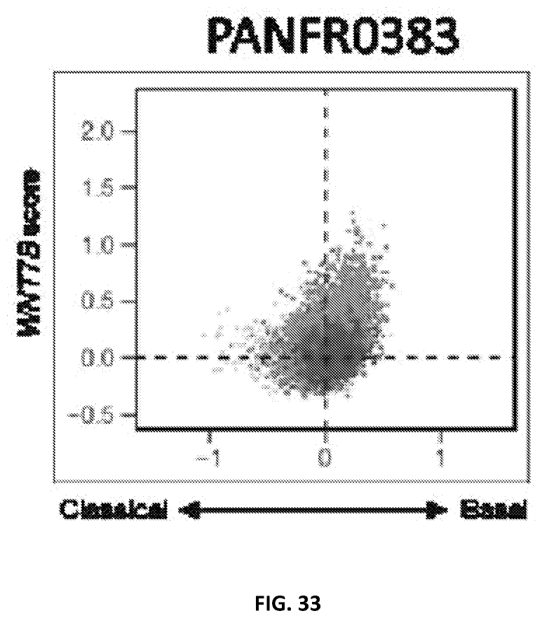

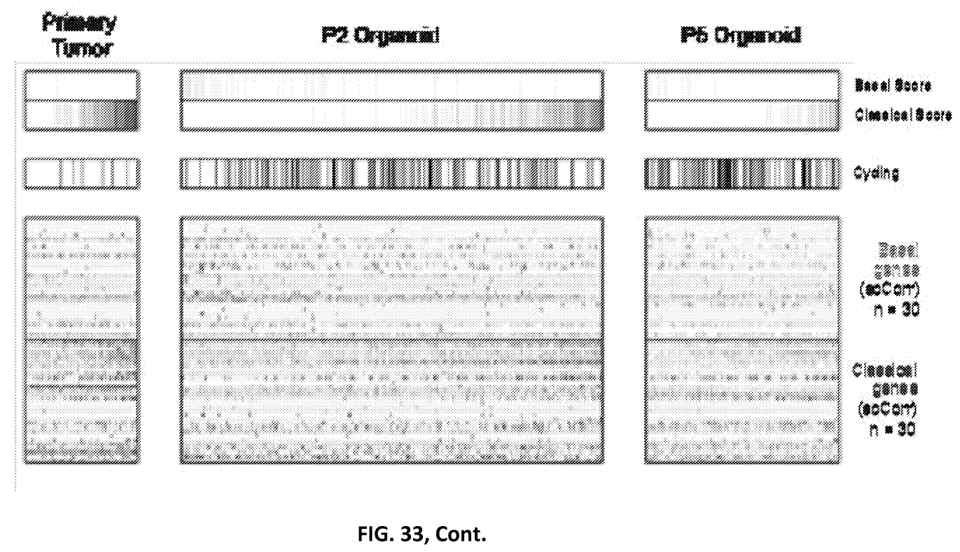

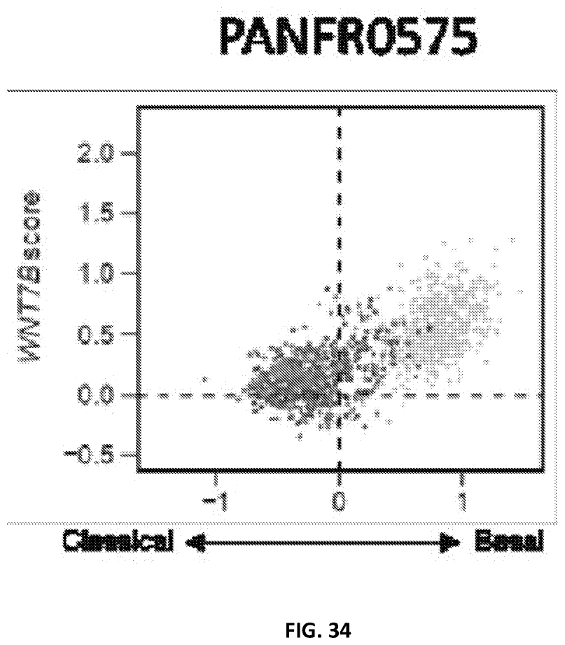

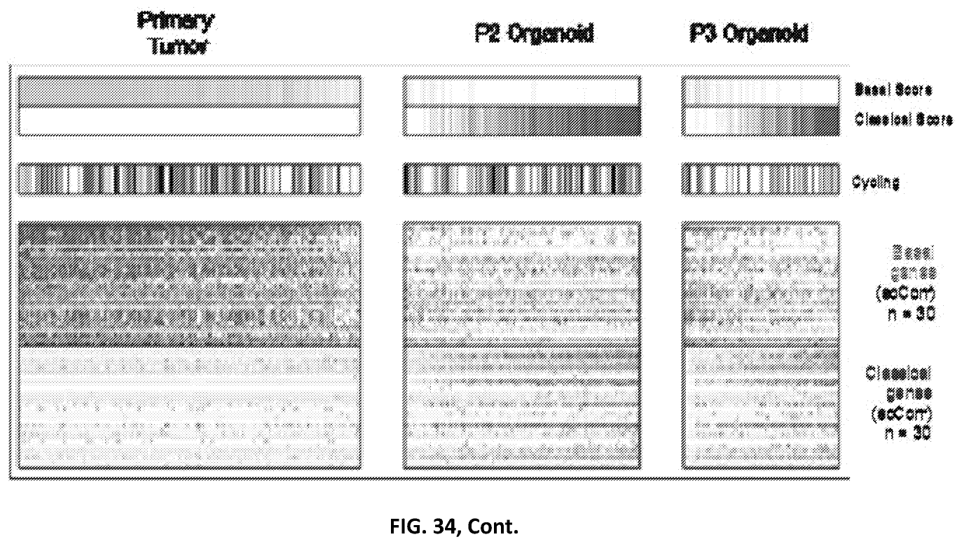

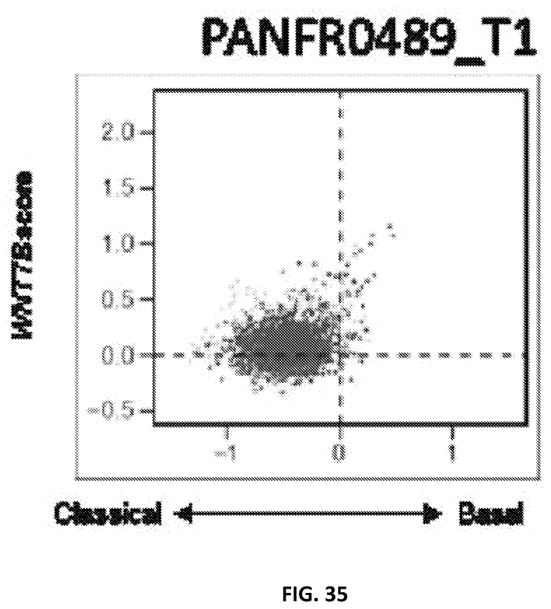

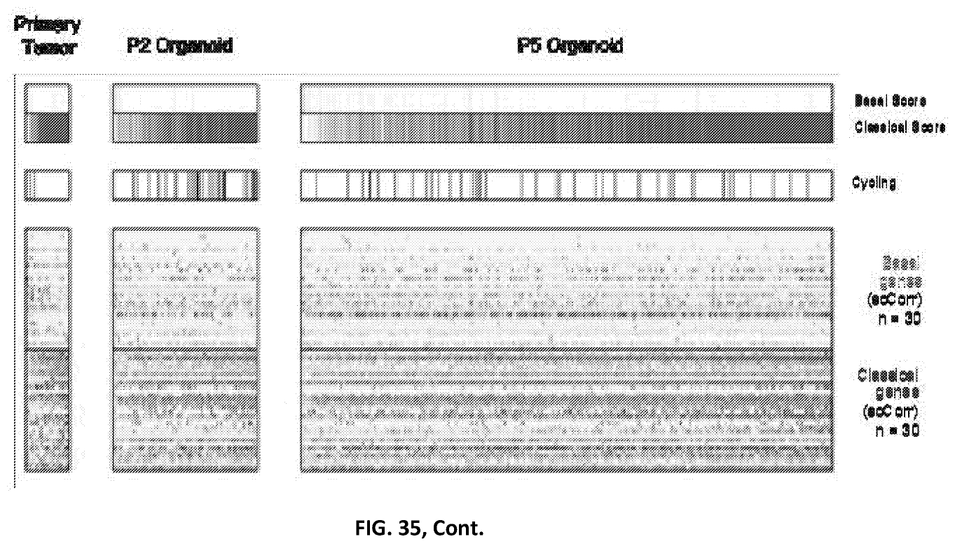

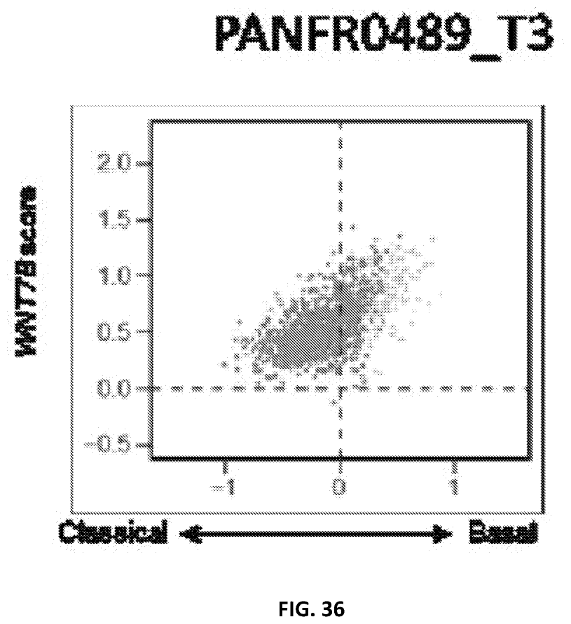

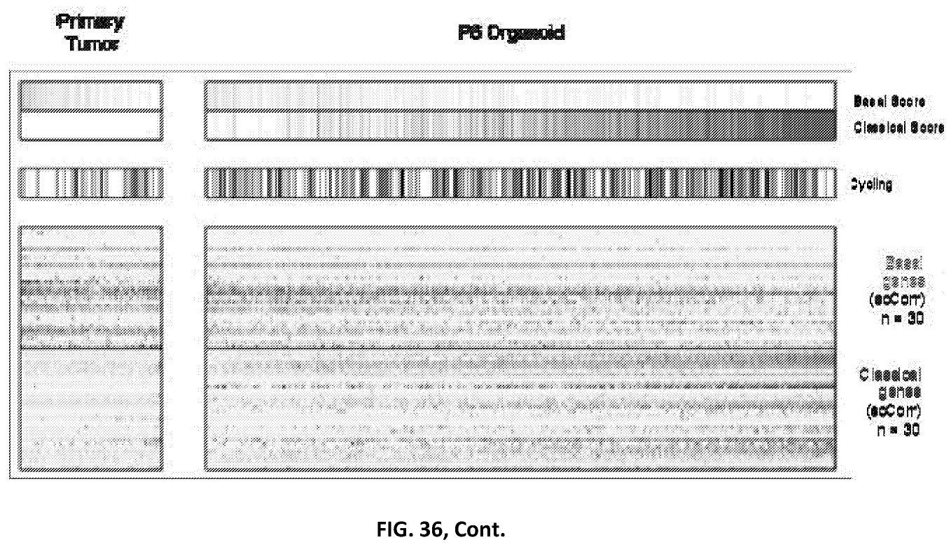

FIGS. 32-34 show changes along the Wnt axis in organoid models, with FIGS. 33 and 34 showing evolution from classical and basal tumors, respectively. Serial sampling revealed dynamic transcriptional changes (FIG. 14). To address the question of whether basal and classical classifications change as a function of therapeutic treatment, Applicants serially sampled organoid models both before and after treatment (FIGS. 35 and 36, respectively) and revealed dynamic transcriptional changes (FIG. 14). Sampling of the same lesion pre- and post-treatment showed that lesions had high spatial heterogeneity and that lesions partially responded to treatment. Overall, Applicants found that classifications did change across a limited sample set. There are ongoing experiments to increase sample number.

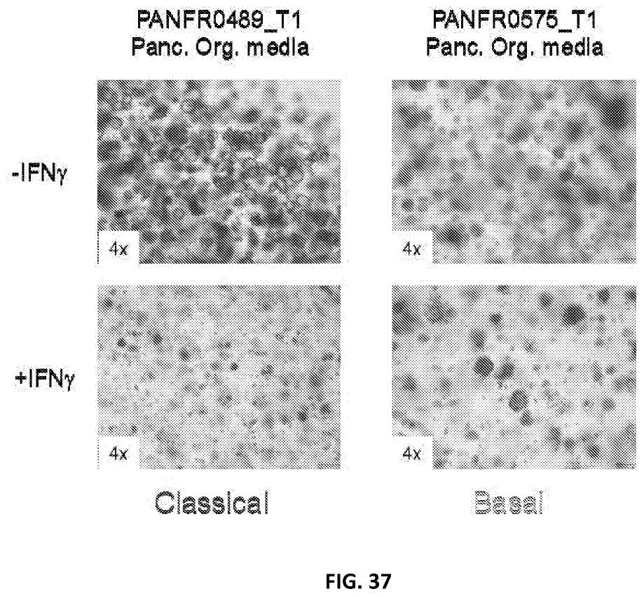

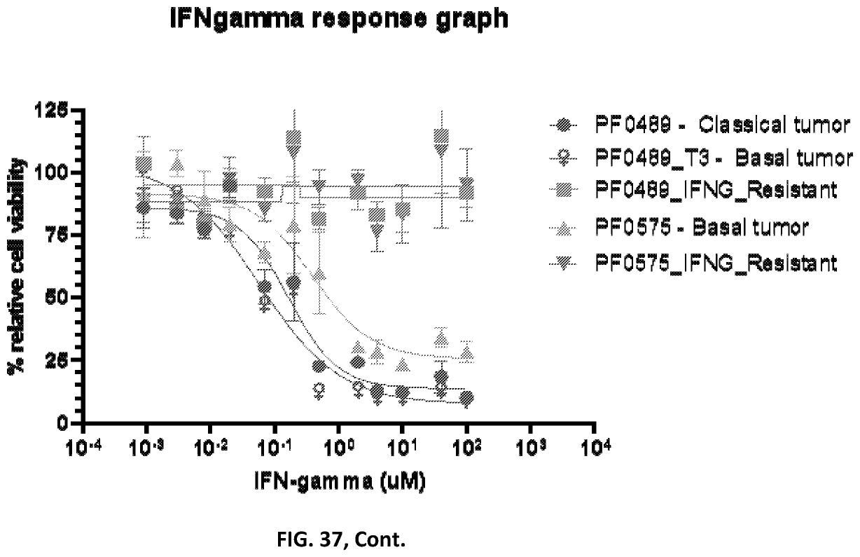

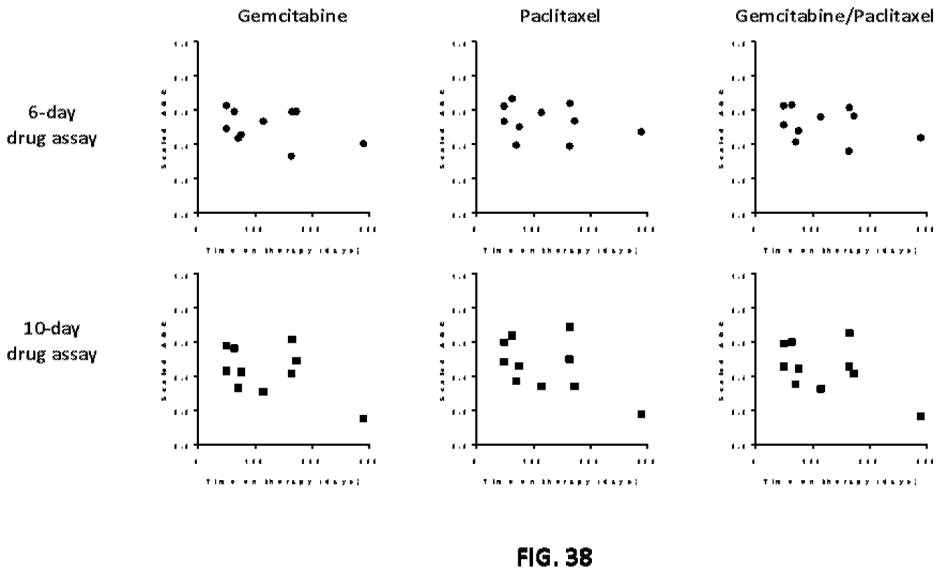

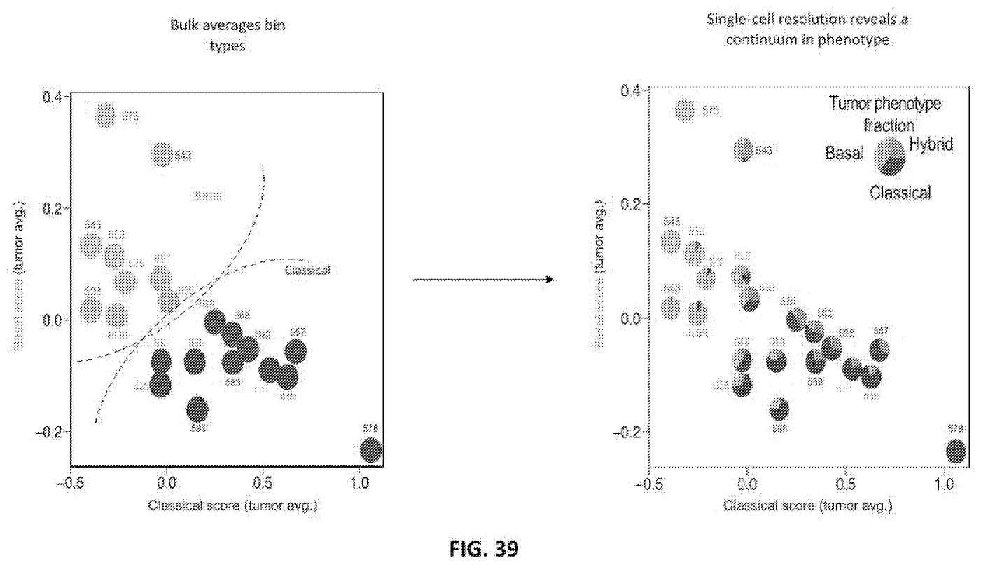

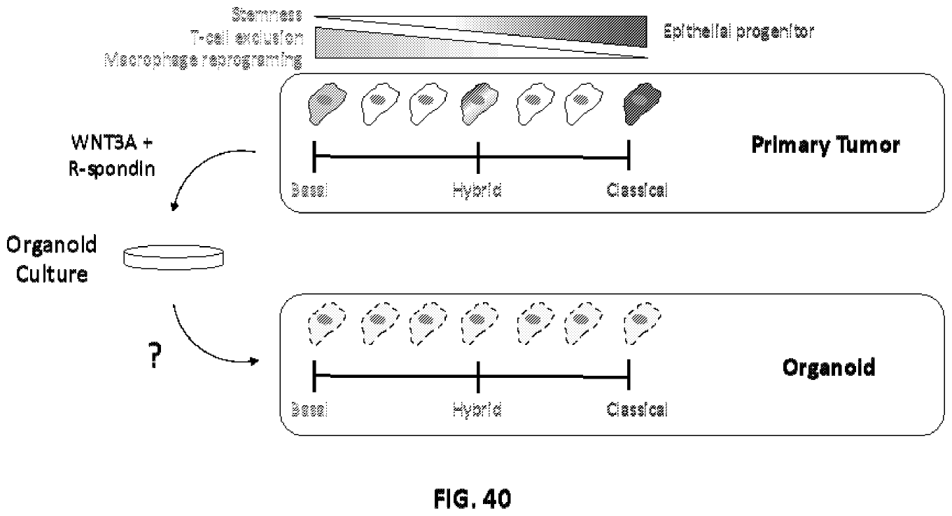

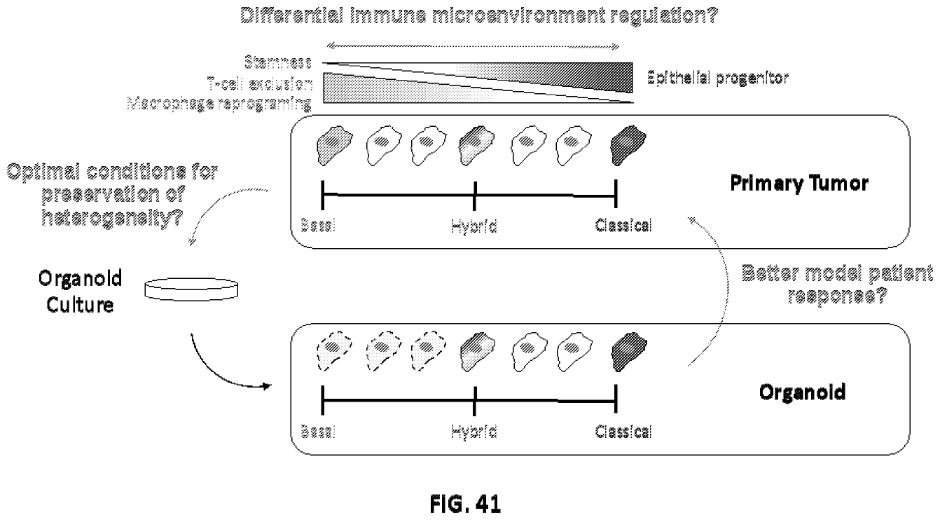

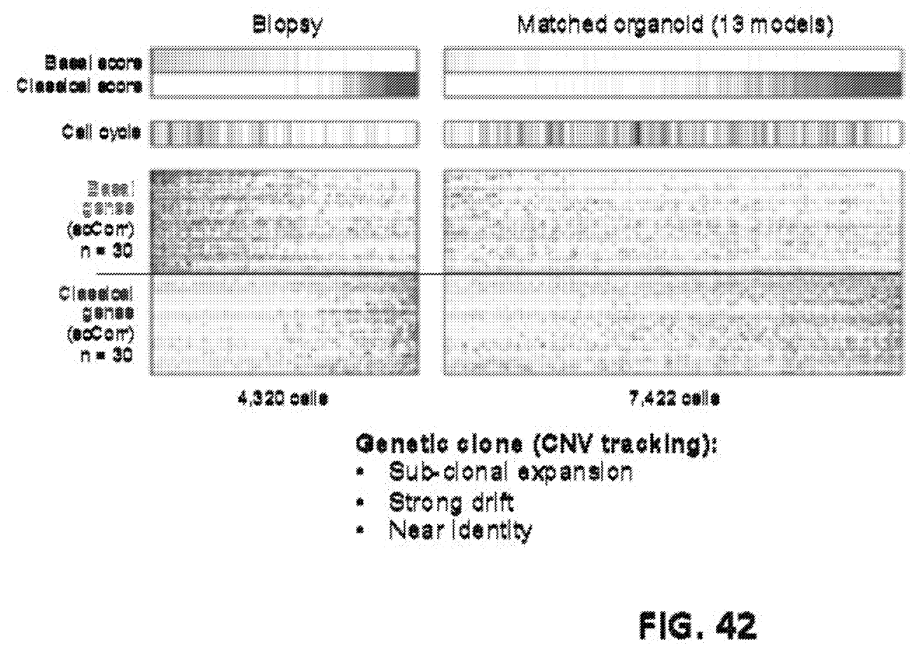

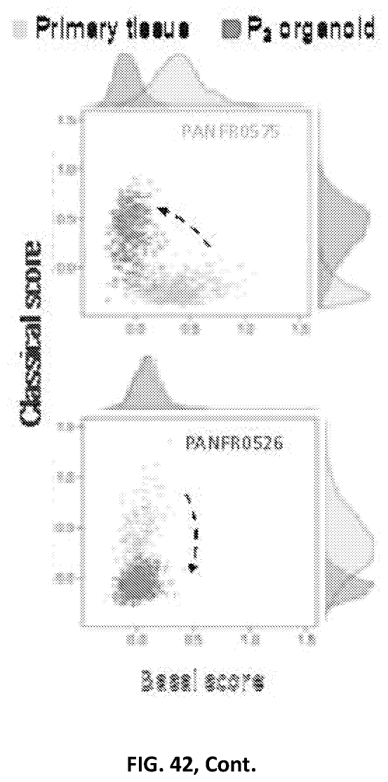

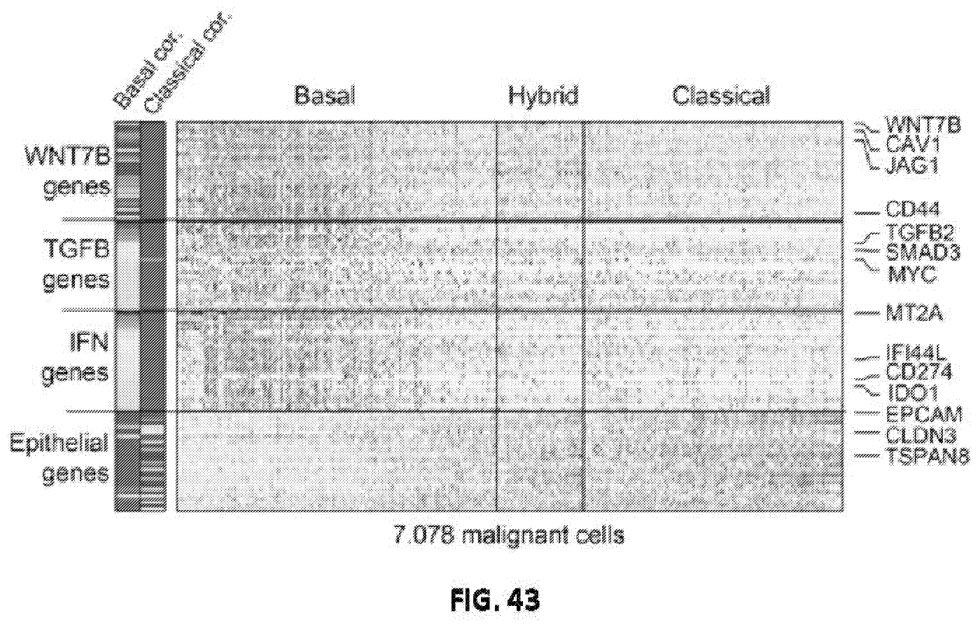

Applicants next asked whether organoids from classical versus basal-like tumors respond differently to addition of IFNγ. Preliminary results show that grown inhibition by IFNγ is more pronounced in organoids derived from classical tumors. IFNγ appears to select against the classical expression state in organoids and may not be sufficient to drive complete return to a basal state (FIG. 37).

In conclusion, Applicants established a robust pipeline for single cell sequencing of liver metastases and matched organoids from low cellular input biopsies. The basal-like transcriptional state is strongly correlated with IFN responses in tumor cells and corresponding IFNγ secretion by T and NK cells from the tumor microenvironment.

Validation of these transcriptional states and correlated modules in archival specimens is ongoing. Some tumors may transition to a basal-like high IFNγ immuno-resistant state after immunotherapy. Organoid models exhibit transcriptional shifts relative to matched tumor specimens after ex vivo culture.

### Example 4—Transcriptional Subtype-Specific Microenvironmental Crosstalk and Tumor Cell Plasticity in Metastatic Pancreatic Cancer

While classification of human malignancies by genotype has provided critical structure for understanding tumor biology, tumors can also harbor clinically relevant variation in transcriptional phenotypes (1). Indeed, for several malignancies such as pancreatic ductal adenocarcinoma (PDAC), classification based on RNA expression has emerged as a genotype-independent predictor of chemotherapy sensitivity and patient survival (2-6). In PDAC, bulk transcriptional profiling has defined two major transcriptional subtypes, basal-like/squamous (hereafter referred to as “basal”) and classical, where the former is associated with worse prognosis and greater treatment resistance (3-5, 7-14). However, classification based on bulk expression profiling can obscure clinically relevant cellular attributes because it reduces signals from multiple cell types to a single, whole sample average. In reality, PDAC tumors, like many other cancers, are complex multicellular ecosystems shaped by both malignant and microenvironmental features. Unlike in DNA sequencing where mutant and normal reads can be precisely separated, malignant and non-malignant signals in bulk RNA profiles are not easily disentangled, making conclusions about their relationships challenging.

The recent application of single-cell RNA-sequencing (scRNA-seq) to human cancers has revealed that the tumor ecosystem is highly heterogeneous and often consists of continuous phenotypes within both malignant and non-malignant populations (15-21). The precise cellular characterization this method affords has enabled the re-examination of transcriptional taxonomies and reframed our understanding of the summaries provided by bulk measurements in multiple cancers (15, 21-26). Such enhanced resolution may be particularly useful in PDAC, where neoplastic cellularity is generally low and stromal content is high. Understanding the distribution and plasticity of malignant and non-malignant states within individual PDAC tumors has important implications for the interpretation of transcriptional subtypes, directing therapy, and monitoring tumor evolution. However, few single cell studies have been conducted in human PDAC, and these have largely focused on stromal cell types or provided a limited analysis of malignant cells (11, 27-29). We therefore lack a harmonized view of the interplay between malignant transcriptional subtypes and their associated tumor microenvironment (TME).

Our current understanding of PDAC is largely derived from resected primary tumors (12, 13, 30). However, the majority of patients with PDAC present with, and succumb to, metastatic disease, which occurs most commonly in the liver (30). At present, there is little information about the cellular phenotypes and microenvironmental interactions in metastatic lesions. Tissue availability has been a key barrier to enhanced understanding of metastatic disease, as needle biopsies provide an important but cell-limited window into the biology of the metastatic niche.

In conjunction with detailed molecular analysis of patient samples, reliable ex vivo models are needed to functionally test clinical and molecular observations. For this purpose, human cancer cell line models are frequently utilized, as is the case in PDAC. However, the methods to generate new cell lines from human tissue are generally inefficient, which limits their utility in personalized medicine (31). Moreover, once established, cell lines can display significant drift in culture (32). To address these limitations, several groups have established efficient methods for generating patient-derived organoid cultures from PDAC tissue with the goal of modeling an individual patient's disease (10, 33-35). However, few studies have examined the fidelity and evolution of organoid phenotype and genotype relative to the parental patient tissue.

Here, Applicants developed and employed an optimized translational workflow to perform both high-resolution profiling of patient tissue using scRNA-seq via Seq-Well (36) and derivation of matched organoid models from the same metastatic core needle biopsy. Through this approach Applicants refrained bulk classifications by clarifying the underlying distribution of malignant phenotypes, revealed how microenvironmental heterogeneity is distributed in a transcriptional subtype-dependent manner, and systematically evaluated the ex vivo evolution and plasticity of malignant phenotypes.

### Results

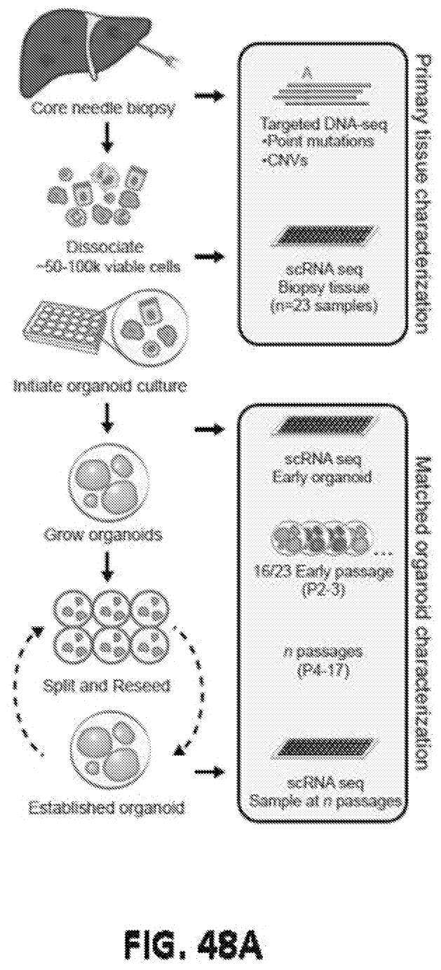

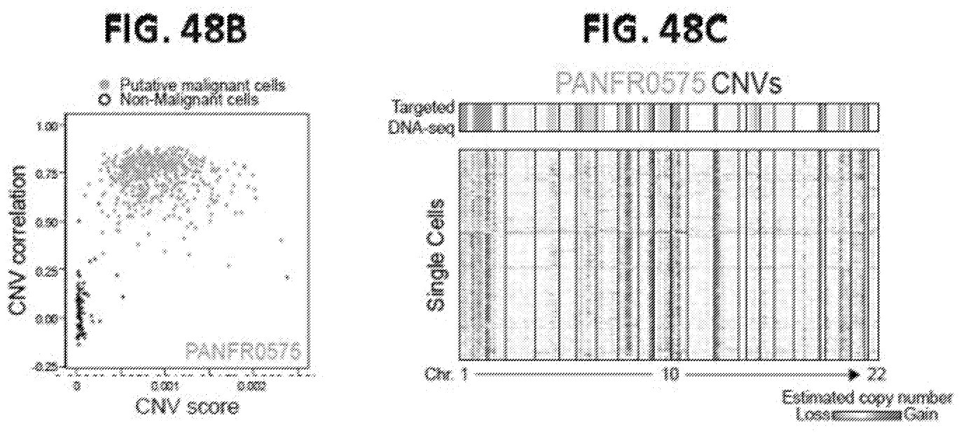

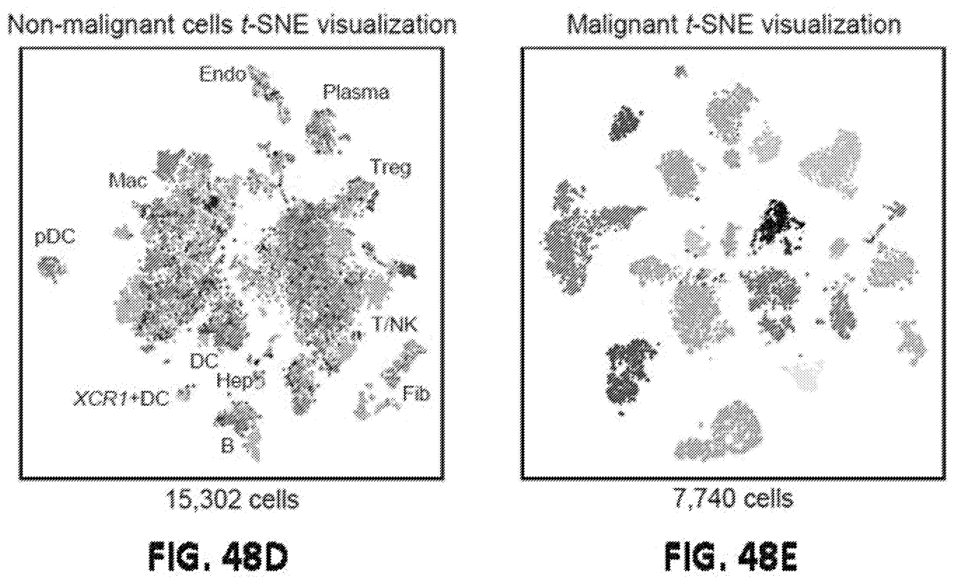

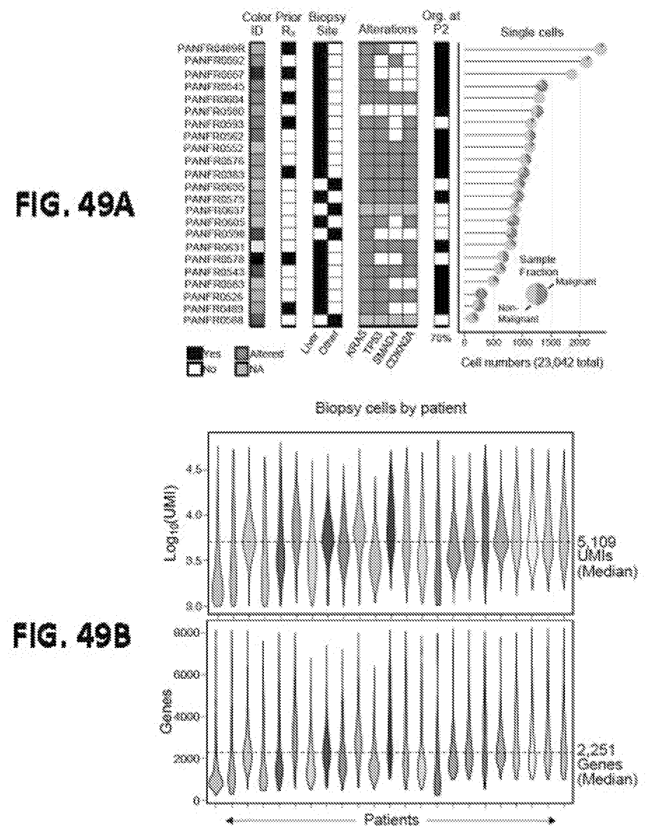

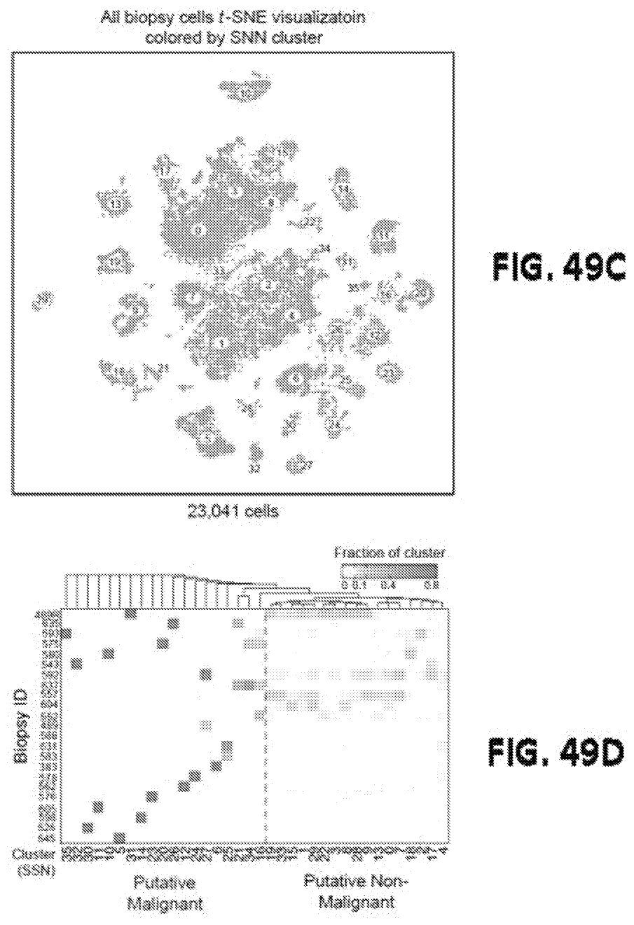

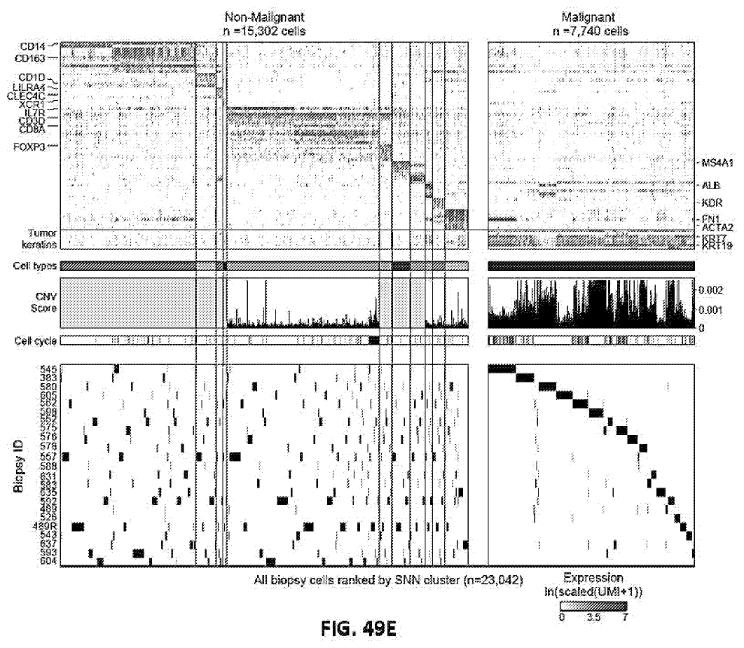

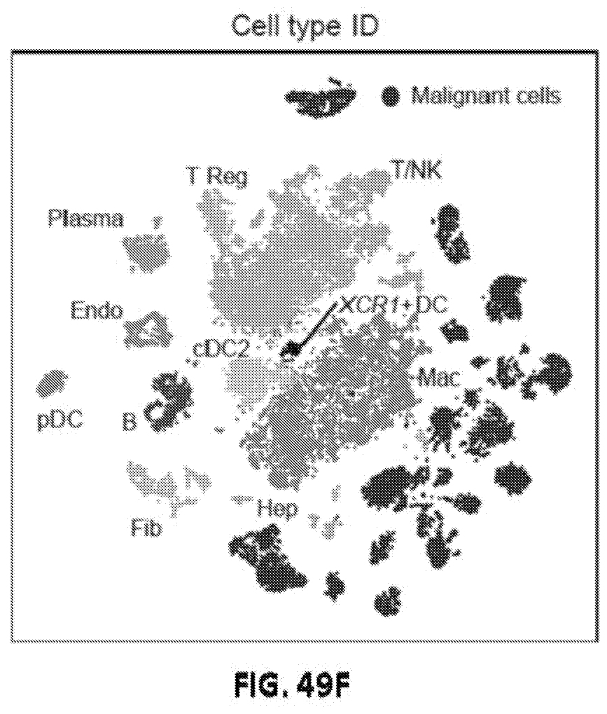

A clinical pipeline for matched single-cell profiling and organoid model generation. Applicants established a pipeline for collecting needle biopsies from patients with metastatic PDAC (n=23) to generate matched scRNA-seq profiles and organoid models (FIG. 48A, 49A, Table 1). Most samples were obtained from metastatic lesions residing in the liver (19/23), and the majority (21/23) were analyzed by targeted DNA-sequencing which yielded the expected mutational pattern for this disease (FIG. 49A) (4, 12, 13). After tissue dissociation, Applicants used 10,000-20,000 viable cells for scRNA-seq via Seq-Well, and the remainder were seeded for organoid culture (FIG. 48A). This pipeline yielded approximately 1,000 high-quality single cells per biopsy (n=23,042 total cells) and successful early-passage organoid cultures from 70% (16/23) of patient tumor samples (FIG. 49A, 49B). Dimensionality reduction and shared nearest neighbor (SNN) clustering of the biopsy cells revealed substantial heterogeneity at the single-cell level (FIG. 49C; Methods). The fractional representation from each biopsy readily split the data into two groups, clusters of admixed cells from multiple patients and distinct patient-specific clusters (FIG. 49D). This pattern suggested both malignant and non-malignant cells within each biopsy, with patient-specific clusters driven by specific copy number variations (CNVs). To confirm malignant cell identity, Applicants inferred transcriptome-wide CNVs from single-cell data as previously described (21, 26). CNV alteration scores separated putative malignant and non-malignant cells in each biopsy and demonstrated high concordance with reference targeted DNA-seq (FIG. 48B, 48C, 50A, 50B). CNV analysis paired with manual inspection of expression patterns for known markers across single cells supported the identification of malignant cells as well as unique non-malignant cell types (FIG. 49D-49F, 48D, 48E, Table 2). Thus, Applicants established a robust workflow capable of recovering high quality malignant (n=7,740) and non-malignant (n=15,302) populations from metastatic PDAC needle biopsies with low neoplastic cellularity while also enabling simultaneous generation of matched organoid models.

TABLE 1
Cohort patient characteristics. This table details the demographic and clinical
characteristics of patients whose biopsy samples were used in this study.

| | Patient | | | | | | |
|---|---|---|---|---|---|---|---|
| Sample | age at | | | Stage at | | | Number |
| (scRNA- | initial | | | initial | | Site of | of cores |
| seq ID) | diagnosis | Gender | Ethnicity | diagnosis | Histology | biopsy | collected |
| PANFR0383 | 76 | Male | Caucasian | Metastatic | PDAC | Liver | 7 |
| PANFR0489 | 67 | Female | Caucasian | Localized | PDAC | Liver | 6 |
| and | | | | | | | |
| PANFR0489R2 | | | | | | | |
| PANFR0526 | 72 | Male | Caucasian | Metastatic | PDAC | Liver | 5 |
| PANFR0543 | 65 | Male | Caucasian | Metastatic | PDAC | Liver | 7 |
| PANFR0545 | 75 | Female | Caucasian | Metastatic | PDAC | Liver | 5 |
| PANFR0552 | 49 | Female | Caucasian | Localized | PDAC | Liver | 4 |
| PANFR0557 | 44 | Female | Caucasian | Localized | PDAC | Liver | 4 |
| PANFR0562 | 76 | Female | Caucasian | Localized | PDAC | Liver | 5 |
| PANFR0575 | 79 | Female | Asian | Metastatic | PDAC | Liver | 5 |
| PANFR0576 | 63 | Male | Caucasian | Metastatic | PDAC | Liver | 5 |
| PANFR0578 | 62 | Female | Caucasian | Metastatic | PDAC | Liver | 4 |
| PANFR0580 | 53 | Male | Caucasian | Metastatic | Pan | Liver | 6 |
| | | | | | NET | | |
| PANFR0583 | 61 | Male | Caucasian | Metastatic | PDAC | Liver | 5 |
| PANFR0588 | 71 | Female | Caucasian | Metastatic | PDAC | Peritoneum | 6 |
| PANFR0592 | 37 | Male | Caucasian | Locally | PDAC | Liver | 4 |
| | | | | advanced | | | |
| PANFR0593 | 63 | Male | Caucasian | Localized | PDAC | Liver | 4 |
| PANFR0598 | 75 | Male | Caucasian | Metastatic | PDAC | Omentum | 7 |
| PANFR0604 | 67 | Female | Caucasian | Metastatic | PDAC | Liver | 8 |
| PANFR0605 | 75 | Male | Caucasian | Metastatic | PDAC | Liver | 8 |
| PANFR0631 | 66 | Male | Caucasian | Metastatic | PDAC | Liver | 12 |
| PANFR0635 | 76 | Female | Caucasian | Metastatic | PDAC | Omentum | 7 |

| | Number | | | | Survival |
|---|---|---|---|---|---|
| | of cores | | Metastatic | Patient | time from |
| Sample | for scRN | Treatment | treatments | status | initial |
| (scRNA- | Aseq/ | of primary | prior to | (alive/ | diagnosis |
| seq ID) | organoids | disease | biopsy | deceased) | (days)1 |
| PANFR0383 | 2 | None | Gemcitabine/ | Deceased | 302 |
| | | | Nabpaclilaxel/ | | |
| | | | Antihepatocyte | | |
| | | | growth factor | | |
| | | | antibody | | |
| PANFR0489 | 2 | Whipple; | Gemcitabine/ | Deceased | 2068 |
| and | | FOLFOX/ | Nabnaclitaxel; | | |
| PANFR0489R2 | | Nabpaclitaxel; | Gemcitabine; | | |
| | | Capecitabine + | 5-FU/Liposomal | | |
| | | radiation | irinotecan; | | |
| | | | FOLFOX | | |
| PANFR0526 | 2 | None | None | Deceased | 10 |
| PANFR0543 | 5 | None | None | Deceased | 120 |
| PANFR0545 | 2 | None | None | Deceased | 94 |
| PANFR0552 | 2 | FOLFIRINOX; | None | Deceased | 536 |
| | | Whipple; | | | |
| | | Gemcitabine/ | | | |
| | | Nabpaclitaxel | | | |
| PANFR0557 | 3 | Distal | FOLFIRINOX; | Alive | 1154 |
| | | pancreatectomy; | FOLFIRI | | |
| | | Gemcitabine/ | | | |
| | | Capecitabine | | | |
| PANFR0562 | 2 | None | None | Deceased | 111 |
| PANFR0575 | 2 | None | None | Deceased | 32 |
| PANFR0576 | 2 | None | None | Deceased | 159 |
| PANFR0578 | 1 | None | FOLFIRINOX; | Alive | 1653 |
| | | | Lung | | |
| | | | metastatectomy; | | |
| | | | FOLFIRINOX; | | |
| | | | 5-FU + | | |
| | | | radiation; | | |
| | | | Olaparib; | | |
| | | | Whipple | | |
| PANFR0580 | 2 | None | None | Alive | 454 |
| PANFR0583 | 2 | None | None | Deceased | 52 |
| PANFR0588 | 2 | None | None | Deceased | 227 |
| PANFR0592 | 2 | FOLFIRINOX; | None | Deceased | 376 |
| | | Whipple | | | |
| PANFR0593 | 2 | Distal | Gemcitabine/ | Alive | 146 (lost |
| | | pancreatectomy; | Nabpaclitaxel/ | | to follow |
| | | FOLFIRINOX | Trastuzumab | | up) |
| PANFR0598 | 2 | None | None | Alive | 392 |
| PANFR0604 | 2 | None | FOLFIRINOX | Alive | 854 |
| PANFR0605 | 3 | None | Norte | Deceased | 322 |
| PANFR0631 | 2 | None | None | Alive | 308 |
| PANFR0635 | 3 | None | None | Alive | 279 |

footnotes
1Patient survival was calculated as time from initial diagnosis to death or last documented follow-up for patients who are alive.
2This patient had two samples, PANTR0489 and PANFR0489R, collected at different time points along their treatment course.

### Table 2. Normal Cell Type Markers (See Pages 145-162).

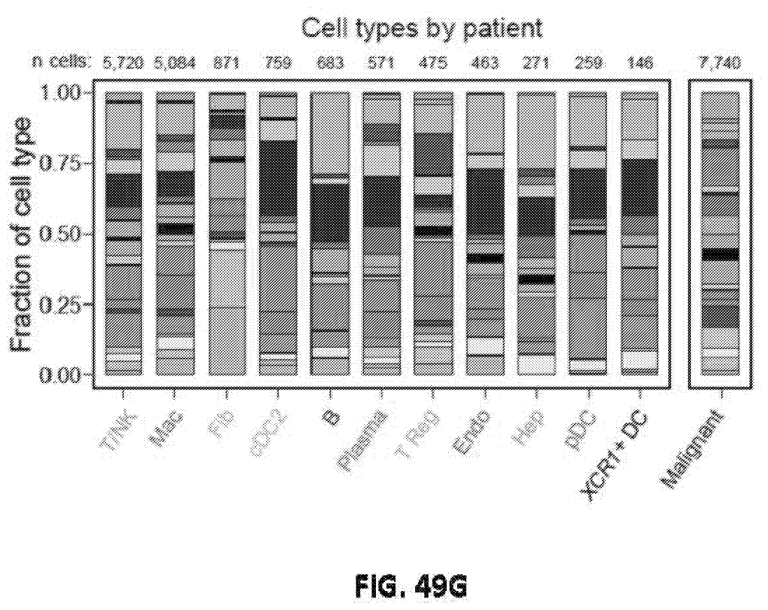

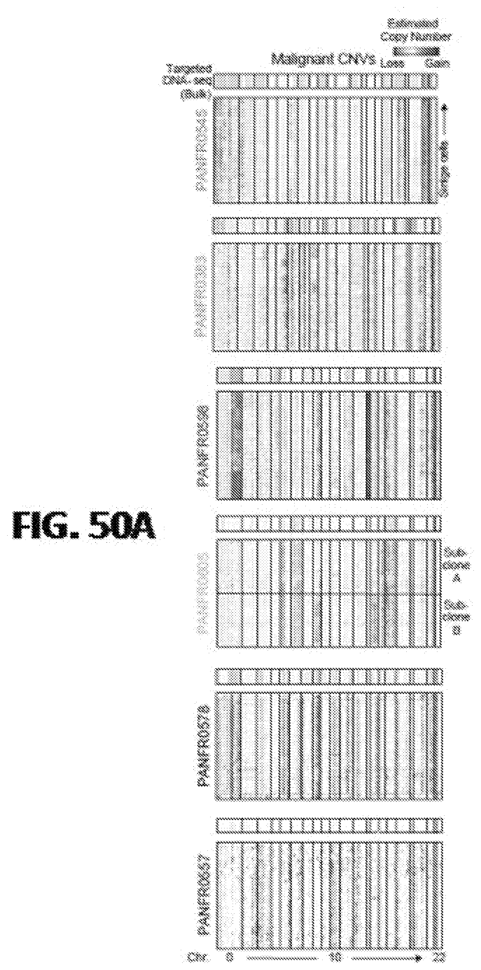

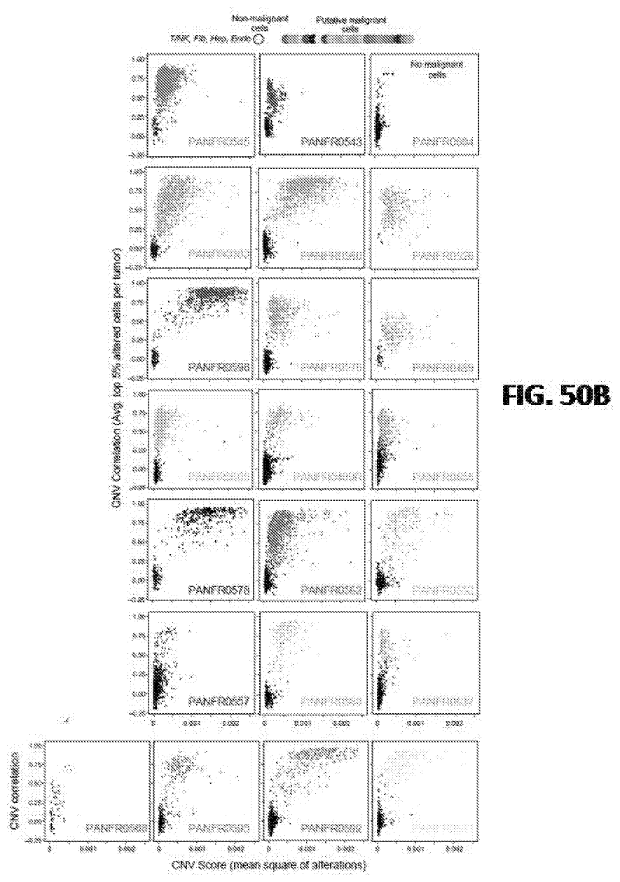

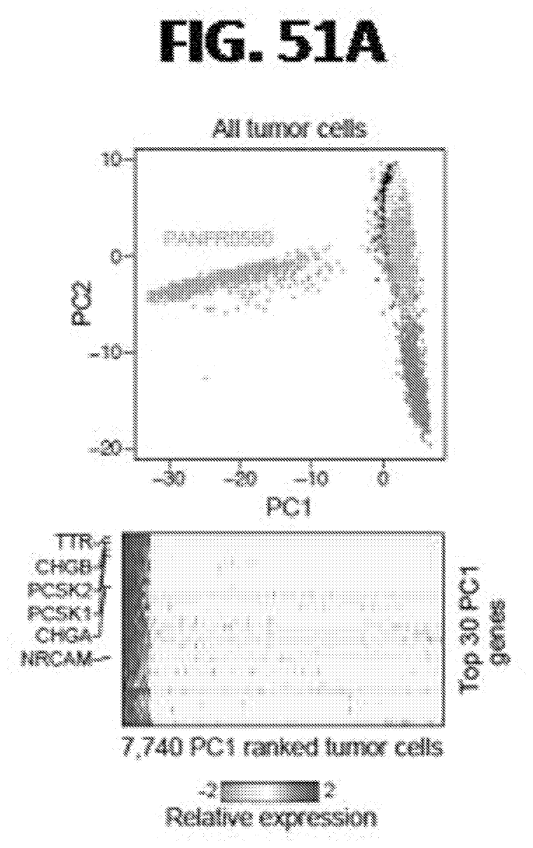

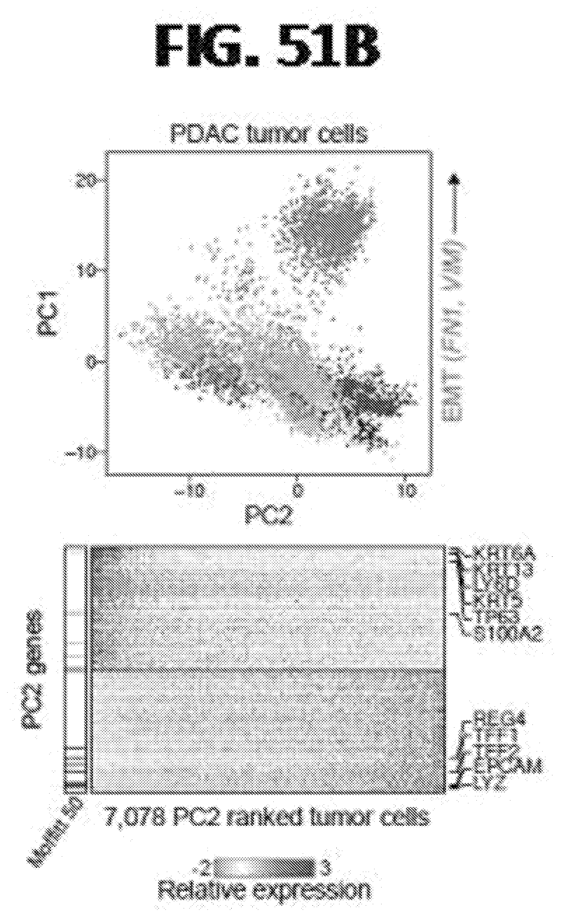

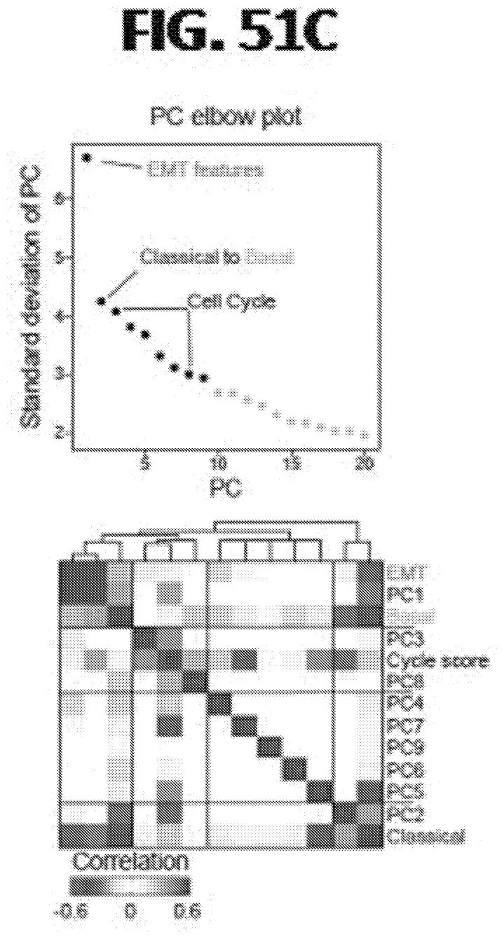

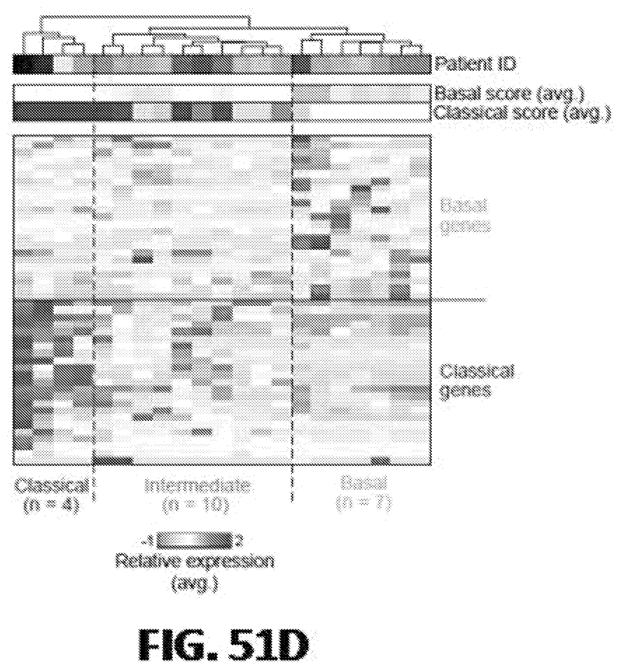

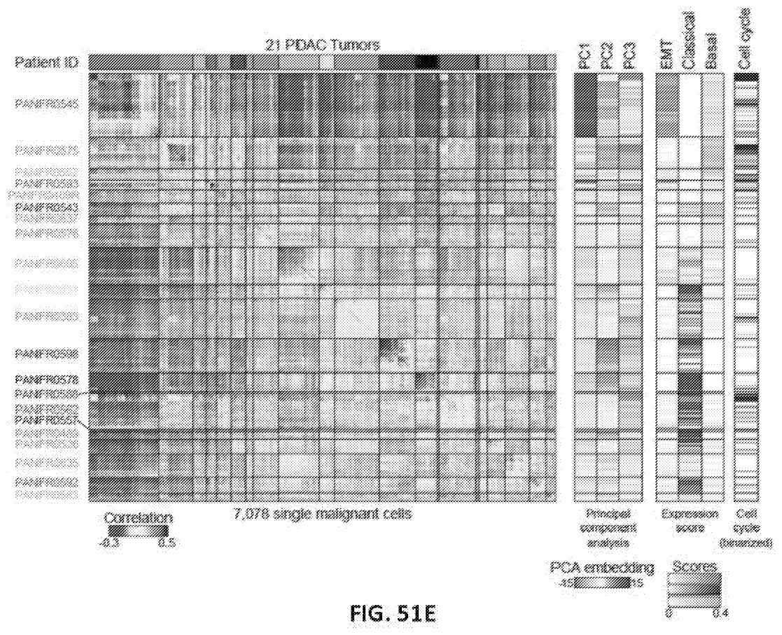

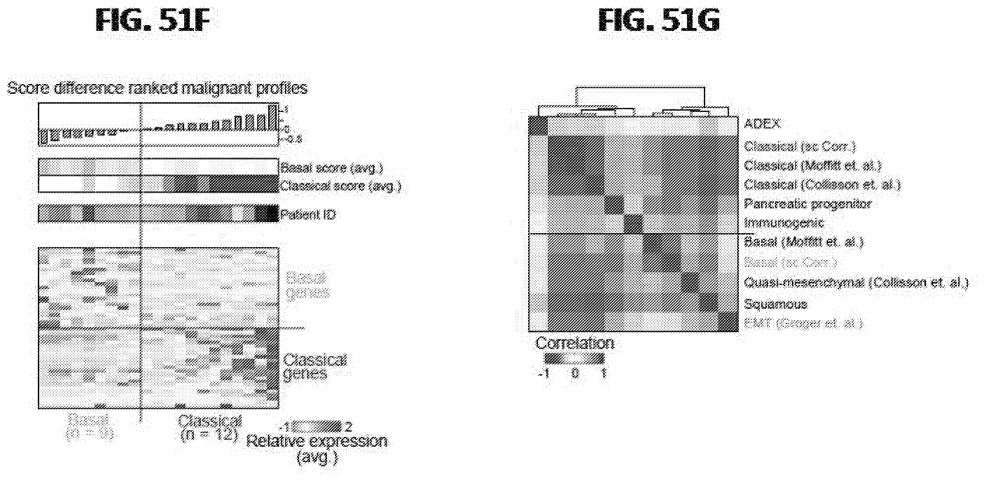

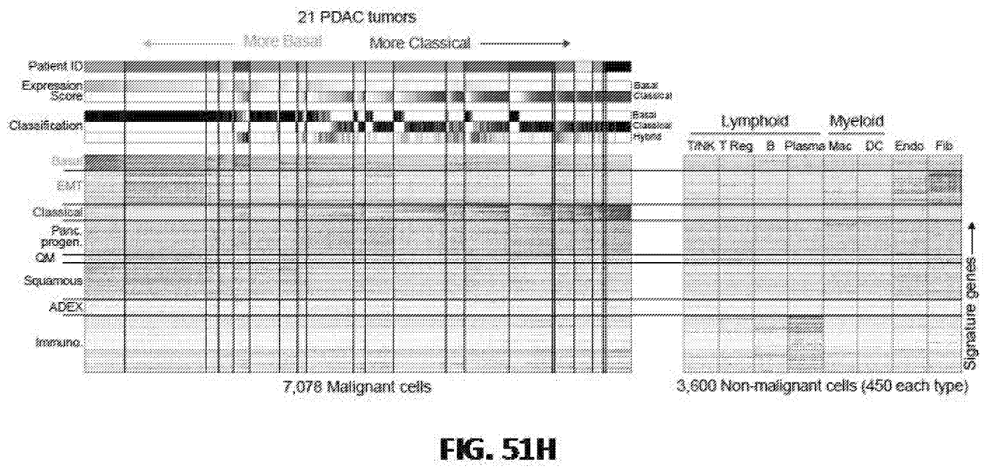

PDAC transcriptional subtypes exist on a continuum and include hybrid expression states. Applicants first applied principal component analysis (PCA) to examine major axes of transcriptional variation across malignant cells from all biopsy samples. Notably, Applicants failed to identify canonical driver mutations typically observed in PDAC in one patient sample obtained prior to a pathologically confirmed clinical diagnosis, PANFR0580 (FIG. 49A); however, Applicants detected a significant fraction of putative malignant cells (n=662) in this biopsy (FIG. 50B). Principal component 1 (PC1) separated PANFR0580 from all other tumors in the cohort (FIG. 51A, top). Genes with the strongest negative loading on PC1 were indicative of a neuroendocrine phenotype (TTR, CHGA, CHGB; FIG. 51A, bottom) and subsequent pathological evaluation confirmed that this sample was a pancreatic neuroendocrine tumor (PanNET). To focus on transcriptional heterogeneity among PDAC samples, Applicants removed the PanNET cells and performed a new PCA on the remaining 7,078 malignant cells. Inspection of the genes driving the first 3 PCs within PDAC cells revealed separation along previously characterized transcriptional phenotypes (epithelial/mesenchymal transition (EMT) (37), PC1; basal/classical (5), PC2; cell cycle (16), PC3; FIG. 51B, 51C), confirming that the main axes of variation in the data align with established transcriptional subtypes.

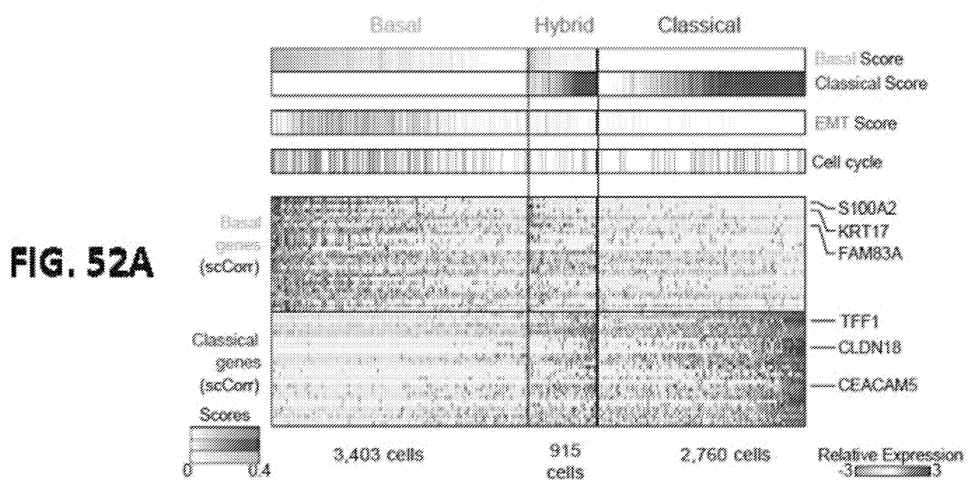

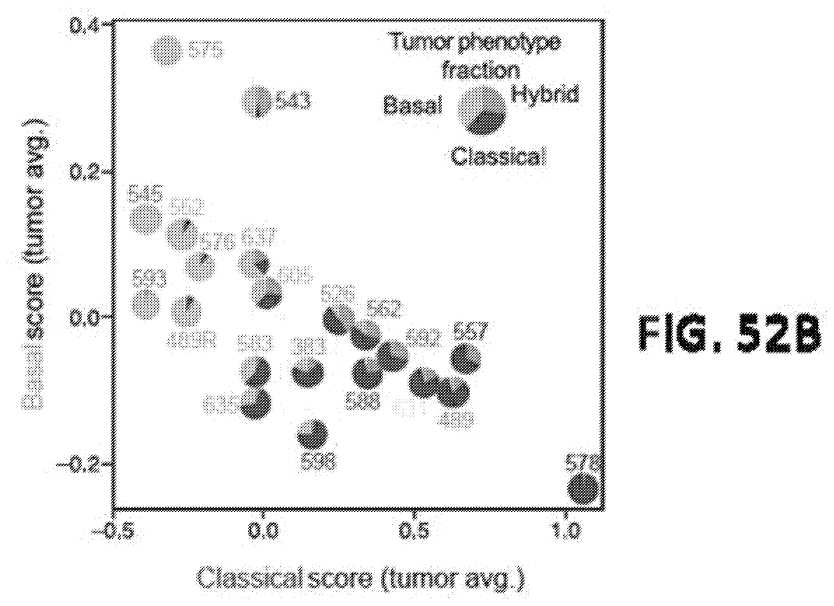

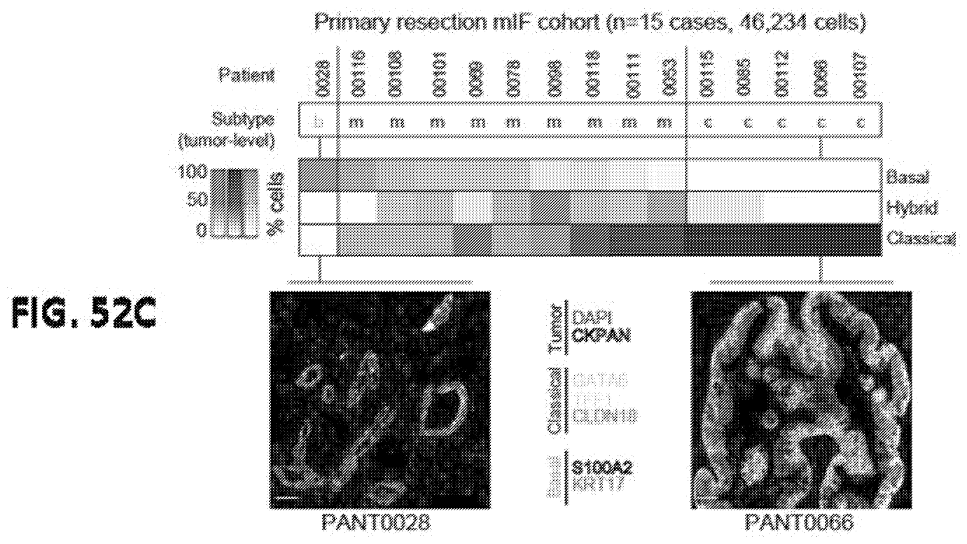

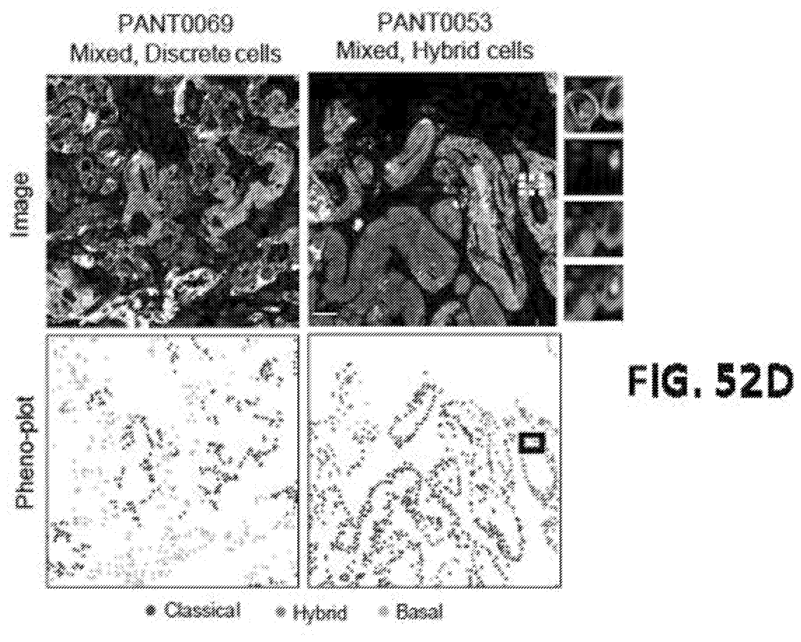

Previous studies using bulk RNA-seq data have converged on two main tumor subtypes, basal and classical (3-5, 11-13). Collapsing the malignant cells from each sample into a pseudo-bulk averaged transcriptome split the cohort into 3 groups: those that exhibit predominately basal character (n=7), those with more classical features (n=4), and those that are intermediate (n=10; FIG. 51D). Examination of basal and classical phenotypes within each biopsy at single-cell resolution suggested that tumors are comprised of a heterogenous mixture of states, likely driving the ambiguous classification of weakly polarized tumors when using hierarchical clustering (FIG. 51D, 51E). Applicants also observed a significant fraction of malignant cells co-expressing both basal and classical phenotypes, hereafter referred to as “hybrid” cells (˜13% of malignant cells, FIG. 52A, see Methods), suggesting that these phenotypes exist on a continuum rather than as discrete states. Classification of each single cell as basal, classical, or hybrid revealed substantial heterogeneity across individual tumors for these phenotypes (FIG. 52B). These observations underscore the difficulty in assigning intermediate tumors exclusively to basal or classical groups (11). Thus, where discrete binning was necessary, Applicants employed a basal-classical “score difference” to stratify samples and preserve the polarization for each tumor along this continuum (FIG. 51F).

Applicants also used their single-cell data to examine signatures proposed by other bulk RNA sequencing studies to clarify their inter-relationships. Pairwise correlation of all established signatures in malignant cells revealed that many contribute overlapping information and reflect similar underlying biology (FIG. 51G). Applicants observed that cells with higher basal expression were also classified as squamous and quasimesenchymal, while cells with classical signatures were correlated with the pancreatic progenitor subtype (FIG. 51G, 51H) (3, 4). By contrast, Applicants did not observe evidence for expression of the immunogenic, ADEX, or exocrine-like transcriptional signatures in malignant cells (3, 4). While the absence of these signatures might represent differences between primary and metastatic disease, these bulk RNA profiles also likely incorporate signals from non-malignant cells in the TME. In support of the latter hypothesis, Applicants find evidence of immunogenic signature expression originating from plasma cells as well as EMT signature expression from both malignant cells and fibroblasts (FIG. 51H). These patterns underscore the need for single-cell resolution to dissect malignant and non-malignant contributions to transcriptional signatures.

Applicants next confirmed the presence of basal, classical, and hybrid cells using a novel subtype-specific single cell multiplexed immunofluorescence (mIF) panel in a cohort of primary resected PDAC (n=15 cases, 46,234 cells, Methods; FIG. 52C; FIG. 53A-53C, Tables 3 and 4). This orthogonal approach confirmed the intratumoral heterogeneity observed in Applicants' scRNA-seq cohort and revealed that PDAC transcriptional subtype diversity occurs on two levels: (i) “mixed” tumors comprised of discrete cells with differing subtype identity, and (ii) hybrid cells which co-express basal and classical programs. These observations indicate that PDAC transcriptional subtypes exist on a continuum, with mixed and hybrid phenotypes occurring even within a single tumor gland (FIG. 52D).

TABLE 3
mIF marker combinations. This table indicates how
each individual cell was subtyped based on different
combinations of mIF marker positivity.

| Cell subtype | KRT17 | S100A2 | TFF1 | CLDN18.2 | GATA6 |
|---|---|---|---|---|---|
| Negative/Excluded | − | − | − | − | − |
| Basal | + | + | − | − | − |
| Basal | + | − | − | − | − |
| Basal | − | + | − | − | − |
| Classical | − | − | + | + | + |
| Classical | − | − | + | + | − |
| Classical | − | − | + | − | + |
| Classical | − | − | + | − | − |
| Classical | − | − | − | + | + |
| Classical | − | − | − | + | − |
| Classical | − | − | − | − | + |
| Hybrid | + | + | + | + | + |
| Hybrid | + | + | + | + | − |
| Hybrid | + | + | + | − | + |
| Hybrid | + | + | + | − | − |
| Hybrid | + | + | − | + | + |
| Hybrid | + | + | − | + | − |
| Hybrid | + | + | − | − | + |
| Hybrid | + | − | + | + | + |
| Hybrid | + | − | + | + | − |
| Hybrid | + | − | + | − | + |
| Hybrid | + | − | + | − | − |
| Hybrid | + | − | − | + | + |
| Hybrid | + | − | − | + | − |
| Hybrid | + | − | − | − | + |
| Hybrid | − | + | + | + | + |
| Hybrid | − | + | + | + | − |
| Hybrid | − | + | + | − | + |
| Hybrid | − | + | + | − | − |
| Hybrid | − | + | − | + | + |
| Hybrid | − | + | − | + | − |
| Hybrid | − | + | − | − | + |

TABLE 4
mIF fields of view and subtyping cell counts. This table
demonstrates the number of fields of view and subtyped cells
quantified for each sample in FIG. 52C, 52D, and 53.

| | | | | | Total | Total |
|---|---|---|---|---|---|---|
| | | Num- | Num- | Num- | cells | cells |
| | Fields | ber | ber | ber | (ex- | (in- |
| | of view | basal | classical | hybrid | cluding | cluding |
| Case ID | (FOV) | cells | cells | cells | negatives) | negatives) |
| PANT00101 | 8 | 580 | 267 | 318 | 1165 | 3124 |
| PANT00107 | 6 | 5 | 3753 | 9 | 3767 | 4571 |
| PANT00108 | 9 | 1002 | 397 | 436 | 1835 | 4235 |
| PANT00111 | 14 | 499 | 1668 | 711 | 2878 | 6334 |
| PANT00112 | 11 | 79 | 5680 | 143 | 5902 | 10813 |
| PANT00115 | 10 | 119 | 4028 | 512 | 4659 | 6853 |
| PANT00116 | 9 | 831 | 237 | 10 | 1078 | 5609 |
| PANT00118 | 10 | 1289 | 2115 | 1336 | 4740 | 6522 |
| PANT0028 | 3 | 729 | 15 | 10 | 754 | 1746 |
| PANT0053 | 8 | 537 | 4595 | 3198 | 8330 | 9452 |
| PANT0066 | 5 | 1 | 2038 | 25 | 2064 | 2560 |
| PANT0069 | 7 | 2585 | 2029 | 622 | 5236 | 7821 |
| PANT0078 | 4 | 253 | 125 | 161 | 539 | 2244 |
| PANT0085 | 6 | 21 | 2454 | 333 | 2808 | 3205 |
| PANT0098 | 7 | 105 | 140 | 234 | 479 | 1003 |
| Total | 117 | 8635 | 29541 | 8058 | 46234 | 76092 |

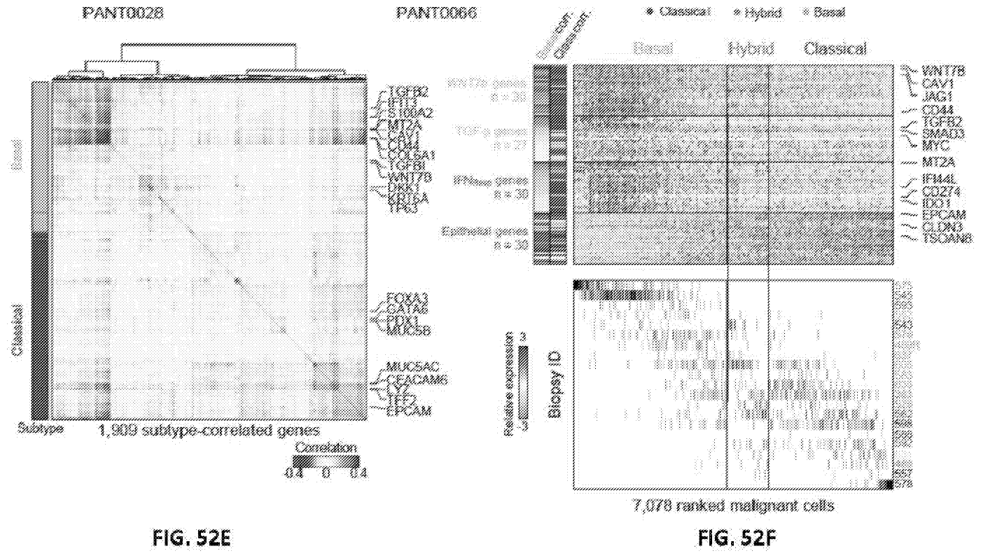

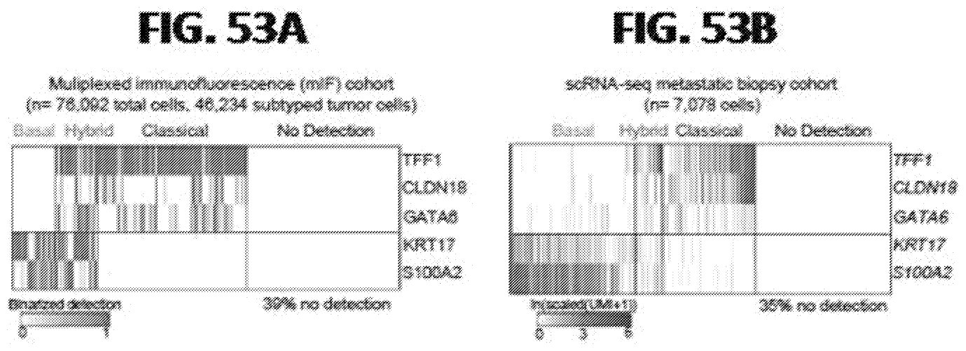

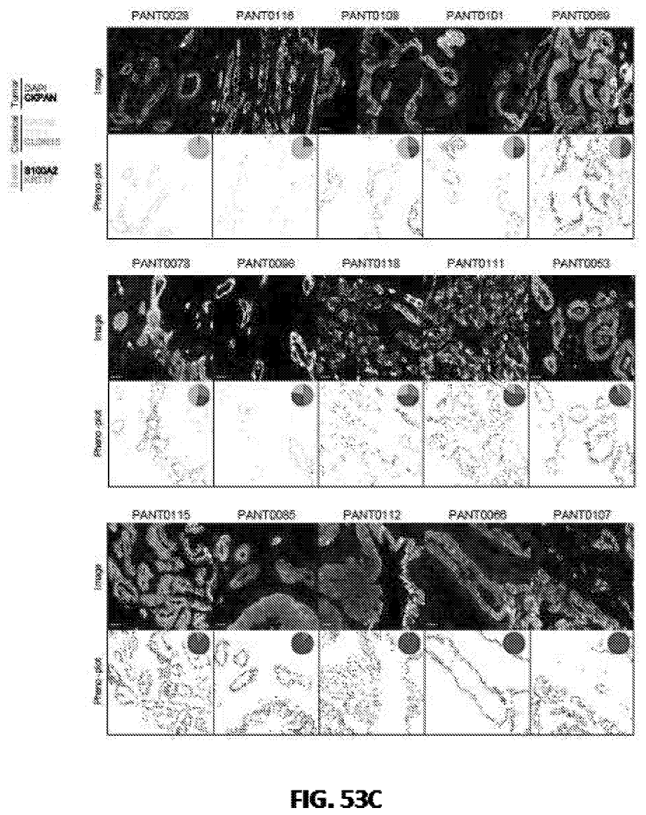

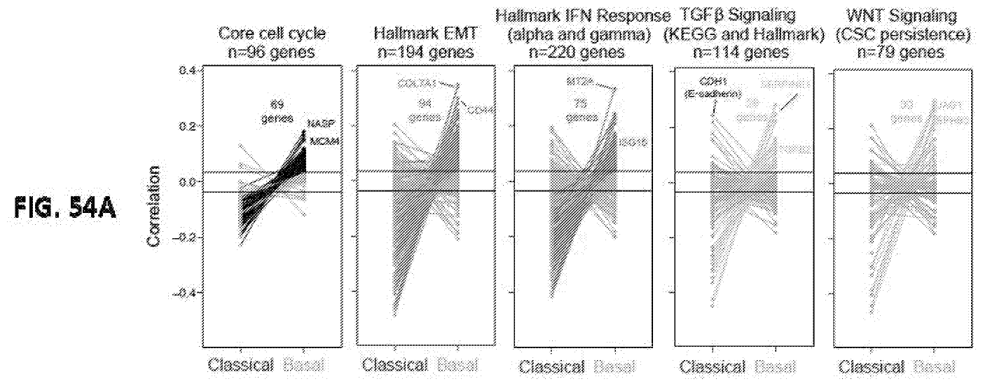

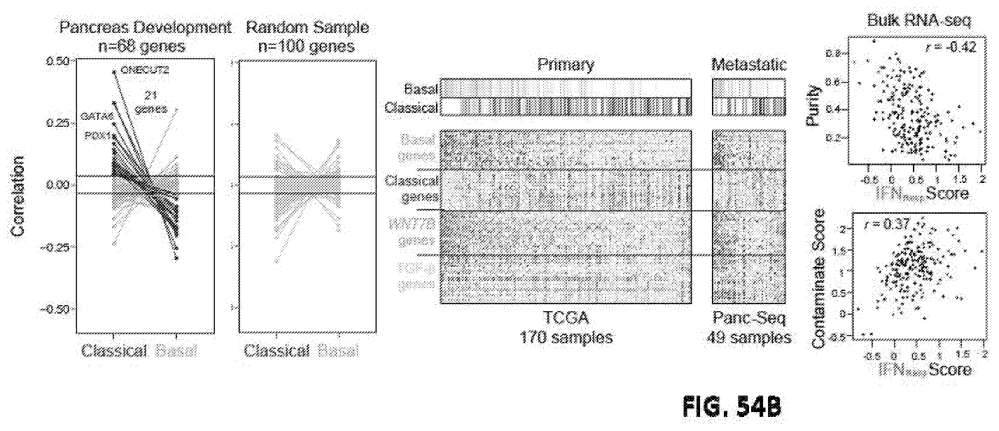

Basal and classical cells exhibit subtype-specific expression programs. Applicants next leveraged their single-cell resolution to examine whether specific tumor cell gene expression programs were correlated with either the basal or classical phenotypes. This correlation analysis across malignant cells revealed 1,909 genes significantly associated with either basal or classical expression scores (FIG. 52E; Tables 5, 6; Methods). Inspection of these genes revealed basal cells are defined by more mesenchymal features and co-express programs associated with transforming growth factor beta (TGFB2, SERPINE1; TGF-β) signaling, interferon response (IFI44L, ISG15; IFN_{Rep}), WNT signaling (WNT71B, FZD6, EPHB2; WNT), and cell cycle progression (NASP, TOP2A) (37-40). Notably, these patterns are concordant with larger bulk RNA-seq cohorts from primary and metastatic patient samples (FIG. 52E, 52F, 54A, 54B) (12, 13). While WNT ligands are included in organoid culture media and thought to be necessary to support tumor cell growth ex vivo, Applicants consistently detected only the WNT ligands WNT7B and WNT10A, which are enriched in malignant basal cells in vivo (FIG. 54C) (33, 41). Conversely, epithelial and pancreatic progenitor transcriptional programs are enriched in classical PDAC cells (FIG. 52E, 52F, 54A). Together, these expression patterns suggest a developmental continuum within PDAC tumors from higher cycling (FIG. 52A), de-differentiated basal cells to more committed classical epithelial pancreatic progenitors that mirror phenotypes seen in the early developing pancreas.

### Table 5. Basal Single-Ell Gene Correlates (See Pages 163-330).

### Table 6. Classical Single-Cell Gene Correlates (See Pages 331-492).

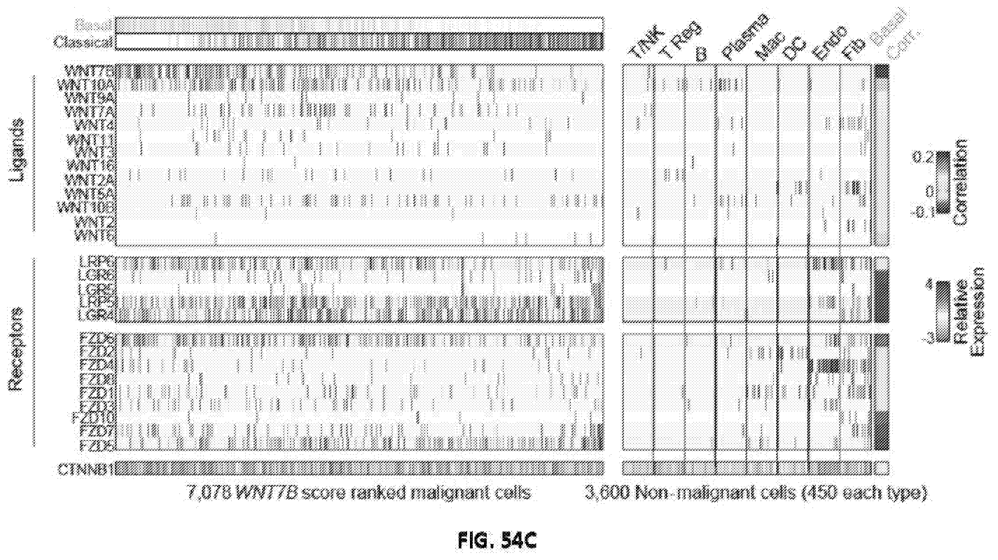

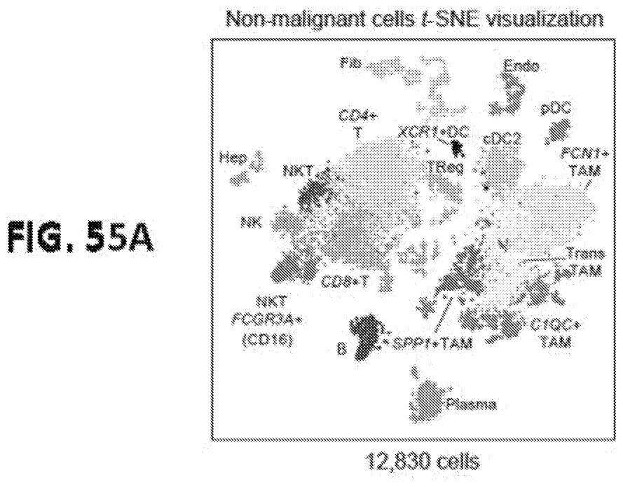

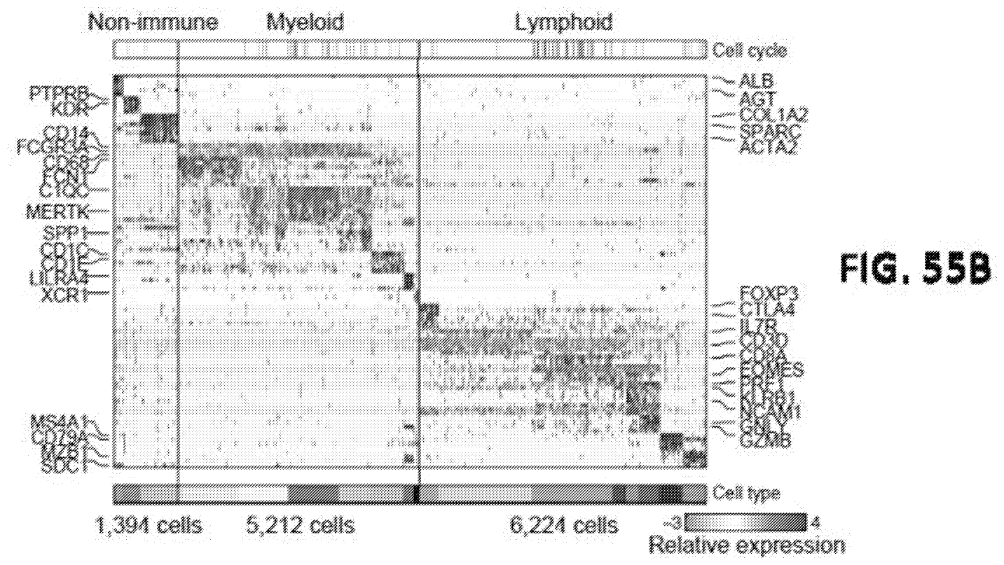

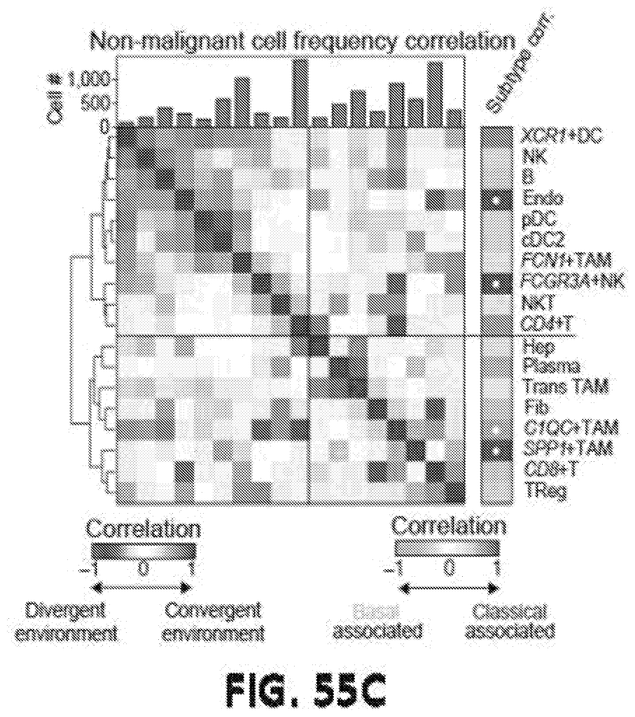

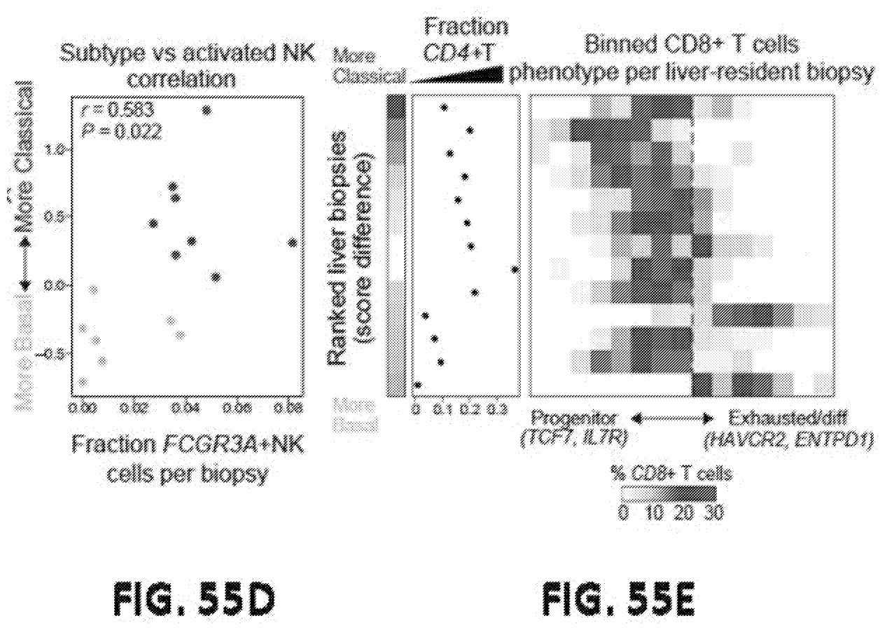

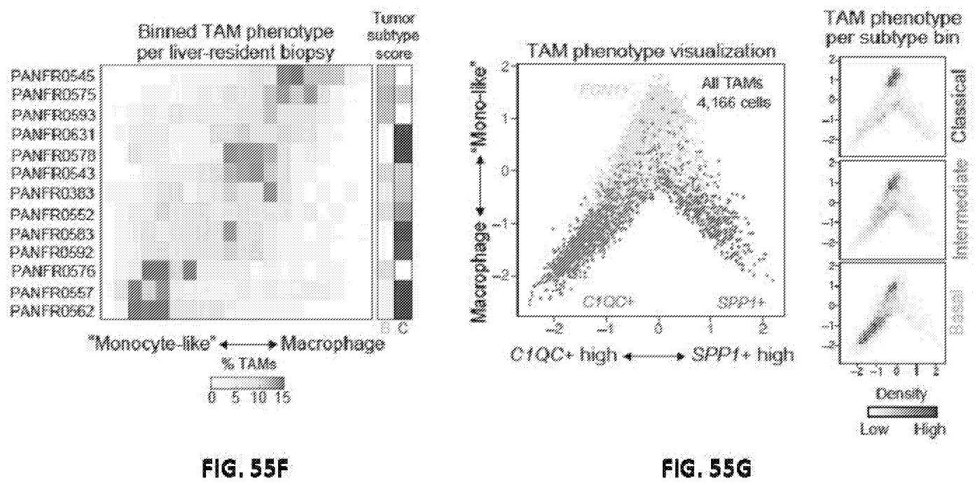

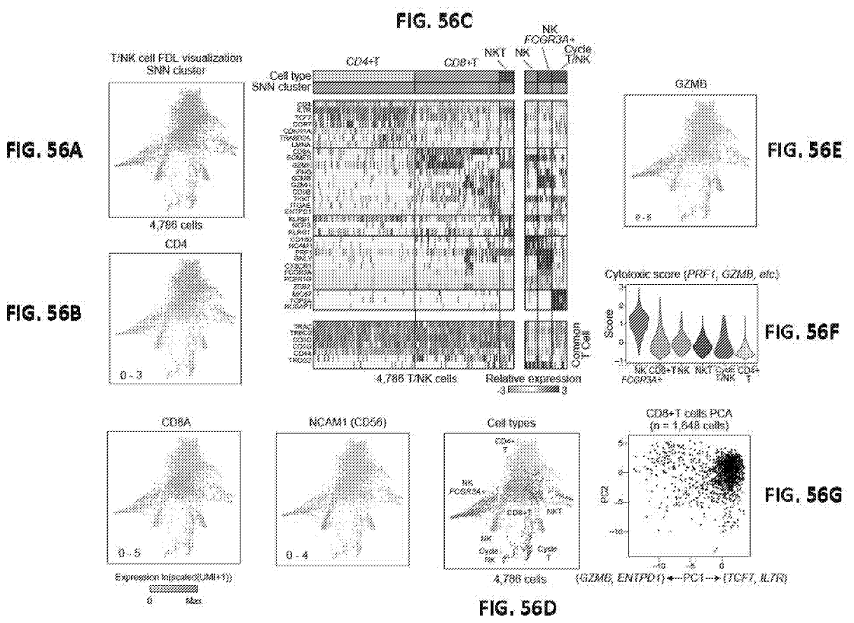

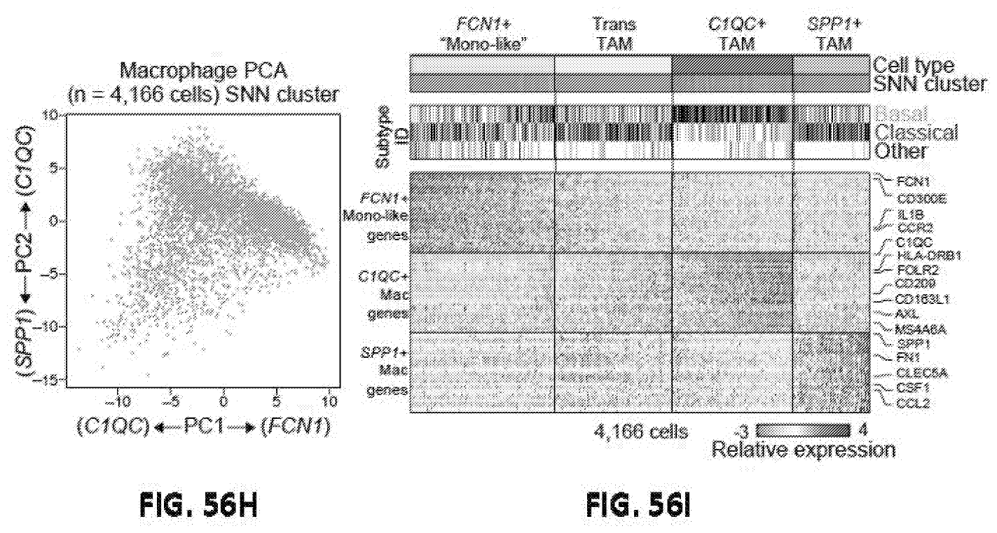

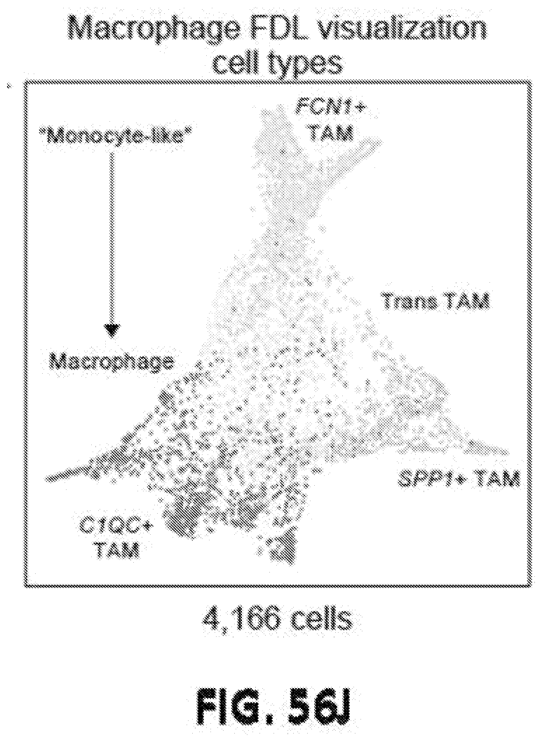

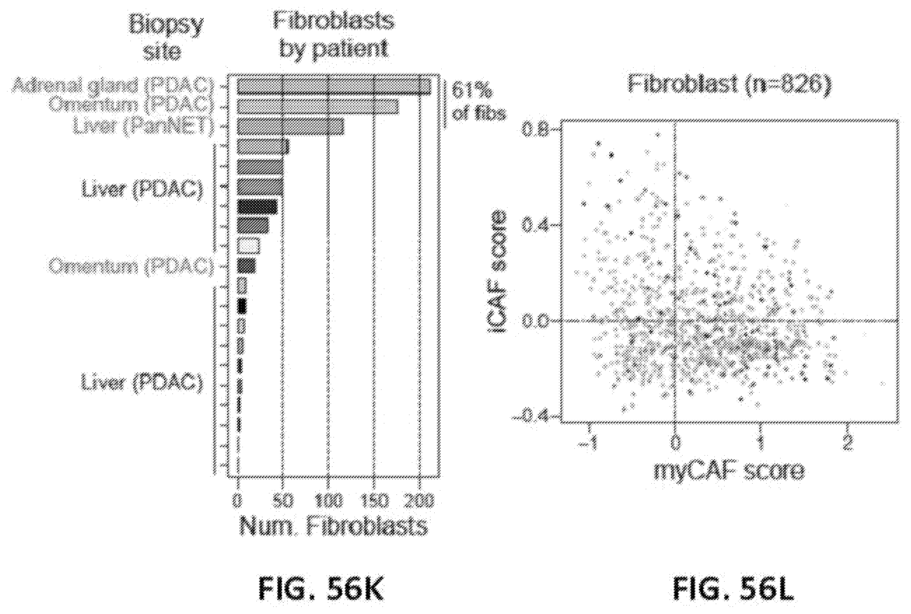

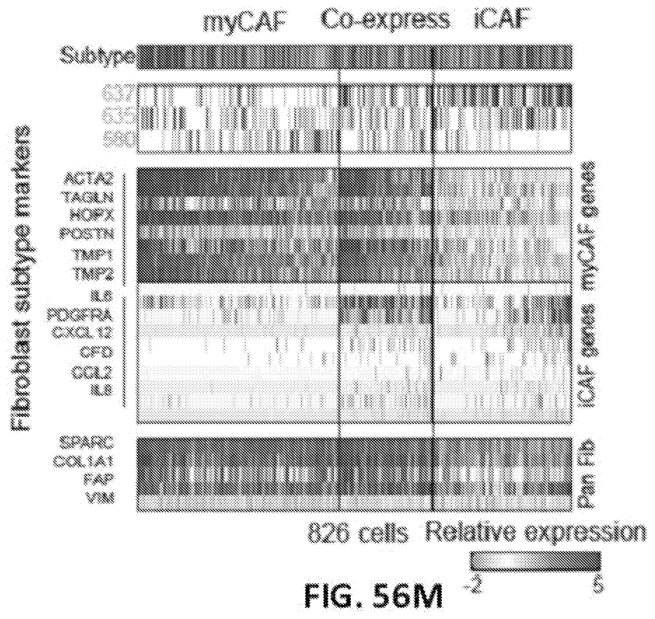

Transcriptional subtypes associate with distinct immune microenvironments. Relatively little is known about the structure and composition of the metastatic microenvironment in PDAC, and, more specifically, about how non-malignant heterogeneity associates with the basal to classical continuum. To characterize the cell types in the metastatic niche, we analyzed the non-malignant cells (n=12,830) and refined our broad cell-typing scheme from FIG. 48D by further subdividing the T/NK cells, monocytes/macrophages, and fibroblasts (FIG. 55A, 55B). First, a closer analysis of the T/NK cell cluster revealed 5 cell types-CD4+ T, CD8+ T, NKT, NK, and CD16+ (FCGR3A+) NK cells—each expressing the corresponding established markers (FIG. 56A-56D). Similarly, an unsupervised examination within the monocyte/macrophage compartment revealed a tumor associated macrophage (TAM) continuum similar to one recently described in colorectal cancer (42, 43). The first two PCs readily identified 3 TAM subsets: “monocyte-like” FCN1+, C1QC+, and SPP1+ macrophages (FIG. 56H). FCN1+“monocyte-like” cells expressed high levels of IL1B and CCR2 and shared some features with CD14+ blood monocytes (CD300E, S100A8) (42). C1QC+ TAMs resembled a phagocytic phenotype (CD163, MERTK), but also demonstrated preferentially high expression of antigen presentation genes (HLA-DRB1, CD74) and genes described in anti-inflammatory macrophage subsets (FOLR2, CD209, AXL, CSF1R). Conversely, SPP1+ TAMs expressed gene programs associated with angiogenesis (SPP1, FLT1) and inflammatory response (CCL2, CCL7, CSF1, CLEC5A). A fourth subset was positioned as intermediate between these three phenotypes and likely represents a population of actively transitioning/differentiating TAMs (Trans TAM; FIG. 56H-56J) (42). Finally, although several scRNA-seq studies in primary resected PDAC have focused on fibroblast phenotypes, Applicants observed few fibroblasts per tumor (Methods), with the outliers coming from sampling sites other than the liver (PANFR0637 and PANFR0635) or from a different disease etiology (PANFR0580, PanNET; FIG. 56K) (27-29). Still, in the fibroblasts recovered Applicants noted evidence of previously identified subtypes including myofibroblastic and inflammatory cancer-associated fibroblasts (myCAFs and iCAFs, respectively) in this metastatic setting (FIG. 56L, 56M). Taken together, Applicants identified 18 unique cell types/states in the PDAC metastatic microenvironment (FIG. 55A).

Applicants next determined whether the 18 non-malignant cell types/states were represented evenly across the malignant basal-to-classical transcriptional continuum described in FIG. 52. For this analysis, Applicants computed two quantities: 1) the fractional representation of each non-malignant cell type per biopsy and 2) the correlation of each non-malignant cell type's capture frequency to the average “score difference” (basal/classical polarization; FIG. 51F) derived from the malignant cells in the same biopsy. Cross-correlation of each cell type's fractional representation revealed two distinct patterns that largely diverged by malignant transcriptional subtype association (FIG. 55C). Overall, cell types traditionally believed to facilitate a more immune-responsive microenvironment were frequently captured together. For example, DC subsets, NK, B, CD4+ T and inflammatory FCN1+ TAMs derive from shared microenvironments (hereafter “immune-infiltrated”) and tend to associate with more classical tumors (FIG. 55C). Activated, mature NK cells (FCGR3A+NK) were captured most often from these immune-infiltrated biopsies and showed a strong correlation with classical tumors (FIG. 55D). Interestingly, FCGR3A+NK cells showed the highest expression of cytotoxic markers in Applicants' metastatic dataset, even compared to CD8+ T cells (FIG. 56E, 56F). Examination of the T cell compartment revealed that CD4+ T cells were captured more frequently in classical tumors (FIG. 55C, 55E), whereas CD8+ T cells were captured less frequently in immune-infiltrated biopsies and associated more often with an increased basal score. PCA within the CD8+ compartment revealed a progenitor (TCF7, IL7R) to differentiated/exhausted (HAVCR2, ENTPD1) continuum previously associated with differential outcomes to immune checkpoint blockade (FIG. 56G) (20, 44). Scoring each CD8+ T cell over this axis, we observed a progenitor-restricted distribution in most tumors, with only two outlier basal tumors skewing toward more differentiated/exhausted phenotypes (FIG. 55E). In sum, these findings indicate that much of the cytotoxic activity in the metastatic niche may originate from the innate immune system by way of activated NK cells in the microenvironment of classical tumors.

Along with differences in lymphocyte content, the myeloid compartment, specifically TAM phenotypes, showed strong subtype-specific associations. First, Applicants noted selective skewing for the types of TAMs originating from basal versus classical tumors (FIG. 56I, P<2.2×10-16, Chi-squared test; FIG. 55C, C1QC+ TAM, r=−0.59, basal association and SPP1+ TAM, r=0.52, classical association). Indeed, when examining the monocyte-like to macrophage distribution for TAMs from individual liver biopsies, the most basal-polarized tumors were associated with more macrophage-committed phenotypes (FIG. 55F). Moreover, by scoring each macrophage using TAM subtype-specific signatures and visualizing them with respect to the likely differentiation trajectory inferred from recent studies (Tables 7-9; Methods) (42), Applicants confirm a preferential association between C1QC+ TAMs and basal tumors and, conversely, an enrichment for the inflammatory FCN1+ monocyte-like and SPP1+ TAM subsets in tumors with intermediate and classical phenotypes (FIG. 55G). In addition to demonstrating that classical tumors are relatively more immune infiltrated, this analysis also identifies distinct microenvironmental phenotypes that co-vary with each PDAC transcriptional subtype and suggests opportunities to direct microenvironmental therapies in a subtype-specific manner.

### Table 7. FCN1+ Macrophage Subtype Markers (See Pages 493-548).

### Table 8. C1QC+ Macrophage Subtype Markers (See Pages 549-661).

### Table 9. SPP1+ Macrophage Subtype Markers (See Pages 662-759).

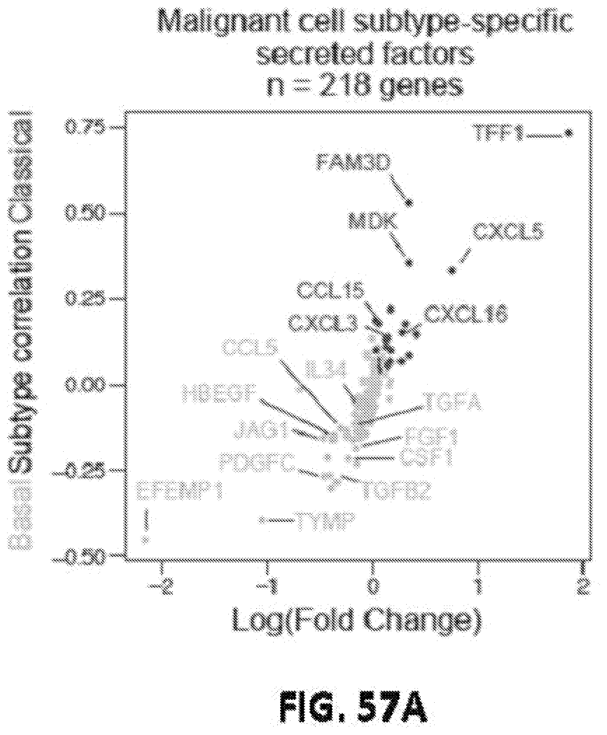

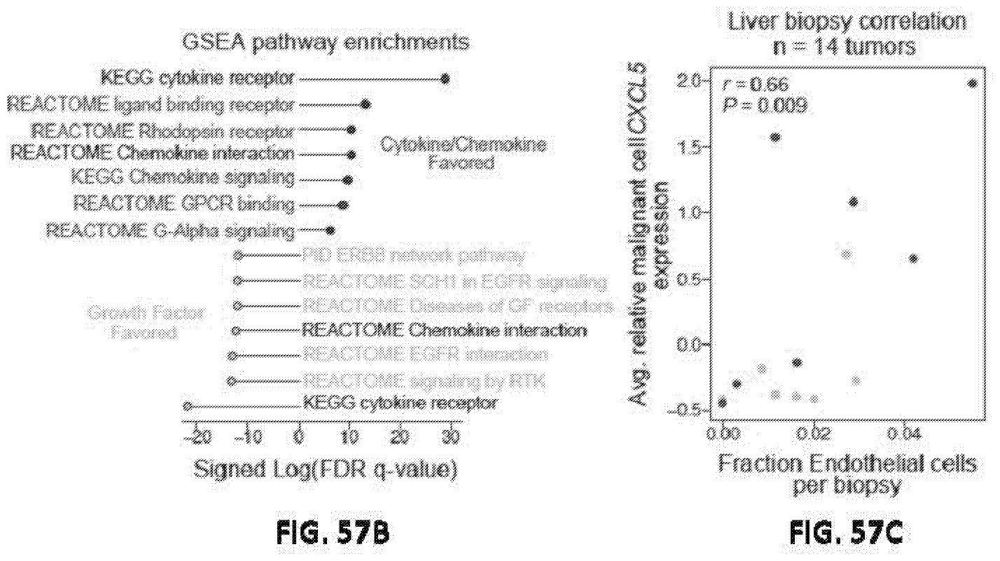

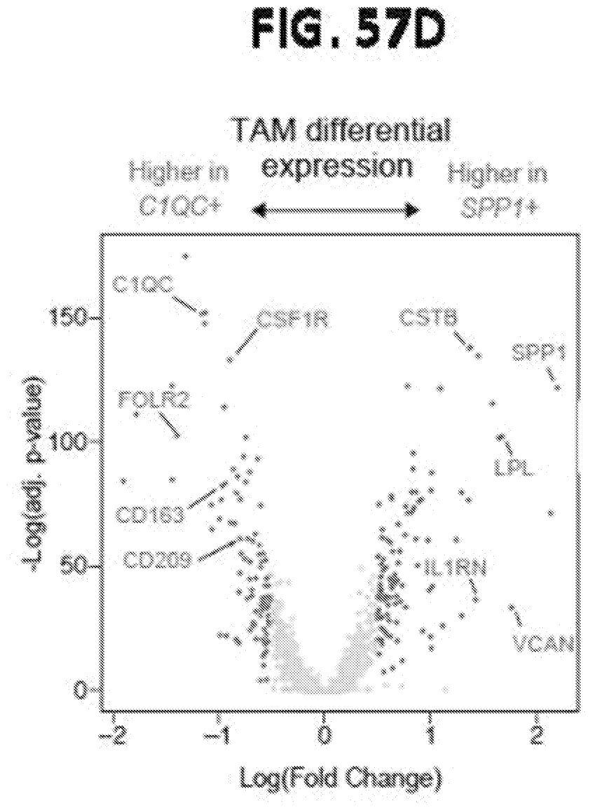

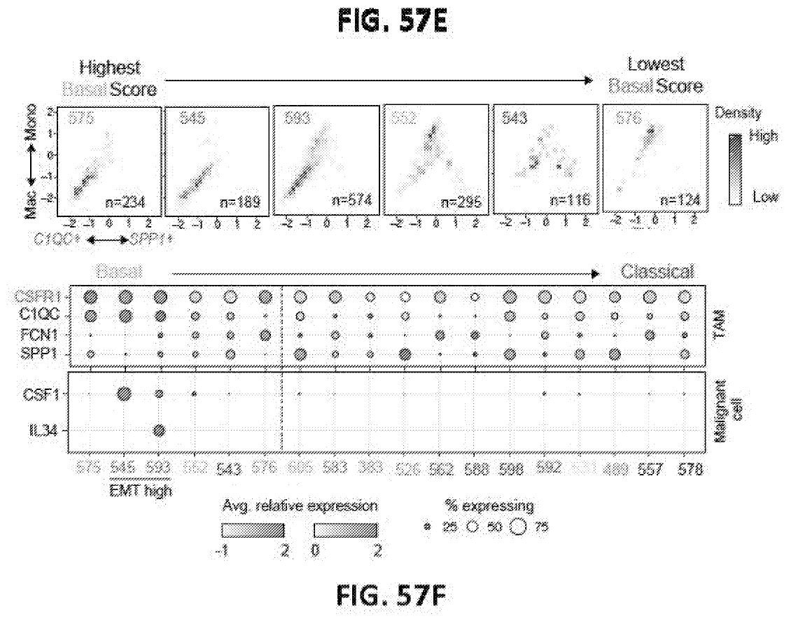

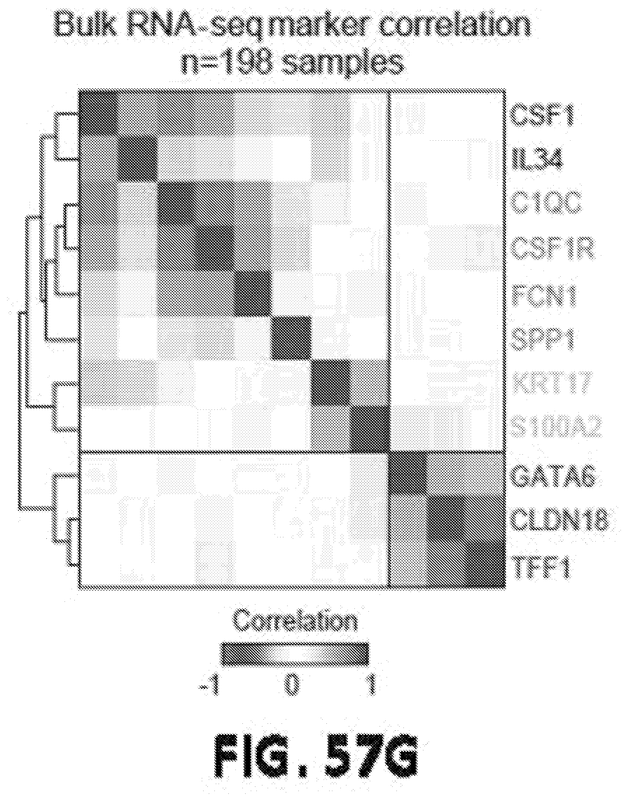

Differential microenvironmental signaling shapes subtype-specific metastatic niches. Given the striking compositional differences Applicants observed in the immune microenvironment across the basal to classical axis, Applicants next searched for tumor-secreted factors that might influence the structure of the local metastatic niche. Specifically, Applicants analyzed subtype-specific expression patterns for genes detected in malignant cells that were annotated as secreted factors (cytokines, chemokines, growth factors by Gene Ontology; n=218 genes). This analysis nominated 57 basal (orange) and 23 classical-associated (blue) secreted factors (FIG. 57A). Gene set enrichment analysis (GSEA) demonstrated that basal tumors were enriched for genes associated with growth factor secretion, while classical tumors were enriched for cytokine/chemokine signaling (FIG. 57B). Applicants observed expression of multiple TGF ligands secreted by basal tumors, consistent with the association of increased TGF-β signaling in basal tumors (FIG. 52F) and local immune suppression/exclusion. Conversely, several chemokines (CXCL5, CXCL3) were enriched in classical tumors in agreement with their overall higher degree of immune infiltration and higher fraction of endothelial cells (FIG. 55C). As such, classical tumors expressed higher levels of CXCL5 which plays a documented role in enhancing tumor-supportive angiogenesis (45, 46). Consistent with this finding, Applicants observed a strong positive correlation between high average malignant cell expression of CXCL5 and the fraction of endothelial cells recovered (FIG. 57C). In basal tumors, Applicants noted increased expression of the ligands CSF1 and IL34 (FIG. 57A) and concomitant expression of their receptor, CSF1R, in the basal-associated C1QC+ TAMs (FIG. 55C, 55G, 57D). Per-tumor analysis revealed a continuum of C1QC+ TAM distribution within basal tumors that correlated with high CSF1R expression (FIG. 57E, 57F, top). Malignant cells with strong EMT features (PANFR0545, PANFR0593) expressed the highest levels of CSF1 and IL34, consistent with a role for tumor cells in shaping their local macrophage phenotypes (FIG. 57F, bottom). To extend this finding in larger cohorts, Applicants analyzed bulk RNA-sequencing of primary and metastatic PDAC tumors for markers of transcriptional subtype, TAM, and tumor secretion phenotypes (12, 13). Consistent with Applicants' single-cell observations, macrophage markers and the ligands CSF1 and IL34 were associated with basal but not classical markers in these samples (n=198, FIG. 57G). Together, these data provide evidence that subtype-specific intercellular crosstalk shapes distinct niches in the metastatic microenvironment.

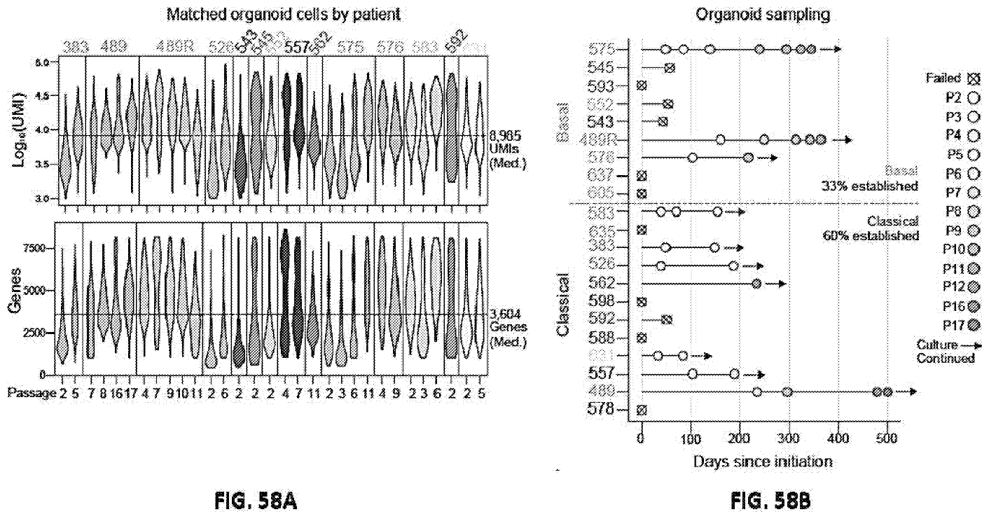

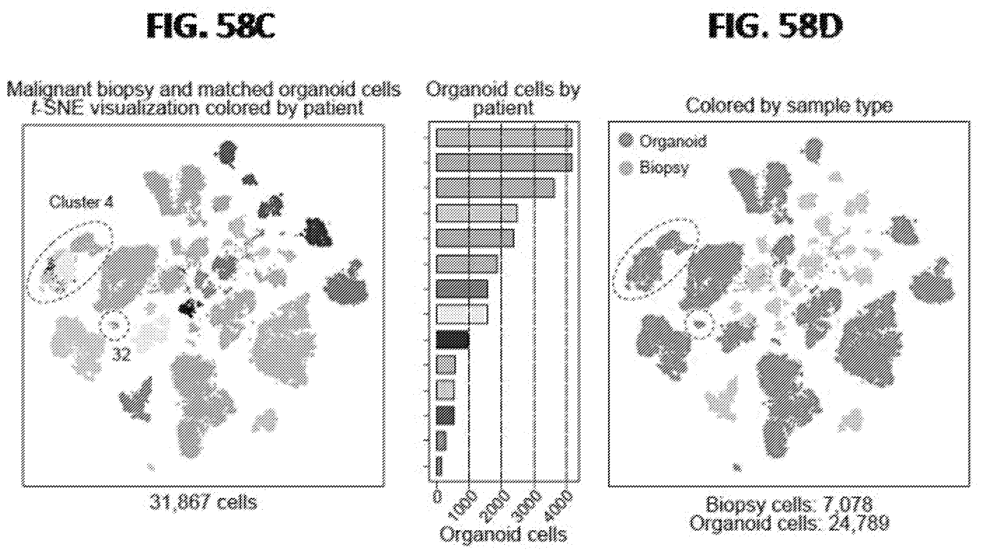

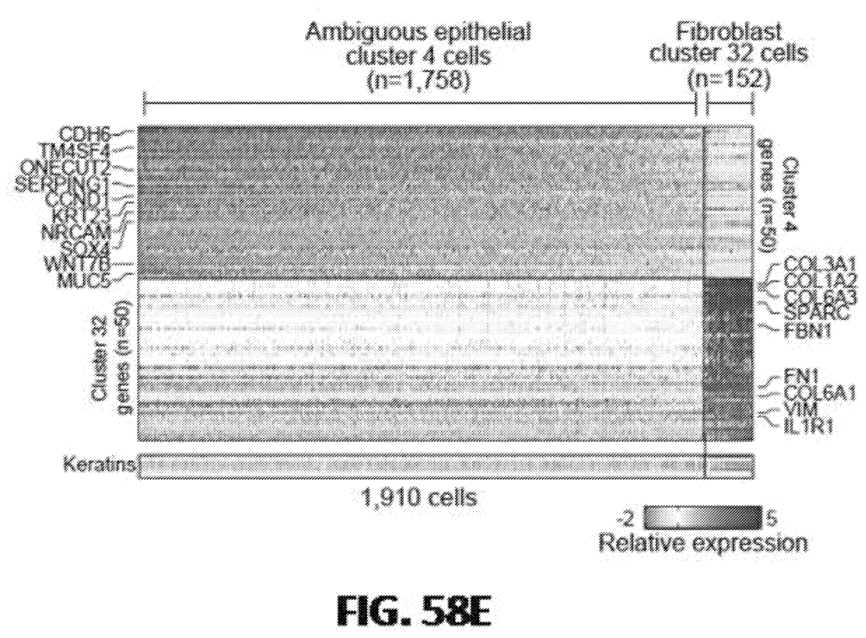

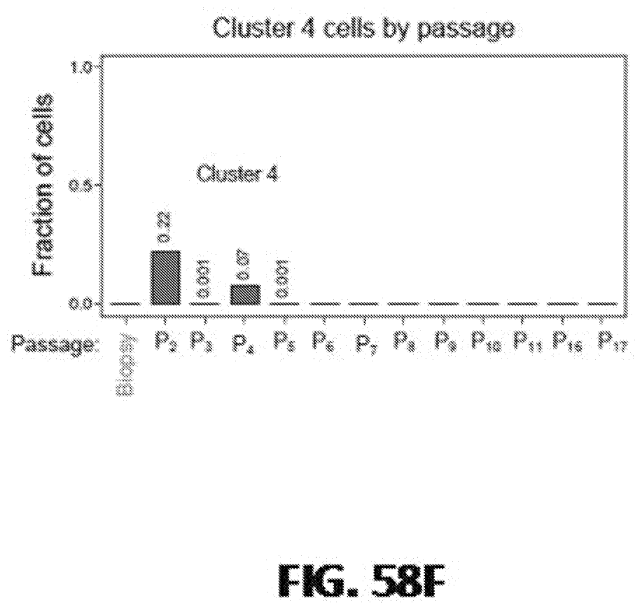

Genotype and phenotype evolution of matched patient-derived organoid models. Applicants' observations indicate that basal and classical phenotypes exist along a continuum and exhibit distinct patterns of reciprocal interaction with their local microenvironments. To examine how tumor cell phenotypes adapt and evolve in ex vivo microenvironments, Applicants utilized the matched organoid models generated from their metastatic biopsy cohort (Methods). For most models, Applicants obtained scRNA-seq samples at the earliest passage possible, typically passage 2 (P2), and again at a later passage (FIG. 58A, 58B). Notably, only 33% of models derived from basal tumors propagated beyond passage 2, whereas 60% of models derived from classical tumors established long-term cultures (FIG. 58B). Globally, unbiased analysis of malignant biopsy (7,078 cells) and organoid cells (n=14 models, 24,789 cells) revealed that biopsy cells clustered separately from their matched organoid counterparts (FIG. 58C, 58D). Only two clusters were admixed by donor and originated from early passage organoids (clusters 4 and 32; FIG. 58C). These clusters were defined by expression patterns consistent with fibroblasts (cluster 32) and poorly differentiated epithelial cells (cluster 4), and were not seen in samples from later passages (FIG. 58E, 58F).

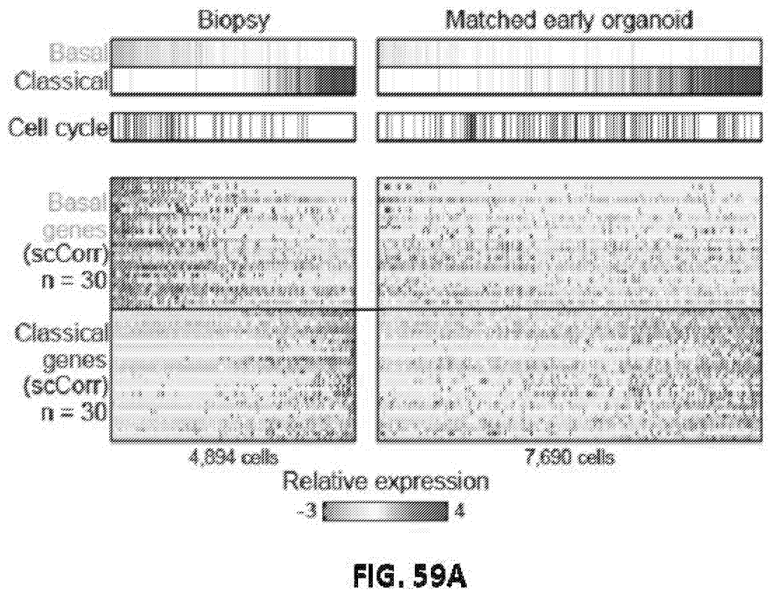

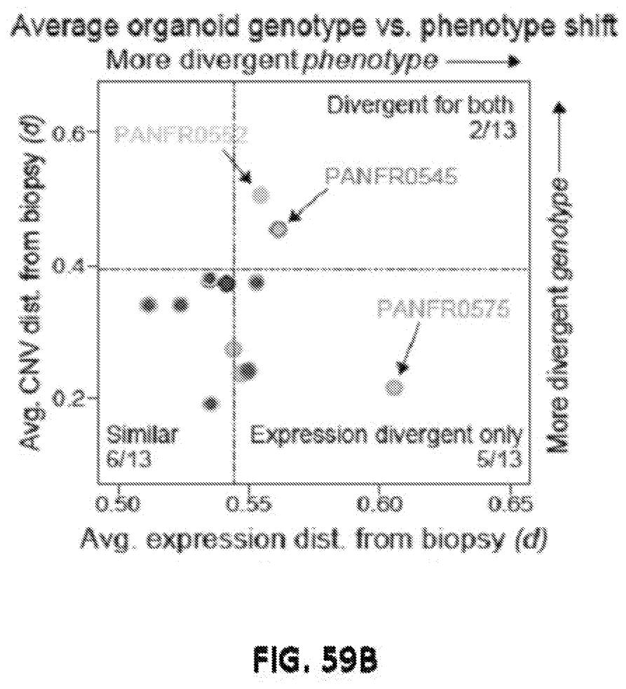

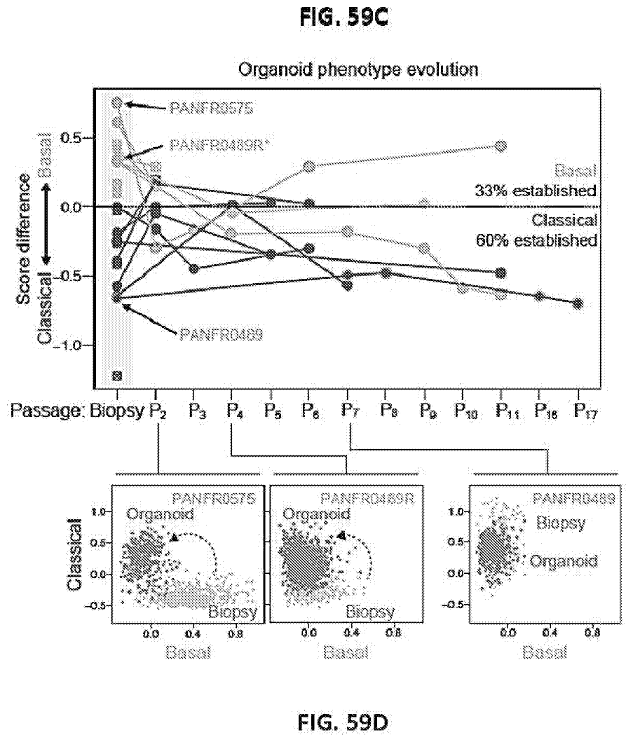

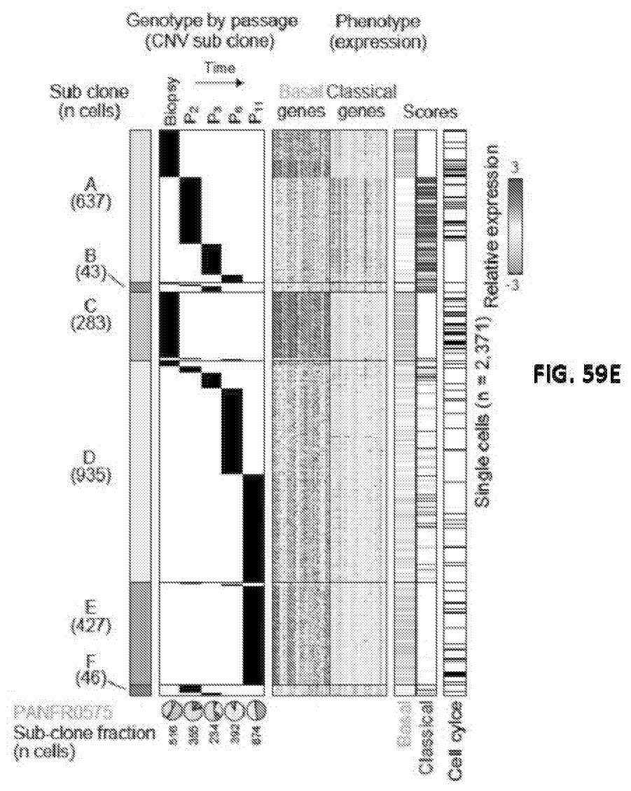

Comparison of transcriptional phenotypes revealed a striking selection against the basal subtype in organoid culture despite it being the higher cycling subset in vivo (FIGS. 52A & 59A). To understand the relative contribution of genotype versus phenotype to this bottleneck, Applicants computed the average single-cell genotype (CNV) and phenotype (basal versus classical) correlation distance (d) between each biopsy and its matched early passage organoid (FIG. 59B; Methods). Six models, all classical, did not significantly deviate along either the CNV or transcriptional axes outside the expected distance for highly similar samples (intra-biopsy d across the cohort; dotted line, P<0.05 for both metrics). Another group, largely basal (right of x-axis dotted line), deviated significantly from their original biopsies along the transcriptional but not the CNV axis. Finally, two basal models (PANFR0545 and PANFR0552) exhibited the strongest deviation from their parent biopsies along both axes (FIG. 59B, upper right). This analysis demonstrated that early passage organoid models largely maintain genomic features observed in parental tumor tissue, but over half of these models, and in particular models derived from basal tumors, were significantly divergent in phenotype compared to their matched tissue-of-origin.

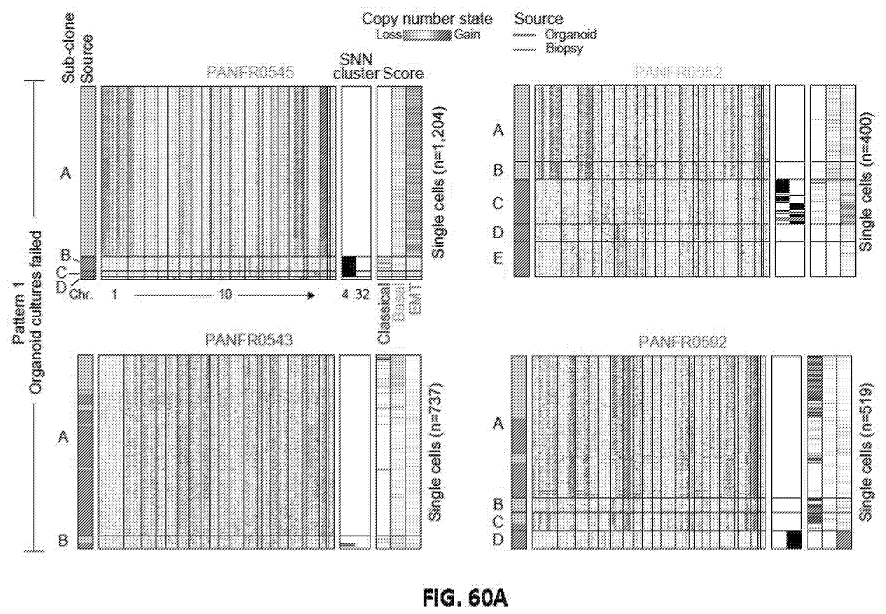

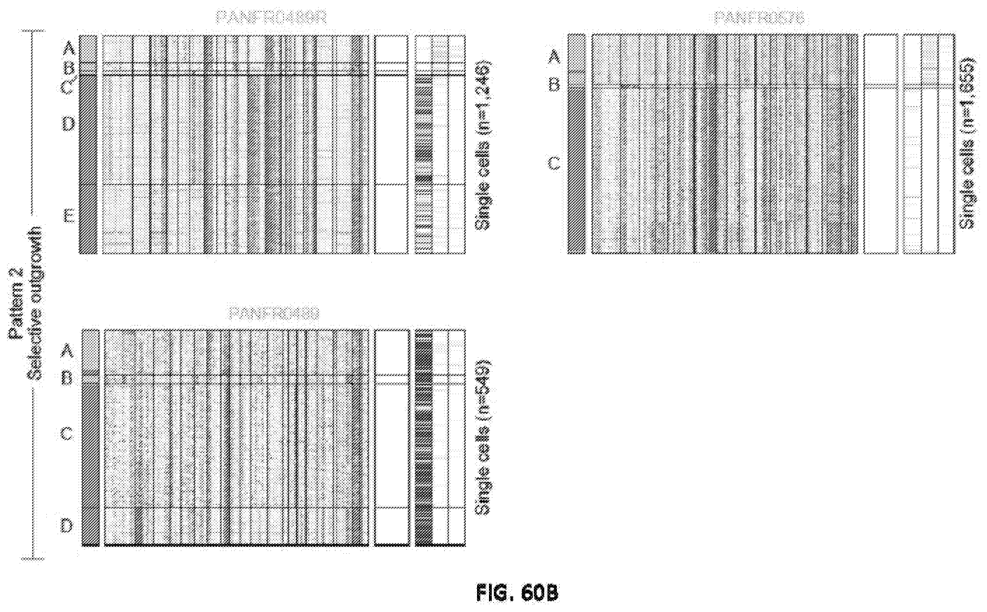

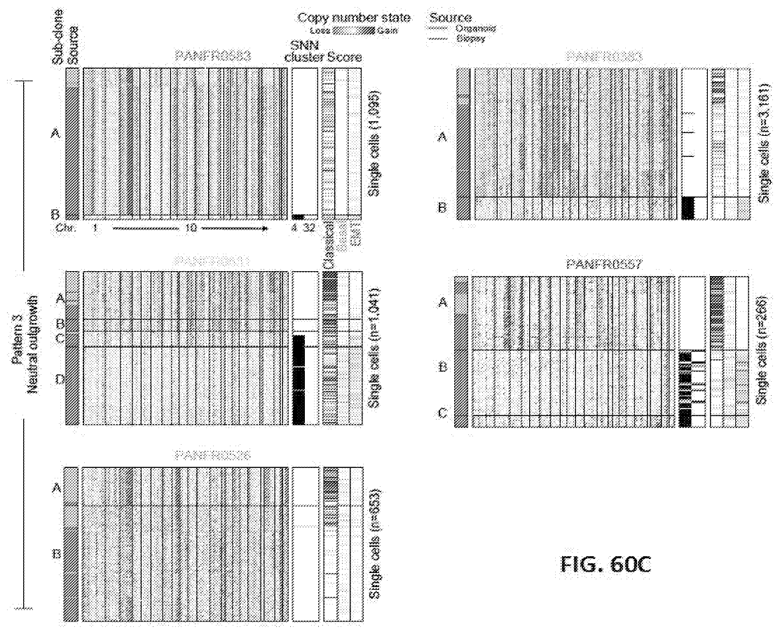

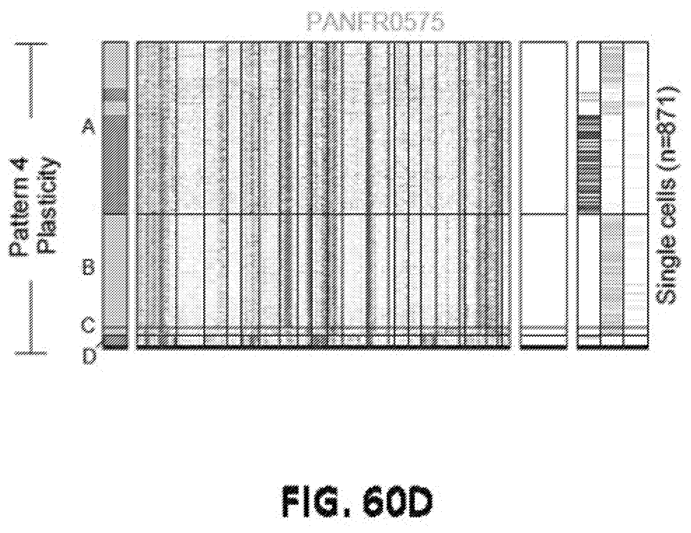

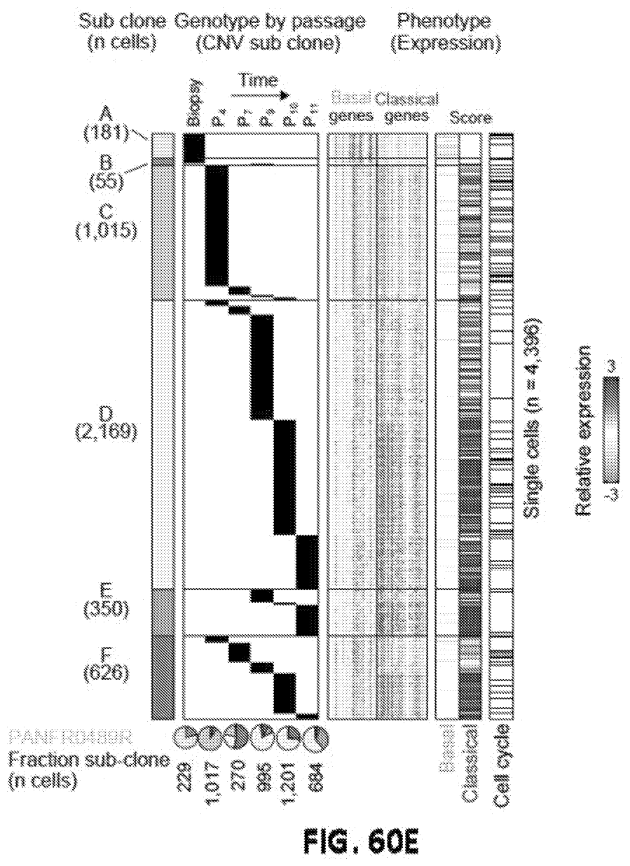

Applicants next examined the subclonal hierarchies within each biopsy-organoid pair. This single-cell comparative analysis identified 4 broad patterns of drift/selection. Pattern 1 consisted of tumors (n=4) where the organoids failed to grow beyond P2; the majority of these were derived from basal tumors (3/4 models), and included the two models (PANFR0545 and PANFR0552) that deviated the most genotypically and phenotypically from their parent biopsies (FIG. 60A). Of the models that propagated beyond P2, Pattern 2 models (n=3) showed evidence of selective outgrowth wherein models derived from basal tumors enriched rare subclones tied to more classical or less basal phenotypes (FIG. 60B). In contrast, models within Pattern 3 (n=5) were typified by neutral outgrowth (no overt selection) where the dominant clone(s) in the biopsy grew out in the organoid (FIG. 60C). These models expressed predominantly classical phenotypes and had the least overall deviation from their parent biopsies (FIG. 59B); none of the models derived from basal tumors displayed this pattern. Finally, Pattern 4 comprised one basal biopsy-organoid pair (PANFR0575) that demonstrated phenotypic plasticity with nearly identical CNVs but a divergent transcriptional phenotype in organoid culture (FIG. 60D). These data illustrate the dramatic adaptation that organoid models undergo ex vivo via transcriptional and clonal selection at early passages, especially when derived from basal tumors.

When Applicants serially sampled and assessed organoid phenotypes over time, they observed that each model assumed a more classical phenotype regardless of its parent tumor's transcriptional identity, and only the Pattern 4 plastic model, PANFR0575, re-acquired its basal phenotype at a later passage (FIG. 59C, 59D). Linked genotype and phenotype assessment from iterative passages provided evidence for significant evolution along both CNV and transcriptional axes over time in culture (FIG. 59E, 60E). After identifying CNV10 defined subclones in the parental biopsy and its associated serial organoid samples (FIG. 59E; clones A-F; 4 Methods), Applicants related cells that were similar in genotype (e.g., all cells within clone A) to their corresponding transcriptional phenotype. In sample PANFR0575 (FIG. 59E), Applicants observed examples of transcriptional plasticity at early passages within clone A. Cells derived from the parental biopsy were basal, but all other cells in this subclone derived from organoids had classical phenotypes. Interestingly, with successive passaging, several subclones emerged with hybrid and basal phenotypes (clones D and E). While model PANFR0575 is a unique case, it highlights the various ways organoids can evolve in culture, including via transcriptional plasticity (clone A) and the late emergence of rare subclones (clones D and E). In contrast, PANFR0489R was initially basal, but Applicants observed clonal selection and phenotypic drift toward classical states, as seen in most other models (FIG. 59C, 60E). Together, these findings demonstrate that multiple mechanisms underlie organoid evolution and divergence from the parental tumor, highlight that transcriptional variation is a key contributor to these differences, and emphasize the importance of deep molecular characterization of patient-derived models prior to functional application.

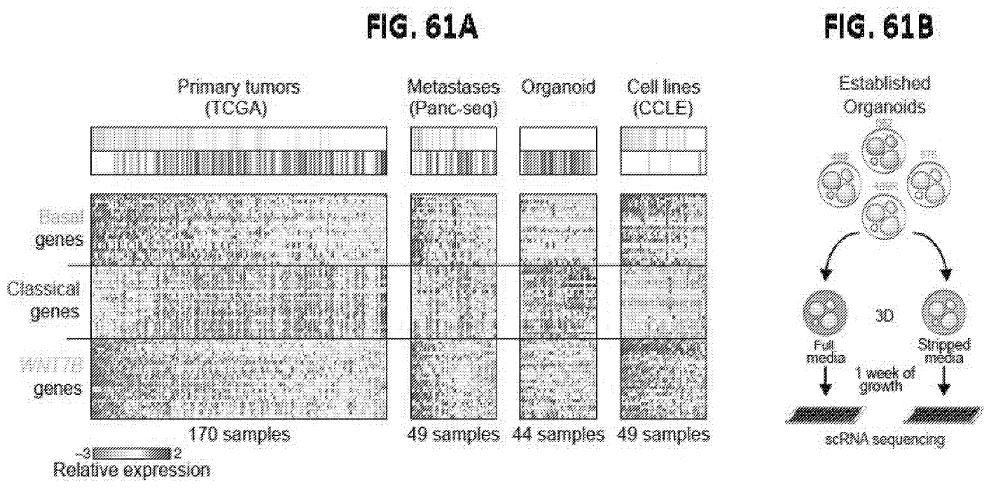

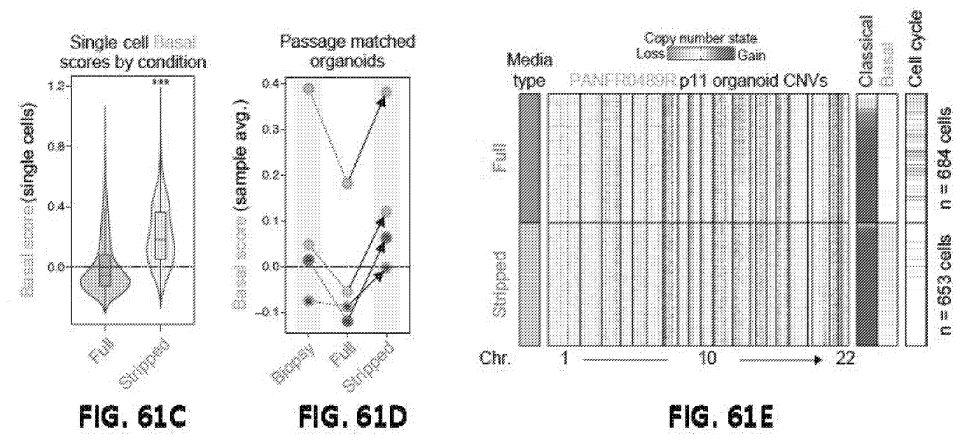

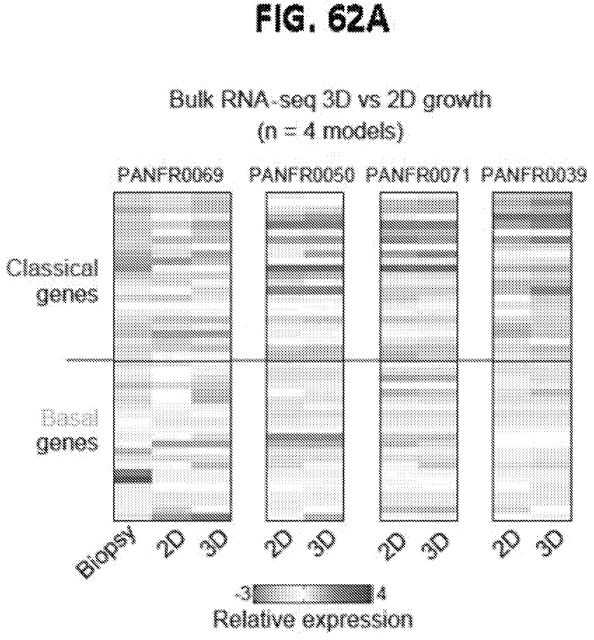

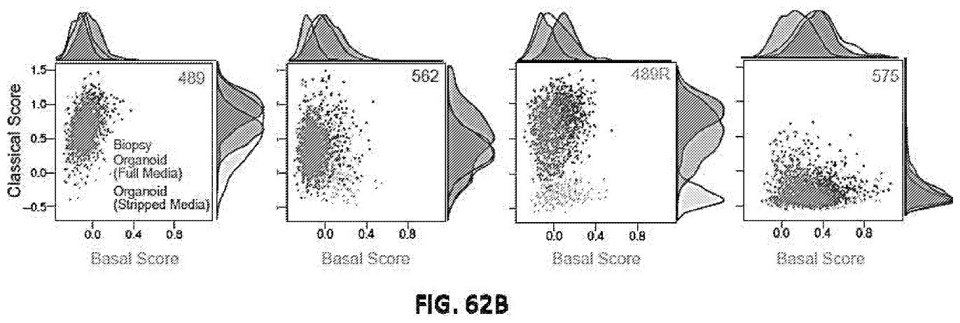

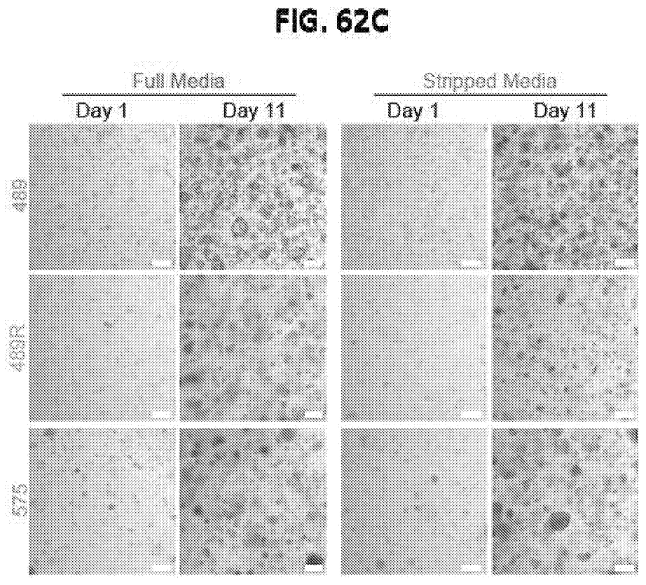

Alterations to the ex vivo culture environment revive the basal state in organoids. Having demonstrated that distinct expression states as well as the local microenvironment co-vary across the basal to classical axis, we reasoned that different conditions may be needed to preserve basal versus classical transcriptional heterogeneity. Comparing bulk RNA expression data from patient tumors (n=219), organoids 351 (n=44) and cell lines (n=49, CCLE) provided evidence that culture conditions can profoundly influence transcriptional state (12, 13, 47). Indeed, most organoid models recapitulate the classical phenotype while cell lines mirror basal expression patterns (FIG. 61A). To isolate the effects of extracellular matrix dimensionality from media formulation, Applicants cultured established 3-dimensional (3D) PDAC organoid models (n=4) as 2-dimensional (2D) cell lines on tissue culture plastic in the same organoid media and noted that this had little effect on transcriptional subtype across the models tested (FIG. 62A). Applicants then hypothesized that multiple components within standard organoid media (10, 33), including WNT3A, R-SPONDIN-1, FGF10, and TGF and BMP pathway inhibitors such as NOGGIN and A-8301, may drive tumor cells toward more classical phenotypes in organoid culture. When established organoid models (n=4) were grown for 1 week in reduced medium without any additives (“stripped” media, containing only Glutamax, anti-microbials, HEPES buffer, and Advanced DMEM/F12 media; FIG. 61B; see Methods), Applicants observed a significant increase in basal gene expression across single cells (FIG. 61C; P<0.0001), as well as coordinated sample-level shifts to a more basal phenotype in each model, in some cases returning to levels observed in the parental biopsy (FIG. 61D). This shift was less pronounced in the model derived from the most classical tumor (PANFR0489, pink outline; FIG. 61D; FIG. 62B). Although there was an appreciable effect on the fraction of cycling cells in the stripped media (FIG. 61E, far right), the organoids continued to grow under these conditions (FIG. 62C). These responses were unlikely to be driven by acute selection since the CNV profiles between the conditions remained stable within this timeframe (FIG. 61E). Collectively, these observations provide evidence for significant ex vivo tumor cell plasticity in response to microenvironmental cues and suggest that organoid and cell line culture conditions can be further optimized to recapitulate clinically relevant in vivo tumor cell states.

### Discussion

This study demonstrates the precision afforded by scRNA-seq for categorizing and phenotyping relevant malignant and non-malignant cell populations in metastatic PDAC. In the malignant compartment, Applicants confirmed the basal-like and classical transcriptional subtyping framework; however, Applicants found that these subtypes exist on a continuum and include a newly identified “hybrid” phenotype. Applicants show at both the RNA and protein level that most tumors are comprised of all three phenotypes and exhibit notable intratumoral heterogeneity in two ways: (i) basal or classical phenotypes in discrete cells but co-occurring in the same tumor, consistent with a recent report (11), and (ii) co-expression of both states in the same single cell (hybrid cells). Importantly, the identification of these hybrid cells in human tumor biopsies suggests that interconversion may be possible between the classical and basal subtypes. Basal tumor cells exhibit mesenchymal and stem-like features, including TGF-β pathway activation and evidence for WNT signaling. In this tissue context, WNT signaling is likely mediated through the expression of WNT7B and/or WNT10A as these were the only ligands consistently expressed in the cells captured. WNT7B is a key developmental signal for pancreatic progenitor proliferation, normal morphogenesis, and mesenchymal expansion, and its expression evokes the possibility that basal tumor cells may share similarities with a discrete subset of early pancreatic progenitors (48). Several studies have suggested a role for WNT signaling in supporting proliferation and cell state specification in PDAC models, but more experimentation is needed to clarify its impact (41, 49, 50). Given that PDAC transcriptional subtypes have been associated with differential response to chemotherapy (3, 7-10), these new insights into PDAC subtype heterogeneity and their associated biologies have important implications for understanding therapy response in clinical trials.

In coordination with malignant cell phenotypes, non-malignant cells establish subtype-specific local immune microenvironments within the PDAC metastatic niche. Applicants' observations support a model wherein classical tumors exhibit greater chemokine signaling and concomitant immune infiltration. Although this has been hypothesized previously (2, 4, 6), the specific cell types and their phenotypes have remained elusive. Applicants' single-cell dataset clarifies this relationship and identifies a classical TME enriched for endothelial cells and specific myeloid and lymphoid cell types. In the lymphoid compartment, surprisingly, Applicants observed cytotoxic signaling that originates primarily from activated NK cells, suggesting a dominant role for innate immune function in the classical metastatic niche. Conversely, the basal microenvironment is optimally tuned for immune suppression/evasion, which may contribute to the overall lower survival seen in this subtype. The relative paucity of CD4+ T cells found in basal tumors suggests exclusion, possibly driven by the higher levels of TGF gene expression in basal contexts. Somewhat unexpectedly, Applicants found evidence of terminally exhausted CD8+ T cells in only two basal tumors, and, in most cases, both basal and classical tumors exist in a CD8+ T-cell progenitor-restricted state. Basal tumor cells exhibited higher levels of IFN response gene expression compared with classical tumors, suggesting exposure to, and potential tolerance of, the presence of activated T cells (39, 40). Basal tumor cells also shape the myeloid compartment by secreting CSF1 and IL34, with concomitant microenvironmental increase in C1QC+ TAM populations that skew towards a tumor supportive, anti-inflammatory phenotype. Notably, even within basal tumors, those with the most mesenchymal characteristics possessed the most potent immune-evasive phenotypes, suggestive of additional layers of variation even within the basal subtype.

Comparison of matched biopsies and organoids revealed relative preservation of genomic features in most organoid models, as has previously been demonstrated (10, 35), but significant deviation in basal/classical transcriptional state. While classical phenotypes were relatively better preserved, Applicants note strong selection against the basal state under standard organoid media conditions. Serial sampling of organoid models across successive passages demonstrated both phenotypic drift and subclonal outgrowth, such that the dominant clones in some later passage models were only present at low frequencies in the parent tumors. Despite the bias toward classical phenotypes in organoid culture, the rare emergence of basal clones at late passages (PANFR0575; FIG. 59E) suggests that genotype, in addition to microenvironment, may influence transcriptional plasticity. However, resolution is an important limitation of clonal tracing, as one cannot comment directly on variation/selection for single mutations. While Applicants' findings may explain some of the limitations observed when using PDAC organoid models to predict clinical responses (10, 35), they also highlight the significant phenotypic plasticity and adaptability of PDAC cells and, moreover, the utility of primary tissue and matched model comparisons for understanding these features of tumor biology. Interestingly, established PDAC cell lines exhibit predominantly basal phenotypes, but changing matrix dimensionality (2D versus 3D culture) alone did not alter malignant organoid transcriptional phenotypes along the basal-classical axis, implying that variation in adhesive context may affect some but not all biologic behaviors. Encouragingly, the basal phenotype could be recovered by removing exogenous factors from the standard culture media, setting the stage for further optimization of these conditions to adequately support intratumoral heterogeneity and growth (51). These results highlight that ex vivo model growth may not necessarily equate to model fidelity and suggest that experimental conditions, heterogeneity, and plasticity all influence the phenotype of patient-derived organoids (32, 52).

In sum, Applicants show how scRNA-seq can be employed to clarify the structure of the PDAC metastatic niche and uncover formerly unappreciated relationships between tumor transcriptional phenotype and the local immune microenvironment. Although traditionally thought of as a uniformly “immune-cold” tumor, Applicants' findings highlight that the immune microenvironment in PDAC harbors a layer of unappreciated complexity closely linked to tumor cell transcriptional subtype that may provide new avenues for therapeutic targeting. Specifically, TAM-directed therapies, such as anti-CSF1R antibodies, could be employed to selectively target transcriptional-subtype-associated populations (42, 53-55). However, while basal tumors associate with a potentially sensitive CSF1R-expressing population (C1QC+ TAM), classical tumors harbor TAMs that are resistant to such therapies (SPP1+ TAM) (42). Thus, just as one considers combinations to target malignant states, the TME will also likely require tailored combination therapies. These findings provide rationale for future clinical trials to employ high-resolution phenotyping of malignant and non-malignant cells to stratify patients and track tumor evolution in response to therapy. While organoid platforms represent a transformative technology to develop patient-specific tumor models, Applicants demonstrate that some organoid models show a high degree of plasticity and that both their genotype and transcriptional phenotype must be understood to enable their optimal use in personalized medicine. Finally, Applicants provide a framework for relating malignant cells, the TME, and patient-derived model systems that may be applicable in other tumor types with clinically relevant transcriptional variation across the malignant and microenvironmental landscape.

### REFERENCES

1. Hyman, D. M., Taylor, B. S. & Baselga, J. Implementing Genome-Driven Oncology. Cell 168, 584-599, doi:10.1016/j.cell.2016.12.015 (2017).
2. Collisson, E. A., Bailey, P., Chang, D. K. & Biankin, A. V. Molecular subtypes of pancreatic cancer. Nat 454 Rev Gastroenterol Hepatol 16, 207-220, doi:10.1038/s41575-019-0109-y (2019).
3. Collisson, E. A. et al. Subtypes of pancreatic ductal adenocarcinoma and their differing responses to therapy. Nat Med 17, 500-503, doi:10.1038/nm.2344 (2011).
4. Bailey, P. et al. Genomic analyses identify molecular subtypes of pancreatic cancer. Nature 531, 47-52, 458 doi:10.1038/nature16965 (2016).
5. Moffitt, R. A. et al. Virtual microdissection identifies distinct tumor- and stroma-specific subtypes of pancreatic ductal adenocarcinoma. Nat Genet 47, 1168-1178, doi:10.1038/ng.3398 (2015).
6. Puleo, F. et al. Stratification of Pancreatic Ductal Adenocarcinomas Based on Tumor and Microenvironment Features. Gastroenterology 155, 1999-2013 e1993, doi:10.1053/j.gastro.2018.08.033 (2018).
7. Porter, R. L. et al. Epithelial to mesenchymal plasticity and differential response to therapies in pancreatic ductal adenocarcinoma. Proc Natl Acad Sci USA, doi:10.1073/pnas. 1914915116 (2019).
8. Aung, K. L. et al. Genomics-Driven Precision Medicine for Advanced Pancreatic Cancer: Early Results from the COMPASS Trial. Clin Cancer Res 24, 1344-1354, doi:10.1158/1078-0432.CCR-17-2994 (2018).
9. O'Kane, G. M. et al. GATA6 Expression Distinguishes Classical and Basal-like Subtypes in Advanced Pancreatic Cancer. Clin Cancer Res, doi:10.1158/1078-0432.CCR-19-3724 (2020).
10. Tiriac, H. et al. Organoid Profiling Identifies Common Responders to Chemotherapy in Pancreatic Cancer. Cancer Discov 8, 1112-1129, doi:10.1158/2159-8290.CD-18-0349 (2018).
11. Chan-Seng-Yue, M. et al. Transcription phenotypes of pancreatic cancer are driven by genomic events during tumor evolution. Nat Genet 52, 231-240, doi:10.1038/s41588-019-0566-9 (2020).
12. Cancer Genome Atlas Research Network. Electronic address, a. a. d. h. e. & Cancer Genome Atlas Research, N. Integrated Genomic Characterization of Pancreatic Ductal Adenocarcinoma. Cancer Cell 32, 185-203 e113, doi: 10.1016/j.ccell.2017.07.007 (2017).
13. Aguirre, A. J. et al. Real-time Genomic Characterization of Advanced Pancreatic Cancer to Enable Precision Medicine. Cancer Discov 8, 1096-1111, doi:10.1158/2159-8290.CD-18-0275 (2018).
14. Connor, A. A. et al. Integration of Genomic and Transcriptional Features in Pancreatic Cancer Reveals 480 Increased Cell Cycle Progression in Metastases. Cancer Cell 35, 267-282 e267, 481 doi:10.1016/j.ccell.2018.12.010 (2019).
15. Venteicher, A. S. et al. Decoupling genetics, lineages, and microenvironment in IDH-mutant gliomas by single-cell RNA-seq. Science 355, doi:10.1126/science.aai8478 (2017).
16. Tirosh, I. et al. Dissecting the multicellular ecosystem of metastatic melanoma by single-cell RNA-seq. Science 352, 189-196, doi:10.1126/science.aad0501 (2016).
17. Puram, S. V. et al. Single-Cell Transcriptomic Analysis of Primary and Metastatic Tumor Ecosystems in Head and Neck Cancer. Cell 171, 1611-1624 e1624, doi:10.1016/j.cell.2017.10.044 (2017).
18. van Galen, P. et al. Single-Cell RNA-Seq Reveals AML Hierarchies Relevant to Disease Progression and Immunity. Cell 176, 1265-1281 e1224, doi:10.1016/j.cell.2019.01.031 (2019).
19. Kim, C. et al. Chemoresistance Evolution in Triple-Negative Breast Cancer Delineated by Single-Cell Sequencing. Cell 173, 879-893 e813, doi:10.1016/j.cell.2018.03.041 (2018).
20. Sade-Feldman, M. et al. Defining T Cell States Associated with Response to Checkpoint Immunotherapy in Melanoma. Cell 176, 404, doi:10.1016/j.cell.2018.12.034 (2019).
21. Patel, A. P. et al. Single-cell RNA-seq highlights intratumoral heterogeneity in primary glioblastoma. Science 344, 1396-1401, doi:10.1126/science.1254257 (2014).
22. Filbin, M. G. et al. Developmental and oncogenic programs in H3K27M gliomas dissected by single-cell RNA-seq. Science 360, 331-335, doi:10.1126/science.aao4750 (2018).
23. Hovestadt, V. et al. Resolving medulloblastoma cellular architecture by single-cell genomics. Nature 572, 74-79, doi:10.1038/s41586-019-1434-6 (2019).
24. Neftel, C. et al. An Integrative Model of Cellular States, Plasticity, and Genetics for Glioblastoma. Cell 178, 835-849 e821, doi:10.1016/j.cell.2019.06.024 (2019).
25. Tirosh, I. & Suva, M. L. Dissecting human gliomas by single-cell RNA sequencing. Neuro Oncol 20, 37-503 43, doi:10.1093/neuonc/nox126 (2018).
26. Tirosh, I. et al. Single-cell RNA-seq supports a developmental hierarchy in human oligodendroglioma. Nature 539, 309-313, doi:10.1038/nature20123 (2016).
27. Elyada, E. et al. Cross-Species Single-Cell Analysis of Pancreatic Ductal Adenocarcinoma Reveals Antigen-Presenting Cancer-Associated Fibroblasts. Cancer Discov 9, 1102-1123, doi:10.1158/2159-508 8290.CD-19-0094 (2019).
28. Bernard, V. et al. Single-Cell Transcriptomics of Pancreatic Cancer Precursors Demonstrates Epithelial and Microenvironmental Heterogeneity as an Early Event in Neoplastic Progression. Clin Cancer Res 25, 2194-2205, doi:10.1158/1078-0432.CCR-18-1955 (2019).
29. Ligorio, M. et al. Stromal Microenvironment Shapes the Intratumoral Architecture of Pancreatic Cancer. Cell 178, 160-175 e127, doi:10.1016/j.cell.2019.05.012 (2019).
30. Siegel, R. L., Miller, K. D. & Jemal, A. Cancer statistics, 2020. CA Cancer J Clin 70, 7-30, doi:10.3322/caac.21590 (2020).
31. Drost, J. & Clevers, H. Organoids in cancer research. Nat Rev Cancer 18, 407-418, doi:10.1038/s41568-517 018-0007-6 (2018).
32. Ben-David, U. et al. Genetic and transcriptional evolution alters cancer cell line drug response. Nature 560, 325-330, doi:10.1038/s41586-018-0409-3 (2018).
33. Boj, S. F. et al. Organoid models of human and mouse ductal pancreatic cancer. Cell 160, 324-338, doi:10.1016/j.cell.2014.12.021 (2015).
34. Huang, L. et al. Ductal pancreatic cancer modeling and drug screening using human pluripotent stem cell- and patient-derived tumor organoids. Nat Med 21, 1364-1371, doi:10.1038/nm.3973 (2015).
35. Driehuis, E. et al. Pancreatic cancer organoids recapitulate disease and allow personalized drug screening. Proc Natl Acad Sci USA, doi:10.1073/pnas.1911273116 (2019).
36. Gierahn, T. M. et al. Seq-Well: portable, low-cost RNA sequencing of single cells at high throughput. Nat Methods 14, 395-398, doi:10.1038/nmeth.4179 (2017).
37. Groger, C. J., Gubinger, M., Waldhor, T., Vierlinger, K. & Mikulits, W. Meta-analysis of gene expression signatures defining the epithelial to mesenchymal transition during cancer progression. PLoS One 7, e51136, doi:10.1371/journal.pone.0051136 (2012).
38. Kim, J. H., Park, S. Y., Jun, Y., Kim, J. Y. & Nam, J. S. Roles of Wnt Target Genes in the Journey of Cancer Stem Cells. Int J Mol Sci 18, doi:10.3390/ijms18081604 (2017).
39. Benci, J. L. et al. Tumor Interferon Signaling Regulates a Multigenic Resistance Program to Immune Checkpoint Blockade. Cell 167, 1540-1554 el512, doi:10.1016/j.cell.2016.11.022 (2016).
40. Benci, J. L. et al. Opposing Functions of Interferon Coordinate Adaptive and Innate Immune Responses to Cancer Immune Checkpoint Blockade. Cell 178, 933-948 e914, doi:10.1016/j.cell.2019.07.019 (2019).
41. Seino, T. et al. Human Pancreatic Tumor Organoids Reveal Loss of Stem Cell Niche Factor Dependence during Disease Progression. Cell Stem Cell 22, 454-467 e456, doi:10.1016/j.stem.2017.12.009 (2018).
42. Zhang, L. et al. Single-Cell Analyses Inform Mechanisms of Myeloid-Targeted Therapies in Colon Cancer. Cell 181, 442-459 e429, doi:10.1016/j.cell.2020.03.048 (2020).
43. Zilionis, R. et al. Single-Cell Transcriptomics of Human and Mouse Lung Cancers Reveals Conserved Myeloid Populations across Individuals and Species. Immunity 50, 1317-1334 e1310, 543 doi:10.1016/j.immuni.2019.03.009 (2019).
44. Miller, B. C. et al. Subsets of exhausted CD8(+) T cells differentially mediate tumor control and respond to checkpoint blockade. Nat Immunol 20, 326-336, doi:10.1038/s41590-019-0312-6 (2019).
45. Chen, C. et al. CXCL5 induces tumor angiogenesis via enhancing the expression of FOXD1 mediated by the AKT/NF-kappaB pathway in colorectal cancer. Cell Death Dis 10, 178, doi:10.1038/s41419-019-548 1431-6 (2019).
46. Li, A. et al. Overexpression of CXCL5 is associated with poor survival in patients with pancreatic cancer. 550 Am J Pathol 178, 1340-1349, doi:10.1016/j.ajpath.2010.11.058 (2011).
47. Ghandi, M. et al. Next-generation characterization of the Cancer Cell Line Encyclopedia. Nature 569, 503-508, doi:10.1038/s41586-019-1186-3 (2019).
48. Afelik, S., Pool, B., Schmerr, M., Penton, C. & Jensen, J. Wnt7b is required for epithelial progenitor growth and operates during epithelial-to-mesenchymal signaling in pancreatic development. Dev Biol 399, 204-217, doi:10.1016/j.ydbio.2014.12.031 (2015).
49. Arensman, M. D. et al. WNT7B mediates autocrine Wnt/beta-catenin signaling and anchorage-independent growth in pancreatic adenocarcinoma. Oncogene 33, 899-908, doi:10.1038/onc.2013.23 558 (2014).
50. Steinhart, Z. et al. Genome-wide CRISPR screens reveal a Wnt-FZD5 signaling circuit as a druggable vulnerability of RNF43-mutant pancreatic tumors. Nat Med 23, 60-68, doi:10.1038/nm.4219 (2017).
51. Mead, B. E. et al. Harnessing single-cell genomics to improve the physiological fidelity of organoid-derived cell types. BMC Biol 16, 62, doi:10.1186/s12915-018-0527-2 (2018).
52. Ben-David, U., Beroukhim, R. & Golub, T. R. Genomic evolution of cancer models: perils and opportunities. Nat Rev Cancer 19, 97-109, doi:10.1038/s41568-018-0095-3 (2019).
53. Balachandran, V. P., Beatty, G. L. & Dougan, S. K. Broadening the Impact of Immunotherapy to Pancreatic Cancer: Challenges and Opportunities. Gastroenterology 156, 2056-2072, doi:10.1053/j.gastro.2018.12.038 (2019).
54. Zhu, Y. et al. CSF1/CSF1R blockade reprograms tumor-infiltrating macrophages and improves response to T-cell checkpoint immunotherapy in pancreatic cancer models. Cancer Res 74, 5057-5069, 570 doi:10.1158/0008-5472.CAN-13-3723 (2014).
55. Candido, J. B. et al. CSF1R(+) Macrophages Sustain Pancreatic Tumor Growth through T Cell Suppression and Maintenance of Key Gene Programs that Define the Squamous Subtype. Cell Rep 23, 1448-1460, doi:10.1016/j.celrep.2018.03.131 (2018).

### Methods

Tissue collection and dissociation. Investigators obtained written, informed consent from patients with pancreatic cancer for Dana-Farber/Harvard Cancer Center Institutional Review Board (IRB)-approved protocols 11-104, 17-000, 03-189, and/or 14-408 for tissue collection, molecular analysis, and organoid generation. Core needle biopsy specimens were collected and the first core was sent for pathologic analysis. One or more additional cores were then allocated for scRNA-seq and organoid generation.

Samples were minced into small portions using a scalpel and then digested at 37° C. for 15 minutes using digest medium that consisted of human complete organoid medium (see below), 1 mg/ml collagenase XI (Sigma Aldrich), 10 μg/ml DNase (Stem Cell Technologies), and 10 μM Y27632 (Selleck) (1). In the initial process optimization, Applicants found that dissociation times below 30 minutes, while not always completely digesting all biopsy material and potentially affecting the representation of difficult to dissociate cell types (e.g., fibroblasts), resulted in greater cell viability and improved RNA quality downstream. After digestion, cells were washed, treated with ACK lysing buffer (Gibco) to lyse red blood cells, washed again, and counted using a hemocytometer with 0.4% Trypan blue (Gibco) added at 1:1 dilution for viability assessment. Applicants allowed residual tissue chunks to settle before selecting a predominance of single cells for counting and Seq-Well processing. Applicants allocated between 10,000 and 15,000 viable cells per Seq-Well array based upon total cell counts, and where possible Applicants prepared two arrays per sample. Most samples were processed and loaded onto Seq-Well arrays within 2-3 hours of biopsy acquisition.

Organoid generation and sampling. Cells remaining after scRNA-seq allocation were initiated and maintained as patient-derived organoid cultures as previously described (1, 2). In brief, digested cells were seeded in 3-dimensional (3D) Growth-factor Reduced Matrigel (Corning) and fed with human complete organoid medium containing advanced DMEM/F12 (Gibco), 10 mM HEPES (Gibco), 1× GlutaMAX (Gibco), 500 nM A83-0l (Tocris), 50 ng/mL mEGF (Peprotech), 100 ng/mL mNoggin (Peprotech), 100 ng/mL hFGF10 (Peprotech), 10 nM hGastrin I (Sigma), 1.25 mM N-acetylcysteine (Sigma), 10 mM Nicotinamide (Sigma), 1×B27 supplement (Gibco), R-spondin1 conditioned media 10% final, Wnt3A conditioned media 500% final, 100 U/ml penicillin/streptomycin (Gibco), and 1× Primocin (Invivogen). 10 μM Y27632 (Selleck) was included in the culture medium of newly initiated samples until the first media exchange. For propagation, organoids were dissociated with TrypLE (Gibco) before re-seeding into fresh Matrigel and culture medium.

After initial processing of fresh tissue specimens, Applicants monitored samples closely for organoid growth. Applicants did not passage organoids at set time intervals, as there was significant variability in the time needed to establish relatively robust growth of organoids (FIG. 58B). Instead, Applicants maintained early passage organoids until they reached relative confluence, and then passaged them at low split ratios (1:1, 1:1.5, or 1:2 dilutions) in full organoid media to promote continued growth. In one case, PANFR0489R, cells persisted as individuals and small organoids after initiation in full organoid media, but did not grow and expand cell numbers significantly. Approximately 15 weeks after initiation, Applicants switched a portion of the surviving cells to organoid media without A83-01 or mNoggin, and observed renewed growth of organoids under these media conditions but not of those that remained in full organoid media. Consequently, Applicants expanded this sample in media without A83-01 or mNoggin, including performing early passage scRNA-seq. After several additional passages, once the organoids were robustly growing, Applicants were able to transition back to full organoid media with no apparent change in growth rate, morphology, or transcriptional phenotype. All other serially sampled organoids were maintained and assessed by scRNA-seq in full media.

For scRNA-seq of organoid samples, Applicants passaged organoids and allowed them to grow for 6 days before then dissociating, counting, and allocating 15,000 viable cells for Seq-Well. By standardizing the collection of organoid scRNA-seq samples at 6 days after passaging, Applicants tried to minimize bias arising from cell cycle differences in samples at different degrees of confluence.

Testing organoid phenotypes under different matrix and media conditions. For adaptation of patient derived organoids onto 2-dimensional (2D) culture surfaces as patient-derived cell lines, tissue culture plates were pre-coated with 100 μg/ml Matrigel suspended in basal media for 2 hours at 37° C. before washing with PBS. Established organoid models were dissociated and seeded onto these Matrigel-coated culture wells in standard organoid media. In parallel, a portion of these passage-matched organoid cells were re-seeded into Matrigel droplets as above. Cells were cultured in both matrix conditions in standard organoid media until they were confluent, approximately 2-3 weeks. Cells were collected and lysed using Trizol before snap freezing. RNA was isolated and purified as below using chloroform extraction, aqueous phase isolation, and processing using the Qiagen AllPrep DNA/RNA/miRNA Universal kit before being submitted for sequencing.

For scRNA-seq assessment of organoid phenotypes under different media conditions, established organoid models were passaged as above by dissociating and reseeding into Matrigel droplets. A portion of the cells were cultured with standard organoid media (“Full”). A distinct portion of passage-matched cells were cultured in “Stripped” media, which consisted of advanced DMEM/F12 (Gibco), 10 mM HEPES (Gibco), 1× GlutaMAX (Gibco), 100 U/ml penicillin/streptomycin (Gibco), and 1× Primocin (Invivogen). Cells were cultured for 6 days before being collected, dissociated, and aliquoted for scRNA-seq. Images were taken with an Olympus XM10 camera mounted to an Olympus CKX41 microscope 1 day after seeding and again after 11 days in culture to assess organoid growth in both conditions.

Single-cell RNA-seq (scRNA-seq) data library generation, sequencing, and alignment. ScRNA-seq processing followed the Seq-Well protocol, uniquely compatible with low-input samples (3). Briefly, arrays were preloaded with RNA capture beads (ChemGenes) and stored in quenching buffer until used. Prior to cell loading, arrays were resuspended in 5 mL RPMI medium with 10% fetal bovine serum (both from Gibco, hereafter referred to as RP-10). After dissociation, single-cell suspensions were manually counted and diluted to 15,000 cells per 200 μL of RP-10 when cell numbers allowed. Excess RP-10 was aspirated from the array and cells were loaded onto arrays. Excess cells were washed off with PBS (4×5 mL, Gibco), briefly left in RPMI (5 mL) and cell+bead pairs were sealed for 40 minutes at 37° C. using a polycarbonate membrane (Fisher Scientific NC1421644). Arrays were rocked in lysis buffer for 20 minutes and RNA was hybridized onto the beads for 40 minutes. Beads were removed and reverse transcription was performed overnight using Maxima H Minus Reverse Transcriptase (Thermo Fisher EP0753). Prior to sequencing, the beads underwent an exonuclease treatment (NewEngland Biolabs M0293L) and second strand synthesis en masse followed by whole transcriptome amplification (WTA, Kapa Biosystems KK2602) in 1,500 bead reactions (50 μL). cDNA was isolated using Agencourt AMPure XP beads (Beckman Coulter, A63881) at 0.6×SPRI (solid-phase reversible immobilization) followed by a IX SPRI and quantified using a Qubit dsDNA High Sensitivity assay kit (Thermo Fisher Q32854). Library preparation was performed using Nextera XT DNA tagmentation (Illumina FC-131-1096) and N700 and N500 indices specific to a given sample. Tagmented and amplified sequences were purified with a 0.6×SPRI. cDNA was loaded onto either an Illumina Nextseq (75 Cycle NextSeq500/550v2 kit) or Novaseq (100 Cycle NovaSeq6000S kit, Broad Institute Genomics Platform) at 2.4 μM. Regardless of platform, the paired end read structure was 21 bases (cell 100 barcode and UMI) by 50 bases (transcriptomic information) with an 8 base pair (bp) custom read one primer. The demultiplex and alignment protocol was followed as previously described (4). While Novaseq data were directly output as FASTQs, Nextseq BCL files were converted to FASTQs using bcl2fastq2. The resultant Nextseq and Novaseq FASTQs were demultiplexed by sample based on Nextera N700 and N500 indices. Reads were then aligned to the hg19 transcriptome using the cumulus/dropseq_tools pipeline on Terra maintained by the Broad Institute using standard settings.

Bulk RNA-sequencing of organoids. RNA was obtained for bulk RNA-sequencing from established organoids using one of two approaches. Dissociated organoids were resuspended into cold Matrigel, added as droplets to tissue culture plates (Greiner BioOne), and allowed to polymerize for 30 minutes before addition of media. Organoids were grown for 14-21 days (until confluent) under these conditions with regular media changes. At the time of harvest, cells were washed with cold phosphate buffered saline (PBS) 111 at 4° C., then lysed with Trizol (Invitrogen) before snap-freezing. To isolate RNA, Applicants performed chloroform extraction with isolation of the aqueous phase before processing RNA as per protocols outlined in the Qiagen AllPrep DNA/RNA/miRNA Universal kit.

In the second approach, dissociated organoids were resuspended in a 10% Matrigel in organoid media suspension (volume/volume) and cultured in ultra-low-attachment culture flasks (Corning). Organoids were grown for 14-21 days (until confluent) before pelleting, washing with cold PBS at 4° C. until most Matrigel was dissipated, and then snap frozen. For RNA isolation, cell pellets were homogenized using buffer RLT Plus (Qiagen) and a Precellys homogenizer. Samples were then processed for both DNA extraction and RNA isolation as per the Qiagen AllPrep DNA/RNA/miRNA Universal kit. Purified RNA was then submitted for sequencing by the Broad Institute Genomics Platform.

In brief, total RNA was quantified using the Quant-iT RiboGreen RNA Assay Kit (Thermo Fisher R11490) and normalized to 5 ng/μl. Following plating, 2 μL of a 1:1000 dilution of ERCC RNA controls (Thermo Fisher 4456740) were spiked into each sample. An aliquot of 200 ng for each sample was transferred into library preparation which uses an automated variant of the Illumina TruSeq Stranded mRNA Sample Preparation Kit. This method preserves strand orientation of the RNA transcript, and uses oligo dT beads to select mRNA from the total RNA sample followed by heat fragmentation and cDNA synthesis from the RNA template. The resultant 400 bp cDNA then goes through dual-indexed library preparation: ‘A’ base addition, adapter ligation using P7 adapters, and PCR enrichment using P5 adapters. After enrichment, the libraries were quantified using Quant-iT PicoGreen (1:200 dilution; Thermo Fisher P11496). After normalizing samples to 5 ng/μL, the set was pooled and quantified using the KAPA Library Quantification Kit for Illumina Sequencing Platforms. The entire process was performed in 96-well format and all pipetting was done by either Agilent Bravo or Hamilton Starlet.

Pooled libraries were normalized to 2 nM and denatured using 0.1 N NaOH prior to sequencing. Flowcell cluster amplification and sequencing were performed according to the manufacturer's protocols using the NovaSeq 6000. Each run was a 101 bp paired-end with an eight-base index barcode read. Data were analyzed using the Broad Picard Pipeline which includes de-multiplexing and data aggregation (https://broadinstitute.github.io/picard/). FASTQ files were then processed as described below (see Bulk RNA-sequencing analysis).

Multiplex immunofluorescence imaging. A multi-marker panel was developed to characterize tumor cell subtype in formalin-fixed paraffin-embedded (FFPE) 4 μm tissue sections using multiplex immunofluorescence. The panel comprises markers associated with either a basal (Keratin-17: Thermo Fisher MA513539 and s100a2: Abcam 109494) or classical (cldn18.2: Abcam 241330, GATA6: CST 5851 and TFF1: Abcam 92377) subtype. Additionally, DAPI (Akoya Biosciences FP1490) was included for identification of nuclei and pan-cytokeratin (AE1/AE3: DAKO M3515; C11: CST 4545) for identification of epithelial cells. Secondary Opal Polymer HRP mouse and rabbit (ARH1001EA), Tyramide signal amplification and Opal fluorophores (Akoya Biosciences) were used to detect primary antibodies (Keratin-17, Opal 520; s100a2, Opal 650; GATA6, Opal 540; cldn18.2, Opal 570; TFF1, Opal 690; panCK, Opal 620). Prior to use in multiplex staining, primary antibodies were first optimized via immunohistochemistry on control tissue to confirm contextual specificity. Monoplex immunofluorescence and iterative multiplex fluorescent staining were then used to optimize staining order, antibody-fluorophore assignments and fluorophore concentrations. Multiplex staining was performed using a Leica BOND RX Research Stainer (Leica Biosystems, Buffalo, Ill.) with sequential cycles of antigen retrieval, protein blocking, primary antibody incubation, secondary antibody incubation, and fluorescent labeling. Overview images of stained slides were acquired at 10× magnification using a Vectra 3.0 Automated Quantitative Imaging System (Perkin Elmer, Waltham, Mass.) and regions of interest (ROIs) were selected for multispectral image acquisition at 20×. After unmixing using a spectral library of single-color references, each image was inspected to ensure uniform staining quality and adequate tumor representation.

### Data Analysis.

Mutation and CNV identification from tissue DNA-sequencing. For targeted DNA-sequencing of clinical samples, next-generation sequencing using a custom-designed hybrid capture library preparation was performed on an Illumina HiSeq 2500 with 2×100 paired-end reads, as previously described (5, 6). Sequence reads were aligned to reference sequence b37 edition from the Human Genome Reference Consortium using bwa, and further processed using Picard (version 1.90, http://broadinstitute.github.io/picard/) to remove duplicates and Genome Analysis Toolkit (GATK, version 1.6-5-g557da77) to perform localized realignment around indel sites. Single nucleotide variants were called using MuTect v1.1.45, insertions and deletions were called using GATK Indelocator. Copy number variants and structural variants were called using the internally-developed algorithms RobustCNV and BreaKmer followed by manual review (7). RobustCNV calculates copy ratios by performing a robust linear regression against a panel of normal samples. The data were segmented using circular binary segmentation, and event identification was performed based on the observed variance of the data points (8).

Applicants computed the cytoband-level copy number calls and weighted (by length) average segment means across the covered regions of each cytoband using an in-house pipeline. Briefly, cytobands were considered amplified/deleted if more than 70% of the covered regions had a log 2 copy ratio of greater than 0.2/less than −0.2, and were considered neutral if more than 70% of the covered regions had a log 2 copy ratio between −0.2 and 0.2.

Single-cell data quality pre-processing and initial cell-type discovery. All single-cell data analysis was performed using the R language for Statistical Computing (v3.5.1). Each biopsy sample's digital gene expression (DGE) matrix (cells×genes) was trimmed to exclude low quality cells (<400 genes detected; <1,000 UMIs; >50% mitochondrial reads) before being merged together (preserving all unique genes) to create the larger biopsy dataset. The merged dataset was further trimmed to remove cells with >8,000 genes which represent outliers and likely doublet cells. Applicants also removed genes that were not detected in at least 50 cells. The same metrics were applied to the organoid single-cell cohort (see below). On a per cell basis, UMI count data was divided by total transcripts captured and multiplied by a scaling factor of 10,000. These normalized values were then natural log transformed for downstream analysis (i.e. log-normalized cell×gene matrix). Initial exploration of the data was performed using the R package Seurat (v2.3.4) and followed two steps: 1) SNN-guided quality assessment and 2) cell-type composition determination. In step 1, Applicants intentionally left cells in the DGE matrix of dubious quality (e.g. % mitochondrial reads >25% but <500%), performed principal component analysis (PCA) over the variable genes (n=1,070 genes), and input the first 50 PCs (determined by Jackstraw analysis implemented through Seurat) to build an SNN graph and cluster the cells (res=1; k.param=40). The inclusion of poor-quality cells essentially acts as a variance “sink” for other poor-quality cells and they cluster together based on their shared patterns in quality-associated gene expression. This method helped to nominate additional low quality (e.g. defined exclusively by mitochondrial genes) or likely doublet cells (e.g. clusters defined by co-expression of divergent lineage markers) which were removed from the dataset (n=1,678 cells).

Using the trimmed dataset, Applicants proceeded to step 2 using a very similar workflow as above but with slightly altered input conditions for defining clusters. Here Applicants used PCs 1-45 and their associated statistically significant genes for building the SNN graph and determining cluster membership (resolution=1.2; k.param=40). This identified the 36 clusters shown (visualized using t-SNE; perplexity, 40; iterations, 2,500) in FIG. 49C. The expression of known markers was used to collapse clusters containing shared lineage information. For example, clusters 1, 2, and 4 all express high levels of macrophage markers-CD14, FCGR3A (CD16), CD68—and were accordingly collapsed for this first pass analysis (FIG. 49C, 49E). To aid the cell-type identification, Applicants performed a ROC test implemented in Seurat to confirm the specificity (power >0.6 for cell type specificity) of the top marker genes used to discern the cell types. Combined with inferred CNV information (see below), this analysis confirmed the presence of 11 broad non-malignant cell types in the biopsy dataset (Table 2). Variation in the SNN graph parameters above did not strongly affect cell type identification.

Single-cell CNV identification. To confirm the identity of the putative malignant clusters identified in FIG. 49D, Applicants estimated single-cell CNVs as previously described by computing the average expression in a sliding window of 100 genes within each chromosome after sorting the detected genes by their chromosomal coordinates (9, 10). Applicants used all T/NK, Fib, Hep, and Endo cells identified above as reference normal populations for this analysis. To compare with bulk targeted DNA-sequencing, Applicants collapsed individual probes to cytoband-level information (weighted average of log 2 ratios across each cytoband, see above) within each sample. ScRNA-seq-inferred CNVs showed high concordance across samples with the bulk measurements and suggests that, at least by this metric, Applicants are likely sampling the same dominant clones within sequential but distinct cores from each needle biopsy procedure. For plotting CNV profiles in putative malignant versus normal cells (FIG. 50B), Applicants computed the average CNV signal for the top 5% of altered cells in each biopsy and correlated all cells in that biopsy to the averaged profile as has been previously described (11). Relation of this correlation coefficient to the CNV score (mean square deviation from diploidy) in the single cells from each biopsy shows consistent separation of malignant from non-malignant cells, and, combined with membership in patient-specific SNN clusters, substantiates the identification of malignant cells in the dataset.

Subclustering of malignant and non-malignant cells. Detailed phenotyping required splitting the dataset into malignant and non-malignant fractions. After subsetting to only the malignant cells, Applicants re-scaled the data and ran PCA including the first 35 PCs for SNN clustering and t-SNE visualization (FIG. 48E). This PCA was used to determine the PanNET identity for PANFR0580 (FIG. 51A). After removing PANFR0580, Applicants repeated the steps above and used this new PCA for the remainder of PDAC malignant cell analysis. Subsequent phenotyping for malignant cells is discussed below (Generation of expression signatures/scores). A similar approach was used for the non-malignant cells in FIG. 55A. Here, Applicants excluded cells from PANFR0489R since this patient was the only one in our cohort recently exposed to immunomodulatory agents on a clinical trial and would likely not represent a comparable distribution of steady-state phenotypes in the metastatic niche—these cells cluster together in comparison to all others within cell types in the dataset (grey-blue dots, FIG. 48D). After removing these cells, Applicants re-scaled the data and ran PCA including the first 40 PCs as input for SNN clustering and t-SNE embedding (FIG. 55A). To determine the specific phenotypes within T/NK and macrophage populations, Applicants separately subclustered these groups using PCs 1-20 and a resolution of 0.6 in both cases. Of note, subclustering within the macrophages revealed a distinct cluster of cells co-expressing markers of both T/NK cells and macrophages (n=357 cells). Applicants discarded these cells as doublets, as have others, and re-ran the macrophage PCA and clustering (12, 13). Each unbiased analysis helped to define the non-malignant phenotypes summarized in FIGS. 55A & 55B and 56. For visualization, Applicants utilized a force-directed layout (FDL, using SPRING14) of the k-nearest-neighbor (KNN) graph representing each cell's connectivity to its 10 nearest neighbors. Each graph was initialized with either the variable genes for the T/NK cells or the top 100 cell-type specific genes (see Generation of expression signatures, below) for the macrophage populations.

Generation of expression signatures scores. All expression scores were computed as previously described by taking a given input set of genes and comparing their average relative expression to that of a control set (n=100 genes) randomly sampled to mirror the expression distribution of the genes used for the input (10). While all scores were computed in the same way, choosing the genes for input varied. Applicants have outlined the relevant approaches below. Where correlations (Pearson's r) are performed over genes, Applicants used the log-transformed UMI count data for each case. Unless otherwise noted, Applicants selected the top 30 statistically significant genes for each signature (>3 s.d. above the mean for shuffled data) for visualization and scoring.

Cell cycle. Applicants utilized previously established signatures for GUS (n=43 genes) and G2/M (n=55 genes) to place each cell along this dynamic process (11). After inspecting the distribution of scores in the complete dataset, Applicants considered any cell >1.5 s.d. above the mean (15) for either the GUS or the G2/M scores to be cycling.

Malignant programs. Applicants started by scoring each malignant single cell for the basal-like and classical genes identified by Moffitt et al (16) as these were well described by unbiased analysis in their data (PCA). Applicants used these scores to classify each single cell as basal, classical, or hybrid. Single cells were classified based on their maximal expression of either basal or classical scores. Hybrid cells are those that have greater than average expression (i.e. scaled and centered expression score >0) for both the basal and classical programs. Applicants used these classifications to summarize overall tumor composition and visualize the groups. Heterogeneity measures were not significantly affected by changing the hybrid cutoff. Applicants also used these scores to determine each cell's basal versus classical polarization or “score difference”, simply the difference of the two scores.

To determine programs associated with basal and classical phenotypes, Applicants correlated the aforementioned basal and classical scores to the entire gene expression matrix containing malignant cells and selected the 1,909 genes significantly associated with either subtype (r>0.1; >3 s.d. above the mean for shuffled data, full data in Tables 5 & 6). For visualization, Applicants use the “scCorr” basal and classical genes (top 30 correlated genes for each). Selection of genes for programs associated with basal and classical phenotypes took two approaches. For top correlated programs that have established gene sets (IFNResp, TGFB, EMT; Hallmark/Reactome gene sets), Applicants selected the genes for inclusion whose expression was significantly correlated with the appropriate state in our dataset (17). Since existing epithelial gene sets are derived from comparisons of “epithelial” and “not” in the non-malignant setting, they were ill-suited to describe the data. For “epithelial genes”, Applicants correlated the expression of EPCAM (co-correlated with classical) and selected the top 30 statistically significant genes for the signature (>3 s.d. above the mean for shuffled data). Applicants followed a similar method for the WNT7B-associated genes (co-correlated with basal) as there was no prior gene-set specific to this biology.

Non-Malignant programs. The cytotoxic score was chosen after consulting the literature and includes PRF1, GNLY, GZMB, TNF, and IFNG (18, 19). The CD8+ T cell progenitor versus differentiated/exhausted continuum was defined in two steps and largely mirrors the phenotypes seen in previous work (18, 19). First, PC1 nominated the stereotyped markers for each end of the continuum (TCF7, IL7R, progenitor; HAVCR2, GZMB, exhausted/diff.). Second, we correlated expression within the CD8+ T cells to top loaded markers of each state on either end of PC1 (TCF7, progenitor; GZMB, exhausted/diff.; FIG. 56G), again selecting the top significant genes for each score (n=30). Finally, for plotting, Applicants computed each CD8+ T cell's separation across this continuum by subtracting the two signatures and binned all CD8+ T cells/tumor at a regular interval to show their distribution (20).

TAM signatures were determined in a slightly different manner, similar to previous work (15). Again, using PCA as an anchor, Applicants correlated expression within the TAM compartment to either FCN1, SPP1, or C1QC (top loaded genes on each relevant PC) and merged the resultant correlation coefficients for every detected gene to the 3 subtypes into one matrix (i.e. a 16,920×3 matrix). For each TAM type (i.e. vector of correlation coeffects to each marker), Applicants first ranked the matrix by decreasing correlation coefficient, selected only the most significantly associated genes to that type (r>0.1; >3 s.d. above the mean for shuffled data), subtracted the second highest correlation coefficient for each subtype-associated gene, and then re-ranked the matrix by this corrected value. Applicants repeated this procedure for each TAM subtype independently. This ensures that the genes selected are specific to a given TAM subset and do not describe general TAM features. The top 30 genes for each were used for scoring and visualization (Tables 7-9; FIG. 56I). To visualize these dynamic programs in 2-dimensions, Applicants computed two polarization scores for each TAM (10). Applicants first calculated the committed TAM phenotype polarization score (SPP1 versus C1QC) for each cell by inverting the sign for the C1QC score and adding this to the SPP1 score such that negative scores now represent strongly C1QC-polarized cells (x-axis, FIG. 55G). Next, previous work suggests FCN1+ TAMs are likely recent infiltrates to the tumor microenvironment (TME), share some features with CD14+ blood monocytes, and can give rise to other committed TAM phenotypes (12, 13). To reflect this, Applicants calculated a “mono-like” to macrophage polarization score for each TAM by subtracting the absolute value of the SPP1 versus C1QC polarization score from the FCN1+ score (“mono-like” to macrophage score, FIG. 55F; y-axis FIG. 55G) and situated FCN1+ TAMs at the top of a putative developmental hierarchy within the TME. Density visualizations are made over this backbone by placing a 25×25 grid over the plot and computing the relative density of TAMs within each bin.

Basal-classical TME associations. Applicants determined the transcriptional-subtype-dependent composition of the TME (FIG. 55C) following two steps. First, Applicants computed the fractional representation for every non-malignant cell type in each core needle biopsy and determined their pairwise correlation distance (Pearson's r) followed by hierarchical clustering using Ward's method. For this analysis Applicants only used samples derived from liver metastases that had >200 non-malignant cells captured. In the main heatmap (FIG. 55C), yellow to red heat indicates cell types captured at similar frequencies across samples (i.e. cell types existing in convergent microenvironments). Light to dark blue in the same heatmap reflects cell types that are anti-correlated with each other in our cohort (i.e. cell types that originate from divergent microenvironments). Second, to incorporate information on how the tumor's overall malignant transcriptional phenotype relates to these capture patterns, Applicants correlated each tumor's average malignant score difference (basal-classical polarization; Pearson's r) to each cell type's capture frequency. The blue (classical-associated) to orange (basal-associated) heat in the right offset column for each cell type indicates its preferential subtype association. These values were not included in the original “convergent to divergent” clustering and do not affect the ordering of the dendrogram. This analysis avoids using absolute cell numbers captured from each array, assumes the same capture biases are present in each sample, and is tuned to relate patterns of cell type representation across the basal-classical axis.

Matched organoid clustering and cell-typing. After applying similar quality metrics as above, Applicants performed PCA, SNN clustering, and t-SNE embedding for 31,867 cells including organoid cells and all malignant cells from primary PDAC biopsies (PCs 1-50; resolution=1.2; k.param=45; perplexity=45; max_iter=2,500), and identified 39 total clusters. Organoids clustered separately from their matched biopsies, suggesting expression and/or CNV related drift in culture. Only two SNN clusters-clusters 4 and 32-were admixed by sample. Applicants determined the specific gene expression programs in these two clusters via differential expression testing by Wilcoxon rank sum test (P<0.05, Bonferroni correction; log(fold change)>0.5). These comparisons were done in a “I versus rest” fashion, testing for genes defining each cluster (4 or 332 32) compared to the entire dataset. Their expression profiles were consistent with fibroblasts (cluster 32) and epithelial cells (cluster 4; FIG. 58E).

Correlation distances for genotype and phenotype. To generate correlation distances for genotype and phenotype, each single cell in a biopsy-organoid pair was represented by two vectors of information: (i) a phenotype vector containing expression values for basal and classical genes (scCorr basal and classical genes, n=60 genes) and (ii) a genotype vector containing the average CNV score for each cytoband. The phenotype and genotype distances between every single cell within a biopsy/early organoid pair was computed from these vectors using a correlation-based (Pearson's r) distance metric of the form d=(1−r)/2. This resulted in two distance matrices of n×n dimension where n is the total number of cells from each biopsy/early organoid sample pair. Points in FIG. 59B are computed by averaging the values for d between only early organoid and matched biopsy cells. P-value thresholds (dotted lines on each axis) represent P<0.05 considering the distribution of intra-biopsy cell-cell distances across all the parent biopsies (i.e. averaging all d for only biopsy cell vs. biopsy cell comparisons) for both metrics. Crossing this threshold thus indicates greater than expected divergence from the distribution of expected “highly similar” samples.

Sunbclonal analysis with single-cell inferred CNVs. The inferCNV workflow can be used to call subclonal genetic variation with high sensitivity and is comprehensively outlined here https://github.com/broadinstitute/inferCNV/wiki.21 Briefly, Applicants used a six-state Hidden Markov Model (i6-HMM) to predict copy number status (complete loss to >3× gain) across putative altered regions in each cell. A Bayesian latent mixture model then evaluated the posterior probability that a given copy number alteration is a true positive. Applicants set a relatively stringent cutoff for this step (BayesMaxPNormal=0.2) to only include high probability alterations for downstream clustering. The results of this filtered i6-HMM output were then used to cluster the single cells using Ward's method. We used inferCNV's “random trees” method to test for statistical significance (P<0.05, 100 random permutations for each split) at each tree bifurcation and only retained subclusters that had statistical evidence underlying the presumed heterogeneity. To track subclonal heterogeneity between biopsy and matched organoid cells in FIG. 59E and FIG. 60A-60E, the above workflow was implemented within each biopsy and the relevant matched organoid samples, essentially treating all cells as the same “tumor” and allowing the CNVs to determine cell sorting agnostic to sample-of-origin.

Bulk RNA-sequencing analysis. FASTQs for bulk RNA expression profiles were downloaded from the relevant repository (TCGA, https://toil.xenahubs.net; PDAC Cell lines, https://portals.broadinstitute.org/ccle), available in house (Panc-Seq, metastatic PDAC), or generated for this study (organoid cohort) (22-24). All were processed using the same pipeline. Briefly, each sample's sequences were marked for duplicates and then mapped to hg38 using STAR. After running QC checks using RNAseqQC, gene-level count matrices were generated using RSEM. Instructions to run the pipeline are given in the Broad CCLE github repository https://github.com/broadinstitute/ccle_processing. Length-normalized 369 values (TPM) were then transformed according to log 2(TPM+1) for downstream analysis. The entire dataset was scaled and centered to allow relative comparisons across sample types (e.g. tumors, organoids, and cell lines). Signature scores were computed as above (e.g. basal and classical; Generation of expression signatures/scores) (25). The “Contaminate Score” used in FIG. 54B represents an average of top marker genes from our single cell dataset specific for non-malignant cell types (Fibroblasts, T cells, Macrophages, B cells, Endothelial, Plasma; Table 2). ABSOLUTE purity scores were provided with each relevant dataset (TCGA and Panc-Seq). Hierarchical clustering was performed using Ward's method.

Tumor phenotyping from mIF data. Supervised machine learning algorithms were applied for tissue and cell segmentation (inForm 2.4.1, Akoya Biosciences). Single-cell-level imaging data were exported and further processed and analyzed using R (v3.6.2). To assign phenotypes to individual tumor epithelial cells, mean expression intensity in the relevant subcellular compartment was first used to classify cells as positive or negative for each of the 5 markers. Combinatorial expression patterns for the five markers were then used to phenotypically classify cells as basal, classical, hybrid or marker negative (3 combinations of 2 basal markers, 7 combinations of 3 classical markers, 1 pan-marker negative, 1 pan-marker positive, 20 combinations of co-expression of basal and classical markers, Table 3; FIG. 53). Tumor subtype composition was assessed by calculating the fraction of total tumor cells positive for each cell phenotype (Table 4, excluding pan-marker negative cells).

### REFERENCES

1. Tiriac, H. et al. Organoid Profiling Identifies Common Responders to Chemotherapy in Pancreatic Cancer. Cancer Discov 8, 1112-1129, doi:10.1158/2159-8290.CD-18-0349 (2018).
2. Boj, S. F. et al. Organoid models of human and mouse ductal pancreatic cancer. Cell 160, 324-338, doi:10.1016/j.cell.2014.12.021 (2015).
3. Gierahn, T. M. et al. Seq-Well: portable, low-cost RNA sequencing of single cells at high throughput. Nat Methods 14, 395-398, doi:10.1038/nmeth.4179 (2017).
4. Macosko, E. Z. et al. Highly Parallel Genome-wide Expression Profiling of Individual Cells Using Nanoliter Droplets. Cell 161, 1202-1214, doi:10.1016/j.cell.2015.05.002 (2015).
5. Sholl, L. M. et al. Institutional implementation of clinical tumor profiling on an unselected cancer population. JCI Insight 1, e87062, doi:10.1172/jci.insight.87062 (2016).
6. Garcia, E. P. et al. Validation of OncoPanel: A Targeted Next-Generation Sequencing Assay for the Detection of Somatic Variants in Cancer. Arch Pathol Lab Med 141, 751-758, doi:10.5858/arpa.2016-0527-OA (2017).
7. Abo, R. P. et al. BreaKmer: detection of structural variation in targeted massively parallel sequencing data using kmers. Nucleic Acids Res 43, e19, doi:10.1093/nar/gkul211 (2015).
8. Bi, W. L. et al. Clinical Identification of Oncogenic Drivers and Copy-Number Alterations in Pituitary Tumors. Endocrinology 158, 2284-2291, doi:10.1210/en.2016-1967 (2017).
9. Patel, A. P. et al. Single-cell RNA-seq highlights intratumoral heterogeneity in primary glioblastoma. Science 344, 1396-1401, doi:10.1126/science.1254257 (2014).
10. Tirosh, I. et al. Single-cell RNA-seq supports a developmental hierarchy in human oligodendroglioma. Nature 539, 309-313, doi:10.1038/nature20123 (2016).
11. Tirosh, I. et al. Dissecting the multicellular ecosystem of metastatic melanoma by single-cell RNA-seq. Science 352, 189-196, doi:10.1126/science.aad0501 (2016).
12. Zhang, L. et al. Single-Cell Analyses Inform Mechanisms of Myeloid-Targeted Therapies in Colon Cancer. Cell 181, 442-459 e429, doi:10.1016/j.cell.2020.03.048 (2020).
13. Zilionis, R. et al. Single-Cell Transcriptomics of Human and Mouse Lung Cancers Reveals Conserved Myeloid Populations across Individuals and Species. Immunity 50, 1317-1334 el310, 470 doi:10.1016/j.immuni.2019.03.009 (2019).
14. Weinreb, C., Wolock, S. & Klein, A. M. SPRING: a kinetic interface for visualizing high dimensional single-cell expression data. Bioinformatics 34, 1246-1248, doi:10.1093/bioinformatics/btx792 (2018).
15. van Galen, P. et al. Single-Cell RNA-Seq Reveals AML Hierarchies Relevant to Disease Progression and Immunity. Cell 176, 1265-1281 e1224, doi:10.1016/j.cell.2019.01.031 (2019).
16. Moffitt, R. A. et al. Virtual microdissection identifies distinct tumor- and stroma-specific subtypes of pancreatic ductal adenocarcinoma. Nat Genet 47, 1168-1178, doi:10.1038/ng.3398 (2015).
17. Groger, C. J., Grubinger, M., Waldhor, T., Vierlinger, K. & Mikulits, W. Meta-analysis of gene expression signatures defining the epithelial to mesenchymal transition during cancer progression. PLoS One 7, e51136, doi:10.1371/journal.pone.0051136 (2012).
18. Sade-Feldman, M. et al. Defining T Cell States Associated with Response to Checkpoint Immunotherapy in Melanoma. Cell 176, 404, doi:10.1016/j.cell.2018.12.034 (2019).
19. Miller, B. C. et al. Subsets of exhausted CD8(+) T cells differentially mediate tumor control and respond to checkpoint blockade. Nat Immunol 20, 326-336, doi:10.1038/s41590-019-0312-6 (2019).
20. Venteicher, A. S. et al. Decoupling genetics, lineages, and microenvironment in IDH-mutant gliomas by single-cell RNA-seq. Science 355, doi: 10.1126/science.aai8478 (2017).
21. Filbin, M. G. et al. Developmental and oncogenic programs in H3K27M gliomas dissected by single-cell RNA-seq. Science 360, 331-335, doi:10.1126/science.aao4750 (2018).
22. Cancer Genome Atlas Research Network. Electronic address, a. a. d. h. e. & Cancer Genome Atlas Research, N. Integrated Genomic Characterization of Pancreatic Ductal Adenocarcinoma. Cancer Cell 32, 185-203 e113, doi:10.1016/j.ccell.2017.07.007 (2017).
23. Aguirre, A. J. et al. Real-time Genomic Characterization of Advanced Pancreatic Cancer to Enable Precision Medicine. Cancer Discov 8, 1096-1111, doi:10.1158/2159-8290.CD-18-0275 (2018).
24. Ghandi, M. et al. Next-generation characterization of the Cancer Cell Line Encyclopedia. Nature 569, 494 503-508, doi:10.1038/s41586-019-1186-3 (2019).
25. Puram, S. V. et al. Single-Cell Transcriptomic Analysis of Primary and Metastatic Tumor Ecosystems in Head and Neck Cancer. Cell 171, 1611-1624 e1624, doi:10.1016/j.cell.2017.10.044 (2017).
26. Bailey, P. et al. Genomic analyses identify molecular subtypes of pancreatic cancer. Nature 531, 47-52, doi:10.1038/nature16965 (2016).
27. Collisson, E. A. et al. Subtypes of pancreatic ductal adenocarcinoma and their differing responses to therapy. Nat Med 17, 500-503, doi:10.1038/nm.2344 (2011).
28. Elyada, E. et al. Cross-Species Single-Cell Analysis of Pancreatic Ductal Adenocarcinoma Reveals Antigen-Presenting Cancer-Associated Fibroblasts. Cancer Discov 9, 1102-1123, doi:10.1158/2159-8290.CD-19-0094 (2019).

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

Lengthy table referenced here
US20220396777A1-20221215-T00001
Please refer to the end of the specification for access instructions.

Lengthy table referenced here
US20220396777A1-20221215-T00002
Please refer to the end of the specification for access instructions.

Lengthy table referenced here
US20220396777A1-20221215-T00003
Please refer to the end of the specification for access instructions.

Lengthy table referenced here
US20220396777A1-20221215-T00004
Please refer to the end of the specification for access instructions.

Lengthy table referenced here
US20220396777A1-20221215-T00005
Please refer to the end of the specification for access instructions.

Lengthy table referenced here
US20220396777A1-20221215-T00006
Please refer to the end of the specification for access instructions.

LENGTHY TABLES
The patent application contains a lengthy table section. A copy of the table is available in electronic form from the USPTO web site (https://seqdata.uspto.gov/?pageRequest=docDetail&DocID=US20220396777A1). An electronic copy of the table will also be available from the USPTO upon request and payment of the fee set forth in 37 CFR 1.19(b)(3).

What is claimed is:

1. A method of generating an ex vivo cell-based system comprising
dissociating an original tissue sample obtained from a subject into a single cell population;
determining an in vivo phenotype of the tissue sample by conducting single-cell RNA analysis on a first portion of the single cells;
establishing an ex vivo cell-based system from a second portion of the single cells; and
culturing the ex vivo cell-based system in a medium or conditions selected to maintain the in vivo phenotype.

2. The method of claim 1, wherein the original tissue sample is a tumor tissue sample.

3. The method of claim 2, wherein the tumor tissue sample is a metastatic tumor tissue sample.

4. The method of any of claims 1 to 3, further comprising;
conducting a second single-cell RNA analysis on single cells derived from the established ex vivo cell-based system to determine a current phenotype; and
if the phenotype has changed, modifying the culture medium or conditions to revert to or decrease the expression space between the current phenotype and the in vivo phenotype.

5. The method of any one of the preceding claims, wherein selecting or modifying the medium or conditions comprises the addition or subtraction of one or more growth factors or cell signaling molecules, inducing changes in intra-cellular signaling between one or more cell types in the ex vivo cell-based model, inducing changes in cell state of one or more cell types, or changing cellular composition of the ex vivo cell-based model.

6. The method of claim 5, wherein the ex vivo cell-based model is co-cultured with fibroblasts in depleted media.

7. The method of claim 1, wherein the medium comprises one or more growth factors or cell signaling molecules.

8. The method of claim 7, wherein the cell signaling molecules comprise WNT7B, WNT10A, or a combination thereof.

9. The method of any of claims 5 to 8, further comprising culturing the cells in a medium which does not contain TGF beta inhibitor.

10. The method any one of claims 2 to 9, wherein the tumor is a pancreatic ductal adenocarcinoma (PDAC) tumor.

11. The method of claim 10, wherein the PDAC is the basal-like subtype, the classical subtype, or a hybrid sub-type including transcriptional phenotypes from both.

12. The method of any one of claims 2 to 9, wherein the tumor is a breast cancer tumor.

13. The method of any one of claims 2 to 9, wherein the tumor is a bladder cancer tumor.

14. The method of any of claims 10 to 13, wherein the organoid is cultured in a medium comprising IFNγ if the phenotype is a basal phenotype and/or IFNγ phenotype.

15. An ex vivo cell-based system derived by the method of any one of claims 1 to 14.

16. The ex vivo cell-based system of claim 15, wherein the ex vivo cell-based system comprises a tumor microenvironment cell.

17. The ex vivo cell-based system of claim 16, wherein the tumor microenvironment cell is a tumor infiltrating lymphocyte (TIL) and/or natural killer (NK) cell.

18. The ex vivo cell-based system of any of claims 15 to 17, wherein the ex vivo cell-based system simulates a phenotype from a subject who is responsive to cancer treatment.

19. The ex vivo cell-based system of any of claims 15 to 17, wherein the ex vivo cell-based system simulates a phenotype from a subject who is non-responsive to cancer treatment.

20. The ex vivo cell-based system of claim 19, wherein the treatment is chemotherapy.

21. The ex vivo cell-based system of claim 19, wherein the treatment is immunotherapy.

22. The ex vivo cell-based system of claim 21, wherein the treatment is checkpoint blockade (CPB) therapy.

23. The ex vivo cell-based system of claim 22, wherein the phenotype is a basal phenotype and/or IFNγ phenotype.

24. The ex vivo cell-based system of any of claims 15 to 23, wherein the system is an organoid.

25. A method for screening therapeutic agents comprising;
exposing the ex vivo cell-based model system of any one of claims 15 to 24 to one or more therapeutic agents,
measuring responsiveness of the ex vivo model to the one or more therapeutic agents; and
classifying the one or more therapeutic agents as indicated if the ex vivo model exhibits a responsive phenotype indicating a susceptibility of the model to the one or more therapeutic agents, or contraindicated if the ex vivo model exhibits a non-responsive phenotype indicating a lack of susceptibility of the model to the one or more therapeutic agents.

26. The method of claim 25, wherein the responsive phenotype is measured by a change in one or more cell types or cell states of the model indicating reduced fitness of the model or cell death of one or more target cell types in the model.

27. The method of claim 25, wherein the non-responsive phenotype is measured by no change in model phenotype or a change in one or more cell types or cell states indicating increased growth or fitness of the model or one or more cell types in the model.

28. The method of claim 27, further comprising clonally expanding the one or more cell types exhibiting increased growth or fitness and performing single cell RNA analysis of the clonally expanded cells to identify cell type and/or cell state.

29. The method of any one of claims 25 to 28, wherein the ex vivo cell-based model is derived from a subject to be treated.

30. The method of claim 29, further comprising administering the indicated one or more therapeutic agents to the subject.

31. The method of claim 29, further comprising administering one or more therapeutic agents based on the identified cell type and/or cell state of the clonally expanded cells.

32. The method of any of claims 25 to 30, wherein the ex vivo cell-based model system is a tumor system.

33. The method of claim 32, wherein the tumor system is derived from a pancreatic ductal adenocarcinoma (PDAC) tumor.

34. The method of claim 32 or 33, wherein the therapeutic agent is a chemotherapy.

35. The method of claim 34, wherein the therapeutic agent is a combination therapy comprising an agent predicted to shift the ex vivo cell model to have increased responsiveness to a chemotherapy and a chemotherapy.

36. The method of claim 32 or 33, wherein the therapeutic agent is an immunotherapy.

37. The method of claim 36, wherein the immunotherapy is one or more T cells expressing a chimeric antigen receptor (CAR) or T cell receptor (TCR).

38. The method of claim 36, wherein the immunotherapy is checkpoint blockade (CPB) therapy.

39. The method of any of claims 36 to 38, wherein the therapeutic agent is a combination therapy comprising an agent predicted to shift the ex vivo cell model to have increased responsiveness to an immunotherapy and an immunotherapy.

40. The method of claim 32 or 33, wherein the therapeutic agent is a targeted therapy.

41. The method of claim 40, wherein the therapeutic agent is a combination therapy comprising an agent predicted to shift the ex vivo cell model to have increased responsiveness to a targeted therapy and a targeted therapy.

42. A method of treating PDAC tumors comprising administering one or more agents that reduce IFNγ expression or interferon response gene expression in the tumor microenvironment.

43. A method of treating PDAC tumors comprising administering one or more agents that shift tumor cell phenotype from a basal or IFNγ phenotype to a classical phenotype.

44. A method of treating PDAC tumors comprising tumor cells expressing a basal subtype phenotype comprising administering one or more agents capable of interfering with intracellular crosstalk between tumor cells and basal associated tumor associated macrophages (TAM).

45. The method of claim 44, wherein the one or more agents interfere with CSF1 and/or IL34 from binding to CSF1R.

46. The method of claim 45, wherein the one or more agents bind to CSF1, IL34, and/or CSF1R.

47. The method of claim 46, wherein CSF1R antibodies are administered.

48. The method of any of claims 42 to 47, further comprising administering an immunotherapy, chemotherapy and/or targeted therapy.

49. The method of any of claims 40 to 47, wherein the PDAC is the basal-like subtype the classical subtype, or a hybrid sub-type including transcriptional phenotypes from both.
